SEQUENCE LISTING (246 sequences)

SEQ ID NO: 1 - protein, 18 aa, synthetic construct
EGRGSLLTCGDVEENPGP

SEQ ID NO: 2 - protein, 19 aa, synthetic construct
ATNFSLLKQAGDVEENPGP

SEQ ID NO: 3 - protein, 20 aa, synthetic construct
QCTNYALLKLAGDVESNPGP

SEQ ID NO: 4 - protein, 22 aa, synthetic construct
VKQTLNFDLLKLAGDVESNPGP

SEQ ID NO: 5 - protein, 10 aa, synthetic construct
TSTLLMENSS

SEQ ID NO: 6 - protein, 11 aa, synthetic construct
PRSSLYPALTP

SEQ ID NO: 7 - protein, 8 aa, synthetic construct
VQALVLTQ

SEQ ID NO: 8 - protein, 12 aa, synthetic construct
PLQVLTLNIERR

SEQ ID NO: 9 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 10 - protein, 515 aa, Homo sapiens
MNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 11 - skipped

SEQ ID NO: 12 - protein, 658 aa, synthetic construct
MAFEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 13 - protein, 756 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 14 - protein, 755 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFER

SEQ ID NO: 15 - protein, 753 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 16 - protein, 753 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 17 - protein, 335 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLL

SEQ ID NO: 18 - protein, 335 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLL

SEQ ID NO: 19 - protein, 342 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLPKKKRKV

SEQ ID NO: 20 - protein, 19 aa, synthetic construct
MKRTADGSEFESPKKKRKV

SEQ ID NO: 21 - protein, 30 aa, synthetic construct
MDSLLMNRRKFLYQFKNVRWAKGRRETYLC

SEQ ID NO: 22 - protein, 20 aa, synthetic construct
AVKRPAATKKAGQAKKKKLD

SEQ ID NO: 23 - protein, 25 aa, synthetic construct
MSRRRKANPTKLSENAKKLAKEVEN

SEQ ID NO: 24 - protein, 9 aa, synthetic construct
PAAKRVKLD

SEQ ID NO: 25 - protein, 9 aa, synthetic construct
KLKIKRPVK

SEQ ID NO: 26 - protein, 8 aa, synthetic construct
VSRKRPRP

SEQ ID NO: 27 - protein, 10 aa, synthetic construct
EGAPPAKRAR

SEQ ID NO: 28 - protein, 12 aa, synthetic construct
PPQPKKKPLDGE

SEQ ID NO: 29 - protein, 21 aa, synthetic construct
SGGSKRTADGSEFEPKKKRKV

SEQ ID NO: 30 - protein, 7 aa, synthetic construct
PKKKRKV

SEQ ID NO: 31 - protein, 18 aa, synthetic construct
KRTADGSEFESPKKKRKV

SEQ ID NO: 32 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 33 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 34 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 35 - protein, 33 aa, synthetic construct
SPEDEIQQLEEEIAQLEQKNAALKEKNQALKYG

SEQ ID NO: 36 - protein, 33 aa, synthetic construct
SPEDKIAQLKQKIQALKQENQQLEEENAALEYG

SEQ ID NO: 37 - protein, 1368 aa, unidentified [sequence omitted]

SEQ ID NO: 38 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 39 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 40 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 41 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 42 - protein, 1404 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAG

SEQ ID NO: 43 - protein, 1378 aa, Lactobacillus fermentum
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPTKNQAFIGKLLFPDLLKKNERGYPTLIKMRDELPVEQRAHYPVMNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKERGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKASDVASLVMAKTRGVNKPSVEGLSDPKKFNSNLATYLDLKSIVGDKVDDNRYQTDLENIIEWRSVFEDGEIFADKLTEVEWLTDEQRSALVKKRYKGWGRLSKKLLTGIVDENGQRIIDLMWNTDQNFKEIVDQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDNSLDNRVLTSRKENNKKSDQVPAKLYAAKMKPYWNQLLKQGLITQRKFENLTKDVDQNIKYRSLGFVKRQLVETRQVIKLTANILGSMYQEAGTEIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVAKSFDRLLNMKYMLVSKEVHDRSDQLYGATIVTAKESGKLTSPIEIKKNRLVDLYGAYTNGTSAFMTIIKFTGNKPKYKVIGIPTTSAASLKRAGKPGSESYNQELHRIIKSNPKVKKGFEIVVPHVSYGQLIVDGDCKFTLASPTVQHPATQLVLSKKSLETISSGYKILKDKPAIANERLIRVFDEVVGQMNRYFTIFDQRSNRQKVADARDKFLSLPTESKYEGAKKVQVGKTEVITNLLMGLHANATQGDLKVLGLATFGFFQSTTGLSLSEDTMIVYQSPTGLFERRICLKDI

SEQ ID NO: 44 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 45 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 46 - protein, 1402 aa, Lactobacillus crispatus
MKIKNYNLALTPSTSAVGHVEVDDDLNILEPVHHQKAIGVAKFGEGETAEARRLARSARRTTKRRANRINHYFNEIMKPEIDKVDPLMFDRIKQAGLSPLDERKEFRTVIFDRPNIASYYHNQFPTIWHLQKYLMITDEKADIRLIYWALHSLLKHRGHFFNTTPMSQFKPGKLNLKDDMLALDDYNDLEGLSFAVANSPEIEKVIKDRSMHKKEKIAELKKLIVNDVPDKDLAKRNNKIITQIVNAIMGNSFHLNFIFDMDLDKLTSKAWSFKLDDPELDTKFDAISGSMTDNQIGIFETLQKIYSAISLLDILNGSSNVVDAKNALYDKHKRDLNLYFKFLNTLPDEIAKTLKAGYTLYIGNRKKDLLAARKLLKVNVAKNFSQDDFYKLINKELKSIDKQGLQTRFSEKVGELVAQNNFLPVQRSSDNVFIPYQLNAITFNKILENQGKYYDFLVKPNPAKKDRKNAPYELSQLMQFTIPYYVGPLVTPEEQVKSGIPKTSRFAWMVRKDNGAITPWNFYDKVDIEATADKFIKRSIAKDSYLLSELVLPKHSLLYEKYEVFNELSNVSLDGKKLSGGVKQILFNEVFKKTNKVNTSRILKALAKHNIPGSKITGLSNPEEFTSSLQTYNAWKKYFPNQIDNFAYQQDLEKMIEWSTVFEDHKILAKKLDEIEWLDDDQKKFVANTRLRGWGRLSKRLLTGLKDNYGKSIMQRLETTKANFQQIVYKPEFREQIDKISQAAAKNQSLEDILANSYTSPSNRKAIRKTMSVVDEYIKLNHGKEPDKIFLMFQRSEQEKGKQTEARSKQLNRILSQLKADKSANKLFSKQLADEFSNAIKKSKYKLNDKQYFYFQQLGRDALTGEVIDYDELYKYTVLHIIPRSKLTDDSQNNKVLTKYKIVDGSVALKFGNSYSDALGMPIKAFWTELNRLKLIPKGKLLNLTTDFSTLNKYQRDGYIARQLVETQQIVKLLATIMQSRFKHTKIIEVRNSQVANIRYQFDYFRIKNLNEYYRGFDAYLAAVVGTYLYKVYPKARRLFVYGQYLKPKKTNQENQDMHLDSEKKSQGFNFLWNLLYGKQDQIFVNGTDVIAFNRKDLITKMNTVYNYKSQKISLAIDYHNGAMFKATLFPRNDRDTAKTRKLIPKKKDYDTDIYGGYTSNVDGYMLLAEIIKRDGNKQYGFYGVPSRLVSELDTLKKTRYTEYEEKLKEIIKPELGVDLKKIKKIKILKNKVPFNQVIIDKGSKFFITSTSYRWNYRQLILSAESQQTLMDLVVDPDFSNHKARKDARKNADERLIKVYEEILYQVKNYMPMFVELHRCYEKLVDAQKTFKSLKISDKAMVLNQILILLHSNATSPVLEKLGYHTRFTLGKKHNLISENAVLVTQSITGLKENHVSIKQML

SEQ ID NO: 47 - protein, 1385 aa, unidentified
  source 1..1385: note = Pedicoccus damnosus
MTNEKYSIGLDIGTSSIGFAVVNDNNRVIRVKGKNAIGVRLFDEGKAAADRRSFRTTRRSFRTTRRRLSRRRWRLKLLREIFDAYITPVDEAFFIRLKESNLSPKDSKKQYSGDILFNDRSDKDFYEKYPTIYHLRNALMTEHRKFDVREIYLAIHHIMKFRGHFLNATPANNFKVGRLNLEEKFEELNDIYQRVFPDESIEFRTDNLEQIKEVLLDNKRSRADRQRTLVSDIYQSSEDKDIEKRNKAVATEILKASLGNKAKLNVITNVEVDKEAAKEWSITFDSESIDDDLAKIEGQMTDDGHEIIEVLRSLYSGITLSAIVPENHTLSQSMVAKYDLHKDHLKLFKKLINGMTDTKKAKNLRAAYDGYIDGVKGKVLPQEDFYKQVQVNLDDSAEANEIQTYIDQDIFMPKQRTKANGSIPHQLQQQELDQIIENQKAYYPWLAELNPNPDKKRQQLAKYKLDELVTFRVPYYVGPMITAKDQKNQSGAEFAWMIRKEPGNITPWNFDQKVDRMATANQFIKRMTTTDTYLLGEDVLPAQSLLYQKFEVLNELNKIRIDHKPISIEQKQQIFNDLFKQFKNVTIKHLQDYLVSQGQYSKRPLIEGLADEKRFNSSLSTYSDLCGIFGAKLVEENDRQEDLEKIIEWSTIFEDKKIYRAKLNDLTWLTDDQKEKLATKRYQGWGRLSRKLLVGLKNSEHRNIMDILWITNENFMQIQAEPDFAKLVTDANKGMLEKTDSQDVINDLYTSPQNKKAIRQILLVVHDIQNAMHGQAPAKIHVEFARGEERNPRRSVQRQRQVEAAYEKVSNELVSAKVRQEFKEAINNKRDFKDRLFLYFMQGGIDIYTGKQLNIDQLSSYQIDHILPQAFVKDDSLTNRVLTNENQVKADSVPIDIFGKKMLSVWGRMKDQGLISKGKYRNLTMNPENISAHTENGFINRQLVETRQVIKLAVNILADEYGDSTQIISVKADLSHQMREDFELLKNRDVNDYHHAFDAYLAAFIGNYLLKRYPKLESYFVYGDFKKFTQKETKMRRFNFIYDLKHCDQVVNKETGEILWTKDEDIKYIRHLFAYKKILVSHEVREKRGALYNQTIYKAKDDKGSGQESKKLIRIKDDKETKIYGGYSGKSLAYMTIVQITKKNKVSYRVIGIPTLALARLNKLENDSTENNGELYKIIKPQFTHYKVDKKNGEIIETTDDFKIVVSKVRFQQLIDDAGQFFMLASDTYKNNAQQLVISNNALKAINNTNITDCPRDDLERLDNLRLDSAFDEIVKKMDKYFSAYDANNFREKIRNSNLIFYQLPVEDQWENNKITELGKRTVLTRILQGLHANATTTDMSIFKIKTPFGQLRQRSGISLSENAQLIYQSPTGLFERRVQLNKIK

SEQ ID NO: 48 - protein, 1367 aa, unidentified
  source 1..1367: note = Fusobaterium nucleatum
MKKQKFSDYYLGFDIGTNSVGWCVTDLDYNVLRFNKKDMWGSRLFEEAKTAAERRVQRNSRRRLKRRKWRLNLLEEIFSNEILKIDSNFFRRLKESSLWLEDKSSKEKFTLFNDDNYKDYDFYKQYPTIFHLRNELIKNPEKKDIRLVYLAIHSIFKSRGHFLFEGQNLKEIKNFETLYNNLIAFLEDNGINKIIDKNNIEKLEKIVCDSKKGLKDKEKEFKEIFNSDKQLVAIFKLSVGSSVSLNDLFDTDEYKKGEVEKEKISFREQIYEDDKPIYYSILGEKIELLDIAKTFYDFMVLNNILADSQYISEAKVKLYEEHKKDLKNLKYIIRKYNKGNYDKLFKDKNENNYSAYIGLNKEKSKKEVIEKSRLKIDDLIKNIKGYLPKVEEIEEKDKAIFNKILNKIELKTILPKQRISDNGTLPYQIHEAELEKILENQSKYYDFLNYEENGIITKDKLLMTFKFRIPYYVGPLNSYHKDKGGNSWIVRKEEGKILPWNFEQKVDIEKSAEEFIKRMTNKCTYLNGEDVIPKDTFLYSEYVILNELNKVQVNDEFLNEENKRKIIDELFKENKKVSEKKFKEYLLVKQIVDGTIELKGVKDSFNSNYISYIRFKDIFGEKLNLDIYKEISEKSILWKCLYGDDKKIFEKKIKNEYGDILTKDEIKKINTFKFNNWGRLSEKLLTGIEFINLETGECYSSVMDALRRTNYNLMELLSSKFTLQESINNENKEMNEASYRDLIEESYVSPSLKRAIFQTLKIYEEIRKITGRVPKKVFIEMARGGDESMKNKKIPARQEQLKKLYDSCGNDIANFSIDIKEMKNSLISYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEKKGQLFDLNPIKKGETSNEIISIKPKVYNGKDDKLNEKYGYYKSLNPAYFLYVEHKEKNKRIKSFERVNLVDVNNIKDEKSLVKYLIENKKLVEPRVIKKVYKRQVILINDYPYSIVTLDSNKLMDFENLKPLFLENKYEKILKNVIKFLEDNQGKSEENYKFIYLKKKDRYEKNETLESVKDRYNLEFNEMYDKFLEKLDSKDYKNYMNNKKYQELLDVKEKFIKLNLFDKAFTLKSFLDLFNRKTMADFSKVGLTKYLGKIQKISSNVLSKNELYLLEESVTGLFVKKIKL

SEQ ID NO: 49 - protein, 1286 aa, Enterococcus cecorum
RRKQRIQILQELLGEEVLKTDPGFFHRMKESRYVVEDKRTLDGKQVELPYALFVDKDYTDKEYYKQFPTINHLIVYLMTTSDTPDIRLVYLALHYYMKNRGNFLHSGDINNVKDINDILEQLDNVLETFLDGWNLKLKSYVEDIKNIYNRDLGRGERKKAFVNTLGAKTKAEKAFCSLISGGSTNLAELFDDSSLKEIETPKIEFASSSLEDKIDGIQEALEDRFAVIEAAKRLYDWKTLTDILGDSSSLAEARVNSYQMHHEQLLELKSLVKEYLDRKVFQEVFVSLNVANNYPAYIGHTKINGKKKELEVKRTKRNDFYSYVKKQVIEPIKKKVSDEAVLTKLSEIESLIEVDKYLPLQVNSDNGVIPYQVKLNELTRIFDNLENRIPVLRENRDKIIKTFKFRIPYYVGSLNGVVKNGKCTNWMVRKEEGKIYPWNFEDKVDLEASAEQFIRRMTNKCTYLVNEDVLPKYSLLYSKYLVLSELNNLRIDGRPLDVKIKQDIYENVFKKNRKVTLKKIKKYLLKEGIITDDDELSGLADDVKSSLTAYRDFKEKLGHLDLSEAQMENIILNITLFGDDKKLLKKRLAALYPFIDDKSLNRIATLNYRDWGRLSERFLSGITSVDQETGELRTIIQCMYETQANLMQLLAEPYHFVEAIEKENPKVDLESISYRIVNDLYVSPAVKRQIWQTLLVIKDIKQVMKHDPERIFIEMAREKQESKKTKSRKQVLSEVYKKAKEYEHLFEKLNSLTEEQLRSKKIYLYFTQLGKCMYSGEPIDFENLVSANSNYDIDHIYPQSKTIDDSFNNIVLVKKSLNAYKSNHYPIDKNIRDNEKVKTLWNTLVSKGLITKEKYERLIRSTPFSDEELAGFIARQLVETRQSTKAVAEILSNWFPESEIVYSKAKNVSNFRQDFEILKVRELNDCHHAHDAYLNIVVGNAYHTKFTNSPYRFIKNKANQEYNLRKLLQKVNKIESNGVVAWVGQSENNPGTIATVKKVIRRNTVLISRMVKEVDGQLFDLTLMKKGKGQVPIKSSDERLTDISKYGGYNKATGAYFTFVKSKKRGKVVRSFEYVPLHLSKQFENNNELLKEYIEKDRGLTDVEILIPKVLINSLFRYNGSLVRITGRGDTRLLLVHEQPLYVSNSFVQQLKSVSSYKLKKSENDNAKLTKTATEKLSNIDELYDGLLRKLDLPIYSYWFSSIKEYLVESRTKYIKLSIEEKALVIFEILHLFQSDAQVPNLKILGLSTKPSRIRIQKNLKDTDKMSIIHQSPSGIFEHEIELTSL

SEQ ID NO: 50 - protein, 1312 aa, unidentified
  source 1..1312: note = Anaerostipes hadrus
MQNGFLGITVSSEQVGWAVTNPKYELERASRKDLWGVRLFDKAETAEDRRMFRTNRRLNQRKKNRIHYLRDIFHEEVNQKDPNFFQQLDESNFCEDDRTVEFNFDTNLYKNQFPTVYHLRKYLMETKDKPDIRLVYLAFSKFMKNRGHFLYKGNLGEVMDFENSMKGFCESLEKFNIDFPTLSDEQVKEVRDILCDHKIAKTVKKKNIITITKVKSKTAKAWIGLFCGCSVPVKVLFQDIDEEIVTDPEKISFEDASYDDYIANIEKGVGIYYEAIVSAKMLFDWSILNEILGDHQLLSDAMIAEYNKHHDDLKRLQKIIKGTGSRELYQDIFINDVSGNYVCYVGHAKTMSSADQKQFYTFLKNRLKNVNGISSEDAEWIDTEIKNGTLLPKQTKRDNSVIPHQLQLREFELILDNMQEMYPFLKENREKLLKIFNFVIPYYVGPLKGVVRKGESTNWMVPKKDGVIHPWNFDEMVDKEASAECFISRMTGNCSYLFNEKVLPKNSLLYETFEVLNELNPLKINGEPISVELKQRIYEQLFLTGKKVTKKSLTKYLIKNGYDKDIELSGIDNEFHSNLKSHIDFEDYDNLSDEEVEQIILRITVFEDKQLLKDYLNREFVKLSEDERKQICSLSYKGWGNLSEMLLNGITVTDSNGVEVSVMDMLWNTNLNLMQILSKKYGYKAEIEHYNKEHEKTIYNREDLMDYLNIPPAQRRKVNQLITIVKSLKKTYGVPNKIFFKISREHQDDPKRTSSRKEQLKYLYKSLKSEDEKHLMKELDELNDHELSNDKVYLYFLQKGRCIYSGKKLNLSRLRKSNYQNDIDYIYPLSAVNDRSMNNKVLTGIQENRADKYTYFPVDSEIQKKMKGFWMELVLQGFMTKEKYFRLSRENDFSKSELVSFIEREISDNQQSGRMIASVLQYYFPESKIVFVKEKLISSFKRDFHLISSYGHNHLQAAKDAYITIVVGNVYHTKFTMDPAIYFKNHKRKDYDLNRLFLENISRDGQIAWESGPYGSIQTVRKEYAQNHIAVTKRVVEVKGGLFKQMPLKKGHGEYPLKTNDPRFGNIAQYGGYTNVTGSYFVLVESMEKGKKRISLEYVPVYLHERLEDDPGHKLLKEYLVDHRKLNHPKILLAKVRKNSLLKIDGFYYRLNGRSGNALILTNAVELIMDDWQTKTANKISGYMKRRAIDKKARVYQNEFHIQELEQLYDFYLDKLKNGVYKNRKNNQAELIHNEKEQFMELKTEDQCVLLTEIKKLFVCSPMQADLTLIGGSKHTGMIAMSSNVTKADFAVIAEDPLGLRNKVIYSHKGEK

SEQ ID NO: 51 - protein, 1344 aa, Kandleria vitulina
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 52 - protein, 1288 aa, Enterococcus faecalis
MRLFEEGHTAEDRRLKRTARRRISRRRNRLRYLQAFFEEAMTDLDENFFARLQESFLVPEDKKWHRHPIFAKLEDEVAYHETYPTIYHLRKKLADSSEQADLRLIYLALAHIVKYRGHFLIEGKLSTENTSVKDQFQQFMVIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLVKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQTFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSPTGLYETRRKVVD

SEQ ID NO: 53 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 54 - protein, 1082 aa, unidentified
  source 1..1082: note = Bacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 55 - protein, 1375 aa, Streptococcus canis
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 56 - DNA, 4104 bp, Streptococcus pyogenes
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 57 - DNA, 4212 bp, Streptococcus pyogenes
  source 1..4212: mol_type = genomic DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 58 - DNA, 4107 bp, Streptococcus pyogenes
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 59 - protein, 677 aa, unidentified
  source 1..677: note = moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 60 - protein, 677 aa, unidentified
  source 1..677: note = moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 61 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 62 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 63 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 64 - protein, 677 aa, synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 65 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 66 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 67 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 68 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 69 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 70 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 71 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 72 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 73 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 74 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 75 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 76 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 77 - protein, 17 aa, synthetic construct
KRTADGSEFEPKKKRKV

SEQ ID NO: 78 - protein, 19 aa, synthetic construct
NLSKRPAAIKKAGQAKKKK

SEQ ID NO: 79 - protein, 11 aa, synthetic construct
RQRRNELKRSF

SEQ ID NO: 80 - protein, 38 aa, synthetic construct
NQSSNFGPMKGGNFGGRSSGPYGGGGQYFAKPRNQGGY

SEQ ID NO: 81 - protein, 16 aa, unidentified
  source 1..16: note = Xenopus nucleoplasmin
KRXXXXXXXXXXKKKL

SEQ ID NO: 82 - protein, 14 aa, synthetic construct
MEELSQALASSFSV

SEQ ID NO: 83 - protein, 12 aa, synthetic construct
PLQLPPLERLTL

SEQ ID NO: 84 - protein, 12 aa, synthetic construct
NELALKLAGLDI

SEQ ID NO: 85 - protein, 15 aa, synthetic construct
ERFEMFRELNEALEL

SEQ ID NO: 86 - protein, 15 aa, synthetic construct
DHAEKVAEKLEALSV

SEQ ID NO: 87 - protein, 15 aa, synthetic construct
QLVEELLKIICAFQL

SEQ ID NO: 88 - protein, 15 aa, synthetic construct
TNLEALQKKLEELEL

SEQ ID NO: 89 - protein, 15 aa, synthetic construct
DVKEEMTSALATMRV

SEQ ID NO: 90 - protein, 15 aa, synthetic construct
STNGSLAAEFRHLQL

SEQ ID NO: 91 - protein, 15 aa, synthetic construct
PSVQELTEQIHRLLM

SEQ ID NO: 92 - protein, 15 aa, synthetic construct
MNFKELKDFLKELNI

SEQ ID NO: 93 - protein, 15 aa, synthetic construct
ENFEILMKLKESLEL

SEQ ID NO: 94 - protein, 14 aa, synthetic construct
FETVYELTKMCTIR

SEQ ID NO: 95 - protein, 15 aa, synthetic construct
SGKASSSLGLQDFDL

SEQ ID NO: 96 - protein, 15 aa, synthetic construct
PKYSDIDVDGLCSEL

SEQ ID NO: 97 - protein, 15 aa, synthetic construct
VDLACTPTDVRDVDI

SEQ ID NO: 98 - protein, 15 aa, synthetic construct
YGEKTTQRDLTELEI

SEQ ID NO: 99 - protein, 15 aa, synthetic construct
RRIYDITNVLEGIGL

SEQ ID NO: 100 - protein, 15 aa, synthetic construct
AKIIPYSGLLLVITV

SEQ ID NO: 101 - protein, 15 aa, synthetic construct
LRSEEVHWLHVDMGV

SEQ ID NO: 102 - protein, 15 aa, synthetic construct
LQSEEVHWLHLDMGV

SEQ ID NO: 103 - protein, 15 aa, synthetic construct
LQVRKYSLDLASLIL

SEQ ID NO: 104 - protein, 15 aa, synthetic construct
AGVEAIIRILQQLLF

SEQ ID NO: 105 - protein, 15 aa, synthetic construct
TGVEALIRILQQLLF

SEQ ID NO: 106 - protein, 15 aa, synthetic construct
IVLNQLCVRFFGLDL

SEQ ID NO: 107 - protein, 15 aa, synthetic construct
SLGGFEITPPVVLRL

SEQ ID NO: 108 - protein, 15 aa, synthetic construct
EAIQDLCLAVEEVSL

SEQ ID NO: 109 - protein, 15 aa, synthetic construct
DELLQVLRMMVGVNI

SEQ ID NO: 110 - protein, 15 aa, synthetic construct
SVMLAVQEGIDLLTF

SEQ ID NO: 111 - protein, 10 aa, synthetic construct
LSSHFQELSI

SEQ ID NO: 112 - protein, 15 aa, synthetic construct
QSTHVDIRTLEDLLM

SEQ ID NO: 113 - protein, 15 aa, synthetic construct
ESSAEDLRTLQQLFL

SEQ ID NO: 114 - protein, 15 aa, synthetic construct
EFSLPTHHTVRLIRV

SEQ ID NO: 115 - protein, 15 aa, synthetic construct
MSSGYYLGEILRLAL

SEQ ID NO: 116 - protein, 15 aa, synthetic construct
DTVLDILRDFFELRL

SEQ ID NO: 117 - protein, 15 aa, synthetic construct
NSVNEILSEFYYVRL

SEQ ID NO: 118 - protein, 15 aa, synthetic construct
CAFLSVKKQFEELTL

SEQ ID NO: 119 - protein, 15 aa, synthetic construct
ISPEHVIQALESLGF

SEQ ID NO: 120 - protein, 15 aa, synthetic construct
AHWMRQLVSFQKLKL

SEQ ID NO: 121 - protein, 15 aa, synthetic construct
ATRELDELMASLSDF

SEQ ID NO: 122 - protein, 15 aa, synthetic construct
YQNIELITFINALKL

SEQ ID NO: 123 - protein, 15 aa, synthetic construct
FNATAVVRHMRKLQL

SEQ ID NO: 124 - protein, 15 aa, synthetic construct
SGIFGLVTNLEELEV

SEQ ID NO: 125 - protein, 15 aa, synthetic construct
EESYTLNSDLARLGV

SEQ ID NO: 126 - protein, 15 aa, synthetic construct
EESYDLTSHLARLGV

SEQ ID NO: 127 - protein, 15 aa, synthetic construct
GIQQAHAEQLANMRI

SEQ ID NO: 128 - protein, 15 aa, synthetic construct
AAEPVILDLRDLFQL

SEQ ID NO: 129 - protein, 10 aa, synthetic construct
MEGCVSNLMV

SEQ ID NO: 130 - protein, 9 aa, synthetic construct
EGCVSNLMV

SEQ ID NO: 131 - protein, 15 aa, synthetic construct
DMDFLRNLFSQTLSL

SEQ ID NO: 132 - protein, 15 aa, synthetic construct
EQLLEIVHDLENLSL

SEQ ID NO: 133 - protein, 15 aa, synthetic construct
NVMKYFTDLFDYLPL

SEQ ID NO: 134 - protein, 15 aa, synthetic construct
KVYPIILRLGSNLSL

SEQ ID NO: 135 - protein, 15 aa, synthetic construct
YAGFSLPHAILRIDL

SEQ ID NO: 136 - protein, 15 aa, synthetic construct
EIVRDIKEKLCYVAL

SEQ ID NO: 137 - protein, 15 aa, synthetic construct
EAINKLESNLRELQI

SEQ ID NO: 138 - protein, 15 aa, synthetic construct
EAINKLENNLRELQI

SEQ ID NO: 139 - protein, 15 aa, synthetic construct
SDQKQEQLLLKKMYL

SEQ ID NO: 140 - protein, 15 aa, synthetic construct
KQVLWDRTFSLFQQL

SEQ ID NO: 141 - protein, 15 aa, synthetic construct
AQLQNLTKRIDSLPL

SEQ ID NO: 142 - protein, 15 aa, synthetic construct
NDENEHQLSLRTVSL

SEQ ID NO: 143 - protein, 15 aa, synthetic construct
ISFTEFVKVLEKVDV

SEQ ID NO: 144 - protein, 14 aa, synthetic construct
MESAITLWQFLLQL

SEQ ID NO: 145 - protein, 15 aa, synthetic construct
VPKELMQQIENFEKI

SEQ ID NO: 146 - protein, 15 aa, synthetic construct
QARFILEKIDGKIII

SEQ ID NO: 147 - protein, 15 aa, synthetic construct
QVKFIKMIIEKELTV

SEQ ID NO: 148 - protein, 15 aa, synthetic construct
NHRMKNLREISQLGI

SEQ ID NO: 149 - protein, 15 aa, synthetic construct
NHRVKKLNEISKLGI

SEQ ID NO: 150 - protein, 15 aa, synthetic construct
TEKHLQKYLRQDLRL

SEQ ID NO: 151 - protein, 15 aa, synthetic construct
RQERKRPLLDLHIEL

SEQ ID NO: 152 - protein, 15 aa, synthetic construct
ANMRIQDLKVSLKPL

SEQ ID NO: 153 - protein, 15 aa, synthetic construct
ATMRVDYEQIKIKKI

SEQ ID NO: 154 - protein, 15 aa, synthetic construct
LQGEEFVCLKSIILL

SEQ ID NO: 155 - protein, 15 aa, synthetic construct
THYGQKAILFLPLPV

SEQ ID NO: 156 - protein, 15 aa, synthetic construct
PSAHEITGLADSLQL

SEQ ID NO: 157 - protein, 15 aa, synthetic construct
VRLHDVLHSDKKLTL

SEQ ID NO: 158 - protein, 15 aa, synthetic construct
LINRNGELKLANFGL

SEQ ID NO: 159 - protein, 15 aa, synthetic construct
LEPLKKLECLKSLDL

SEQ ID NO: 160 - protein, 34 aa, synthetic construct
SGGSSGGSKRTADGSEFESPKKKRKVSGGSSGGS

SEQ ID NO: 161 - protein, 33 aa, synthetic construct
SGGSSGGSSGSETPGTSESATPESSGGSSGGSS

SEQ ID NO: 162 - protein, 4 aa, synthetic construct
SGGS

SEQ ID NO: 163 - protein, 8 aa, synthetic construct
SGGSSGGS

SEQ ID NO: 164 - protein, 150 aa, synthetic construct
  VARIANT 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 165 - protein, 30 aa, synthetic construct
  VARIANT 2..30: note = may be absent
GGGGGGGGGGGGGGGGGGGGGGGGGGGGGG

SEQ ID NO: 166 - protein, 150 aa, synthetic construct
  VARIANT 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 167 - protein, 90 aa, synthetic construct
  VARIANT 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 168 - protein, 120 aa, synthetic construct
  VARIANT 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 169 - protein, 60 aa, synthetic construct
  VARIANT 3..60: note = may be absent
XPXPXPXPXPXPXPXPXPXPXPXPXPXPXPXPXPXPXPXPXPXPXPXPXPXPXPXPXPXP

SEQ ID NO: 170 - protein, 16 aa, synthetic construct
SGSETPGTSESATPES

SEQ ID NO: 171 - protein, 32 aa, synthetic construct
SGGSSGGSSGSETPGTSESATPESSGGSSGGS

SEQ ID NO: 172 - protein, 10 aa, synthetic construct
SGGSGGSGGS

SEQ ID NO: 173 - protein, 60 aa, synthetic construct
SGGSSGGSSGSETPGTSESATPESAGSYPYDVPDYAGSAAPAAKKKKLDGSGSGGSSGGS

SEQ ID NO: 174 - protein, 6 aa, synthetic construct
GGSGGS

SEQ ID NO: 175 - protein, 9 aa, synthetic construct
GGSGGSGGS

SEQ ID NO: 176 - protein, 380 aa, Homo sapiens
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 177 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVRAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 178 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGAPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 179 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSARRKEPEPKGSTKKKAKTGAAGKFKRGK

SEQ ID NO: 180 - protein, 908 aa, synthetic construct
MGVNDLWQILEPVKQHIPLRNLGGKTIAVDLSLWVCEAQTVKKMMGSVMKPHLRNLFFRISYLTQMDVKLVFVMEGEPPKLKADVISKRNQSRYGSSGKSWSQKTGRSHFKSVLRECLHMLECLGIPWVQAAGEAEAMCAYLNAGGHVDGCLTNDGDTFLYGAQTVYRNFTMNTKDPHVDCYTMSSIKSKLGLDRDALVGLAILLGCDYLPKGVPGVGKEQALKLIQILKGQSLLQRFNRWNETSCNSSPQLLVTKKLAHCSVCSHPGSPKDHERNGCRLCKSDKYCEPHDYEYCCPCEWHRTEHDRQLSEVENNIKKKACCCEGFPFHEVIQEFLLNKDKLVKVIRYQRPDLLLFQRFTLEKMEWPNHYACEKLLVLLTHYDMIERKLGSRNSNQLQPIRIVKTRIRNGVHCFEIEWEKPEHYAMEDKQHGEFALLTIEEESLFEAAYPEIVAVYQKQKLEIKGKKQKRIKPKENNLPEPDEVMSFQSHMTLKPTCEIFHKQNSKLNSGISPDPTLPQESISASLNSLLLPKNTPCLNAQEQFMSSLRPLAIQQIKAVSKSLISESSQPNTSSHNISVIADLHLSTIDWEGTSFSNSPAIQRNTFSHDLKSEVESELSAIPDGFENIPEQLSCESERYTANIKKVLDEDSDGISPEEHLLSGITDLCLQDLPLKERIFTKLSYPQDNLQPDVNLKTLSILSVKESCIANSGSDCTSHLSKDLPGIPLQNESRDSKILKGDQLLQEDYKVNTSVPYSVSNTVVKTCNVRPPNTALDHSRKVDMQTTRKILMKKSVCLDRHSSDEQSAPVFGKAKYTTQRMKHSSQKHNSSHFKESGHNKLSSPKIHIKETEQCVRSYETAENEESCFPDSTKSSLSSLQCHKKENNSGTCLDSPLPLRQRLKLRFQST

SEQ ID NO: 181 - protein, 1186 aa, synthetic construct
MGVQGLWKLLECSGRQVSPEALEGKILAVDISIWLNQALKGVRDRHGNSIENPHLLTLFHRLCKLLFFRIRPIFVFDGDAPLLKKQTLVKRRQRKDLASSDSRKTTEKLLKTFLKRQAIKTAFRSKRDEALPSLTQVRRENDLYVLPPLQEEEKHSSEEEDEKEWQERMNQKQALQEEFFHNPQAIDIESEDFSSLPPEVKHEILTDMKEFTKRRRTLFEAMPEESDDFSQYQLKGLLKKNYLNQHIEHVQKEMNQQHSGHIRRQYEDEGGFLKEVESRRVVSEDTSHYILIKGIQAKTVAEVDSESLPSSSKMHGMSFDVKSSPCEKLKTEKEPDATPPSPRTLLAMQAALLGSSSEEELESENRRQARGRNAPAAVDEGSISPRTLSAIKRALDDDEDVKVCAGDDVQTGGPGAEEMRINSSTENSDEGLKVRDGKGIPFTATLASSSVNSAEEHVASTNEGREPTDSVPKEQMSLVHVGTEAFPISDESMIKDRKDRLPLESAVVRHSDAPGLPNGRELTPASPTCTNSVSKNETHAEVLEQQNELCPYESKFDSSLLSSDDETKCKPNSASEVIGPVSLQETSSIVSVPSEAVDNVENVVSFNAKEHENFLETIQEQQTTESAGQDLISIPKAVEPMEIDSEESESDGSFIEVQSVISDEELQAEFPETSKPPSEQGEEELVGTREGEAPAESESLLRDNSERDDVDGEPQEAEKDAEDSLHEWQDINLEELETLESNLLAQQNSLKAQKQQQERIAATVTGQMFLESQELLRLFGIPYIQAPMEAEAQCAILDLTDQTSGTITDDSDIWLFGARHVYRNFFNKNKFVEYYQYVDFHNQLGLDRNKLINLAYLLGSDYTEGIPTVGCVTAMEILNEFPGHGLEPLLKFSEWWHEAQKNPKIRPNPHDTKVKKKLRTLQLTPGFPNPAVAEAYLKPVVDDSKGSFLWGKPDLDKIREFCQRYFGWNRTKTDESLFPVLKQLDAQQTQLRIDSFFRLAQQEKEDAKRIKSQRLNRAVTCMLRKEKEAAASEIEAVSVAMEKEFELLDKAKRKTQKRGITNTLEESSSLKRKRLSDSKRKNTCGGFLGETCLSESSDGSSSEDAESSSLMNVQRRTAAKEPKTSASDSQNSVKEAPVKNGGATTSSSSDSDDDGGKEKMVLVTARSVFGKKRRKLRRARGRKRKT

SEQ ID NO: 182 - protein, 236 aa, Homo sapiens
MSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 183 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 184 - protein, 236 aa, Rattus norvegicus
MSEPLRAETFVFLDLEATGLPNMDPEIAEISLFAVHRSSLENPERDDSGSLVLPRVLDKLTLCMCPERPFTAKASEITGLSSEGLMNCRKAAFNDAVVRTLQGFLSRQEGPICLVAHNGFDYDFPLLCTELQRLGAHLPRDTVCLDTLPALRGLDRVHSHGTRAQGRKSYSLASLFHRYFQAEPSAAHSAEGDVNTLLLIFLHRAPELLAWADEQARSWAHIEPMYVPPDGPSLEA

SEQ ID NO: 185 - protein, 803 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 186 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 187 - protein, 845 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 188 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 189 - protein, 413 aa, synthetic construct
CLAEGTRIFDPVTGTTHRIEDVVDGRKPIHVVAVAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYTNVVPLYDLLLEMLDAHRLHAGGSGASRVQAFADALDDKFLHDMLAEELRYSVIREVLPTRRARTFDLEVEELHTLVAEGVVVHNC

SEQ ID NO: 190 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 191 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 192 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 193 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYNPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 194 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 195 - protein, 413 aa, synthetic construct
CLAEGTRIFDPVTGTTHRIEDVVDGRKPIHVVAVAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 196 - protein, 33 aa, synthetic construct
GCCAACATGAGGATCACCCATGTCTGCAGGGCC

SEQ ID NO: 197 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 198 - protein, 84 aa, synthetic construct
MTNLSDIIEKETGKQLVIQESILMLPEEVEEVIGNKPESDILVHTAYDESTDENVMLLTSDAPEYKPWALVIQDSNGENKIKML

SEQ ID NO: 199 - protein, 345 aa, Homo sapiens
MPARALLPRRMGHRTLASTPALWASIPCPRSELRLDLVLPSGQSFRWREQSPAHWSGVLADQVWTLTQTEEQLHCTVYRGDKSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVLFSADLRQSRHAQEPPAKRRKGSKGPEG

SEQ ID NO: 200 - protein, 298 aa, Homo sapiens
MVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 201 - protein, 580 aa, Homo sapiens
MGTTGLESLSLGDRGAAPTVTSSERLVPDPPNDLRKEDVAMELERVGEDEEQMMIKRSSECNPLLQEPIASAQFGATAGTECRKSVPCGWERVVKQRLFGKTAGRFDVYFISPQGLKFRSKSSLANYLHKNGETSLKPEDFDFTVLSKRGIKSRYKDCSMAALTSHLQNQSNNSNWNLRTRSKCKKDVFMPPSSSSELQESRGLSNFTSTHLLLKEDEGVDDVNFRKVRKPKGKVTILKGIPIKKTKKGCRKSCSGFVQSDSKRESVCNKADAESEPVAQKSQLDRTVCISDAGACGETLSVTSEENSLVKKKERSLSSGSNFCSEQKTSGIINKFCSAKDSEHNEKYEDTFLESEEIGTKVEVVERKEHLHTDILKRGSEMDNNCSPTRKDFTGEKIFQEDTIPRTQIERRKTSLYFSSKYNKEALSPPRRKAFKKWTPPRSPFNLVQETLFHDPWKLLIATIFLNRTSGKMAIPVLWKFLEKYPSAEVARTADWRDVSELLKPLGLYDLRAKTIVKFSDEYLTKQWKYPIELHGIGKYGNDSYRIFCVNEWKQVHPEDHKLNKYHDWLWENHEKLSLS

SEQ ID NO: 202 - protein, 410 aa, Homo sapiens
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 203 - protein, 760 aa, Mus musculus
MAFVAGVIRRLDETVVNRIAAGEVIQRPANAIKEMIENCLDAKSTNIQVVVKEGGLKLIQIQDNGTGIRKEDLDIVCERFTTSKLQTFEDLASISTYGFRGEALASISHVAHVTITTKTADGKCAYRASYSDGKLQAPPKPCAGNQGTLITVEDLFYNIITRRKALKNPSEEYGKILEVVGRYSIHNSGISFSVKKQGETVSDVRTLPNATTVDNIRSIFGNAVSRELIEVGCEDKTLAFKMNGYISNANYSVKKCIFLLFINHRLVESAALRKAIETVYAAYLPKNTHPFLYLSLEISPQNVDVNVHPTKHEVHFLHEESILQRVQQHIESKLLGSNSSRMYFTQTLLPGLAGPSGEAARPTTGVASSSTSGSGDKVYAYQMVRTDSREQKLDAFLQPVSSLGPSQPQDPAPVRGARTEGSPERATREDEEMLALPAPAEAAAESENLERESLMETSDAAQKAAPTSSPGSSRKRHREDSDVEMVENASGKEMTAACYPRRRIINLTSVLSLQEEISERCHETLREMLRNHSFVGCVNPQWALAQHQTKLYLLNTTKLSEELFYQILIYDFANFGVLRLSEPAPLFDLAMLALDSPESGWTEDDGPKEGLAEYIVEFLKKKAEMLADYFSVEIDEEGNLIGLPLLIDSYVPPLEGLPIFILRLATEVNWDEEKECFESLSKECAMFYSIRKQYILEESTLSGQQSDMPGSTSKPWKWTVEHIIYKAFRSHLLPPKHFTEDGNVLQLANLPDLYKVFERC

SEQ ID NO: 204 - protein, 757 aa, Rattus norvegicus
MSFVAGVIRRLDETVVNRIAAGEVIQRPANAIKEMTENCLDAKSTNIQVIVREGGLKLIQIQDNGTGIRKEDLDIVCERFTTSKLQTFEDLAMISTYGFRGEALASISHVAHVTITTKTADGKCAYRASYSDGKLQAPPKPCAGNQGTLITVEDLFYNIITRKKALKNPSEEYGKILEVVGRYSIHNSGISFSVKKQGETVSDVRTLPNATTVDNIRSIFGNAVSRELIEVGCEDKTLAFKMNGYISNANYSVKKCIFLLFINHRLVESAALKKAIEAVYAAYLPKNTHPFLYLILEISPQNVDVNVHPTKHEVHFLHEESILERVQQHIESKLLGSNSSRMYFTQTLLPGLAGPSGEAVKSTTGIASSSTSGSGDKVHAYQMVRTDSRDQKLDAFMQPVSRRLPSQPQDPVPGNRTEGSPEKAMQKDQEISELPAPMEAAADSASLERESVIGASEVVAPQRHPSSPGSSRKRHPEDSDVEMMENDSRKEMTAACYPRRRIINLTSVLSLQEEINDRGHETLREMLRNHTFVGCVNPQWALAQHQTKLYLLNTTKLSEELFYQILIYDFANFGVLRLPEPAPLFDFAMLALDSPESGWTEEDGPKEGLAEYIVEFLKKKAKMLADYFSVEIDEEGNLIGLPLLIDSYVPPLEGLPIFILRLATEVNWDEEECFESLSKECAVFYSIRKQYILEESALSGQQSDMPGSPSKPWKWTVEHIIYKAFRSHLLPPKHFTEDGNVLQLANLPDLCKVFERC

SEQ ID NO: 205 - protein, 758 aa, Bos taurus
MSLVAGVIRRLDETVVNRIAAGEVIQRPANAIKEMIENCLDAKSTSIQVVVKEGGLKLIQIQDNGTGIRKEDLEIVCERFTTSKLQSFEDLAHISTYGFRGEALASISHVAHVTITTKTADGKCAYRAHYSDGKLKAPPKPCAGNQGTQITVEDLFYNISTRRKALKNPSEEYGKILEVVGRYAVHNSGIGFSVKKQGETVADVRTLPNATTVDNIRSIFGNAVSRELIEVECEDKTLAFKMNGYISNANYSVKKCIFLLFINHRLVESASLRKAIETVYAAYLPKSTHPFLYLSLEISPQNVDVNVHPTKHEVHFLHEDSILERLQQHIESRLLGSNASRTYFTQTLLPGLPGPSGEAVKSTASVTSSSTAGSGDRVYAHQMVRTDCREQKLDAFLQPVSKALSSQPQAVVPEHRTDASSSGTRQQDEEMLELPAPAAVAAKSQALEDDATMRAADLAEKRGPSSSPENPRKRPREDSDVEMVEDASRKEMTAACTPRRRIINLTSVLSLQEEINERGHETLREMLHNHSFVGCVNPQWALAQHQTKLYLLNTTRLSEELFYQILVYDFANFGVLRLSEPAPLFDLAMLALDSPESGWTEEDGPKEGLAEYIVEFLKKKAEMLADYFSLEIDEEGNLVGLPLLIDNYVPPLEGLPIFILRLATEVNWDEEKECFESLSKECAMFYSIRKQYVSAESTLSGQQSEVPGSTANPWKWTVEHVIYKAFRSHLLPPKHFTEDGNILQLANLPDLYKVFERC

SEQ ID NO: 206 - protein, 256 aa, synthetic construct
INLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 207 - protein, 253 aa, synthetic construct
INLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 208 - protein, 296 aa, synthetic construct
KRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 209 - protein, 293 aa, synthetic construct
KRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 210 - protein, 260 aa, synthetic construct
PKKKRKVINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 211 - protein, 300 aa, synthetic construct
PKKKRKVKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 212 - DNA, 125 bp, synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 213 - DNA, 121 bp, synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 214 - DNA, 109 bp, synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 215 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 216 - DNA, 88 bp, synthetic construct
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 217 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 218 - RNA, 82 nt, synthetic construct
  source 1..82: mol_type = other RNA
gttttagagctagaaatagcaagttaaaataaaggctagtccgttatcaacttgaaaaagtggcaccgagtcggtgcttttt

SEQ ID NO: 219 - DNA, 905 bp, synthetic construct
  source 1..905: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgact

SEQ ID NO: 220 - DNA, 942 bp, synthetic construct
  source 1..942: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 221 - DNA, 1370 bp, synthetic construct
  source 1..1370: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaaacacactgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaatctctctgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 222 - DNA, 807 bp, synthetic construct
  source 1..807: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttgtttcaaaagtagactgtacgctaagggtcatatctttttttgtttggtttgtgtcttggttggcgtcttaaa

SEQ ID NO: 223 - DNA, 743 bp, synthetic construct
  source 1..743: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcagttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttcagcaagttcagagaaatctgaacttgctggatttttggagcagggagatggaataggagcttgctccgtccactccacgcatcgacctggtattgcagtacctccaggaacggtgcacccactttctggagtttcaaaagtagactgtacgctaagggtcatatctttttttgtttggtttgtgtcttggttggcgtcttaaa

SEQ ID NO: 224 - DNA, 141 bp, synthetic construct
  source 1..141: mol_type = other DNA
ggcccagactgagcacgtgagttttagagctagctcatgaaaatgagctagcaagttaaaataaggctagtccgttatcaacttgaaaaagtgggaccgagtcggtcctctgccatcaaagcgtgctcagtctgttttttt

SEQ ID NO: 225 - DNA, 129 bp, synthetic construct
  source 1..129: mol_type = other DNA
ggcccagactgagcacgtgagtttgagagctagaaatagcaagtttaaataaggctagtccgttatcaacttgaaaaagtgggaccgagtcggtcctctgccatcaaagcgtgctcagtctgttttttt

SEQ ID NO: 226 - DNA, 197 bp, synthetic construct
  source 1..197: mol_type = other DNA
ggcccagactgagcacgtgagttttagagctagaaatagcaagttaaaataaggctagtccgttatcaacttgaaaaagtgggaccgagtcggtcctctgccatcaaagcgtgctcagtctgggccggcatggtcccagcctcctcgctggcgccggctgggcaacatgcttcggcatggcgaatgggacttttttt

SEQ ID NO: 227 - DNA, 171 bp, synthetic construct
  source 1..171: mol_type = other DNA
ggtgggagacgtcccaccggcccagactgagcacgtgagttttagagctagaaatagcaagttaaaataaggctagtccgttatcaacttgaaaaagtgggaccgagtcggtcctctgccatcaaagcttcgaccgtgctcagtctggtgggagacgtcccaccttttttt

SEQ ID NO: 228 - DNA, 180 bp, synthetic construct
  source 1..180: mol_type = other DNA
gagcagcatggcgtcgctgctcacggcccagactgagcacgtgagttttagagctagaaatagcaagttaaaataaggctagtccgttatcaacttgaaaaagtgggaccgagtcggtcctctgccatcaaagcttcgaccgtgctcagtctccatcagttgacaccctgaggttttttt

SEQ ID NO: 229 - DNA, 188 bp, synthetic construct
  source 1..188: mol_type = other DNA
  modified_base 15: mod_base = OTHER; note = uracil
  modified_base 61: mod_base = OTHER; note = uracil
  modified_base 101: mod_base = OTHER; note = uracil
gcagacctaagtggtgacatatggtctgggcccagactgagcacgtgagttttagagctatacgtagcaagttaaaataaggctagtccgttatcaactttacgaagtgggaccgagtcggtcctctgccatcaaagcttcgaccgtgctcagtctgcatgcgattagaaataatcgcatgttttttt

SEQ ID NO: 230 - DNA, 125 bp, synthetic construct
  source 1..125: mol_type = other DNA
tctgccatcaaagctgcgaccgtgctcagtctggtgggagacgtcccaccggccggcatggtcccagcctcctcgctggcgccggctgggcaacatgcttcggcatggcgaatgggacttttttt

SEQ ID NO: 231 - DNA, 10 bp, synthetic construct
  source 1..10: mol_type = other DNA
gttttaaaac

SEQ ID NO: 232 - DNA, 112 bp, synthetic construct
  source 1..112: mol_type = other DNA
ttactgaaactcccgacacttccactctgctgattgaaaactcctccccttctggcggctcaaaaagaaccgccgacggcagcgaattcgagtctcccaagaagaagaggaa

SEQ ID NO: 233 - protein, 34 aa, synthetic construct
ITETPDTSTLLIENSSPKRTADGSEFESPKKKRK

SEQ ID NO: 234 - DNA, 112 bp, synthetic construct
  source 1..112: mol_type = other DNA
ttcctcttcttcttgggagactcgaattcgctgccgtcggcggttctttttgagccgccagaaggggaggagttttcaatcagcagagtggaagtgtcgggagtttcagtaa

SEQ ID NO: 235 - DNA, 15 bp, synthetic construct
  source 1..15: mol_type = other DNA
gcacgtgatggcaga

SEQ ID NO: 236 - DNA, 15 bp, synthetic construct
  source 1..15: mol_type = other DNA
gcacgagatggcaga

SEQ ID NO: 237 - DNA, 15 bp, synthetic construct
  source 1..15: mol_type = other DNA
gcacgatccggcaga

SEQ ID NO: 238 - DNA, 15 bp, synthetic construct
  source 1..15: mol_type = other DNA
gcacgatccagcaga

SEQ ID NO: 239 - DNA, 15 bp, synthetic construct
  source 1..15: mol_type = other DNA
ttcgcgctggcatct

SEQ ID NO: 240 - DNA, 15 bp, synthetic construct
  source 1..15: mol_type = other DNA
ttcgccctggcatct

SEQ ID NO: 241 - DNA, 15 bp, synthetic construct
  source 1..15: mol_type = other DNA
ttcgccatggcatct

SEQ ID NO: 242 - DNA, 15 bp, synthetic construct
  source 1..15: mol_type = other DNA
ttcgccaacgcatct

SEQ ID NO: 243 - protein, 8 aa, synthetic construct
ASPRAGGK

SEQ ID NO: 244 - protein, 8 aa, synthetic construct
YEADSLEE

SEQ ID NO: 245 - protein, 6 aa, synthetic construct
YQYRAL

SEQ ID NO: 246 - protein, 6 aa, synthetic construct
LGVLIV

(19) United States
(12) Patent Application Publication
(10) Pub. No.: US 2025/0064979 A1
(43) Pub. Date: Feb. 27, 2025

(54) SELF-ASSEMBLING VIRUS-LIKE PARTICLES FOR DELIVERY OF PRIME EDITORS AND METHODS OF MAKING AND USING SAME

(71) Applicants: The Broad Institute, Inc., Cambridge, MA (US); President and Fellows of Harvard College, Cambridge, MA (US)

(72) Inventors: David R. Liu, Cambridge, MA (US); Aditya Raguram, Cambridge, MA (US); Samagya Banskota, Cambridge, MA (US); Meirui An, Cambridge, MA (US)

(73) Assignees: The Broad Institute, Inc., Cambridge, MA (US); President and Fellows of Harvard College, Cambridge, MA (US)

(21) Appl. No.: 18/715,587

(22) PCT Filed: Dec. 2, 2022
(86) PCT No.: PCT/US2022/080836

§ 371 Date: May 31, 2024

Related U.S. Application Data
(60) Provisional application No. 63/423,372, filed on Nov. 7, 2022.
(60) Provisional application No. 63/298,626, filed on Jan. 11, 2022.
(60) Provisional application No. 63/285,995, filed on Dec. 3, 2021.

(51) Int. Cl. A61K 48/00; C12N 7/00; C12N 9/12; C12N 9/22; C12N 15/11; C12N 15/86

(52) U.S. Cl. CPC A61K 48/0058; C12N 7/00; C12N 9/1276; C12N 9/22; C12N 15/11; C12N 15/86; C12Y 207/07049; C12N 2310/20; C12N 2740/13022; C12N 2740/13023; C12N 2740/13043; C12N 2740/13052; C12N 2760/20222

(57) ABSTRACT

The present disclosure provides virus-like particles (VLPs) for delivering prime editors, and systems comprising such prime editor (PE) VLPs. The present disclosure also provides polynucleotides encoding the PE-VLPs described herein, which may be useful for producing said PE-VLPs. Also provided herein are methods for editing the genome of a target cell by introducing the presently described PE-VLPs into the target cell. The present disclosure also provides fusion proteins that make up a component of the PE-VLPs described herein, as well as polynucleotides, vectors, cells, and kits.

### RELATED APPLICATIONS

This application claims priority under 35 U.S.C. § 119(e) to U.S. Provisional application, U.S. Ser. No. 63/285,995, filed Dec. 3, 2021, U.S. Provisional application, U.S. Ser. No. 63/298,626, filed Jan. 11, 2022, and U.S. Provisional application, U.S. Ser. No. 63/423,372, filed Nov. 7, 2022, each of which is incorporated herein by reference.

### GOVERNMENT SUPPORT

This invention was made with government support under Grant Nos. UG3AI150551, U01AI142756, R35GM118062, RM1HG009490, R01EY009339, and T32GM095450 awarded by the National Institutes of Health. The government has certain rights in the invention.

### BACKGROUND OF THE INVENTION

Recently developed gene editing agents enable the precise manipulation of genomic DNA in living organisms and raise the possibility of treating the root cause of many genetic diseases (Anzalone et al., 2020; Doudna, 2020). The recent development of prime editing enables the insertion, deletion, or replacement of genomic DNA sequences without requiring error-prone double-strand DNA breaks. See Anzalone et al., “Search-and-replace genome editing without double-strand breaks or donor DNA,” Nature, 2019, Vol. 576, pp. 149-157, the contents of which are incorporated herein by reference. Prime editing uses an engineered Cas9 nickase-reverse transcriptase fusion protein paired with an engineered prime editing guide RNA (pegRNA) that not only directs Cas9 to a target genomic site, but also encodes the information for installing the desired edit. Prime editing proceeds through a multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

The broad therapeutic application of in vivo prime editing requires safe and efficient methods for delivering prime editors (PEs) to multiple tissues and organs. Adeno-associated viruses (AAVs) and lentivirus (LV) have been used to deliver gene editing agent-encoding DNA to target tissues (Levy et al., 2020; Newby and Liu, 2021). However, viral delivery of DNA encoding editing agents leads to prolonged expression in transduced cells, which increases the frequency of off-target editing (Akcakaya et al., 2018; Davis et al., 2015; Wang et al., 2020; Yeh et al., 2018). In addition, viral delivery of DNA raises the possibility of viral vector integration into the genome of transduced cells, both of which can promote oncogenesis or other adverse effects (Anzalone et al., 2020; Chandler et al., 2017). Further, in spite of the constant evolution of transfection methods and performances of viral delivery vectors (e.g., AAV or LV), the efficiency of these approaches can vary dramatically, especially in primary cells that are highly sensitive to modifications of their environment and may be altered in response to transfection agents and/or vectors.

One alternate method for delivering gene editing agents (e.g., PEs) in vivo would be to directly deliver proteins (e.g., a PE) or ribonucleoproteins (RNPs) (e.g., a PE complexed with a pegRNA) instead of DNA. The short lifespan of RNPs in cells limits opportunities for off-target editing. No generalizable strategy for delivering PE RNPs to multiple tissues and organs in vivo has been reported previously. Accordingly, there is a need for a method that effectively delivers PE ribonucleoproteins (RNPs) into cells, tissues, or organs of subjects in need, and in a manner which improves the overall safety by limiting and/or avoiding off-target editing without sacrificing target edits.

### SUMMARY OF THE INVENTION

The present disclosure describes the engineering of virus-like particles (VLPs) to package prime editors (PE), the associated prime editor guide RNAs (pegRNAs), and other components to enable efficient prime editing. In one aspect, the present disclosure provides virus-like particles (referred to herein as either “VLPs” or “eVLPs” (“engineered virus-like particles”) interchangeably) comprising a group-specific antigen (gag) protease (pro) polyprotein and one or more fusion proteins, wherein the gag-pro polyprotein and the one or more fusion proteins are encapsulated by a lipid membrane and a viral envelope glycoprotein, and wherein each of the one or more fusion proteins comprises: (i) a gag nucleocapsid protein; (ii) a nuclear export sequence (NES); (iii) a cleavable linker; and (iv) a nucleic acid programmable DNA binding protein (napDNAbp) and/or a domain comprising an RNA-dependent DNA polymerase activity. In some embodiments, the fusion protein comprises both a napDNAbp and a domain comprising an RNA-dependent DNA polymerase activity. In some embodiments, a VLP comprises a first fusion protein comprising the napDNAbp and a second fusion protein comprising the domain comprising an RNA-dependent DNA polymerase activity. In certain embodiments, the first and the second fusion proteins each comprise a portion of a split intein to facilitate fusion of the napDNAbp and the domain comprising an RNA-dependent DNA polymerase activity to one another following delivery of the VLP into a target cell. Without being bound by theory, the components of the VLPs provided herein self-assemble at the cell membrane and bud out in accordance with the naturally occurring mechanism of budding (e.g., retroviral budding or the budding mechanism of other envelope viruses) in order to release from the cell fully-matured VLPs. Once formed, the Gag-Pol-Pro cleaves the protease-sensitive linker of the Gag-cargo (i.e., [Gag]-[cleavable linker]-[cargo], wherein the cargo can be, for example, PE-RNP) thereby releasing the PE RNP within the VLP. Thus, in various embodiments, the present disclosure also provides VLPs in which the protease-sensitive linker has been cleaved (e.g., producing two cleavage products comprising (i) a fusion protein comprising a gag nucleocapsid protein and a nuclear export sequence, and (ii) a prime editor). For example, the present disclosure provides VLPs comprising (i) a group-specific antigen (gag) protease (pro) polyprotein, (ii) a prime editor protein comprising a nucleic acid programmable DNA binding protein (napDNAbp) and a domain comprising an RNA-dependent DNA polymerase activity (e.g., a reverse transcriptase), and (iii) a fusion protein comprising a gag nucleocapsid protein and a nuclear export sequence (NES), encapsulated by a lipid membrane and a viral envelope glycoprotein. In some embodiments, the present disclosure provides VLPs comprising a mixture of cleaved and uncleaved products (i.e., some of the prime editors have been cleaved from the gag proteins and are free, while some have not yet been cleaved from the gag proteins). In some embodiments, more than 50%, more than 60%, more than 70%, more than 80%, or more than 90% of the prime editor has been cleaved from the gag protein inside the VLP. Once the VLP is administered to a recipient cell and taken up by said recipient cell, the contents of the VLP are released, e.g., released PE RNP. Once in the cell, the RNPs may translocate to the nucleus of the cell (in particular, where nuclear localization signals (NLSs) are linked to the RNPs), where DNA editing may occur at target sites specified by the guide RNA. The present disclosure also provides polynucleotides and vectors encoding various components of the VLPs described herein.

In another aspect, the present disclosure provides pluralities of polynucleotides comprising: (i) a first polynucleotide comprising a nucleic acid sequence encoding a viral envelope glycoprotein; (ii) a second polynucleotide comprising a nucleic acid sequence encoding a group-specific antigen (gag) protease (pro) polyprotein; (iii) a third polynucleotide comprising a nucleic acid sequence encoding one or more fusion proteins, wherein each of the one or more fusion protein comprises: (a) a gag nucleocapsid protein; (b) a nuclear export sequence (NES); (c) a cleavable linker; and (d) a nucleic acid programmable DNA binding protein (napDNAbp) and/or a domain comprising an RNA-dependent DNA polymerase activity; and (iv) a fourth polynucleotide comprising a nucleic acid sequence encoding a guide RNA (gRNA), wherein the gRNA binds to the napDNAbp of the fusion protein encoded by the third polynucleotide. In some embodiments, a pharmaceutical composition comprises a VLP comprising (i) a group-specific antigen (gag) protease (pro) polyprotein, (ii) a prime editor protein comprising a nucleic acid programmable DNA binding protein (napDNAbp) and a domain comprising an RNA-dependent DNA polymerase activity (e.g., a reverse transcriptase), and (iii) a fusion protein comprising a gag nucleocapsid protein and a nuclear export sequence (NES), encapsulated by a lipid membrane and a viral envelope glycoprotein.

In another aspect, the present disclosure provides pharmaceutical compositions comprising a virus-like particle (VLP) comprising a group-specific antigen (gag) protease (pro) polyprotein and one or more fusion proteins, wherein the gag-pro polyprotein and the one or more fusion proteins are encapsulated by a lipid membrane and a viral envelope glycoprotein, and wherein each of the one or more fusion proteins comprises: (i) a gag nucleocapsid protein; (ii) a nuclear export sequence (NES); (iii) a cleavable linker; and (iv) a nucleic acid programmable DNA binding protein (napDNAbp) and/or a domain comprising an RNA-dependent DNA polymerase activity.

In another aspect, the present disclosure provides methods for editing a nucleic acid molecule in a target cell by prime editing comprising contacting the target cell with any of the compositions provided herein, thereby installing one or more modifications to the nucleic acid molecule at a target site. In some embodiments, the cell is a mammalian cell (e.g., a human cell). In some embodiments, the cell is a cell from an animal relevant for veterinary or agricultural use. In some embodiments, the cell is in a subject. In certain embodiments, the subject is a human. In some embodiments, the one or more modifications to the nucleic acid molecule are associated with reducing, relieving, or preventing the symptoms of a disease or disorder.

In another aspect, the present disclosure provides fusion proteins comprising: (i) a gag nucleocapsid protein; (ii) a nuclear export sequence (NES); (iii) a cleavable linker; and (iv) a nucleic acid programmable DNA binding protein (napDNAbp) and/or a domain comprising an RNA-dependent DNA polymerase activity. In some embodiments, the fusion protein comprises both a napDNAbp and a domain comprising an RNA-dependent DNA polymerase activity. In some embodiments, the present disclosure provides compositions comprising a first fusion protein disclosed herein, wherein the first fusion protein comprises a napDNAbp, and a second fusion protein disclosed herein, wherein the second fusion protein comprises a domain comprising a domain comprising an RNA-dependent DNA polymerase activity. In certain embodiments, the first and the second fusion proteins each comprise a portion of a split intein to facilitate fusion of the napDNAbp and the domain comprising an RNA-dependent DNA polymerase activity to one another (e.g., following delivery of the fusion proteins in a VLP disclosed herein into a target cell).

In other aspects, the present disclosure also provides methods for making the PE-VLPs described herein, and methods for prime editing comprising delivering the PE-VLPs described herein to a target cell. Polynucleotides, vectors, cells, and kits comprising the PE-VLPs and fusion proteins described herein are also provided.

In another aspect, the present disclosure provides VLPs produced by transfecting, transducing, electroporating, or otherwise inserting any of the polynucleotides or vectors disclosed herein into a cell and expressing the components of the VLPs from the polynucleotides or vectors, thereby allowing the virus-like particle to spontaneously assemble in the cell. In some embodiments, any of the compositions, methods, or cells described herein may be used to produce the VLPs provided herein.

In another aspect, the present disclosure provides compositions comprising any of the VLPs, polynucleotides, vectors, and fusion proteins provided herein.

In another aspect, the present disclosure provides methods of editing a nucleic acid molecule in a target cell using any of the VLPs, polynucleotides, compositions, and fusion proteins provided herein.

In another aspect, the present dislosure provides cells comprising any of the VLPs, polynucleotides, vectors, compositions, and fusion proteins described herein.

In another aspect, the present disclosure provides kits comprising any of the VLPs, polynucleotides, vectors, compositions, and fusion proteins described herein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1: Summary of previously-developed delivery methods for CRISPR/Cas systems.

FIGS. 2A-2D: Summary of prime editor ribonucleoprotein (PE-RNP) virus-like particle (VLP) delivery strategy.

FIGS. 3A-3B: PE-RNP VLP optimizations of single vs. two-particle system. A single particle system is shown to be more efficient than a two-particle system.

FIGS. 4A-4B: PE-RNP VLP optimizations of 1× vs. 2×NLS system. Incorporation of two NLS is shown to improve editing efficiency.

FIG. 5: Optimizations contribute to packaging of editors into VLPs. Incorporation of an NES promotes export of PE into cytoplasm of producer cells. Gag-fusion directs the packaging of editors into VLPs.

FIG. 6: Efficiency of HEK3+1 T>A edit in HEK293T cells using various concentrations of VLP compared to plasmid transfection.

FIG. 7: Schematic of a pegRNA and a prime editor.

FIGS. 8A-8C: Assessment of pegRNA packaging. Supplementing pegRNAs by plasmid transfection is shown to enhance editing efficiency. In contrast, editing with an adenosine base editor (ABE) is not improved significantly with sgRNA transfection.

FIG. 9: Assessment of pegRNA binding affinity to PE. pegRNAs are shown to have a lower binding affinity to Cas9 compared to sgRNA.

FIGS. 10A-10B: Adoption of F+E scaffold for improved pegRNA binding. The F+E scaffold is shown to modestly improve pegRNA binding to Cas9 in a pegRNA limiting context.

FIGS. 11A-11E: Incorporation of MS2 stem loop for specific packaging of pegRNA.

FIG. 12: Incorporation of PEmax for more robust editing. Delivery of PEmax using VLPs is shown to result in improved editing efficiency.

FIG. 13: Assessment of PE packaging. A qualitative assessment of Cas9 content by dot blot is shown.

FIGS. 14A-14C: Trimming down the polymerase domain to increase cargo space in the VLPs.

FIG. 15: PE3max RNP VLP system. Use of 30% nicking gRNA is shown to lead to the highest editing efficiency. Approximately a 3.5-fold improvement is observed compared to PE2max.

FIGS. 16A-16B: Comparison of PE3max RNP VLP separate-particle system vs. all-in-one particle system. Varying ratios of VLP (editor+ngRNA):VLP (editor+pegRNA) were screened in 50 μl total VLP. The separate-particle system is shown to have comparable editing efficiency to the all-in-one particle system.

FIGS. 17A-17B: PE3max RNP VLP separate-particle system with varying transduction timing. The all-in-one particle system is shown to have increased editing efficiency.

FIG. 18: Mismatch repair-privileged edits are shown to lead to higher overall editing in both PE2 and PE3 RNP VLPs. This suggests that installation of silent mutations to evade MMR may confer improved editing efficiency, especially in a PE-limited context such as the RNP VLP system.

FIG. 19A-19D: PE4max ribonucleoprotein VLP. MLHldn protein was packaged into the VLP using both the all-in-one particle and separate particle systems. Dual transfection-transduction showed that 1) MLHldn plasmid transfection offers significant improvement to PE2 VLP editing efficiency, showing that evading MMR has a significant role in improving PE-VLP editing efficiency; and 2) MLHldn is being packaged in the VLP particle.

FIG. 20: Installing silent mutations improves PE RNP VLP. PE VLP has a similar editing efficiency to plasmid transfection when MMR is sufficiently evaded.

FIG. 21: Assessment of PE assembly. Varying expression of Cas9 and RT halves and inefficient intein trans-splicing may lead to poisoning of the editing site.

FIGS. 22A-22B: Optimization of whole length PE and Cas9 internal split. pmA97 construct (full length PE with RT protease site deletion) showed the highest editing efficiency. At the C-terminus of the RT, a protease cleavage site is present that can be recognized by the MMLV-protease being expressed in the system. If the protease recognizes and cleaves this site, the NLS at the C-terminus of the RT is also cleaved from the prime editor. Thus, deleting the RT protease site improves editing efficiency. In FIG. 22B, sequences shown correspond (top-bottom) to SEQ ID NOs: 232-234.

FIG. 23A-23B: Optimization of full-length PE and Cas9 internal split. Full-length PE shows higher editing efficiency than split PE.

FIGS. 24A-24B: Validation of Cas9-mRNA VLP strategy.

FIGS. 25A-25B: Editing efficiency of PE2max mRNA VLP version 1.

FIGS. 26A-26B: Whole editor construct shows higher editing efficiency than split editor construct. Splitting the editor construct did not improve editing.

FIGS. 27A-27C: Editing efficiency of PE2max mRNA VLP version 2. Psi-signal on the pLV-vector only allows two copies of the viral genome into a particle. MS2-stem loop inserted-pegRNA may increase pegRNA packaging.

FIGS. 28A-28C: Changing the HIV capsid to MMLV capsid in PEmax mRNA VLP design version 2. MMLV capsid leads to higher titer production. pegRNA expression in lentiviral-expression vector enables packaging of more functional pegRNA than in conventional plasmid backbone.

FIGS. 29A-29B: Optimizing the MCP-fusion gag protein in PE2max mRNA VLP version 2. The polymerase domain is important in the viral production process.

FIG. 30: Additional MCP-fusion constructs.

FIG. 31: PE2max mRNA VLP version 2. Features include a 6×MS2 stem loop utilized for packaging of a transgene mRNA.

FIG. 32 shows engineering of split prime editors for more efficient packaging. Full-length editor constructs generally led to higher editing efficiencies. A six amino acid deletion at the C-terminus of the MMLV reverse transcriptase to remove the endogenous protease cleavage site and prevent the NLS on the prime editor from being cleaved off increased editing efficiency in both full-length and split prime editor constructs.

FIG. 33 provides a schematic showing that a fraction of the prime editors delivered by eVLPs may still retain the NES after protease cleavage.

FIGS. 34A-34B show engineering of the NES position to ensure cleavage from the prime editors. Sites with Gag protein that are tolerable to larger insertions were explored.

Insertion of 3×NES in front of the endogenous protease cleavage site between the p12 and the CA domains (NES position 1) resulted in the highest editing efficiencies.

FIGS. 35A-35B show the addition of linkers to better expose the protease cleavage site. SEQ ID NO: 163 (SGGSSGGS) is shown.

FIG. 36 shows combination of the optimized NES positions and linker sequence. V5 eVLP architecture includes these optimized NES position and linker sequence.

FIGS. 37A-37B show that the mismatch repair (MMR) pathway may be especially detrimental to PE-eVLP editing efficiency. MMR-privileged editing leads to higher overall editing in both PE2 and PE3 RNP VLP.

FIGS. 38A-38C show packaging of MLHdn in eVLP. MLHdn-eVLP transduction showed similar editing efficiency to PE2 plasmid transfection. The amount of MLHdn packaged may not be sufficient to suppress MMR.

FIGS. 39A-39B show installation of additional contiguous mutations to evade MMR.

Installation of additional contiguous mutations is a promising strategy for escaping MMR as no additional components need to be packaged in the eVLP. In FIG. 39A, sequences correspond (top-bottom) to SEQ ID NOs: 235-242.

FIGS. 40A-40D show inclusion of the MS2 stem loop for specific packaging of pegRNA. MS2 aptamer insertion in the scaffold region of the pegRNA improves pegRNA packaging via interaction with MCP-Gag-pol.

FIGS. 41A-41C show inclusion of the MS2 stem loop to facilitate nicking guide RNA (ngRNA) packaging for PE3. The MS2 aptamer was shown to improve ngRNA packaging.

An all-in-one particle system including both MS2-pegRNA and MS2-ngRNA was demonstrated to provide the highest PE3 editing efficiency.

FIGS. 42A-42B show that use of the com protein and com aptamer is comparable to the MCP-MS2 aptamer system.

FIGS. 43A-43C show optimization of plasmid ratios for VLP production. In particular, the ratio of Gag-pol to MCP-Gag-pol to Gag-cargo was optimized as shown.

FIGS. 44A-44B show the use of coiled-coil peptides as an additional mechanism for prime editor recruitment in VLPs. In FIG. 44A, when the P4 peptide domain is shown upside down, this indicates an anti-parallel coiled-coil construct design.

FIGS. 45A-45B show that coiled-coil peptide-prime editor constructs improve editing efficiency.

FIGS. 46A-46D provide schematics of coiled-coil peptide-prime editor constructs and show that MCP fusion constructs provide superior editing efficiency over coiled-coil constructs.

FIGS. 47A-47B show testing of PE VLPs in vivo in P0 mice by ICV injection with PE VLP. PE VLPs showed efficient editing in cell populations that are transducible by VSV-g.

FIG. 48 shows testing of PE VLPs in vivo by subretinal injection in rd6 model mice. Correction of the gene encoding the retinal disease-associated membrane-type frizzled-related protein (Mfrp) was observed.

FIGS. 49A-49D show further testing of PE VLPs in vivo by subretinal injection in rd6 model mice. An average of 15% editing with PE3 VLP and protein restoration was observed.

FIGS. 50A-50B show further optimization of PE VLPs for subretinal injection in rd12 model mice using additional silent mutations in the pegRNA and various concentrations of VLP containing either PE2 or PE3.

FIG. 51 shows additional strategies for recruitment of prime editor to eVLPs via coiled-coil peptides.

FIG. 52 shows that evolved small reverse transcriptase (Tf1) can be used in the prime editors delivered by eVLPs.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 37). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 37). In some embodiments, the Cas9 variant comprises a fragment of SEQ ID NO: 37 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 37). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 37).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a PEgRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template may comprise the “edit template” and the “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the PEgRNA molecule to the 3′ end of the edit template. Preferably, the DNA synthesis template excludes the primer binding site (PBS) of PEgRNAs either having a 3′ extension arm or a 5′ extension arm. Certain embodiments described here refer to an “an RT template,” which is inclusive of the edit template and the homology arm, i.e., the sequence of the PEgRNA extension arm that is actually used as a template during DNA synthesis. The term “RT template” is equivalent to the term “DNA synthesis template.”

### Edit Template

The term “edit template” refers to a portion of the extension arm that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here refer to “an RT template,” which refers to both the edit template and the homology arm together, i.e., the sequence of the PEgRNA extension arm that is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Extension Arm

The term “extension arm” refers to a nucleotide sequence component of a PEgRNA which provides several functions, including a primer binding site and an edit template for reverse transcriptase. In some embodiments, the extension arm is located at the 3′ end of the guide RNA. In other embodiments, the extension arm is located at the 5′ end of the guide RNA. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand. Further details, such as the length of the extension arm, are described elsewhere herein.

The extension arm may also be described as comprising generally two regions: a primer binding site (PBS) and a DNA synthesis template, for instance. The primer binding site binds to the primer sequence that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the PEgRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and that ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the PEgRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes fusion of a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which is incorporated herein by reference.

Group-Specific Antigen (gag)

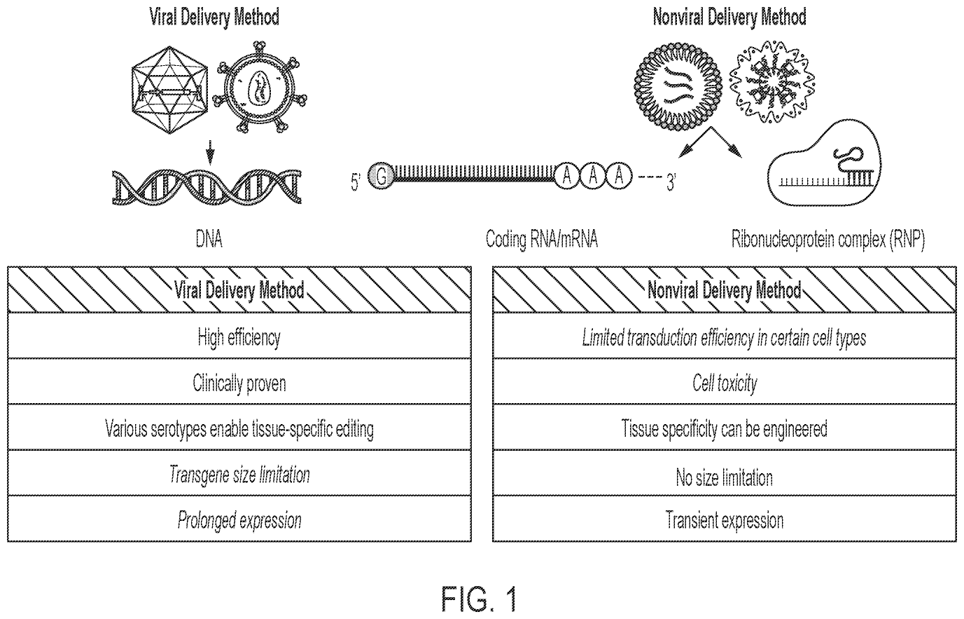

Without being limited by theory, and in the context of typical envelope virus lifecycle, Gag is the primary structural protein responsible for orchestrating the majority of steps in viral assembly, including budding out of fully-formed enveloped virions having an (i) envelope (comprising a lipid membrane formed from cell membrane during budding out, and one or more glycoproteins inserted therein), and (ii) a capsid, which is the internal protein shell. Most of these assembly steps occur via interactions with three Gag subdomains—matrix (MA), capsid (CA), and nucleocapsid (NC; FIG. 1). These three regions have a low level of sequence conservation among the different retroviral genera, which belies the observed high level of structural conservation. Outside of these three domains, Gag proteins can vary widely. For example, HIV-1 Gag additionally codes for a C-terminal p6 protein as well as two spacer proteins, SP1 and SP2, which demarcate the CA-NC and NC-p6 junctions, but HTLV-1 contains no additional sequences outside of MA, CA, and NC (Oroszlan and Copeland, 1985; Henderson et al., 1992).

Gag is also referred to as a “viral structural protein.” As used herein, the term “viral structural protein” refers to viral proteins that contribute to the overall structure of the capsid protein or of the protein core of a virus. The term “viral structural protein” further includes functional fragments or derivatives of such viral protein contributing to the structure of a capsid protein or of protein core of a virus. An example of viral structural protein is MMLV Gag. The viral membrane fusion proteins are not considered as viral structural proteins. Typically, said viral structural proteins are localized inside the core of the virus.

Group-Specific Antigen (gag) Nucleocapsid Protein

The term “group-specific antigen nucleocapsid protein” or “gag nucleocapsid protein” refers to a protein that makes up the core structural component of the inner shell of many viruses. The gag nucleocapsid proteins used in the PE-VLPs of the present disclosure may be an MMLV gag nucleocapsid protein, an FMLV gag nucleocapsid protein, or a nucleocapsid protein from any other virus that produces such proteins.

### Group-Specific Antigen (Sa) Protease (Pro) Polyprotein

A “group-specific antigen (gag) protease (pro) polyprotein” or “gag-pro polyprotein” refers to a gag nucleocapsid protein further comprising a viral protease linked thereto. Gag-pro polyproteins mediate proteolytic cleavage of gag and gag-pol polyproteins or nucleocapsid proteins during or shortly after the release of a virion from the plasma membrane. In the PE-VLPs described herein, the protease of a gag-pro polyprotein is responsible for cleaving a cleavable linker in the fusion protein to release a prime editor following delivery of the PE-VLP to a target cell. In some embodiments, a gag-pro polyprotein is an MMLV gag-pro polyprotein or an FMLV gag-pro polyprotein.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “PEgRNAs”).

Guide RNAs or PEgRNAs may comprise various structural elements that include, but are not limited to:

Spacer sequence—the sequence in the guide RNA or PEgRNA (having about 20 nts in length) which has the same sequence as the protospacer in the target DNA.

gRNA core (or gRNA scaffold or backbone sequence)—the sequence within the gRNA that is responsible for Cas9 binding. It does not include the 20 bp spacer/targeting sequence that is used to guide Cas9 to target DNA.

Extension arm—a single strand extension at the 3′ end or the 5′ end of the PEgRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the guide RNA or PEgRNA may comprise a transcriptional termination sequence at the 3′ of the molecule.

### Linker

The term “linker,” as used herein, refers to a molecule linking two other molecules or moieties. The linker can be an amino acid sequence in the case of a linker joining two fusion proteins. For example, a Cas9 can be fused to a reverse transcriptase by an amino acid linker sequence. The linker can also be a nucleotide sequence in the case of joining two nucleotide sequences together (e.g., in a gRNA). For example, in the instant case, the traditional guide RNA is linked via a spacer or linker nucleotide sequence to the RNA extension of a prime editing guide RNA which may comprise an RT template sequence and an RT primer binding site. In other embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

A “cleavable linker” refers to a linker that can be split or cut by any means. The linker can be an amino acid sequence. In some embodiments, the linker between the NES and the napDNAbp of the PE-VLPs provided herein comprises a cleavable linker. A cleavable linker may comprise a self-cleaving peptide (e.g., a 2A peptide such as EGRGSLLTCGDVEENPGP (SEQ ID NO: 1), ATNFSLLKQAGDVEENPGP (SEQ ID NO: 2), QCTNYALLKLAGDVESNPGP (SEQ ID NO: 3), or VKQTLNFDLLKLAGDVESNPGP (SEQ ID NO: 4)). In some embodiments, a cleavable linker comprises a protease cleavage site that is cut after being contacted by a protease. For example, the present disclosure contemplates the use of cleavable linkers comprising a protease cleavage site of amino acid sequences TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8. In certain embodiments, a cleavable linker comprises an MMLV protease cleavage site of an FMLV protease cleavage site.

### MLH1

The term “MLH1” refers to a gene encoding MLH1 (or MutL Homolog 1), a DNA mismatch repair enzyme. The protein encoded by this gene can heterodimerize with mismatch repair endonuclease PMS2 to form MutL alpha (MutLα), part of the DNA mismatch repair system. MLH1 mediates protein-protein interactions during mismatch recognition, strand discrimination, and strand removal. In mismatch repair, the heterodimer MSH2:MSH6 (MutSα) forms and binds the mismatch. MLH1 then forms a heterodimer with PMS2 (MutLα) and binds the MSH2:MSH6 heterodimer. The MutLα heterodimer then incises the nicked strand 5′ and 3′ of the mismatch, followed by excision of the mismatch from MutLα-generated nicks by EXO1. Finally, POL6 resynthesizes the excised strand, followed by LIG1 ligation.

An exemplary amino acid sequence of MLH1 is human isoform 1, P40692-1: >sp|P40692|MLH1_HUMAN DNA mismatch repair protein Mlh1 OS═Homo sapiens OX=9606 GN=MLH1 PE=1 SV=1:

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLLGSNSSRMYFTQ TLLPGLAGPSGEMVKSTTSLTSSSTSGSSDKVYAHQMVRTDSREQKLDAFLQPLSKP LSSQPQAIVTEDKTDISSGRARQQDEEMLELPAPAEVAAKNQSLEGDTTKGTSEMSE KRGPTSSNPRKRHREDSDVEMVEDDSRKEMTAACTPRRRIINLTSVLSLQEEINEQGH EVLREMLHNHSFVGCVNPQWALAQHQTKLYLLNTTKLSEELFYQILIYDFANFGVLR LSEPAPLFDLAMLALDSPESGWTEEDGPKEGLAEYIVEFLKKKAEMLADYFSLEIDEE GNLIGLPLLIDNYVPPLEGLPIFILRLATEVNWDEEKECFESLSKECAMFYSIRKQYISE ESTLSGQQSEVPGSIPNSWKWTVEHIVYKALRSHILPPKHFTEDGNILQLANLPDLYK VFERC (SEQ ID NO: 9), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 9.

Another exemplary amino acid sequence of MLH1 is human isoform 2, P40692-2 (wherein amino acids 1-241 of isoform 1 are missing): >sp|P40692-2|MLH1_HUMAN Isoform 2 of DNA mismatch repair protein M1h1 OS═Homo sapiens OX=9606 GN=MLH1:

MNGYISNANYSVKKCIFLLFINHRLVESTSLRKAIETVYAAYLPKNTHPFLYLS LEISPQNVDVNVHPTKHEVHFLHEESILERVQQHIESKLLGSNSSRMYFTQTLLPGLA GPSGEMVKSTTSLTSSSTSGSSDKVYAHQMVRTDSREQKLDAFLQPLSKPLSSQPQAI VTEDKTDISSGRARQQDEEMLELPAPAEVAAKNQSLEGDTTKGTSEMSEKRGPTSSN PRKRHREDSDVEMVEDDSRKEMTAACTPRRRIINLTSVLSLQEEINEQGHEVLREML HNHSFVGCVNPQWALAQHQTKLYLLNTTKLSEELFYQILIYDFANFGVLRLSEPAPL FDLAMLALDSPESGWTEEDGPKEGLAEYIVEFLKKKAEMLADYFSLEIDEEGNLIGLP LLIDNYVPPLEGLPIFILRLATEVNWDEEKECFESLSKECAMFYSIRKQYISEESTLSGQ QSEVPGSIPNSWKWTVEHIVYKALRSHILPPKHFTEDGNILQLANLPDLYKVFERC (SEQ ID NO: 10), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 10.

Another exemplary amino acid sequence of MLH1 is human isoform 3, P40692-3 (where amino acids 1-101 (MSFVAGVIRR . . . ASISTYGFRG (SEQ ID NO: 9) is replaced with MAF): >sp|P40692-2|MLH1_HUMAN Isoform 2 of DNA mismatch repair protein Mlh1 OS═Homo sapiens OX=9606 GN=MLH1:

MAFEALASISHVAHVTITTKTADGKCAYRASYSDGKLKAPPKPCAGNQGTQI TVEDLFYNIATRRKALKNPSEEYGKILEVVGRYSVHNAGISFSVKKQGETVADVRTL PNASTVDNIRSIFGNAVSRELIEIGCEDKTLAFKMNGYISNANYSVKKCIFLLFINHRL VESTSLRKAIETVYAAYLPKNTHPFLYLSLEISPQNVDVNVHPTKHEVHFLHEESILER VQQHIESKLLGSNSSRMYFTQTLLPGLAGPSGEMVKSTTSLTSSSTSGSSDKVYAHQ MVRTDSREQKLDAFLQPLSKPLSSQPQAIVTEDKTDISSGRARQQDEEMLELPAPAEV AAKNQSLEGDTTKGTSEMSEKRGPTSSNPRKRHREDSDVEMVEDDSRKEMTAACTP RRRIINLTSVLSLQEEINEQGHEVLREMLHNHSFVGCVNPQWALAQHQTKLYLLNTT KLSEELFYQILIYDFANFGVLRLSEPAPLFDLAMLALDSPESGWTEEDGPKEGLAEYI VEFLKKKAEMLADYFSLEIDEEGNLIGLPLLIDNYVPPLEGLPIFILRLATEVNWDEEK ECFESLSKECAMFYSIRKQYISEESTLSGQQSEVPGSIPNSWKWTVEHIVYKALRSHIL PPKHFTEDGNILQLANLPDLYKVFERC (SEQ ID NO: 12), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 12.

In some embodiments, the present disclosure contemplates delivering using the VLPs described herein an inhibitor of MLH1 and/or MMR pathway components that interact with MLH1, including any wildtype or naturally occurring variant of MLH1, including any amino acid sequence having at least 70%, or 75%, or 80%, or 85%, or 90%, or 95%, or 99% or more sequence identity with any of SEQ ID NOs: 9-19 or 203-211, or nucleic acid molecules encoding any MLH1 or variant of MLH1 (e.g., a dominant negative mutant of MLH1 as described herein), for inhibiting, blocking, or otherwise inactivating the wild type MLH1 function in the MMR pathway, and consequently, inhibiting, blocking, or otherwise inactivating the MMR pathway, e.g., during genome editing with a prime editor.

In some embodiments, inactivation of the MMR pathway involves an inhibitor that disrupts, blocks, interferes with, or otherwise inactivates the wild type function of the MLH1 protein. In some embodiments, inactivation of the MMR pathway involves a mutant of the MLH1 protein, for example, delivering to a target cell using the presently described VLPs an MLH1 mutant protein. In some embodiments, the MLH1 mutant protein interferes with, and thereby inactivates, the function of a wild type MLH1 protein in the MMR pathway. In some embodiments, the MLH1 mutant is a dominant negative mutant. In some embodiments, the MLH mutant protein is capable of binding to an MLH1-interacting protein, for example, MutS.

Without being bound by theory, MLH1 dominant negative mutants function by saturating binding of MutS, thereby blocking MutS-wild type MLH1 binding and interfering with the function of the wild type MLH1 protein in the MMR pathway.

In various embodiments, the dominant negative MLH1 can include, for example, MLH1 E34A, which is based on SEQ ID NO: 13 and has the following amino acid sequence (underline and bolded to show the E34A mutation):

MSFVAGVIRRLDETVVNRIAAGEVIQRPANAIKAMIENCLDAKSTSIQVIVKE GGLKLIQIQDNGTGIRKEDLDIVCERFTTSKLQSFEDLASISTYGFRGEALASISHVAH VTITTKTADGKCAYRASYSDGKLKAPPKPCAGNQGTQITVEDLFYNIATRRKALKNP SEEYGKILEVVGRYSVHNAGISFSVKKQGETVADVRTLPNASTVDNIRSIFGNAVSRE LIEIGCEDKTLAFKMNGYISNANYSVKKCIFLLFINHRLVESTSLRKAIETVYAAYLPK NTHPFLYLSLEISPQNVDVNVHPTKHEVHFLHEESILERVQQHIESKLLGSNSSRMYFT QTLLPGLAGPSGEMVKSTTSLTSSSTSGSSDKVYAHQMVRTDSREQKLDAFLQPLSK PLSSQPQAIVTEDKTDISSGRARQQDEEMLELPAPAEVAAKNQSLEGDTTKGTSEMS EKRGPTSSNPRKRHREDSDVEMVEDDSRKEMTAACTPRRRIINLTSVLSLQEEINEQG HEVLREMLHNHSFVGCVNPQWALAQHQTKLYLLNTTKLSEELFYQILIYDFANFGV LRLSEPAPLFDLAMLALDSPESGWTEEDGPKEGLAEYIVEFLKKKAEMLADYFSLEID EEGNLIGLPLLIDNYVPPLEGLPIFILRLATEVNWDEEKECFESLSKECAMFYSIRKQYI SEESTLSGQQSEVPGSIPNSWKWTVEHIVYKALRSHILPPKHFTEDGNILQLANLPDL YKVFERC (SEQ ID NO: 13), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 13.

In various other embodiments, the dominant negative MLH1 can include, for example, MLH1 Δ756, which is based on SEQ ID NO: 14 and has the following amino acid sequence (underline and bolded to show the A756 mutation at the C terminus of the sequence):

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLLGSNSSRMYFTQ TLLPGLAGPSGEMVKSTTSLTSSSTSGSSDKVYAHQMVRTDSREQKLDAFLQPLSKP LSSQPQAIVTEDKTDISSGRARQQDEEMLELPAPAEVAAKNQSLEGDTTKGTSEMSE KRGPTSSNPRKRHREDSDVEMVEDDSRKEMTAACTPRRRIINLTSVLSLQEEINEQGH EVLREMLHNHSFVGCVNPQWALAQHQTKLYLLNTTKLSEELFYQILIYDFANFGVLR LSEPAPLFDLAMLALDSPESGWTEEDGPKEGLAEYIVEFLKKKAEMLADYFSLEIDEE GNLIGLPLLIDNYVPPLEGLPIFILRLATEVNWDEEKECFESLSKECAMFYSIRKQYISE ESTLSGQQSEVPGSIPNSWKWTVEHIVYKALRSHILPPKHFTEDGNILQLANLPDLYK VFER[-](SEQ ID NO: 14), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 14 (wherein the [-] indicates deleted amino acid residue(s) relative to the parent or wildtype sequence).

In still other embodiments, the dominant negative MLH1 can include, for example, MLH1 Δ754-Δ756, which is based on SEQ ID NO: 15 and has the following amino acid sequence (underline and bolded to show the Δ754-Δ756 mutation at the C terminus of the sequence):

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLLGSNSSRMYFTQ TLLPGLAGPSGEMVKSTTSLTSSSTSGSSDKVYAHQMVRTDSREQKLDAFLQPLSKP LSSQPQAIVTEDKTDISSGRARQQDEEMLELPAPAEVAAKNQSLEGDTTKGTSEMSE KRGPTSSNPRKRHREDSDVEMVEDDSRKEMTAACTPRRRIINLTSVLSLQEEINEQGH EVLREMLHNHSFVGCVNPQWALAQHQTKLYLLNTTKLSEELFYQILIYDFANFGVLR LSEPAPLFDLAMLALDSPESGWTEEDGPKEGLAEYIVEFLKKKAEMLADYFSLEIDEE GNLIGLPLLIDNYVPPLEGLPIFILRLATEVNWDEEKECFESLSKECAMFYSIRKQYISE ESTLSGQQSEVPGSIPNSWKWTVEHIVYKALRSHILPPKHFTEDGNILQLANLPDLYK VF[ - - - ](SEQ ID NO: 15), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 15 (wherein the [ - - - ] indicates deleted amino acid residue(s) relative to the parent or wildtype sequence).

In yet other embodiments, the dominant negative MLH1 can include, for example, MLH1 E34A Δ754-Δ756, which is based on SEQ ID NO: 16 and has the following amino acid sequence (underline and bolded to show the E34A and Δ754-Δ756 mutations):

MSFVAGVIRRLDETVVNRIAAGEVIQRPANAIKAMIENCLDAKSTSIQVIVKE GGLKLIQIQDNGTGIRKEDLDIVCERFTTSKLQSFEDLASISTYGFRGEALASISHVAH VTITTKTADGKCAYRASYSDGKLKAPPKPCAGNQGTQITVEDLFYNIATRRKALKNP SEEYGKILEVVGRYSVHNAGISFSVKKQGETVADVRTLPNASTVDNIRSIFGNAVSRE LIEIGCEDKTLAFKMNGYISNANYSVKKCIFLLFINHRLVESTSLRKAIETVYAAYLPK NTHPFLYLSLEISPQNVDVNVHPTKHEVHFLHEESILERVQQHIESKLLGSNSSRMYFT QTLLPGLAGPSGEMVKSTTSLTSSSTSGSSDKVYAHQMVRTDSREQKLDAFLQPLSK PLSSQPQAIVTEDKTDISSGRARQQDEEMLELPAPAEVAAKNQSLEGDTTKGTSEMS EKRGPTSSNPRKRHREDSDVEMVEDDSRKEMTAACTPRRRIINLTSVLSLQEEINEQG HEVLREMLHNHSFVGCVNPQWALAQHQTKLYLLNTTKLSEELFYQILIYDFANFGV LRLSEPAPLFDLAMLALDSPESGWTEEDGPKEGLAEYIVEFLKKKAEMLADYFSLEID EEGNLIGLPLLIDNYVPPLEGLPIFILRLATEVNWDEEKECFESLSKECAMFYSIRKQYI SEESTLSGQQSEVPGSIPNSWKWTVEHIVYKALRSHILPPKHFTEDGNILQLANLPDL YKVF[ - - - ](SEQ ID NO: 16), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 16.

In certain embodiments, the dominant negative MLH1 can include, for example, MLH1 1-335, which is based on SEQ ID NO: 17 and has the following amino acid sequence (contains amino acids 1-335 of SEQ ID NO: 9):

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLL (SEQ ID NO: 17), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 17.

In other embodiments, the dominant negative MLH1 can include, for example, MLH1 1-335 E34A, which is based on SEQ ID NO: 18 and has the following amino acid sequence (contains amino acids 1-335 of SEQ ID NO: 9 and a E34A mutation relative to SEQ ID NO: 204):

MSFVAGVIRRLDETVVNRIAAGEVIQRPANAIKAMIENCLDAKSTSIQVIVKE GGLKLIQIQDNGTGIRKEDLDIVCERFTTSKLQSFEDLASISTYGFRGEALASISHVAH VTITTKTADGKCAYRASYSDGKLKAPPKPCAGNQGTQITVEDLFYNIATRRKALKNP SEEYGKILEVVGRYSVHNAGISFSVKKQGETVADVRTLPNASTVDNIRSIFGNAVSRE LIEIGCEDKTLAFKMNGYISNANYSVKKCIFLLFINHRLVESTSLRKAIETVYAAYLPK NTHPFLYLSLEISPQNVDVNVHPTKHEVHFLHEESILERVQQHIESKLL (SEQ ID NO: 18), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 18.

In still other embodiments, the dominant negative MLH1 can include, for example, MLH1 1-335 NLS^{SV40} (or referred to as MLH1dn^{NTD}, which is based on SEQ ID NO: 9 and has the following amino acid sequence (contains amino acids 1-335 of SEQ ID NO: 9 and an NLS sequence of SV40):

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLLPKKKRKV (SEQ ID NO: 19), with the underlined and bolded portion referring to the NLS of SV40), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 19.

In still other embodiments, the dominant negative MLH1 can include, for example, MLH1 1-335 NLS^{alternate} (which is based on SEQ ID NO: 9 and having the following amino acid sequence (contains amino acids 1-335 of SEQ ID NO: 9 and an alternate NLS sequence)):

MSFVAGVIRRLDETVVNRIAAGEVIQRPANAIKEMIENCLDAKSTSIQVIVKEG GLKLIQIQDNGTGIRKEDLDIVCERFTTSKLQSFEDLASISTYGFRGEALASISHVAHV TITTKTADGKCAYRASYSDGKLKAPPKPCAGNQGTQITVEDLFYNIATRRKALKNPS EEYGKILEVVGRYSVHNAGISFSVKKQGETVADVRTLPNASTVDNIRSIFGNAVSREL IEIGCEDKTLAFKMNGYISNANYSVKKCIFLLFINHRLVESTSLRKAIETVYAAYLPKN THPFLYLSLEISPQNVDVNVHPTKHEVHFLHEESILERVQQHIESKLL-[alternate NLS sequence](SEQ ID NO: 17)-[alternate NLS sequence], or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 17. The alternate NLS sequence can be any suitable NLS sequence, including but not limited to:

| | | SEQ |
|---|---|---|
| | | ID |
| DESCRIPTION | SEQUENCE | NO: |
| NLS | MKRTADGSEFESPKKKRKV | 20 |
| NLS | MDSLLMNRRKFLYQFKNVRWAKGR | 21 |
| | RETYLC | |
| NLS OF | AVKRPAATKKAGQAKKKKLD | 22 |
| NUCLEOPLASMIN | | |
| NLS OF EGL-13 | MSRRRKANPTKLSENAKKLAKEVEN | 23 |
| NLS OF C-MYC | PAAKRVKLD | 24 |
| NLS OF TUS-PROTEIN | KLKIKRPVK | 25 |
| NLS OF POLYOMA | VSRKRPRP | 26 |
| LARGE T-AG | | |
| NLS OF HEPATITIS D | EGAPPAKRAR | 27 |
| VIRUS ANTIGEN | | |
| NLS OF MURINE P53 | PPQPKKKPLDGE | 28 |
| NLS OF PE1 AND PE2 | SGGSKRTADGSEFEPKKKRKV | 29 |

In still other embodiments, the dominant negative MLH1 can include, for example, MLH1 501-756, which corresponds to a C-terminal fragment of SEQ ID NO: 9 that corresponds to amino acids 501-756 of SEQ ID NO: 9:

INLTSVLSLQEEINEQGHEVLREMLHNHSFVGCVNPQWALAQHQTKLYLLNT TKLSEELFYQILIYDFANFGVLRLSEPAPLFDLAMLALDSPESGWTEEDGPKEGLAEYI VEFLKKKAEMLADYFSLEIDEEGNLIGLPLLIDNYVPPLEGLPIFILRLATEVNWDEEK ECFESLSKECAMFYSIRKQYISEESTLSGQQSEVPGSIPNSWKWTVEHIVYKALRSHIL PPKHFTEDGNILQLANLPDLYKVFERC (SEQ ID NO: 206), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 206.

In still other embodiments, the dominant negative MLH1 can include, for example, MLH1 501-753, which corresponds to a C-terminal fragment of SEQ ID NO: 9 that corresponds to amino acids 501-753 of SEQ ID NO: 9: INLTSVLSLQEEINEQGHEVLREMLHNHSFVGCVNPQWALAQHQTKLYLLNTTKLSE ELFYQILIYDFANFGVLRLSEPAPLFDLAMLALDSPESGWTEEDGPKEGLAEYIVEFL KKKAEMLADYFSLEIDEEGNLIGLPLLIDNYVPPLEGLPIFILRLATEVNWDEEKECFE SLSKECAMFYSIRKQYISEESTLSGQQSEVPGSIPNSWKWTVEHIVYKALRSHILPPKH FTEDGNILQLANLPDLYKVF[ - - - ](SEQ ID NO: 207), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 207.

In still other embodiments, the dominant negative MLH1 can include, for example, MLH1 461-756, which is a C-terminal fragment of SEQ ID NO: 9 that corresponds to amino acids 461-756 of SEQ ID NO: 9: KRGPTSSNPRKRHREDSDVEMVEDDSRKEMTAACTPRRRIINLTSVLSLQEEINEQGH EVLREMLHNHSFVGCVNPQWALAQHQTKLYLLNTTKLSEELFYQILIYDFANFGVLR LSEPAPLFDLAMLALDSPESGWTEEDGPKEGLAEYIVEFLKKKAEMLADYFSLEIDEE GNLIGLPLLIDNYVPPLEGLPIFILRLATEVNWDEEKECFESLSKECAMFYSIRKQYISE ESTLSGQQSEVPGSIPNSWKWTVEHIVYKALRSHILPPKHFTEDGNILQLANLPDLYK VFERC (SEQ ID NO: 208), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 208.

In various embodiments, the dominant negative MLH1 can include, for example, MLH1 461-753, which is a C-terminal fragment of SEQ ID NO: 9 that corresponds to amino acids 461-753 of SEQ ID NO: 9:

KRGPTSSNPRKRHREDSDVEMVEDDSRKEMTAACTPRRRIINLTSVLSLQEEI NEQGHEVLREMLHNHSFVGCVNPQWALAQHQTKLYLLNTTKLSEELFYQILIYDFA NFGVLRLSEPAPLFDLAMLALDSPESGWTEEDGPKEGLAEYIVEFLKKKAEMLADYF SLEIDEEGNLIGLPLLIDNYVPPLEGLPIFILRLATEVNWDEEKECFESLSKECAMFYSI RKQYISEESTLSGQQSEVPGSIPNSWKWTVEHIVYKALRSHILPPKHFTEDGNILQLAN LPDLYKVF[ - - - ](SEQ ID NO: 209), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 209.

In various other embodiments, the dominant negative MLH1 can include, for example, MLH1 461-753, which is a C-terminal fragment of SEQ ID NO: 9 that corresponds to amino acids 461-753 of SEQ ID NO: 9, and which further comprises an N-terminal NLS, e.g., NLS^{SV40}: [NLS]-KRGPTSSNPRKRHREDSDVEMVEDDSRKEMTAACTPRRRIINLTSVLSLQEEINEQGH EVLREMLHNHSFVGCVNPQWALAQHQTKLYLLNTTKLSEELFYQILIYDFANFGVLR LSEPAPLFDLAMLALDSPESGWTEEDGPKEGLAEYIVEFLKKKAEMLADYFSLEIDEE GNLIGLPLLIDNYVPPLEGLPIFILRLATEVNWDEEKECFESLSKECAMFYSIRKQYISE ESTLSGQQSEVPGSIPNSWKWTVEHIVYKALRSHILPPKHFTEDGNILQLANLPDLYK VF[ - - - ](SEQ ID NO: 209), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 209. The NLS sequence can be any suitable NLS sequence, including but not limited to SEQ ID NOs: 20-31 and 77-81

napDNAbp

As used herein, the term “nucleic acid programmable DNA binding protein” or “napDNAbp,” of which Cas9 is an example, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical Cas9 sequence or to an equivalent amino acid position in other Cas9 variants or Cas9 equivalents. In some embodiments, the nickase is a Cas9 that comprises an H840A, N854A, and/or N863A mutation relative to a canonical Cas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

### Nuclear Export Sequence (NES)

The term “nuclear export sequence” or “NES” refers to an amino acid sequence that promotes transport of a protein out of the cell nucleus to the cytoplasm, for example, through the nuclear pore complex by nuclear transport. Nuclear export sequences are known in the art and would be apparent to the skilled artisan. For example, NES sequences are described in Xu, D. et al. Sequence and structural analyses of nuclear export signals in the NESdb database. Mol Biol. Cell. 2012, 23(18) 3677-3693, the contents of which are incorporated herein by reference.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, an NLS comprises the amino acid sequence PKKKRKV (SEQ ID NO: 30).

### Nucleic Acid

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### PEgRNA

As used herein, the terms “prime editing guide RNA” or “PEgRNA” or “extended guide RNA” refer to a specialized form of a guide RNA that has been modified to include one or more additional sequences for implementing the prime editing methods and compositions described herein. As described herein, the prime editing guide RNAs comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the PEgRNAs have in the 5′ to 3′ direction a spacer (1), a gRNA core (2), and an extension arm (3). The extension arm (3) is at the 3′ end of the PEgRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm (3), a spacer (1), and a gRNA core (2). The extension arm (3) is at the 5′ end of the PEgRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

### PE1

As used herein, “PEl” refers to a PE complex comprising a fusion protein comprising Cas9(H840A) and a wild type MMLV RT having the following structure: [NLS]-[Cas9(H840A)]-[linker]-[MMLV_RT(wt)]+a desired PEgRNA, wherein the PE fusion has the amino acid sequence of SEQ ID NO: 32, which is shown as follows;

(SEQ ID NO: 32)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGTAGFCRLWIPGFAEMAAPLYPLTKTG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGLLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS)
TOP:(SEQ ID NO: 20), BOTTOM: (SEQ ID NO: 29)
CAS9(H840A) (SEQ ID NO: 39)
33-AMINO ACID LINKER (SEQ ID NO: 161)
M-MLV reverse transcriptase. (SEQ ID NO: 59)

### PE2

As used herein, “PE2” refers to a PE complex comprising a fusion protein comprising Cas9(H840A) and a variant MMLV RT having the following structure: [NLS]-[Cas9(H840A)]-[linker]-[MMLV_RT(D200N)(T330P)(L603W)(T306K)(W313F)]+a desired PEgRNA, wherein the PE fusion has the amino acid sequence of SEQ ID NO: 33, which is shown as follows:

(SEQ ID NO: 33)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS)
TOP:(SEQ ID NO: 20), BOTTOM: (SEQ ID NO: 29)
CAS9(H840A) (SEQ ID NO: 39)
33-AMINO ACID LINKER (SEQ ID NO: 161)
M-MLV reverse transcriptase. (SEQ ID NO: 60)

### PE3

As used herein, “PE3” refers to PE2 plus a second-strand nicking guide RNA that complexes with the PE2 and introduces a nick in the non-edited DNA strand in order to induce preferential replacement of the edited strand.

### PE3b

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE4

As used herein, “PE4” refers to a system comprising PE2 plus an MLH1 dominant negative protein (i.e., wild-type MLH1 with amino acids 754-756 truncated, which may be referred to herein as “MLH1 Δ754-756” or “MLH1dn”) expressed in trans. In some embodiments, PE4 refers to a fusion protein comprising PE2 and an MLH1 dominant negative protein joined via an optional linker.

### PE5

As used herein, “PE5” refers to a system comprising PE3 plus an MLH1 dominant negative protein (i.e., wild-type MLH1 with amino acids 754-756 truncated, which may be referred to as “MLH1 Δ754-756” or “MLH1dn”) expressed in trans. In some embodiments, PE5 refers to a fusion protein comprising PE3 and an MLH1 dominant negative protein joined via an optional linker.

### PEmax

As used herein, “PEmax” refers to a PE complex comprising a fusion protein comprising Cas9(R221K N39K H840A) and a variant MMLV RT pentamutant (D200N T306K W313F T330P L603W) having the following structure: [bipartite NLS]-[Cas9(R221K)(N394K)(H840A)]-[linker]-[MMLV_RT(D200N)(T330P)(L603W)]-[bipartite NLS]-[NLS]+a desired PEgRNA, wherein the PE fusion has the amino acid sequence of SEQ ID NO: 34, which is shown as follows:

(SEQ ID NO: 34)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRKLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLKREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSKRTADG
SEFESPKKKRKVSGGSSGGSTLNIEDEYRLHETSKEPDVSLGSTWLSDFP
QAWAETGGMGLAVRQAPLIIPLKATSTPVSIKQYPMSQEARLGIKPHIQR
LLDQGILVPCQSPWNTPLLPVKKPGTNDYRPVQDLREVNKRVEDIHPTVP
NPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAA
TSELDCQQGTRALLQTLGNLGYRASAKKAQICQKQVKYLGYLLKEGQRWL
TEARKETVMGQPTPKTPRQLREFLGKAGFCRLFIPGFAEMAAPLYPLTKP
GTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQ
PLVILAPHAVEALVKQPPDRWLSNARMTHYQALLLDTDRVQFGPVVALNP
ATLLPLPEEGLQHNCLDILAEAHGTRPDLTDQPLPDADHTWYTDGSSLLQ
EGQRKAGAAVTTETEVIWAKALPAGTSAQRAELIALTQALKMAEGKKLNV
YTDSRYAFATAHIHGEIYRRRGWLTSEGKEIKNKDEILALLKALFLPKRL
SIIHCPGHQKGHSAEARGNRMADQAARKAAITETPDTSTLLIENSSPSGG
SKRTADGSEFESPKKKRKVGSGPAAKRVKLD
KEY:
BIPARTITE SV40 NUCLEAR LOCALIZATION SEQUENCE (NLS)
TOP: (SEQ ID NO: 20),
CAS9(R221K N39K H840A) (SEQ ID NO: 40)
SGGSx2-BIPARTITE SV40NLS-SGGSx2 LINKER (SEQ ID NO:
160)
M-MLV reverse transcriptase(D200N T306K W313F
T330P L603W) (SEQ ID NO: 60)
Other linker sequence (SEQ ID NO: 162)
BIPARTITE SV40NLS (SEQ ID NO: 31)
Other linker sequence
c-Myc NLS PAAKRVKLD (SEQ ID NO: 24)

### PE4max

As used herein, “PE4max” refers to PE4 but wherein the PE2 component is substituted with PEmax.

### PE5max

As used herein, “PE5max” refers to PE5 but wherein the PE2 component of PE3 is substituted with PEmax.

### Polymerase

As used herein, the term “polymerase” refers to an enzyme that synthesizes a nucleotide strand and that may be used in connection with the prime editor delivery systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotides (i.e., the polymerase activity). Generally, the enzyme will initiate synthesis at the 3′-end of a primer annealed to a polynucleotide template sequence (e.g., such as a primer sequence annealed to the primer binding site of a PEgRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to an approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Prime editing is described in Anzalone, A. V. et al. Search-and-replace genome editing without double-strand breaks or donor DNA. Nature 576, 149-157 (2019), which is incorporated herein by reference in its entirety.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., PEgRNA) containing a spacer sequence that anneals to a complementary protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule, and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In step (d), a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) describe herein, including, but not limited to, the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template, the PEgRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

### Prime Editor

The term “prime editor” refers to fusion constructs comprising a napDNAbp (e.g., Cas9 nickase) and a reverse transcriptase and is capable of carrying out prime editing on a target nucleotide sequence in the presence of a PEgRNA (or “extended guide RNA”). The term “prime editor” may refer to the fusion protein or to the fusion protein complexed with a PEgRNA, and/or further complexed with a second-strand nicking sgRNA. In some embodiments, the prime editor may also refer to the complex comprising a fusion protein (reverse transcriptase fused to a napDNAbp), a PEgRNA, and a regular guide RNA capable of directing the second-site nicking step of the non-edited strand as described herein.

### Primer Binding Site

The term “primer binding site” or “the PBS” refers to the nucleotide sequence located on a PEgRNA as a component of the extension arm (typically at the 3′ end of the extension arm) and serves to bind to the primer sequence that is formed after Cas9 nicking of the target sequence by the prime editor. As detailed elsewhere, when the Cas9 nickase component of a prime editor nicks one strand of the target DNA sequence, a 3′-ended ssDNA flap is formed, which serves a primer sequence that anneals to the primer binding site on the PEgRNA to prime reverse transcription.

### Protease Cleavage Site

The term “protease cleavage site,” as used herein, refers to an amino acid sequence that is recognized and cleaved by a protease, i.e., an enzyme that catalyzes proteolysis and breaks down proteins into smaller polypeptides, or single amino acids. In some embodiments, a protease cleavage site is included in a cleavable linker in a fusion protein, as described herein. In certain embodiments, a protease cleavage site is cleaved by the protease of a gag-pro polyprotein. In some embodiments, a protease cleavage site comprises an MMLV protease cleavage site or an FMLV protease cleavage site. In certain embodiments, a protease cleavage site comprises one of the amino acid sequences TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′, wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes an alternative PAM sequence.

For example, with reference to the canonical SpCas9 amino acid sequence SEQ ID NO: 37, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference is made to Shah et al., “Protospacer recognition motifs: mixed identities and functional diversity,” RNA Biology, 10(5): 891-899 (which is incorporated herein by reference).

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides that are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes. The disclosure provides in some embodiments prime editor fusion proteins comprising MMLV RT.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes that are error-prone in their DNA polymerization activity.

### Spacer Sequence

As used herein, the term “spacer sequence” in connection with a guide RNA or a PEgRNA refers to the portion of the guide RNA or PEgRNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Variant

As used herein, the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, at least 80%, at least 85%, at least 90%, at least 95%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Viral Envelope Glycoprotein

The term “viral envelope glycoprotein” refers to oligosaccharide-containing proteins that form a part of the viral envelope, i.e., the outermost layer of many types of viruses that protects the viral genetic materials when traveling between host cells. Glycoproteins may assist with identification and binding to receptors on a target cell membrane so that the viral envelope fuses with the membrane, allowing the contents of the viral particle (which may comprise, e.g., a PE-VLP as described herein) to enter the host cell. The viral envelope glycoproteins used in the PE-VLPs of the present disclosure may comprise any glycoprotein from an enveloped virus. In some embodiments, a viral envelope glycoprotein is an adenoviral envelope glycoprotein, an adeno-associated viral envelope glycoprotein, a retroviral envelope glycoprotein, or a lentiviral envelope glycoprotein. In certain embodiments, a viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein.

### Virus-Like Particles (VLPs)

As used herein, a virus-like particle consists of a supra-molecular assembly comprising (a) an envelope comprising (i) a lipid membrane (e.g., single-layer or bi-layer membrane) and a (ii) viral envelope glycoprotein and (b) a multi-protein core region comprising (ii) a Gag protein, (ii) a first fusion protein comprising a Gag protein and Pro-Pol, and (iii) a second fusion protein comprising a Gag protein fused to a cargo protein via a protease-cleavable linker. In various embodiments, the cargo protein is a prime editor. In various other embodiments, the multi-protein core region of the VLPs further comprises one or more guide RNA and/or pegRNA molecules which are complexed with the prime editor to form a ribonucleoprotein (RNP). In various embodiments, the VLPs are prepared in a producer cell that is transiently transformed with plasmid DNA that encodes that various protein and nucleic acid (sgRNA) components of the VLPs. The components self-assemble at the cell membrane and bud out in accordance with the naturally occurring mechanism of retroviral budding in order to release from the cell fully-matured VLPs. Once formed, the Pol-Pro cleaves the protease-sensitive linker joining the Gag-cargo linker (e.g., the linker joining a Gag to a PE RNP or a napDNAbp RNP) to release the PE RNP and/or napDNAbp RNA as the case may be within the VLP. Thus, in various embodiments, the present disclosure also provides VLPs in which the prime editor has been cleaved off of the gag protein and released within the VLP. For example, the present disclosure provides VLPs comprising (i) a group-specific antigen (gag) protease (pro) polyprotein, (ii) a prime editor, and (iii) a fusion protein comprising a gag nucleocapsid protein and a nuclear export sequence (NES), encapsulated by a lipid membrane and a viral envelope glycoprotein. In some embodiments, the present disclosure provides VLPs comprising a mixture of cleaved and uncleaved products (i.e., a mixture of prime editors that have been cleaved from the gag protein and that have not yet been cleaved from the gag protein). Once the VLP is administered to a recipient cell and take up by said cell, the contents of the VLP are released, including free PE RNP and/or napDNAbp RNA. Once in the cell, the RNPs may translocate to the nuclease of the cell (in particular, where NLSs are included on the RNPs), where DNA editing may occur at target sites specified by the guide RNA.

In some embodiments, a VLP comprises additional agents for targeting the VLP for delivery to particular cell types. For example, such additional targeting agents may be incorporated into the outer lipid membrane encapsulation layer of the VLP. In some embodiments, the additional targeting agent is a protein. In certain embodiments, the additional targeting agent is an antibody.

Thus, as used herein, a virus-derived particle comprises a virus-like particle formed by one or more virus-derived protein(s), which virus-derived particle is substantially devoid of a viral genome such that the VLP is replication-incompetent when delivered to a recipient cell.

### Wild Type

As used herein the term “wild type” is a term of the art understood by skilled persons and means the typical form of an organism, strain, gene, or characteristic as it occurs in nature as distinguished from mutant or variant forms.

### DETAILED DESCRIPTION

The present disclosure is based on the development and application of an engineered VLP platform for packaging and delivering prime editor ribonucleoproteins in vitro and in vivo, referred to herein as prime editor virus-like proteins (PE-VLPs). These optimized PE-VLPs enable efficient prime editing in a variety of cell types. In particular, the PE-VLPs described herein are based on the surprising discovery that both nuclear-export sequences (NES) and nuclear localization sequences (NLS) may be included on the same fusion protein to promote trafficking of the fusion protein to different parts of a cell during production and during delivery. The presently described PE-VLPs are produced in viral producer cells and exported from the nucleus due to the presence of one or more NES sequences in the fusion proteins inside the PE-VLPs. Following delivery to a target cell, the NES is cleaved from the fusion protein when the prime editor is released from the VLP, allowing the PE (which may comprise one or more NLS sequences) to enter the nucleus of a target cell and edit the genome. The PE-VLPs described herein also include a protease cleavage site which separates the NES and VLP proteins from the rest of the prime editor to promote highly efficient cleavage and delivery of the PE. Finally, the present disclosure also describes the optimization of the ratios of various components of the PE-VLPs, ensuring high efficiency of PE-VLP production.

Accordingly, the present disclosure provides virus-like particles for delivering prime editor fusion proteins (PE-VLPs) and systems comprising such PE-VLPs. The present disclosure also provides polynucleotides encoding the PE-VLPs described herein, which may be useful for producing said VLPs. Also provided herein are methods for editing the genome of a target cell by introducing the presently described PE-VLPs into the target cell. The present disclosure also provides fusion proteins that make up a component of the PE-VLPs described herein, as well as polynucleotides, vectors, cells, and kits.

eVLPs

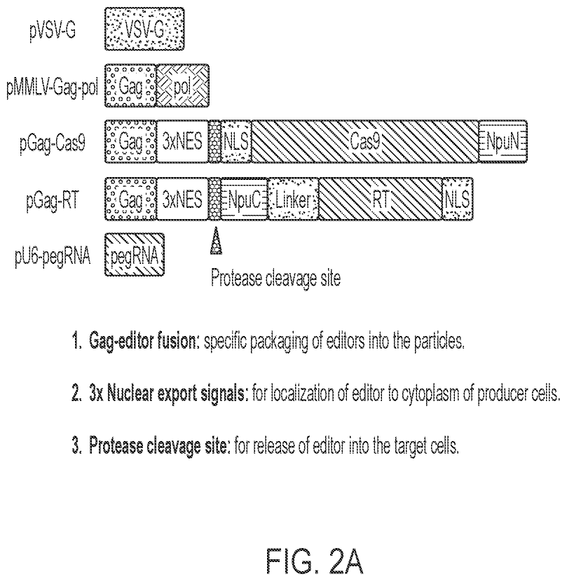

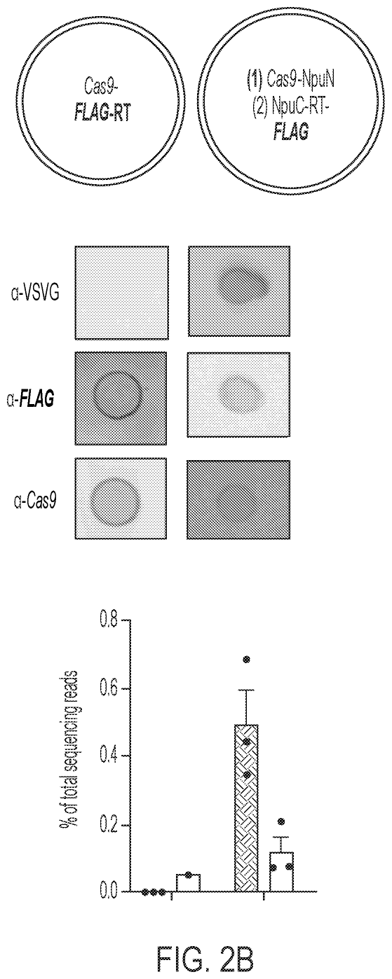

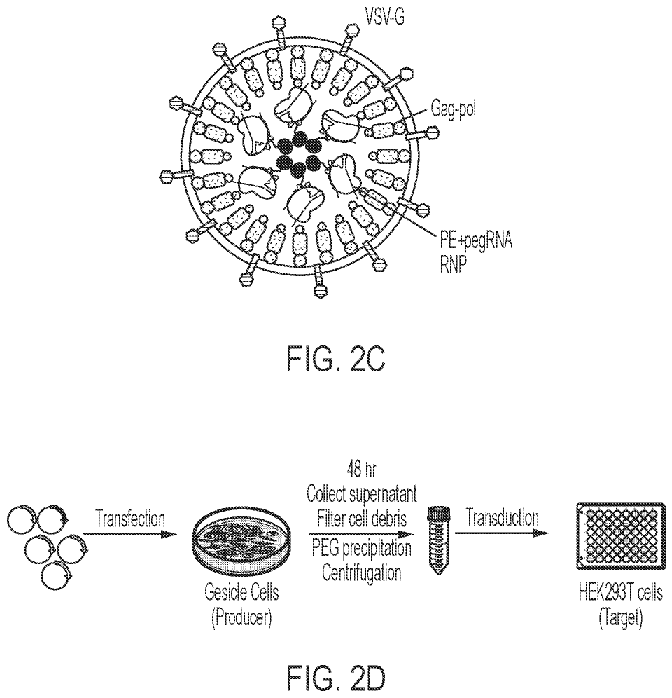

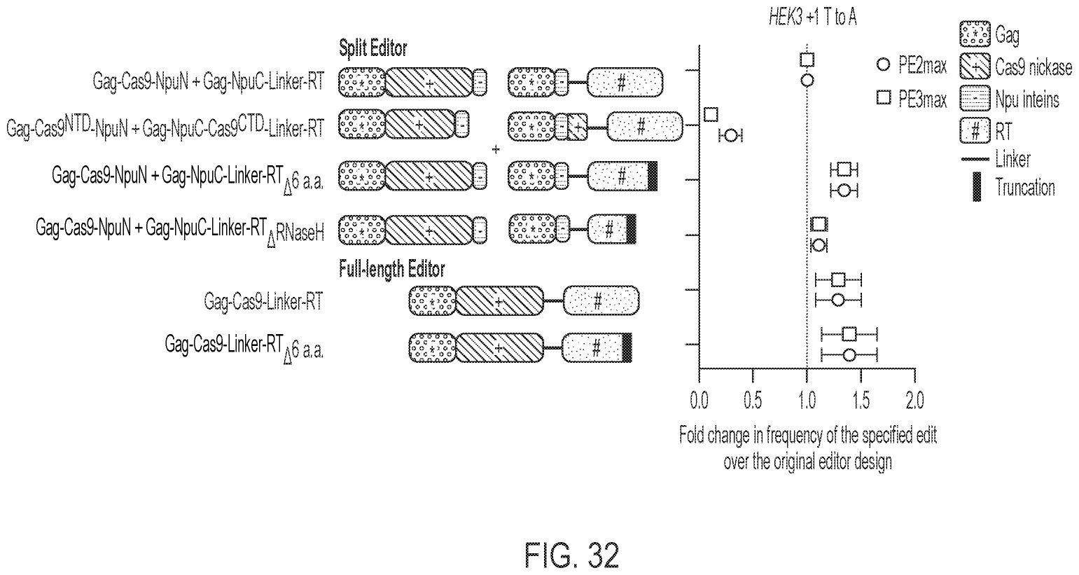

In various embodiments, the eVLPs (e.g., PE-VLPs) comprise a supra-molecular assembly comprising (a) an envelope comprising (i) a lipid membrane (e.g., single-layer or bi-layer membrane) and a (ii) viral envelope glycoprotein (e.g., VSV-G) and (b) a multi-protein core region enclosed by the envelope and comprising (i) a Gag protein, (ii) a Gag-Pro-Pol protein (with the “Pro” component referring to a protease), and (iii) one or more Gag-cargo fusion proteins each comprising a Gag protein fused to a cargo protein (e.g., a napDNAbp or PE or a split PE) via a cleavable linker (e.g., a protease-cleavable linker, e.g., an MMLV protease-cleavable linker). In various embodiments, the cargo protein is a napDNAbp (e.g., Cas9). In other embodiments, the cargo protein is a prime editor. In various embodiments (e.g., FIG. 2A, FIG. 32) the PE may be split into a Cas9 domain and a reverse transcriptase domain as separate fusion proteins each with Gag. In various embodiments, the split domains of PE may comprise split-intein sequences which allows the split domains to re-form a PE once delivered to a cell. In various other embodiments, the multi-protein core region of the VLPs further comprises one or more pegRNA molecules and/or second-site nicking guide RNA which are complexed with the napDNAbp or the prime editor to form a ribonucleoprotein (RNP). In some embodiments, the pegRNAs comprise one or more silent mutations to increase editing efficiency by facilitating evasion of the DNA mismatch repair (MMR) pathway.

In various embodiments, the VLPs are prepared in a producer cell that is transiently transformed with plasmid DNA that encodes the various protein and nucleic acid (pegRNAs and guide RNAs) components of the VLPs. Without being bound by theory, the components self-assemble at the cell membrane and bud out in accordance with the naturally occurring mechanism of budding (e.g., retroviral budding or the budding mechanism of other envelope viruses) in order to release from the cell fully-matured VLPs. Once formed, the Gag-Pol-Pro cleaves the protease-sensitive linker of the Gag-cargo (i.e., [Gag]-[cleavable linker]-[cargo], wherein the cargo can be PE-RNP or a napDNAbp RNP) thereby releasing the PE RNP and/or napDNAbp RNA, as the case may be, within the VLP. Once the VLP is administered to a recipient cell and taken up by said recipient cell, the contents of the VLP are released, e.g., released PE RNP and/or napDNAbp RNP. Once in the cell, the RNPs may translocate to the nuclease of the cell (in particular, where NLSs are included on the RNPs), where DNA editing may occur at target sites specified by the guide RNA. Various embodiments comprise one or more improvements.

In some embodiments, the reverse transcriptase of the prime editors (e.g., full-length prime editors, or split prime editors) delivered by the VLPs disclosed herein is an MMLV reverse transcriptase comprising a C-terminal amino acid truncation to remove the endogenous MMLV protease cleavage site. In some embodiments, the C-terminal amino acid truncation is about 1-180, about 1-170, about 1-160, about 1-150, about 1-140, about 1-130, about 1-120, about 1-110, about 1-100, about 1-90, about 1-80, about 1-70, about 1-60, about 1-50, about 1-40, about 1-30, about 1-20, or about 1-10 amino acids in length. In some embodiments, the C-terminal amino acid truncation is about 1-10 amino acids in length (e.g., about 1, about 2, about 3, about 4, about 5, about 6, about 7, about 8, about 9, or about 10 amino acids in length). In certain embodiments, the C-terminal amino acid truncation is about six amino acids in length. In certain embodiments, the C-terminal amino acid truncation is six amino acids in length.

In one embodiment, the protease-cleavable linker is optimized to improve cleavage efficiency after VLP maturation, as demonstrated herein for v.2 VLPs (or “second generation” VLPs). In some embodiments, one or more additional linkers are inserted N′ and/or C′ to the cleavable linker within the fusion protein(s). Such additional linkers may be useful for better exposing the protease-cleavable linker such that it can be cleaved by a protease at higher rates, thus facilitating release of the cargo protein.

In another embodiment, the Gag-cargo fusion (e.g., Gag-PE) further comprises one or more nuclear export signals at one or more locations along the length of the fusion polypeptide protein which may be joined by a cleavable linker such that during VLP assembly in the producer cell, the Gag-cargo fusions (due to presence of competing NLS signals) do not accumulate in the nucleus of the producer cells but instead are available in the cytoplasm to undergo the VLP assembly process at the cell membrane. Once inside the matured VLPs following release from the producer cell, the NES may be cleaved by Gag-Pro-Pol thereby separating the cargo (e.g., napDNAbp or a PE) from the NES. Upon delivery to a recipient cell, therefore, the cargo (e.g., napDNAbp or PE, typically flanked with one or more NLS elements) will not comprise an NES element, which may otherwise prohibit the transport of the cargo into the nuclease and hinder gene editing activity. This is exemplified as v.3 VLPs described herein (or “third generation” VLPs). In some embodiments, the NES is inserted within the gag nucleocapsid protein portion of the fusion protein. The gag nucleocapsid protein contains multiple endogenous protease sites, and inserting the NES within the gag nucleocapsid protein (rather than, e.g., at one end of the gag nucleocapsid protein) may help ensure that the NES is cleaved from the cargo protein once it has been delivered in the VLP. In certain embodiments, the NES is inserted between the p12 and CA domains of the gag nucleocapsid protein. In certain embodiments, the NES is inserted within the p12 domain of the gag nucleocapsid protein. In certain embodiments, the NES is inserted between the p12 and MA domains of the gag nucleocapsid protein.

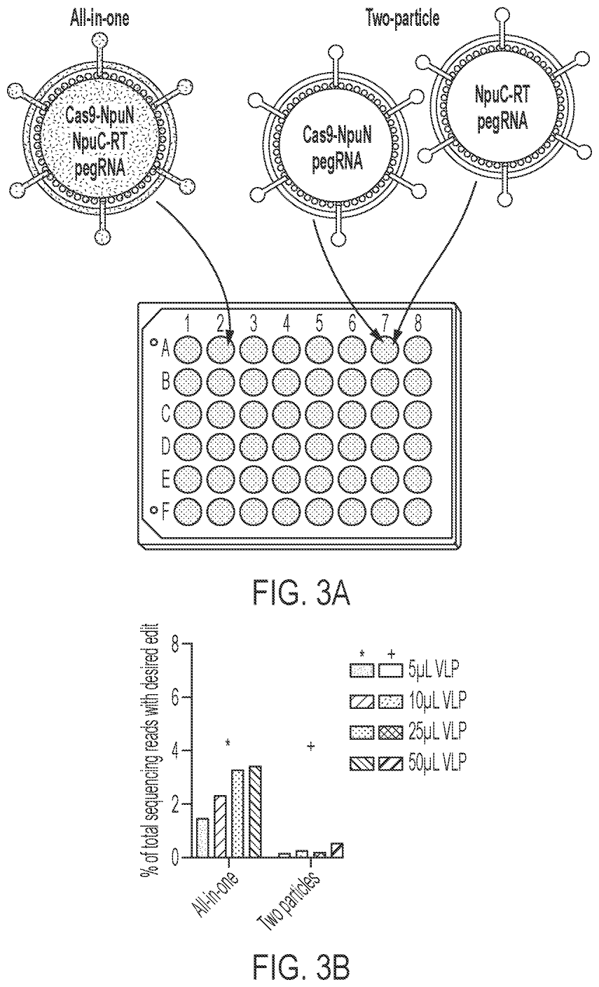

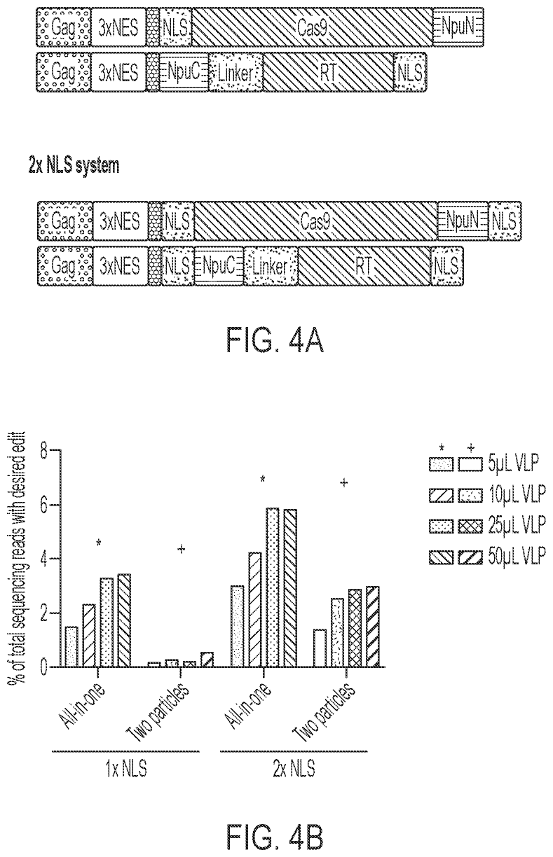

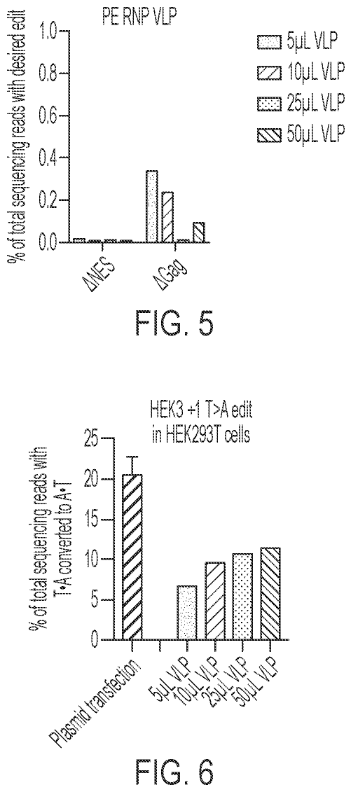

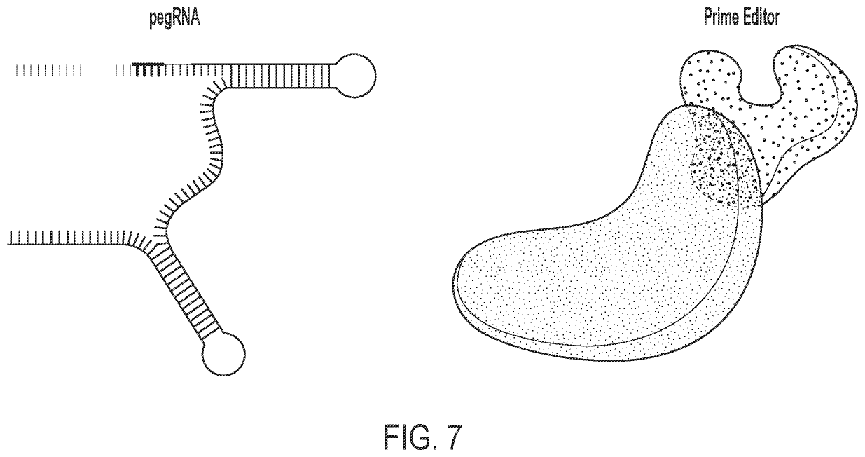

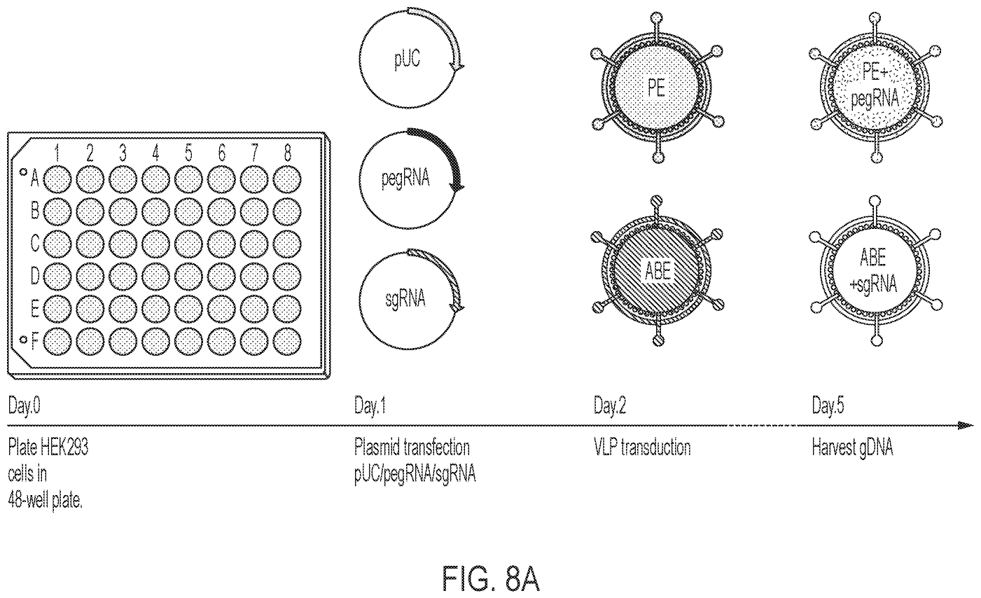

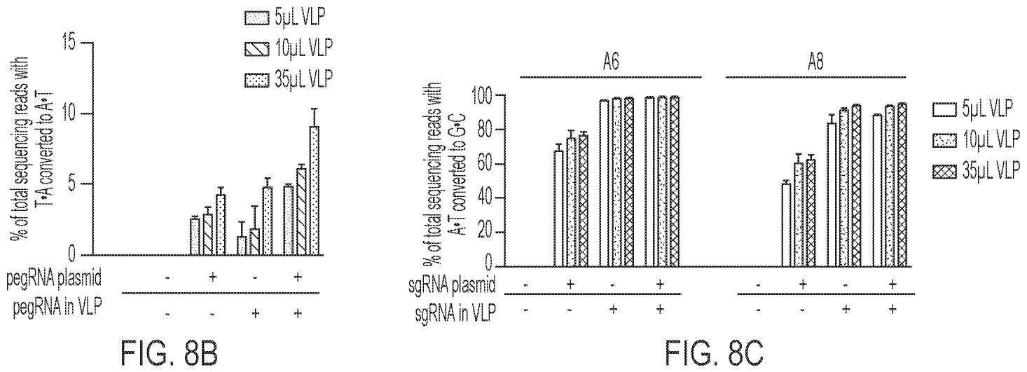

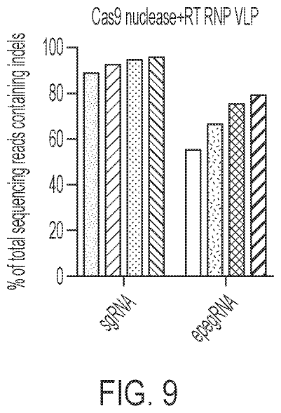

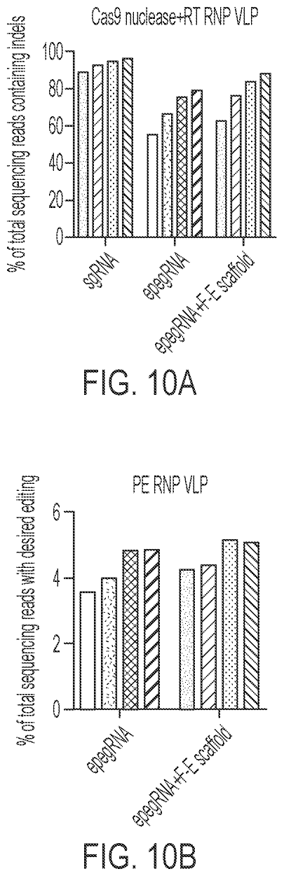

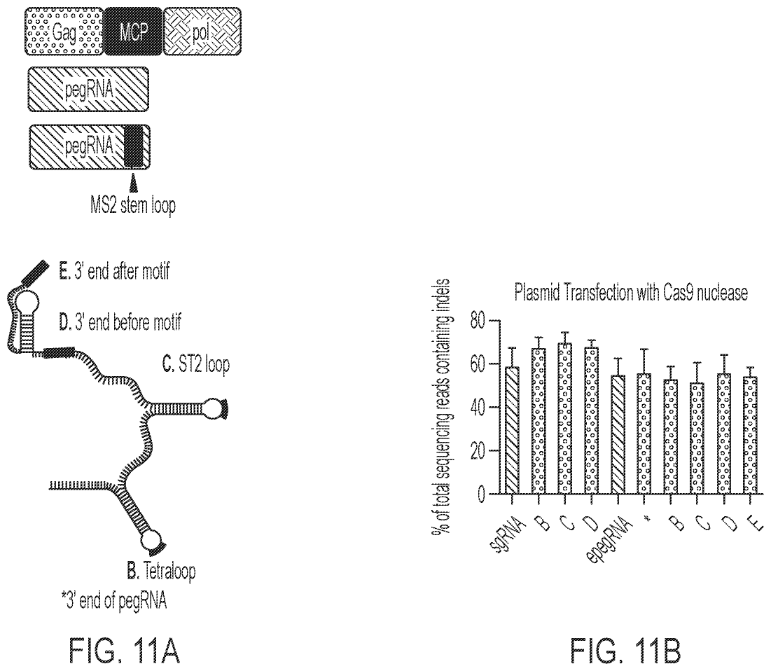

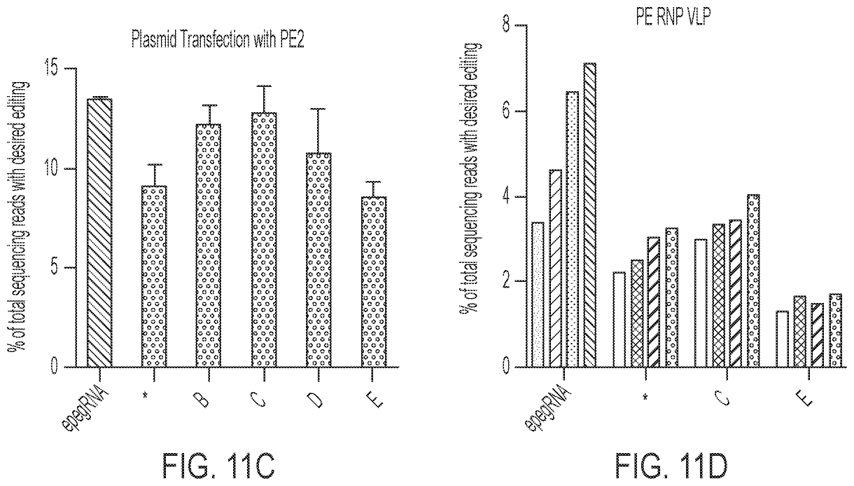

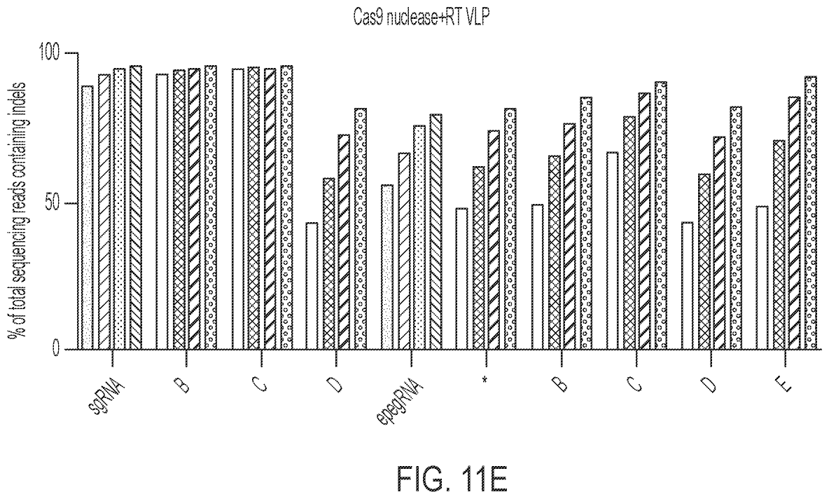

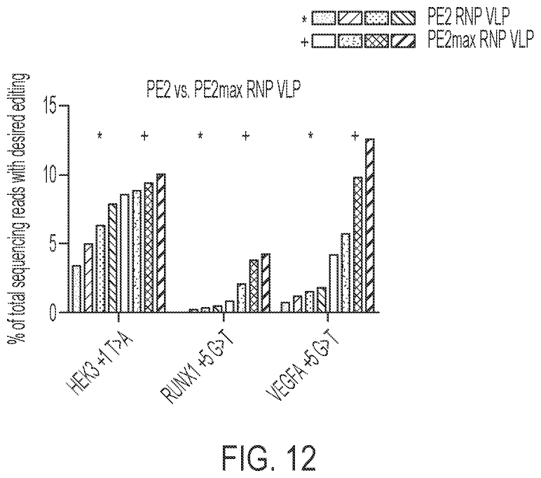

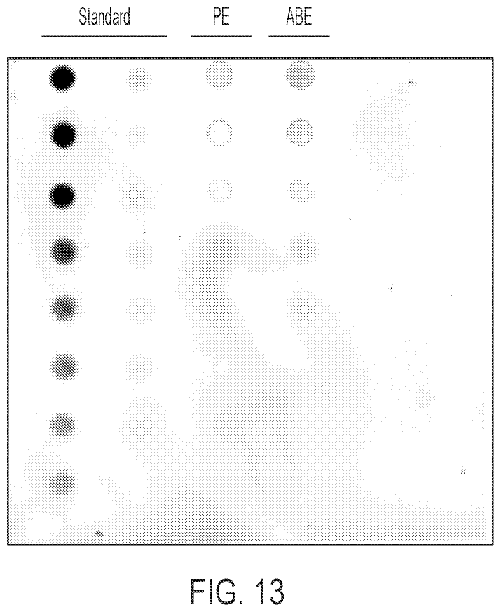

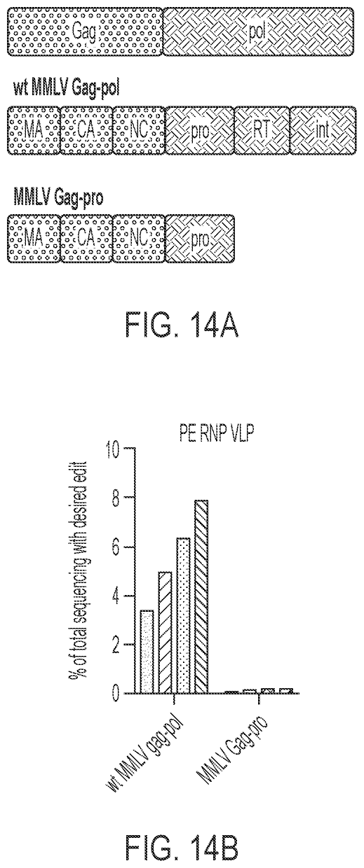

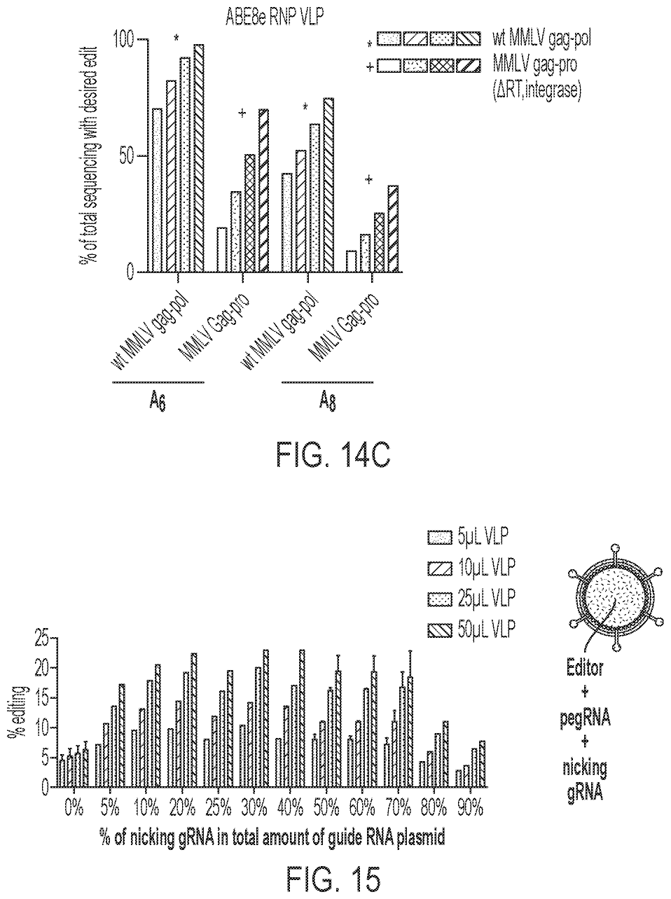

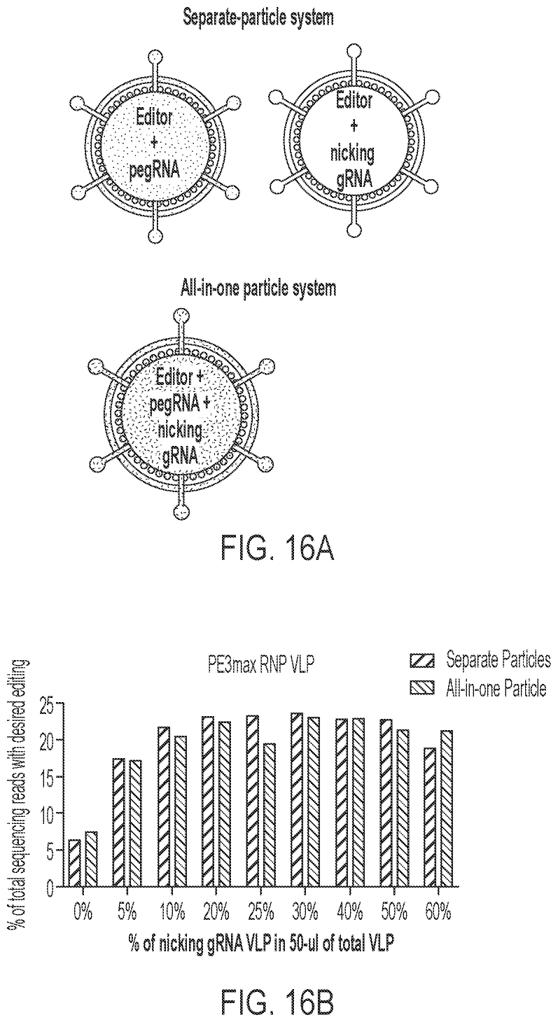

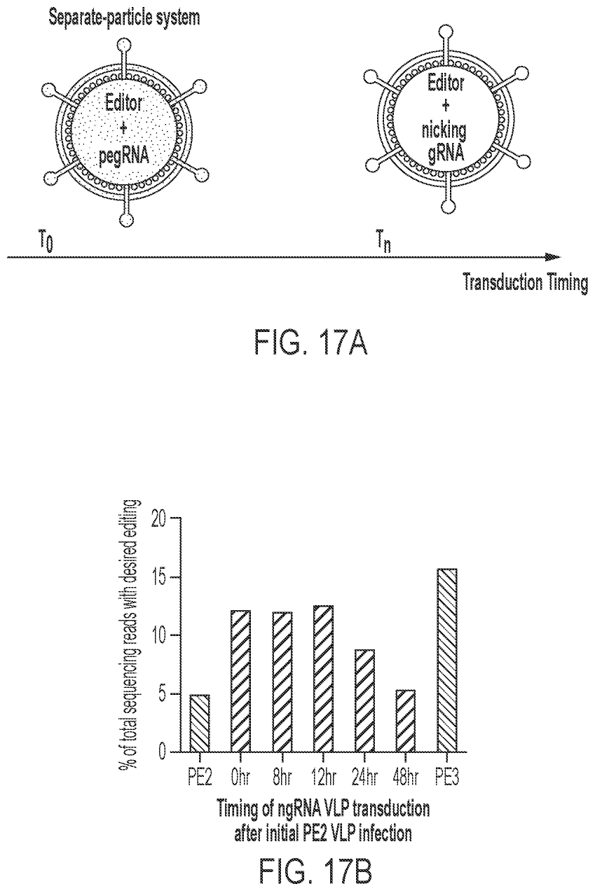

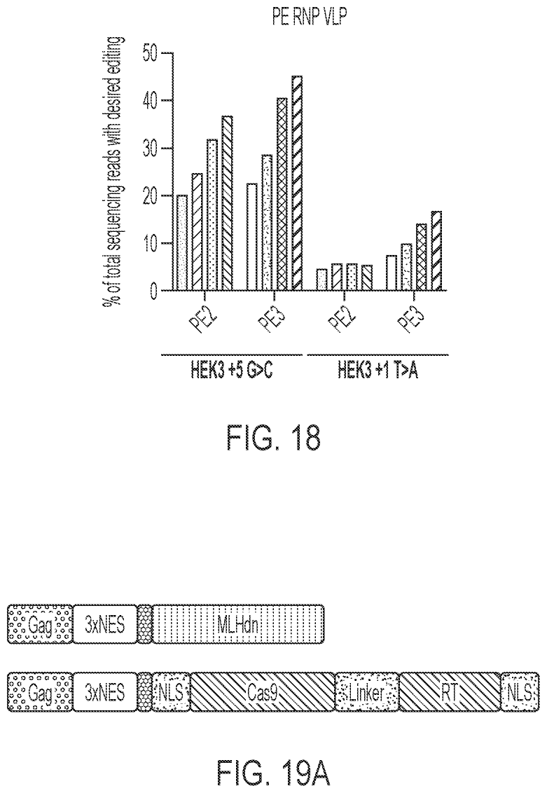

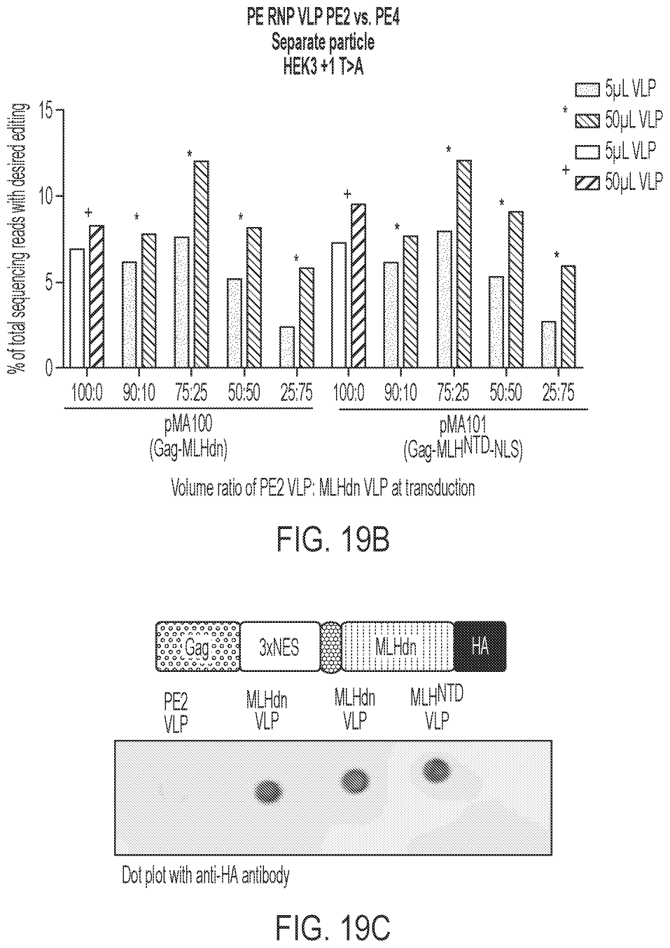

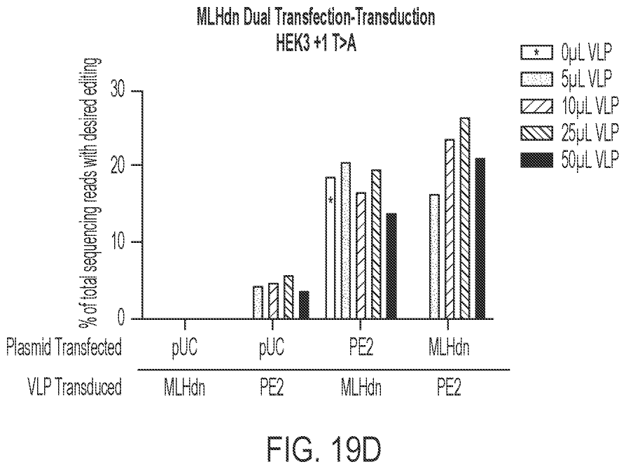

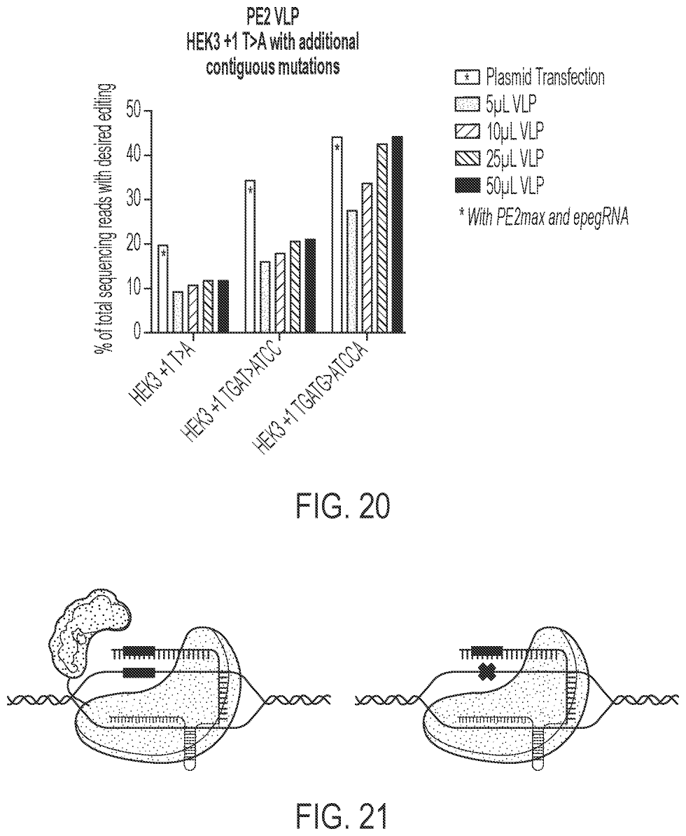

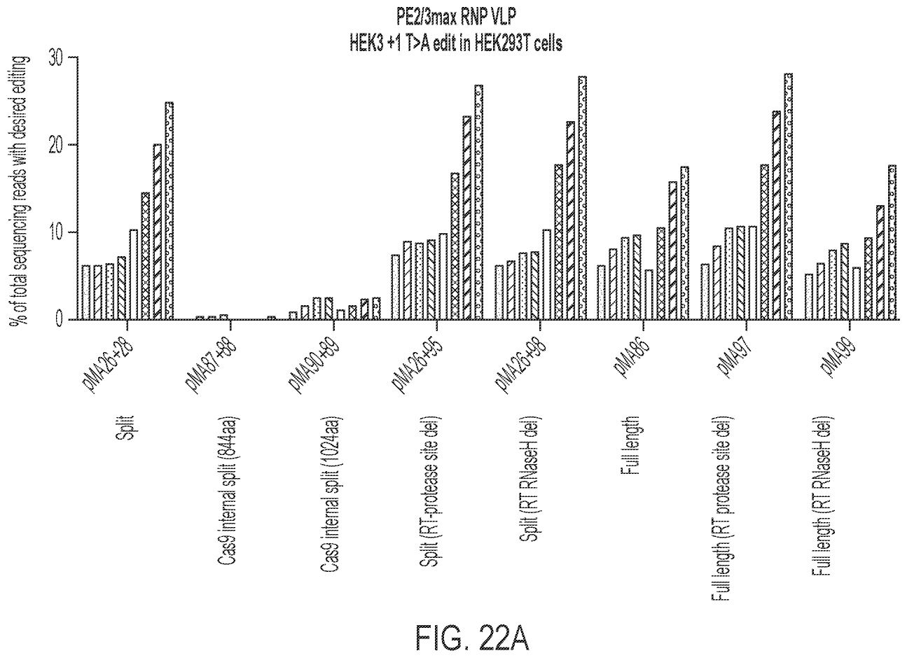

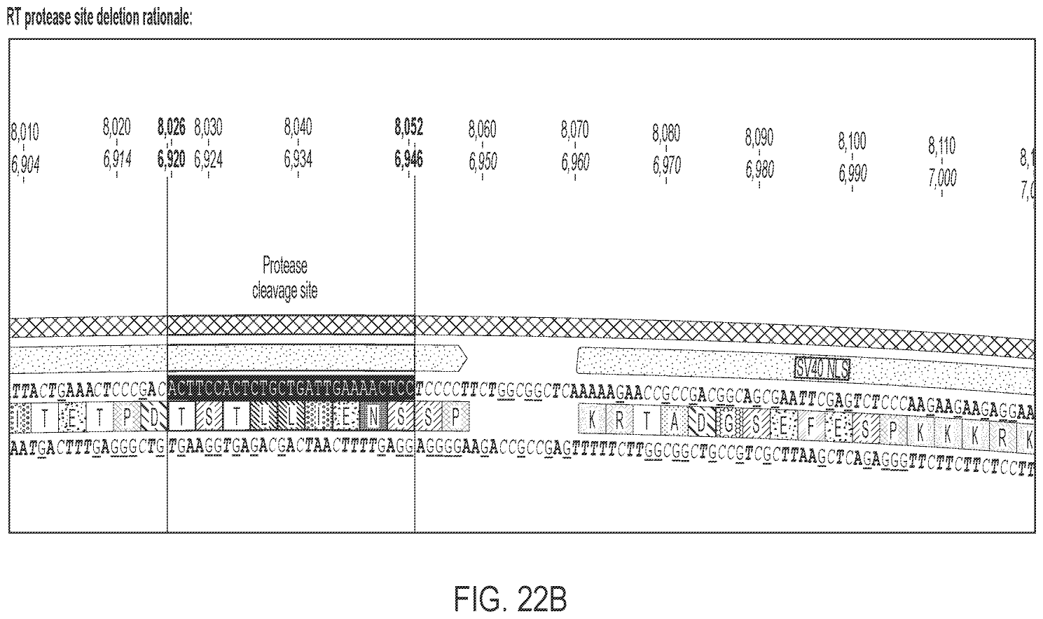

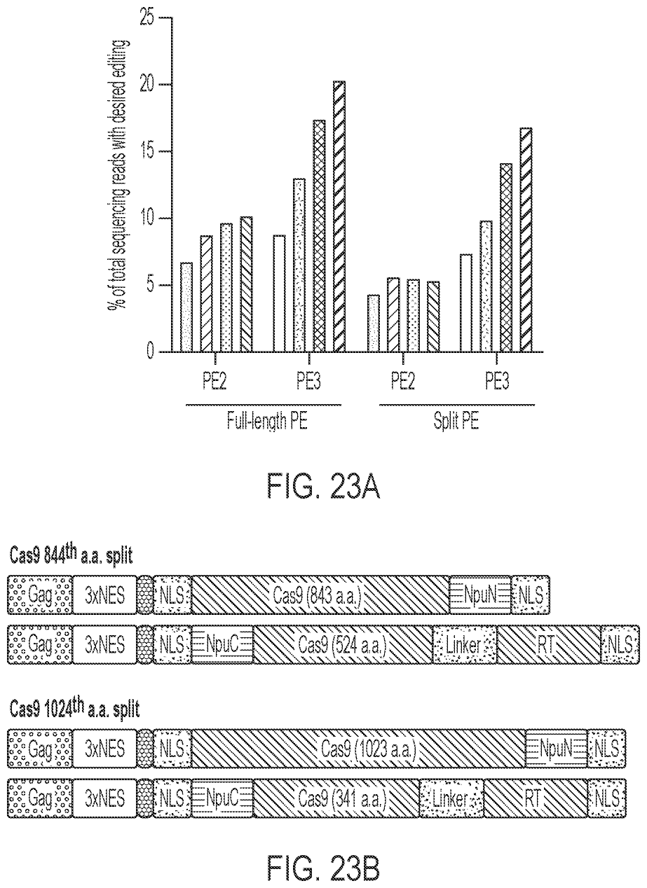

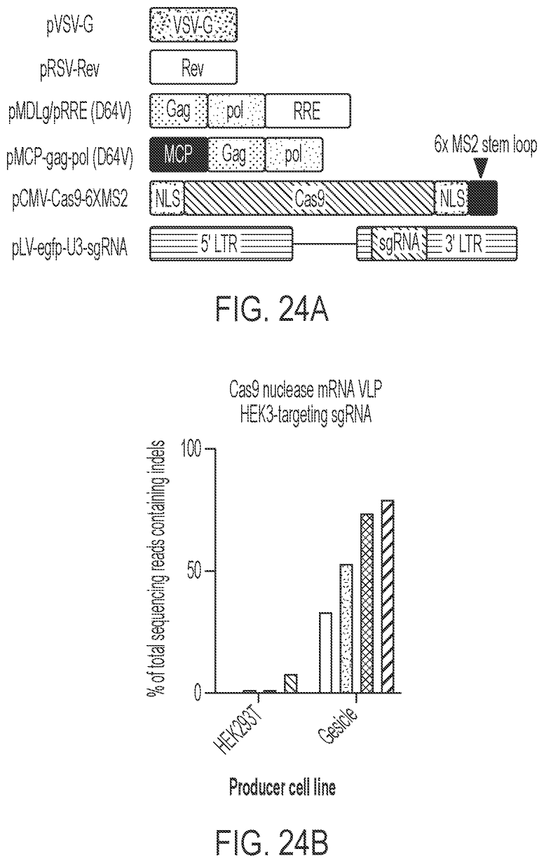

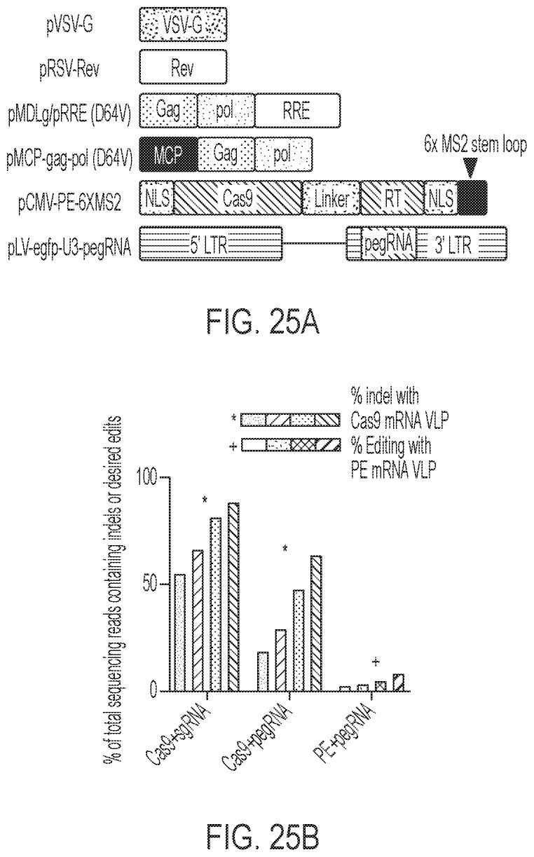

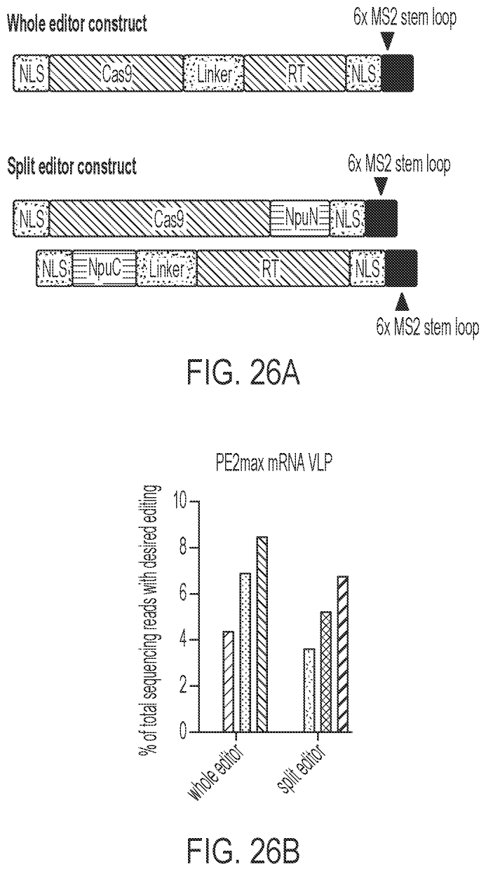

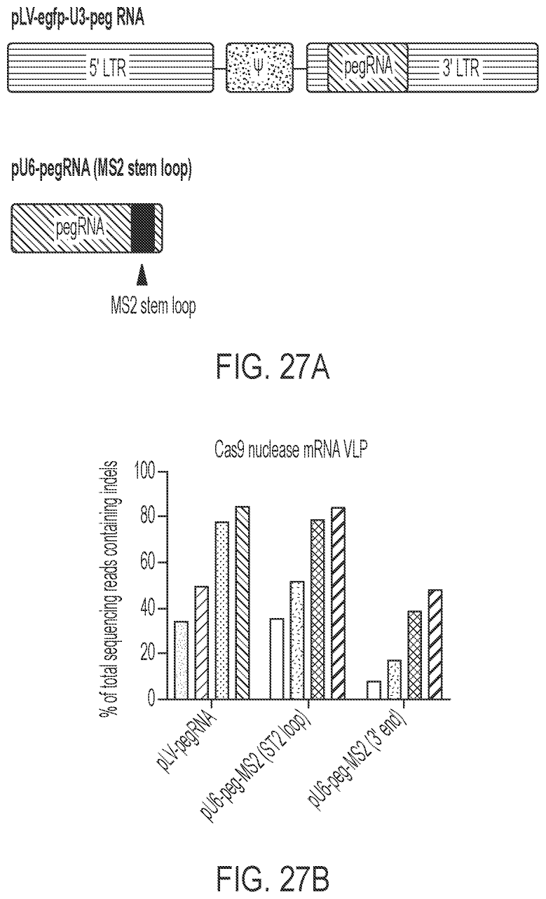

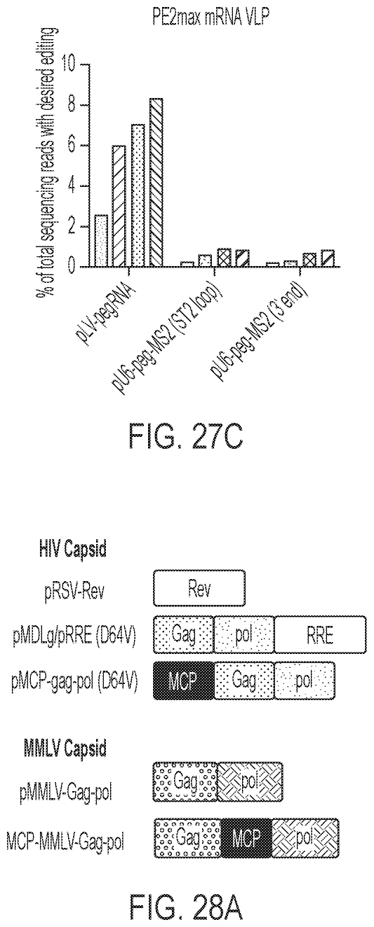

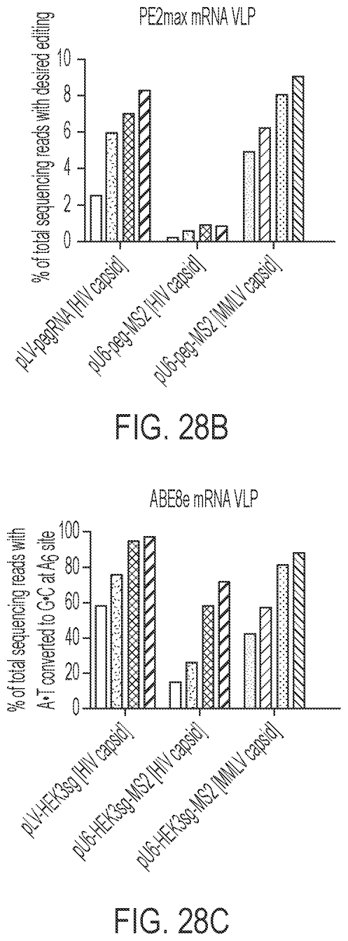

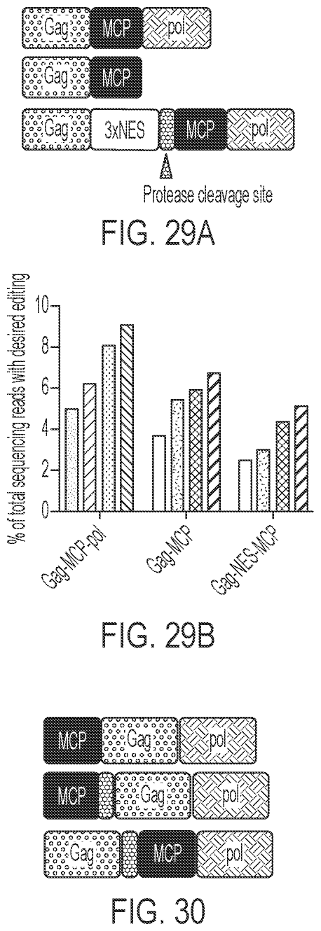

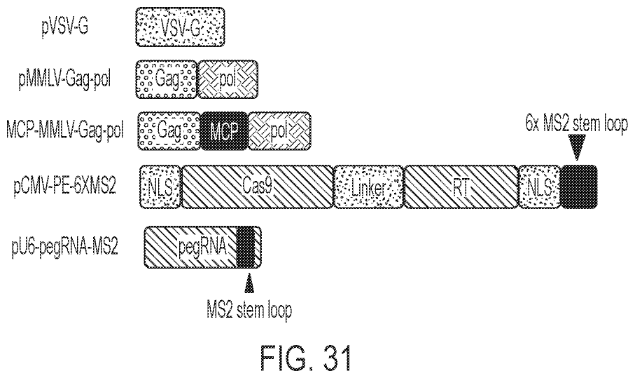

In other embodiments, the eVLPs disclosed herein may comprise split PE domains contained in a single all-in-one VLP system or in a two-particle system whereby each PE half domain is formed in separate VLPs. See FIG. 3A and FIG. 32.

In one aspect, the present disclosure provides a eVLP comprising an (a) envelope and (b) a multi-protein core, wherein the envelope comprises a lipid membrane (e.g., a lipid mono or bi-layer membrane) and a viral envelope glycoprotein and wherein the multi-protein core comprises a Gag (e.g., a retroviral Gag), a group-specific antigen (gag) protease (pro) polyprotein (i.e., “Gag-Pro-Pol”) and one or more fusion proteins comprising a Gag-cargo (e.g., Gag-napDNAbp, Gag-reverse transcriptase, or Gag-PE). In various embodiments, the Gag-cargo may comprise a ribonucleoprotein cargo, e.g., a napDNAbp, a reverse transcriptase, or a PE complexed with a guide RNA. In still further embodiments, the Gag-cargo (e.g., Gag fused to a napDNAbp, a reverse transcriptase, or a PE) may comprise one or more NLS sequences and/or one or more NES sequences to regulate the cellular location of the cargo in a cell. An NLS sequence will facilitate the transport of the cargo into the cell's nuclease to facilitate editing. A NES will do the opposite, i.e., transport the cargo out from the nucleus, and/or prevent the transport of the cargo into the nucleus. In certain embodiments, the NES may be coupled to the fusion protein by a cleavable linker (e.g., a protease linker) such that during assembly in a producer cell, the NES signals operates to keep the cargo in the cytoplasm and available for the packaging process. However, once matured VLPs are budded out or released from a producer cell in a mature form, the cleavable linker joining the NES may be cleaved, thereby removing the association of NES with the cargo. Thus, without an NES, the cargo will translocate to the nuclease with its NLS sequences, thereby facilitating editing. Various napDNAbps may be used in the systems of the present disclosure. In some embodiments, the napDNAbp is a Cas9 protein (e.g., a Cas9 nickase, dead Cas9 (dCas9), or another Cas9 variant as described herein). In some embodiments, the Cas9 protein is bound to a guide RNA (gRNA). The fusion protein may further comprise other protein domains, such as effector domains. In some embodiments, the fusion protein further comprises a deaminase domain (e.g., an adenosine deaminase domain or a cytosine deaminase domain). In certain embodiments, the fusion protein comprises a prime editor, such as PE2, PE3, or PEmax prime editor, or any of the other prime editors described herein or known in the art.

In some embodiments, the fusion protein comprises more than one NES (e.g., two NES, three NES, four NES, five NES, six NES, seven NES, eight NES, nine NES, or ten or more NES). In certain embodiments, the fusion protein further comprises a nuclear localization sequence (NLS), or more than one NLS (e.g., two NLS, three NLS, four NLS, five NLS, six NLS, seven NLS, eight NLS, nine NLS, or ten or more NLS). In certain embodiments, the fusion protein may comprise at least one NES and one NLS.

The Gag-cargo fusion proteins described herein comprise one or more cleavable linkers. In one embodiment, the Gag-cargo fusion proteins comprise a cleavable linker joining the Gag to the cargo, such that once the Gag-cargo fusion has been packaged in mature VLPs (which will also contain the Gag-Pro-Pol, the protease activity can cleave the Gag-cargo cleavable linker, thereby releasing the cargo. In some embodiments, a cleavable linker may also be provided in such a location such that when the cleavable linker is cleaved (e.g., by the Gag-Pro-Pol protein), the NES is separated away from the cargo protein. Such an arrangement of the fusion protein allows the fusion protein to be exported from the nucleus of a producing cell during PE-VLP production, and the NES can later be cleaved from the fusion protein after delivery to a target cell, or prior to delivery to the target cell but after packaging into the VLP, releasing the PE (or release of split PE half domains from the same or a two-particle system) and allowing it to enter the nucleus of the target cell. In some embodiments, the cleavable linker comprises a protease cleavage site (e.g., a Moloney murine leukemia virus (MMLV) protease cleavage site or a Friend murine leukemia virus (FMLV) protease cleavage site). Various protease cleavage sites can be used in the fusion proteins of the present disclosure. In certain embodiments, the protease cleavage site comprises the amino acid sequence TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8. In some embodiments, the protease cleavage site comprises the amino acid sequence of any one of SEQ ID NOs: 5-8 comprising one mutation, two mutations, three mutations, four mutations, five mutations, or more than five mutations relative to one of SEQ ID NOs: 5-8. In some embodiments, the cleavable linker of the fusion protein is cleaved by the protease of the gag-pro polyprotein. In certain embodiments, the cleavable linker of the fusion protein is not cleaved by the protease of the gag-pro polyprotein until the PE-VLP has been assembled and delivered into a target cell.

In some embodiments, one or more additional linkers are inserted N′ and/or C′ to the cleavable linker within the fusion protein(s). Such additional linkers may be useful for better exposing the protease-cleavable linker such that it can be cleaved by a protease at higher rates, thus facilitating release of the cargo protein. In some embodiments, a linker comprising the amino acid sequence G is inserted N′ and/or C′ to the cleavable linker. In certain embodiments, a linker comprising the amino acid sequence G is inserted C′ to the cleavable linker. In some embodiments, a linker comprising the amino acid sequence GGS is inserted N′ and/or C′ to the cleavable linker. In certain embodiments, linkers comprising the amino acid sequence GGS are inserted both N′ and C′ to the cleavable linker. In some embodiments, a linker comprising the amino acid sequence SGGSSGGS (SEQ ID NO: 163) is inserted N′ and/or C′ to the cleavable linker. In certain embodiments, linkers comprising the amino acid sequence SGGSSGGS (SEQ ID NO: 163) are inserted both N′ and C′ to the cleavable linker.

In some embodiments, the gag-pro polyprotein of the PE-VLPs described herein comprises an MMLV gag-pro polyprotein or an FMLV gag-pro polyprotein. In some embodiments, the gag nucleocapsid protein of the fusion protein in the PE-VLPs described herein comprises an MMLV gag nucleocapsid protein or an FMLV gag nucleocapsid protein.

In some embodiments, a fusion protein delivered by the VLP comprises both a napDNAbp and a domain comprising an RNA-dependent DNA polymerase activity (e.g., a reverse transcriptase domain). In certain embodiments, the fusion protein comprises one of the following non-limiting structures:
[gag nucleocapsid protein]-[napDNAbp]-[RT domain], wherein each instance of [-] comprises an optional linker (e.g., an amino acid linker, or any of the linkers provided herein);
[gag nucleocapsid protein]-[1×-3×NES]-[cleavable linker]-[NLS]-[RT domain]-[napDNAbp]-[NLS], wherein each instance of]-[comprises an optional linker (e.g., an amino acid linker, or any of the linkers provided herein);
[1×-3×NES]-[gag nucleocapsid protein]-[cleavable linker]-[NLS]-[RT domain]-[napDNAbp]-[NLS], wherein each instance of]-[comprises an optional linker (e.g., an amino acid linker, or any of the linkers provided herein); or
[gag nucleocapsid protein]-[1×-3×NES]-[cleavable linker]-[NLS]-[RT domain]-[napDNAbp]-[NLS]-[cleavable linker]-[1×-3×NES], wherein each instance of [-[ comprises an optional linker (e.g., an amino acid linker, or any of the linkers provided herein).

In embodiments in which the cleavable linker has been cleaved by the protease within the VLP, the VLP may comprise a fusion protein comprising the structure [gag nucleocapsid protein]-[1×-3×NES], and a free prime editor. In certain embodiments, the prime editor comprises the structure [NLS]-[domain comprising an RNA-dependent DNA polymerase activity]-[napDNAbp]-[NLS].

In some embodiments, any of the constructs above comprise 3×NES.

In some embodiments, the napDNAbp and the domain comprising an RNA-dependent DNA polymerase activity (e.g., a reverse transcriptase domain) are included on two different fusion proteins that are each delivered in a VLP, or are each delivered in separate VLPs. In some embodiments, each of the fusion proteins comprises a split intein to facilitate fusion of the napDNAbp and the domain comprising an RNA-dependent DNA polymerase activity. In certain embodiments, the two fusion proteins, one comprising a napDNAbp and one comprising a domain comprising an RNA-dependent DNA polymerase activity, comprise the following non-limiting structures:
[gag nucleocapsid protein]-[napDNAbp]-[split intein]; and
[gag nucleocapsid protein]-[split intein]-[domain comprising RNA-dependent DNA polymerase activity], wherein each instance of [-] in each fusion protein comprises an optional linker (e.g., an amino acid linker, or any of the linkers provided herein). In certain embodiments, the two fusion proteins, one comprising a napDNAbp and one comprising a domain comprising an RNA-dependent DNA polymerase activity, comprise the following non-limiting structures:
[gag nucleocapsid protein]-[first portion of napDNAbp]-[split intein]; and
[gag nucleocapsid protein]-[split intein]-[second portion of napDNAbp]-[domain comprising RNA-dependent DNA polymerase activity], wherein each instance of [-] in each fusion protein comprises an optional linker (e.g., an amino acid linker, or any of the linkers provided herein).

The eVLPs (e.g., the PE-VLPs) provided by the present disclosure comprise an outer encapsulation layer (or envelope layer) comprising a viral envelope glycoprotein. Any viral envelope glycoprotein described herein, or known in the art, may be used in the PE-VLPs of the present disclosure. In some embodiments, the viral envelope glycoprotein is an adenoviral envelope glycoprotein, an adeno-associated viral envelope glycoprotein, a retroviral envelope glycoprotein, or a lentiviral envelope glycoprotein. In certain embodiments, the viral envelope glycoprotein is a retroviral envelope glycoprotein. In some embodiments, the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein. In some embodiments, the viral envelope glycoprotein targets the system to a particular cell type (e.g., immune cells, neural cells, retinal pigment epithelium cells, etc.). For example, using different envelope glycoproteins in the eVLPs described herein may alter their cellular tropism, allowing the PE-VLPs to be targeted to specific cell types. In some embodiments, the viral envelope glycoprotein is a VSV-G protein, and the VSV-G protein targets the system to retinal pigment epithelium (RPE) cells. In some embodiments, the viral envelope glycoprotein is an HIV-1 envelope glycoprotein, and the HIV-1 envelope glycoprotein targets the system to CD4+ cells. In some embodiments, the viral envelope glycoprotein is a FuG-B2 envelope glycoprotein, and the FuG-B2 envelope glycoprotein targets the system to neurons.

It will be appreciated that general methods are known in the art for producing viral vector particles, which generally contain coding nucleic acids of interest, and such methods may also be used for producing the virus-derived particles according to the present invention, which do not contain coding nucleic acids of interest but instead are designed to deliver a protein cargo (e.g., a PE RNP).

Conventional viral vector particles encompass retroviral, lentiviral, adenoviral and adeno-associated viral vector particles that are well known in the art. For a review of various viral vector particles that may be used, the one skilled in the art may notably refer to Kushnir et al. (2012, Vaccine, Vol. 31: 58-83), Zeltons (2013, Mol Biotechnol, Vol. 53: 92-107), Ludwig et al. (2007, Curr Opin Biotechnol, Vol. 18(no 6): 537-55) and Naskalaska et al. (2015, Vol. 64 (no 1): 3-13). Further, references to various methods using virus-derived particles for delivering proteins to cells are found by the one skilled in the art in the article of Maetzig et al. (2012, Current Gene Therapy, Vol. 12: 389-409) as well as the article of Kaczmarczyk et al. (2011, Proc Natl Acad Sci USA, Vol. 108 (no 41): 16998-17003).

Generally, a virus-like particle that is used according to the present disclosure, which virus-like particle may also be termed “virus-derived particle,” is formed by one or more virus-derived structural protein(s) and/or one more virus-derived envelope protein(s).

A virus-like particle that is used according to the present invention is replication incompetent in a host cell wherein it has entered.

In preferred embodiments, a virus-like particle is formed by one or more retrovirus-derived structural protein(s) and optionally one or more virus-derived envelope protein(s).

In preferred embodiments, the virus-derived structural protein is a retroviral Gag protein or a peptide fragment thereof. As it is known in the art, Gag and Gag/pol precursors are expressed from full length genomic RNA as polyproteins, which require proteolytic cleavage, mediated by the retroviral protease (PR), to acquire a functional conformation. Further, Gag, which is structurally conserved among the retroviruses, is composed of at least three protein units: matrix protein (MA), capsid protein (CA) and nucleocapsid protein (NC), whereas Pol consists of the retroviral protease, (PR), the retrotranscriptase (RT), and the integrase (IN).

In some embodiments, a virus-derived particle comprises a retroviral Gag protein but does not comprise a Pol protein.

As it is known in the art, the host range of retroviral vector, including lentiviral vectors, may be expanded or altered by a process known as pseudotyping. Pseudotyped lentiviral vectors consist of viral vector particles bearing glycoproteins derived from other enveloped viruses. Such pseudotyped viral vector particles possess the tropism of the virus from which the glycoprotein is derived.

In some embodiments, a virus-like particle is a pseudotyped virus-like particle comprising one or more viral structural protein(s) or viral envelope protein(s) imparting a tropism to the said virus-like particle for certain eukaryotic cells. A pseudotyped virus-like particle as described herein may comprise, as the viral protein used for pseudotyping, a viral envelope protein selected in a group comprising VSV-G protein, Measles virus HA protein, Measles virus F protein, Influenza virus HA protein, Moloney virus MLV-A protein, Moloney virus MLV-E protein, Baboon Endogenous retrovirus (BAEV) envelope protein, Ebola virus glycoprotein and foamy virus envelope protein, or a combination of two or more of these viral envelope proteins.

A well-known illustration of pseudotyping viral vector particles consists of the pseudotyping of viral vector particles with the vesicular stomatitis virus glycoprotein (VSV-G). For the pseudotyping of viral vector particles, one skilled in the art may notably refer to Yee et al. (1994, Proc Natl Acad Sci, USA, Vol. 91: 9564-9568) Cronin et al. (2005, Curr Gene Ther, Vol. 5(no 4): 387-398), which are incorporated herein by reference.

For producing virus-like particles, and more precisely VSV-G pseudotyped virus-like particles, for delivering protein(s) of interest into target cells, one skilled in the art may refer to Mangeot et al. (2011, Molecular Therapy, Vol. 19 (no 9): 1656-1666).

In some embodiments, a virus-like particle further comprises a viral envelope protein, wherein either (i) the said viral envelope protein originates from the same virus as the viral structural protein, e.g., originates from the same virus as the viral Gag protein, or (ii) the said viral envelope protein originates from a virus distinct from the virus from which originates the viral structural protein, e.g., originates from a virus distinct from the virus from which originates the viral Gag protein.

As it is readily understood by the one skilled in the art, a virus-like particle that is used according to the disclosure may be selected in a group comprising Moloney murine leukemia virus-derived vector particles, Bovine immunodeficiency virus-derived particles, Simian immunodeficiency virus-derived vector particles, Feline immunodeficiency virus-derived vector particles, Human immunodeficiency virus-derived vector particles, Equine infection anemia virus-derived vector particles, Caprine arthritis encephalitis virus-derived vector particle, Baboon endogenous virus-derived vector particles, Rabies virus-derived vector particles, Influenza virus-derived vector particles, Norovirus-derived vector particles, Respiratory syncytial virus-derived vector particles, Hepatitis A virus-derived vector particles, Hepatitis B virus-derived vector particles, Hepatitis E virus-derived vector particles, Newcastle disease virus-derived vector particles, Norwalk virus-derived vector particles, Parvovirus-derived vector particles, Papillomavirus-derived vector particles, Yeast retrotransposon-derived vector particles, Measles virus-derived vector particles, and bacteriophage-derived vector particles.

In particular, a virus-like particle that is used according to the invention is a retrovirus-derived particle. Such retrovirus may be selected among Moloney murine leukemia virus, Bovine immunodeficiency virus, Simian immunodeficiency virus, Feline immunodeficiency virus, Human immunodeficiency virus, Equine infection anemia virus, and Caprine arthritis encephalitis virus.

In another embodiment, a virus-like particle that is used according to the disclosure is a lentivirus-derived particle. Lentiviruses belong to the retroviruses family, and have the unique ability of being able to infect non-dividing cells.

Such lentivirus may be selected among Bovine immunodeficiency virus, Simian immunodeficiency virus, Feline immunodeficiency virus, Human immunodeficiency virus, Equine infection anemia virus, and Caprine arthritis encephalitis virus.

For preparing Moloney murine leukemia virus-derived vector particles, one skilled in the art may refer to the methods disclosed by Sharma et al. (1997, Proc Nal Acad Sci USA, Vol. 94: 10803+-10808), Guibingua et al. (2002, Molecular Therapy, Vol. 5(no 5): 538-546), which are incorporated herein by reference. Moloney murine leukemia virus-derived (MLV-derived) vector particles may be selected in a group comprising MLV-A-derived vector particles and MLV-E-derived vector particles.

For preparing Bovine Immunodeficiency virus-derived vector particles, one skilled in the art may refer to the methods disclosed by Rasmussen et al. (1990, Virology, Vol. 178(no 2): 435-451), which is incorporated herein by reference.

For preparing Simian immunodeficiency virus-derived vector particles, including VSV-G pseudotyped SIV virus-derived particles, one skilled in the art may notably refer to the methods disclosed by Mangeot et al. (2000, Journal of Virology, Vol. 71(no 18): 8307-8315), Negre et al. (2000, Gene Therapy, Vol. 7: 1613-1623), and Mangeot et al. (2004, Nucleic Acids Research, Vol. 32 (no 12), e102), which are incorporated herein by reference.

For preparing Feline Immunodeficiency virus-derived vector particles, one skilled in the art may notably refer to the methods disclosed by Saenz et al. (2012, Cold Spring Harb Protoc, (1): 71-76; 2012, Cold Spring Harb Protoc, (1): 124-125; 2012, Cold Spring Harb Protoc, (1): 118-123), which are incorporated herein by reference.

For preparing Human immunodeficiency virus-derived vector particles, one skilled in the art may notably refer to the methods disclosed by Jalaguier et al. (2011, PlosOne, Vol. 6(no 11), e28314), Cervera et al. (J Biotechnol, Vol. 166(no 4): 152-165), and Tang et al. (2012, Journal of Virology, Vol. 86(no 14): 7662-7676), which are incorporated herein by reference.

For preparing Equine infection anemia virus-derived vector particles, one skilled in the art may notably refer to the methods disclosed by Olsen (1998, Gene Ther, Vol. 5(no 11): 1481-1487), which are incorporated herein by reference.

For preparing Caprine arthritis encephalitis virus-derived vector particles, one skilled in the art may notably refer to the methods disclosed by Mselli-Lakhal et al. (2006, J Virol Methods, Vol. 136(no 1-2): 177-184), which are incorporated herein by reference.

For preparing Baboon endogenous virus-derived vector particles, one skilled in the art may notably refer to the methods disclosed by Girard-Gagnepain et al. (2014, Blood, Vol. 124(no 8): 1221-1231), which is incorporated herein by reference.

For preparing Rabies virus-derived vector particles, one skilled in the art may notably refer to the methods disclosed by Kang et al. (2015, Viruses, Vol. 7: 1134-1152, doi:10.3390/v7031134) and Fontana et al. (2014, Vaccine, Vol. 32(no 24): 2799-27804), which are incorporated herein by reference, or to the PCT application published under no. WO 2012/0618, which is incorporated herein by reference.

For preparing Influenza virus-derived vector particles, one skilled in the art may notably refer to the methods disclosed by Quan et al. (2012, Virology, Vol. 430: 127-135) and to Latham et al. (2001, Journal of Virology, Vol. 75(no 13): 6154-6155), which are incorporated herein by reference.

For preparing Norovirus-derived vector particles, one skilled in the art may notably refer to the methods disclosed by Tomd-Amat et al., (2014, Microbial Cell Factories, Vol. 13: 134-142), which is incorporated herein by reference.

For preparing Respiratory syncytial virus-derived vector particles, one skilled in the art may notably refer to the methods disclosed by Walpita et al. (2015, PlosOne, DOI: 10.1371/journal.pone.0130755), which is incorporated herein by reference.

For preparing Hepatitis B virus-derived vector particles, one skilled in the art may notably refer to the methods disclosed by Hong et al. (2013, Journal of Virology, Vol. 87(no 12): 6615-6624), which is incorporated herein by reference.

For preparing Hepatitis E virus-derived vector particles, one skilled in the art may notably refer to the methods disclosed by Li et al. (1997, Journal of Virology, Vol. 71(no 10): 7207-7213), which is incorporated herein by reference.

For preparing Newcastle disease virus-derived vector particles, one skilled in the art may notably refer to the methods disclosed by Murawski et al. (2010, Journal of Virology, Vol. 84(no 2): 1110-1123), which is incorporated herein by reference.

For preparing Norwalk virus-derived vector particles, one skilled in the art may notably refer to the methods disclosed by Herbst-Kralovetz et al. (2010, Expert Rev Vaccines, Vol. 9(no 3): 299-307), which is incorporated herein by reference.

For preparing Parvovirus-derived vector particles, one skilled in the art may notably refer to the methods disclosed by Ogasawara et al. (2006, In Vivo, Vol. 20: 319-324), which is incorporated herein by reference.

For preparing Papillomavirus-derived vector particles, one skilled in the art may notably refer to the methods disclosed by Wang et al. (2013, Expert Rev Vaccines, Vol. 12(no 2): doi:10.1586/erv.12.151), which is incorporated herein by reference.

A virus-like particle that is used herein comprises a Gag protein, and most preferably a Gag protein originating from a virus selected from a group consisting of Rous Sarcoma Virus (RSV) Feline Immunodeficiency Virus (FIV), Simian Immunodeficiency Virus (SIV), Moloney Leukemia Virus (MLV), and Human Immunodeficiency Viruses (HIV-1 and HIV-2), especially Human Immunodeficiency Virus of type 1 (HIV-1).

In some embodiments, a virus-like particle may also comprise one or more viral envelope protein(s). The presence of one or more viral envelope protein(s) may impart to the said virus-derived particle a more specific tropism for the cells which are targeted, as it is known in the art. The one or more viral envelope protein(s) may be selected from a group consisting of envelope proteins from retroviruses, envelope proteins from non-retroviral viruses, and chimeras of these viral envelope proteins with other peptides or proteins. An example of a non-lentiviral envelope glycoprotein of interest is the lymphocytic choriomeningitis virus (LCMV) strain WE54 envelope glycoprotein. These envelope glycoproteins increase the range of cells that can be transduced with retroviral derived vectors.

In some embodiments, the prime editing guide RNAs (pegRNAs) and/or the second strand nicking guide RNAs (ngRNAs) delivered by the VLPs disclosed herein comprise an aptamer. In some embodiments, the gag-pro-polyprotein is fused to a target molecule that binds an aptamer inserted into the structure of the pegRNA or ngRNA. The inclusion of such an aptamer and target molecule that binds the aptamer may be useful, for example, for facilitating the packing of the pegRNA and/or ngRNA into the VLP. In some embodiments, the aptamer is inserted into the pegRNA backbone sequence and/or the ngRNA backbone sequence. In some embodiments, the target molecule that binds the aptamer is inserted into the gag-pro polyprotein. In certain embodiments, the aptamer comprises the MS2 stem loop, and the target molecule that binds the aptamer comprises the MS2 coat protein. In certain embodiments, the aptamer comprises the Com aptamer, and the target molecule that binds the aptamer comprises the Com protein. The present disclosure is not limited with respect to the aptamers and target molecules that can be utilized in the VLPs disclosed herein, and any aptamers and their corresponding target molecules known in the art may be incorporated into the VLPs. In some embodiments, the ratio of a wild type gag-pro polyprotein to a target molecule-modified gag-pro polyprotein to one or more fusion proteins in a VLP is approximately 5:2:1. Such a ratio may provide optimal prime editing efficiencies upon delivery of a prime editor cargo protein.

In some embodiments, various components of the VLPs described herein may also be fused to coiled-coil peptides to facilitate the assembly of the VLPs through the interactions of the coiled-coil peptides. For example, in some embodiments, a first coiled-coil peptide may be inserted into the gag-pro polyprotein of the VLPs. In some embodiments, a second coiled-coil peptide may be fused to the one or more fusion proteins of the VLPs (e.g., at the N-terminus, at the C-terminus, or at an internal position within the one or more fusion proteins). In certain embodiments, the coiled-coil peptide is fused to the C-terminus of the one or more fusion proteins.

Any coiled-coil peptide pairs known in the art may be used in the VLPs described herein. For example, in some embodiments, the P3 and P4 peptides may be used:

P3 peptide sequence:
(SEQ ID NO: 35)
SPEDEIQQLEEEIAQLEQKNAALKEKNQALKYG;
P4 peptide sequence:
(SEQ ID NO: 36)
SPEDKIAQLKQKIQALKQENQQLEEENAALEYG.

In some embodiments, one of the first or the second coiled-coil peptides comprises the P3 peptide, and the other of the first or the second coiled-coil peptides comprises the P4 peptide. In certain embodiments, the first coiled-coil peptide comprises the P3 peptide. In certain embodiments, the second coiled-coil peptide comprises the P4 peptide.

napDNAbp

In various embodiments, the PE-VLPs disclosed herein, as well as the prime editor fusion proteins that make up the core component of the presently described PE-VLPs, comprise a nucleic acid programmable DNA binding protein (napDNAbp).

In various embodiments, the PE-VLPs and prime editor fusion proteins may include a napDNAbp domain having a wild type Cas9 sequence, including, for example the canonical Streptococcus pyogenes Cas9 sequence of SEQ ID NO: 37, shown as follows.

| Description | Sequence | SEQ ID NO: |
|---|---|---|
| SpCas9 | MDKKYSIGLDIGTNSVGWAVITDEYKVPSKKFKVLGN | 37 |
| Strepto- | TDRHSIKKNLIGALLFDSGETAEATRLKRTARRRYTRR | |
| coccus | KNRICYLQEIFSNEMAKVDDSFFHRLEESFLVEEDKKH | |
| pyogenes | ERHPIFGNIVDEVAYHEKYPTIYHLRKKLVDSTDKADL | |
| M1 | RLIYLALAHMIKFRGHFLIEGDLNPDNSDVDKLFIQLVQ | |
| SwissProt | TYNQLFEENPINASGVDAKAILSARLSKSRRLENLIAQL | |
| Accession | PGEKKNGLFGNLIALSLGLTPNFKSNFDLAEDAKLQLS | |
| No. | KDTYDDDLDNLLAQIGDQYADLFLAAKNLSDAILLSDI | |
| Q99ZW2 | LRVNTEITKAPLSASMIKRYDEHHQDLTLLKALVRQQL | |
| Wild type | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGN | |
| | SRFAWMTRKSEETITPWNFEEVVDKGASAQSFIERMTN | |
| | FDKNLPNEKVLPKHSLLYEYFTVYNELTKVKYVTEGM | |
| | RKPAFLSGEQKKAIVDLLFKTNRKVTVKQLKEDYFKKI | |
| | ECFDSVEISGVEDRFNASLGTYHDLLKIIKDKDFLDNEE | |
| | NEDILEDIVLTLTLFEDREMIEERLKTYAHLFDDKVMK | |
| | QLKRRRYTGWGRLSRKLINGIRDKQSGKTILDFLKSDG | |
| | FANRNFMQLIHDDSLTFKEDIQKAQVSGQGDSLHEHIA | |
| | NLAGSPAIKKGILQTVKVVDELVKVMGRHKPENIVIEM | |
| | ARENQTTQKGQKNSRERMKRIEEGIKELGSQILKEHPV | |
| | ENTQLQNEKLYLYYLQNGRDMYVDQELDINRLSDYD | |
| | VDHIVPQSFLKDDSIDNKVLTRSDKNRGKSDNVPSEEV | |
| | VKKMKNYWRQLLNAKLITQRKFDNLTKAERGGLSEL | |
| | DKAGFIKRQLVETRQITKHVAQILDSRMNTKYDENDK | |
| | LIREVKVITLKSKLVSDFRKDFQFYKVREINNYHHAHD | |
| | AYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEV | |
| | QTGGFSKESILPKRNSDKLIARKKDWDPKKYGGFDSPT | |
| | VAYSVLVVAKVEKGKSKKLKSVKELLGITIMERSSFEK | |
| | NPIDFLEAKGYKEVKKDLIIKLPKYSLFELENGRKRML | |
| | ASAGELQKGNELALPSKYVNFLYLASHYEKLKGSPED | |
| | NEQKQLFVEQHKHYLDEIIEQISEFSKRVILADANLDKV | |
| | LSAYNKHRDKPIREQAENIIHLFTLTNLGAPAAFKYFDT | |
| | TIDRKRYTSTKEVLDATLIHQSITGLYETRIDLSQLGGD | |

In other embodiments, the PE-VLPs and fusion proteins may include a napDNAbp domain having a modified Cas9 sequence, including, for example the nickase variant of Streptococcus pyogenes Cas9 of SEQ ID NO: 38 having an H840A substitution relative to the wild type SpCas9 (of SEQ ID NO: 37), shown as follows:

| | | |
|---|---|---|
| Cas9 nickase | MDKKYSIGLDIGTNSVGWAVITDEYKVPSKKFKVLGNTD | 38 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H840A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDAIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

The PE-VLPs and prime editor fusion proteins described herein may include any of the modified Cas9 sequences described above, or any variant thereof having at least 80%, at least 85%, at least 90%, at least 95%, or at least 99% sequence identity thereto. In some embodiments, the improved prime editor fusion proteins described herein include any of the following other wild type SpCas9 sequences, which may be modified with one or more of the mutations described herein at corresponding amino acid positions:

| Description | Sequence |
|---|---|
| SpCas9 | ATGGATAAGAAATACTCAATAGGCTTAGATATCGGCACAAATAG |
| Streptococcus | CGTCGGATGGGCGGTGATCACTGATGATTATAAGGTTCCGTCTAA |
| pyogenes | AAAGTTCAAGGTTCTGGGAAATACAGACCGCCACAGTATCAAAA |
| MGAS1882 | AAAATCTTATAGGGGCTCTTTTATTTGGCAGTGGAGAGACAGCGG |
| wild type | AAGCGACTCGTCTCAAACGGACAGCTCGTAGAAGGTATACACGTC |
| NC_017053.1 | GGAAGAATCGTATTTGTTATCTACAGGAGATTTTTTCAAATGAGA |
| | TGGCGAAAGTAGATGATAGTTTCTTTCATCGACTTGAAGAGTCTT |
| | TTTTGGTGGAAGAAGACAAGAAGCATGAACGTCATCCTATTTTTG |
| | GAAATATAGTAGATGAAGTTGCTTATCATGAGAAATATCCAACTA |
| | TCTATCATCTGCGAAAAAAATTGGCAGATTCTACTGATAAAGCGG |
| | ATTTGCGCTTAATCTATTTGGCCTTAGCGCATATGATTAAGTTTCG |
| | TGGTCATTTTTTGATTGAGGGAGATTTAAATCCTGATAATAGTGAT |
| | GTGGACAAACTATTTATCCAGTTGGTACAAATCTACAATCAATTA |
| | TTTGAAGAAAACCCTATTAACGCAAGTAGAGTAGATGCTAAAGC |
| | GATTCTTTCTGCACGATTGAGTAAATCAAGACGATTAGAAAATCT |
| | CATTGCTCAGCTCCCCGGTGAGAAGAGAAATGGCTTGTTTGGGAA |
| | TCTCATTGCTTTGTCATTGGGATTGACCCCTAATTTTAAATCAAAT |
| | TTTGATTTGGCAGAAGATGCTAAATTACAGCTTTCAAAAGATACT |
| | TACGATGATGATTTAGATAATTTATTGGCGCAAATTGGAGATCAA |
| | TATGCTGATTTGTTTTTGGCAGCTAAGAATTTATCAGATGCTATTT |
| | TACTTTCAGATATCCTAAGAGTAAATAGTGAAATAACTAAGGCTC |
| | CCCTATCAGCTTCAATGATTAAGCGCTACGATGAACATCATCAAG |
| | ACTTGACTCTTTTAAAAGCTTTAGTTCGACAACAACTTCCAGAAA |
| | AGTATAAAGAAATCTTTTTTGATCAATCAAAAAACGGATATGCAG |
| | GTTATATTGATGGGGGAGCTAGCCAAGAAGAATTTTATAAATTTA |
| | TCAAACCAATTTTAGAAAAAATGGATGGTACTGAGGAATTATTGG |
| | TGAAACTAAATCGTGAAGATTTGCTGCGCAAGCAACGGACCTTTG |
| | ACAACGGCTCTATTCCCCATCAAATTCACTTGGGTGAGCTGCATG |
| | CTATTTTGAGAAGACAAGAAGACTTTTATCCATTTTTAAAAGACA |
| | ATCGTGAGAAGATTGAAAAAATCTTGACTTTTCGAATTCCTTATT |
| | ATGTTGGTCCATTGGCGCGTGGCAATAGTCGTTTTGCATGGATGA |
| | CTCGGAAGTCTGAAGAAACAATTACCCCATGGAATTTTGAAGAAG |
| | TTGTCGATAAAGGTGCTTCAGCTCAATCATTTATTGAACGCATGA |
| | CAAACTTTGATAAAAATCTTCCAAATGAAAAAGTACTACCAAAAC |
| | ATAGTTTGCTTTATGAGTATTTTACGGTTTATAACGAATTGACAAA |
| | GGTCAAATATGTTACTGAGGGAATGCGAAAACCAGCATTTCTTTC |
| | AGGTGAACAGAAGAAAGCCATTGTTGATTTACTCTTCAAAACAAA |
| | TCGAAAAGTAACCGTTAAGCAATTAAAAGAAGATTATTTCAAAA |
| | AAATAGAATGTTTTGATAGTGTTGAAATTTCAGGAGTTGAAGATA |
| | GATTTAATGCTTCATTAGGCGCCTACCATGATTTGCTAAAAATTAT |
| | TAAAGATAAAGATTTTTTGGATAATGAAGAAAATGAAGATATCTT |
| | AGAGGATATTGTTTTAACATTGACCTTATTTGAAGATAGGGGGAT |
| | GATTGAGGAAAGACTTAAAACATATGCTCACCTCTTTGATGATAA |
| | GGTGATGAAACAGCTTAAACGTCGCCGTTATACTGGTTGGGGACG |
| | TTTGTCTCGAAAATTGATTAATGGTATTAGGGATAAGCAATCTGG |
| | CAAAACAATATTAGATTTTTTGAAATCAGATGGTTTTGCCAATCG |
| | CAATTTTATGCAGCTGATCCATGATGATAGTTTGACATTTAAAGA |
| | AGATATTCAAAAAGCACAGGTGTCTGGACAAGGCCATAGTTTACA |
| | TGAACAGATTGCTAACTTAGCTGGCAGTCCTGCTATTAAAAAAGG |
| | TATTTTACAGACTGTAAAAATTGTTGATGAACTGGTCAAAGTAAT |
| | GGGGCATAAGCCAGAAAATATCGTTATTGAAATGGCACGTGAAA |
| | ATCAGACAACTCAAAAGGGCCAGAAAAATTCGCGAGAGCGTATG |
| | AAACGAATCGAAGAAGGTATCAAAGAATTAGGAAGTCAGATTCT |
| | TAAAGAGCATCCTGTTGAAAATACTCAATTGCAAAATGAAAAGCT |
| | CTATCTCTATTATCTACAAAATGGAAGAGACATGTATGTGGACCA |
| | AGAATTAGATATTAATCGTTTAAGTGATTATGATGTCGATCACAT |
| | TGTTCCACAAAGTTTCATTAAAGACGATTCAATAGACAATAAGGT |
| | ACTAACGCGTTCTGATAAAAATCGTGGTAAATCGGATAACGTTCC |
| | AAGTGAAGAAGTAGTCAAAAAGATGAAAAACTATTGGAGACAAC |
| | TTCTAAACGCCAAGTTAATCACTCAACGTAAGTTTGATAATTTAA |
| | CGAAAGCTGAACGTGGAGGTTTGAGTGAACTTGATAAAGCTGGTT |
| | TTATCAAACGCCAATTGGTTGAAACTCGCCAAATCACTAAGCATG |
| | TGGCACAAATTTTGGATAGTCGCATGAATACTAAATACGATGAAA |
| | ATGATAAACTTATTCGAGAGGTTAAAGTGATTACCTTAAAATCTA |
| | AATTAGTTTCTGACTTCCGAAAAGATTTCCAATTCTATAAAGTAC |
| | GTGAGATTAACAATTACCATCATGCCCATGATGCGTATCTAAATG |
| | CCGTCGTTGGAACTGCTTTGATTAAGAAATATCCAAAACTTGAAT |
| | CGGAGTTTGTCTATGGTGATTATAAAGTTTATGATGTTCGTAAAAT |
| | GATTGCTAAGTCTGAGCAAGAAATAGGCAAAGCAACCGCAAAAT |
| | ATTTCTTTTACTCTAATATCATGAACTTCTTCAAAACAGAAATTAC |
| | ACTTGCAAATGGAGAGATTCGCAAACGCCCTCTAATCGAAACTAA |
| | TGGGGAAACTGGAGAAATTGTCTGGGATAAAGGGCGAGATTTTG |
| | CCACAGTGCGCAAAGTATTGTCCATGCCCCAAGTCAATATTGTCA |
| | AGAAAACAGAAGTACAGACAGGCGGATTCTCCAAGGAGTCAATT |
| | TTACCAAAAAGAAATTCGGACAAGCTTATTGCTCGTAAAAAAGAC |
| | TGGGATCCAAAAAAATATGGTGGTTTTGATAGTCCAACGGTAGCT |
| | TATTCAGTCCTAGTGGTTGCTAAGGTGGAAAAAGGGAAATCGAA |
| | GAAGTTAAAATCCGTTAAAGAGTTACTAGGGATCACAATTATGGA |
| | AAGAAGTTCCTTTGAAAAAAATCCGATTGACTTTTTAGAAGCTAA |
| | AGGATATAAGGAAGTTAAAAAAGACTTAATCATTAAACTACCTA |
| | AATATAGTCTTTTTGAGTTAGAAAACGGTCGTAAACGGATGCTGG |
| | CTAGTGCCGGAGAATTACAAAAAGGAAATGAGCTGGCTCTGCCA |
| | AGCAAATATGTGAATTTTTTATATTTAGCTAGTCATTATGAAAAGT |
| | TGAAGGGTAGTCCAGAAGATAACGAACAAAAACAATTGTTTGTG |
| | GAGCAGCATAAGCATTATTTAGATGAGATTATTGAGCAAATCAGT |
| | GAATTTTCTAAGCGTGTTATTTTAGCAGATGCCAATTTAGATAAA |
| | GTTCTTAGTGCATATAACAAACATAGAGACAAACCAATACGTGAA |
| | CAAGCAGAAAATATTATTCATTTATTTACGTTGACGAATCTTGGA |
| | GCTCCCGCTGCTTTTAAATATTTTGATACAACAATTGATCGTAAAC |
| | GATATACGTCTACAAAAGAAGTTTTAGATGCCACTCTTATCCATC |
| | AATCCATCACTGGTCTTTATGAAACACGCATTGATTTGAGTCAGC |
| | TAGGAGGTGACTGA (SEQ ID NO: 56) |
| SpCas9 | MDKKYSIGLDIGTNSVGWAVITDDYKVPSKKFKVLGNTDRHSIKKN |
| Streptococcus | LIGALLFGSGETAEATRLKRTARRRYTRRKNRICYLQEIFSNEMAKV |
| pyogenes | DDSFFHRLEESFLVEEDKKHERHPIFGNIVDEVAYHEKYPTIYHLRKK |
| MGAS1882 | LADSTDKADLRLIYLALAHMIKFRGHFLIEGDLNPDNSDVDKLFIQLV |
| wild type | QIYNQLFEENPINASRVDAKAILSARLSKSRRLENLIAQLPGEKRNGL |
| NC_017053.1 | FGNLIALSLGLTPNFKSNFDLAEDAKLQLSKDTYDDDLDNLLAQIGD |
| | QYADLFLAAKNLSDAILLSDILRVNSEITKAPLSASMIKRYDEHHQDL |
| | TLLKALVRQQLPEKYKEIFFDQSKNGYAGYIDGGASQEEFYKFIKPIL |
| | EKMDGTEELLVKLNREDLLRKQRTFDNGSIPHQIHLGELHAILRRQE |
| | DFYPFLKDNREKIEKILTFRIPYYVGPLARGNSRFAWMTRKSEETITP |
| | WNFEEVVDKGASAQSFIERMTNFDKNLPNEKVLPKHSLLYEYFTVY |
| | NELTKVKYVTEGMRKPAFLSGEQKKAIVDLLFKTNRKVTVKQLKED |
| | YFKKIECFDSVEISGVEDRFNASLGAYHDLLKIIKDKDFLDNEENEDIL |
| | EDIVLTLTLFEDRGMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGHSLHEQIANLAGSPAIKKGILQTVKIVDELVKVMGHKPEN |
| | IVIEMARENQTTQKGQKNSRERMKRIEEGIKELGSQILKEHPVENTQL |
| | QNEKLYLYYLQNGRDMYVDQELDINRLSDYDVDHIVPQSFIKDDSID |
| | NKVLTRSDKNRGKSDNVPSEEVVKKMKNYWRQLLNAKLITQRKFD |
| | NLTKAERGGLSELDKAGFIKRQLVETRQITKHVAQILDSRMNTKYDE |
| | NDKLIREVKVITLKSKLVSDFRKDFQFYKVREINNYHHAHDAYLNA |
| | VVGTALIKKYPKLESEFVYGDYKVYDVRKMIAKSEQEIGKATAKYF |
| | FYSNIMNFFKTEITLANGEIRKRPLIETNGETGEIVWDKGRDFATVRK |
| | VLSMPQVNIVKKTEVQTGGFSKESILPKRNSDKLIARKKDWDPKKYG |
| | GFDSPTVAYSVLVVAKVEKGKSKKLKSVKELLGITIMERSSFEKNPID |
| | FLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAA |
| | FKYFDTTIDRKRYTSTKEVLDATLIHQSITGLYETRIDLSQLGGD (SEQ |
| | ID NO: 41) |
| SpCas9 | ATGGATAAAAAGTATTCTATTGGTTTAGACATCGGCACTAATTCC |
| Streptococcus | GTTGGATGGGCTGTCATAACCGATGAATACAAAGTACCTTCAAAG |
| pyogenes wild | AAATTTAAGGTGTTGGGGAACACAGACCGTCATTCGATTAAAAAG |
| type | AATCTTATCGGTGCCCTCCTATTCGATAGTGGCGAAACGGCAGAG |
| SWBC2D7W014 | GCGACTCGCCTGAAACGAACCGCTCGGAGAAGGTATACACGTCG |
| | CAAGAACCGAATATGTTACTTACAAGAAATTTTTAGCAATGAGAT |
| | GGCCAAAGTTGACGATTCTTTCTTTCACCGTTTGGAAGAGTCCTTC |
| | CTTGTCGAAGAGGACAAGAAACATGAACGGCACCCCATCTTTGG |
| | AAACATAGTAGATGAGGTGGCATATCATGAAAAGTACCCAACGA |
| | TTTATCACCTCAGAAAAAAGCTAGTTGACTCAACTGATAAAGCGG |
| | ACCTGAGGTTAATCTACTTGGCTCTTGCCCATATGATAAAGTTCCG |
| | TGGGCACTTTCTCATTGAGGGTGATCTAAATCCGGACAACTCGGA |
| | TGTCGACAAACTGTTCATCCAGTTAGTACAAACCTATAATCAGTT |
| | GTTTGAAGAGAACCCTATAAATGCAAGTGGCGTGGATGCGAAGG |
| | CTATTCTTAGCGCCCGCCTCTCTAAATCCCGACGGCTAGAAAACC |
| | TGATCGCACAATTACCCGGAGAGAAGAAAAATGGGTTGTTCGGT |
| | AACCTTATAGCGCTCTCACTAGGCCTGACACCAAATTTTAAGTCG |
| | AACTTCGACTTAGCTGAAGATGCCAAATTGCAGCTTAGTAAGGAC |
| | ACGTACGATGACGATCTCGACAATCTACTGGCACAAATTGGAGAT |
| | CAGTATGCGGACTTATTTTTGGCTGCCAAAAACCTTAGCGATGCA |
| | ATCCTCCTATCTGACATACTGAGAGTTAATACTGAGATTACCAAG |
| | GCGCCGTTATCCGCTTCAATGATCAAAAGGTACGATGAACATCAC |
| | CAAGACTTGACACTTCTCAAGGCCCTAGTCCGTCAGCAACTGCCT |
| | GAGAAATATAAGGAAATATTCTTTGATCAGTCGAAAAACGGGTA |
| | CGCAGGTTATATTGACGGCGGAGCGAGTCAAGAGGAATTCTACA |
| | AGTTTATCAAACCCATATTAGAGAAGATGGATGGGACGGAAGAG |
| | TTGCTTGTAAAACTCAATCGCGAAGATCTACTGCGAAAGCAGCGG |
| | ACTTTCGACAACGGTAGCATTCCACATCAAATCCACTTAGGCGAA |
| | TTGCATGCTATACTTAGAAGGCAGGAGGATTTTTATCCGTTCCTCA |
| | AAGACAATCGTGAAAAGATTGAGAAAATCCTAACCTTTCGCATAC |
| | CTTACTATGTGGGACCCCTGGCCCGAGGGAACTCTCGGTTCGCAT |
| | GGATGACAAGAAAGTCCGAAGAAACGATTACTCCATGGAATTTT |
| | GAGGAAGTTGTCGATAAAGGTGCGTCAGCTCAATCGTTCATCGAG |
| | AGGATGACCAACTTTGACAAGAATTTACCGAACGAAAAAGTATT |
| | GCCTAAGCACAGTTTACTTTACGAGTATTTCACAGTGTACAATGA |
| | ACTCACGAAAGTTAAGTATGTCACTGAGGGCATGCGTAAACCCGC |
| | CTTTCTAAGCGGAGAACAGAAGAAAGCAATAGTAGATCTGTTATT |
| | CAAGACCAACCGCAAAGTGACAGTTAAGCAATTGAAAGAGGACT |
| | ACTTTAAGAAAATTGAATGCTTCGATTCTGTCGAGATCTCCGGGG |
| | TAGAAGATCGATTTAATGCGTCACTTGGTACGTATCATGACCTCC |
| | TAAAGATAATTAAAGATAAGGACTTCCTGGATAACGAAGAGAAT |
| | GAAGATATCTTAGAAGATATAGTGTTGACTCTTACCCTCTTTGAA |
| | GATCGGGAAATGATTGAGGAAAGACTAAAAACATACGCTCACCT |
| | GTTCGACGATAAGGTTATGAAACAGTTAAAGAGGCGTCGCTATAC |
| | GGGCTGGGGACGATTGTCGCGGAAACTTATCAACGGGATAAGAG |
| | ACAAGCAAAGTGGTAAAACTATTCTCGATTTTCTAAAGAGCGACG |
| | GCTTCGCCAATAGGAACTTTATGCAGCTGATCCATGATGACTCTTT |
| | AACCTTCAAAGAGGATATACAAAAGGCACAGGTTTCCGGACAAG |
| | GGGACTCATTGCACGAACATATTGCGAATCTTGCTGGTTCGCCAG |
| | CCATCAAAAAGGGCATACTCCAGACAGTCAAAGTAGTGGATGAG |
| | CTAGTTAAGGTCATGGGACGTCACAAACCGGAAAACATTGTAATC |
| | GAGATGGCACGCGAAAATCAAACGACTCAGAAGGGGCAAAAAA |
| | ACAGTCGAGAGCGGATGAAGAGAATAGAAGAGGGTATTAAAGAA |
| | CTGGGCAGCCAGATCTTAAAGGAGCATCCTGTGGAAAATACCCA |
| | ATTGCAGAACGAGAAACTTTACCTCTATTACCTACAAAATGGAAG |
| | GGACATGTATGTTGATCAGGAACTGGACATAAACCGTTTATCTGA |
| | TTACGACGTCGATCACATTGTACCCCAATCCTTTTTGAAGGACGA |
| | TTCAATCGACAATAAAGTGCTTACACGCTCGGATAAGAACCGAGG |
| | GAAAAGTGACAATGTTCCAAGCGAGGAAGTCGTAAAGAAAATGA |
| | AGAACTATTGGCGGCAGCTCCTAAATGCGAAACTGATAACGCAA |
| | AGAAAGTTCGATAACTTAACTAAAGCTGAGAGGGGGGCTTGTCT |
| | GAACTTGACAAGGCCGGATTTATTAAACGTCAGCTCGTGGAAACC |
| | CGCCAAATCACAAAGCATGTTGCACAGATACTAGATTCCCGAATG |
| | AATACGAAATACGACGAGAACGATAAGCTGATTCGGGAAGTCAA |
| | AGTAATCACTTTAAAGTCAAAATTGGTGTCGGACTTCAGAAAGGA |
| | TTTTCAATTCTATAAAGTTAGGGAGATAAATAACTACCACCATGC |
| | GCACGACGCTTATCTTAATGCCGTCGTAGGGACCGCACTCATTAA |
| | GAAATACCCGAAGCTAGAAAGTGAGTTTGTGTATGGTGATTACAA |
| | AGTTTATGACGTCCGTAAGATGATCGCGAAAAGCGAACAGGAGA |
| | TAGGCAAGGCTACAGCCAAATACTTCTTTTATTCTAACATTATGA |
| | ATTTCTTTAAGACGGAAATCACTCTGGCAAACGGAGAGATACGCA |
| | AACGACCTTTAATTGAAACCAATGGGGAGACAGGTGAAATCGTA |
| | TGGGATAAGGGCCGGGACTTCGCGACGGTGAGAAAAGTTTTGTCC |
| | ATGCCCCAAGTCAACATAGTAAAGAAAACTGAGGTGCAGACCGG |
| | AGGGTTTTCAAAGGAATCGATTCTTCCAAAAAGGAATAGTGATAA |
| | GCTCATCGCTCGTAAAAAGGACTGGGACCCGAAAAAGTACGGTG |
| | GCTTCGATAGCCCTACAGTTGCCTATTCTGTCCTAGTAGTGGCAA |
| | AAGTTGAGAAGGGAAAATCCAAGAAACTGAAGTCAGTCAAAGAA |
| | TTATTGGGGATAACGATTATGGAGCGCTCGTCTTTTGAAAAGAAC |
| | CCCATCGACTTCCTTGAGGCGAAAGGTTACAAGGAAGTAAAAAA |
| | GGATCTCATAATTAAACTACCAAAGTATAGTCTGTTTGAGTTAGA |
| | AAATGGCCGAAAACGGATGTTGGCTAGCGCCGGAGAGCTTCAAA |
| | AGGGGAACGAACTCGCACTACCGTCTAAATACGTGAATTTCCTGT |
| | ATTTAGCGTCCCATTACGAGAAGTTGAAAGGTTCACCTGAAGATA |
| | ACGAACAGAAGCAACTTTTTGTTGAGCAGCACAAACATTATCTCG |
| | ACGAAATCATAGAGCAAATTTCGGAATTCAGTAAGAGAGTCATCC |
| | TAGCTGATGCCAATCTGGACAAAGTATTAAGCGCATACAACAAGC |
| | ACAGGGATAAACCCATACGTGAGCAGGCGGAAAATATTATCCAT |
| | TTGTTTACTCTTACCAACCTCGGCGCTCCAGCCGCATTCAAGTATT |
| | TTGACACAACGATAGATCGCAAACGATACACTTCTACCAAGGAG |
| | GTGCTAGACGCGACACTGATTCACCAATCCATCACGGGATTATAT |
| | GAAACTCGGATAGATTTGTCACAGCTTGGGGGTGACGGATCCCCC |
| | AAGAAGAAGAGGAAAGTCTCGAGCGACTACAAAGACCATGACGG |
| | TGATTATAAAGATCATGACATCGATTACAAGGATGACGATGACAA |
| | GGCTGCAGGA (SEQ ID NO: 57) |
| SpCas9 | MDKKYSIGLDIGTNSVGWAVITDEYKVPSKKFKVLGNTDRHSIKKNL |
| Streptococcus | IGALLFDSGETAEATRLKRTARRRYTRRKNRICYLQEIFSNEMAKVD |
| pyogenes wild | DSFFHRLEESFLVEEDKKHERHPIFGNIVDEVAYHEKYPTIYHLRKKL |
| type | VDSTDKADLRLIYLALAHMIKFRGHFLIEGDLNPDNSDVDKLFIQLV |
| Encoded | QTYNQLFEENPINASGVDAKAILSARLSKSRRLENLIAQLPGEKKNGL |
| product of | FGNLIALSLGLTPNFKSNFDLAEDAKLQLSKDTYDDDLDNLLAQIGD |
| SWBC2D7W014 | QYADLFLAAKNLSDAILLSDILRVNTEITKAPLSASMIKRYDEHHQDL |
| | TLLKALVRQQLPEKYKEIFFDQSKNGYAGYIDGGASQEEFYKFIKPIL |
| | EKMDGTEELLVKLNREDLLRKQRTFDNGSIPHQIHLGELHAILRRQE |
| | DFYPFLKDNREKIEKILTFRIPYYVGPLARGNSRFAWMTRKSEETITP |
| | WNFEEVVDKGASAQSFIERMTNFDKNLPNEKVLPKHSLLYEYFTVY |
| | NELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDIL |
| | EDIVLTLTLFEDREMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKP |
| | ENIVIEMARENQTTQKGQKNSRERMKRIEEGIKELGSQILKEHPVENT |
| | QLQNEKLYLYYLQNGRDMYVDQELDINRLSDYDVDHIVPQSFLKDD |
| | SIDNKVLTRSDKNRGKSDNVPSEEVVKKMKNYWRQLLNAKLITQRK |
| | FDNLTKAERGGLSELDKAGFIKRQLVETRQITKHVAQILDSRMNTKY |
| | DENDKLIREVKVITLKSKLVSDFRKDFQFYKVREINNYHHAHDAYLN |
| | AVVGTALIKKYPKLESEFVYGDYKVYDVRKMIAKSEQEIGKATAKY |
| | FFYSNIMNFFKTEITLANGEIRKRPLIETNGETGEIVWDKGRDFATVR |
| | KVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPI |
| | DFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPA |
| | AFKYFDTTIDRKRYTSTKEVLDATLIHQSITGLYETRIDLSQLGGDGSP |
| | KKKRKVSSDYKDHDGDYKDHDIDYKDDDDKAAG (SEQ ID NO: 42) |
| SpCas9 | ATGGATAAGAAATACTCAATAGGCTTAGATATCGGCACAAATAG |
| Streptococcus | CGTCGGATGGGCGGTGATCACTGATGAATATAAGGTTCCGTCTAA |
| pyogenes | AAAGTTCAAGGTTCTGGGAAATACAGACCGCCACAGTATCAAAA |
| M1GAS wild | AAAATCTTATAGGGGCTCTTTTATTTGACAGTGGAGAGACAGCGG |
| type | AAGCGACTCGTCTCAAACGGACAGCTCGTAGAAGGTATACACGTC |
| NC_002737.2 | GGAAGAATCGTATTTGTTATCTACAGGAGATTTTTTCAAATGAGA |
| | TGGCGAAAGTAGATGATAGTTTCTTTCATCGACTTGAAGAGTCTT |
| | TTTTGGTGGAAGAAGACAAGAAGCATGAACGTCATCCTATTTTTG |
| | GAAATATAGTAGATGAAGTTGCTTATCATGAGAAATATCCAACTA |
| | TCTATCATCTGCGAAAAAAATTGGTAGATTCTACTGATAAAGCGG |
| | ATTTGCGCTTAATCTATTTGGCCTTAGCGCATATGATTAAGTTTCG |
| | TGGTCATTTTTTGATTGAGGGAGATTTAAATCCTGATAATAGTGAT |
| | GTGGACAAACTATTTATCCAGTTGGTACAAACCTACAATCAATTA |
| | TTTGAAGAAAACCCTATTAACGCAAGTGGAGTAGATGCTAAAGC |
| | GATTCTTTCTGCACGATTGAGTAAATCAAGACGATTAGAAAATCT |
| | CATTGCTCAGCTCCCCGGTGAGAAGAAAAATGGCTTATTTGGGAA |
| | TCTCATTGCTTTGTCATTGGGTTTGACCCCTAATTTTAAATCAAAT |
| | TTTGATTTGGCAGAAGATGCTAAATTACAGCTTTCAAAAGATACT |
| | TACGATGATGATTTAGATAATTTATTGGCGCAAATTGGAGATCAA |
| | TATGCTGATTTGTTTTTGGCAGCTAAGAATTTATCAGATGCTATTT |
| | TACTTTCAGATATCCTAAGAGTAAATACTGAAATAACTAAGGCTC |
| | CCCTATCAGCTTCAATGATTAAACGCTACGATGAACATCATCAAG |
| | ACTTGACTCTTTTAAAAGCTTTAGTTCGACAACAACTTCCAGAAA |
| | AGTATAAAGAAATCTTTTTTGATCAATCAAAAAACGGATATGCAG |
| | GTTATATTGATGGGGGAGCTAGCCAAGAAGAATTTTATAAATTTA |
| | TCAAACCAATTTTAGAAAAAATGGATGGTACTGAGGAATTATTGG |
| | TGAAACTAAATCGTGAAGATTTGCTGCGCAAGCAACGGACCTTTG |
| | ACAACGGCTCTATTCCCCATCAAATTCACTTGGGTGAGCTGCATG |
| | CTATTTTGAGAAGACAAGAAGACTTTTATCCATTTTTAAAAGACA |
| | ATCGTGAGAAGATTGAAAAAATCTTGACTTTTCGAATTCCTTATT |
| | ATGTTGGTCCATTGGCGCGTGGCAATAGTCGTTTTGCATGGATGA |
| | CTCGGAAGTCTGAAGAAACAATTACCCCATGGAATTTTGAAGAAG |
| | TTGTCGATAAAGGTGCTTCAGCTCAATCATTTATTGAACGCATGA |
| | CAAACTTTGATAAAAATCTTCCAAATGAAAAAGTACTACCAAAAC |
| | ATAGTTTGCTTTATGAGTATTTTACGGTTTATAACGAATTGACAAA |
| | GGTCAAATATGTTACTGAAGGAATGCGAAAACCAGCATTTCTTTC |
| | AGGTGAACAGAAGAAAGCCATTGTTGATTTACTCTTCAAAACAAA |
| | TCGAAAAGTAACCGTTAAGCAATTAAAAGAAGATTATTTCAAAA |
| | AAATAGAATGTTTTGATAGTGTTGAAATTTCAGGAGTTGAAGATA |
| | GATTTAATGCTTCATTAGGTACCTACCATGATTTGCTAAAAATTAT |
| | TAAAGATAAAGATTTTTTGGATAATGAAGAAAATGAAGATATCTT |
| | AGAGGATATTGTTTTAACATTGACCTTATTTGAAGATAGGGAGAT |
| | GATTGAGGAAAGACTTAAAACATATGCTCACCTCTTTGATGATAA |
| | GGTGATGAAACAGCTTAAACGTCGCCGTTATACTGGTTGGGGACG |
| | TTTGTCTCGAAAATTGATTAATGGTATTAGGGATAAGCAATCTGG |
| | CAAAACAATATTAGATTTTTTGAAATCAGATGGTTTTGCCAATCG |
| | CAATTTTATGCAGCTGATCCATGATGATAGTTTGACATTTAAAGA |
| | AGACATTCAAAAAGCACAAGTGTCTGGACAAGGCGATAGTTTAC |
| | ATGAACATATTGCAAATTTAGCTGGTAGCCCTGCTATTAAAAAAG |
| | GTATTTTACAGACTGTAAAAGTTGTTGATGAATTGGTCAAAGTAA |
| | TGGGGCGGCATAAGCCAGAAAATATCGTTATTGAAATGGCACGT |
| | GAAAATCAGACAACTCAAAAGGGCCAGAAAAATTCGCGAGAGCG |
| | TATGAAACGAATCGAAGAAGGTATCAAAGAATTAGGAAGTCAGA |
| | TTCTTAAAGAGCATCCTGTTGAAAATACTCAATTGCAAAATGAAA |
| | AGCTCTATCTCTATTATCTCCAAAATGGAAGAGACATGTATGTGG |
| | ACCAAGAATTAGATATTAATCGTTTAAGTGATTATGATGTCGATC |
| | ACATTGTTCCACAAAGTTTCCTTAAAGACGATTCAATAGACAATA |
| | AGGTCTTAACGCGTTCTGATAAAAATCGTGGTAAATCGGATAACG |
| | TTCCAAGTGAAGAAGTAGTCAAAAAGATGAAAAACTATTGGAGA |
| | CAACTTCTAAACGCCAAGTTAATCACTCAACGTAAGTTTGATAAT |
| | TTAACGAAAGCTGAACGTGGAGGTTTGAGTGAACTTGATAAAGCT |
| | GGTTTTATCAAACGCCAATTGGTTGAAACTCGCCAAATCACTAAG |
| | CATGTGGCACAAATTTTGGATAGTCGCATGAATACTAAATACGAT |
| | GAAAATGATAAACTTATTCGAGAGGTTAAAGTGATTACCTTAAAA |
| | TCTAAATTAGTTTCTGACTTCCGAAAAGATTTCCAATTCTATAAAG |
| | TACGTGAGATTAACAATTACCATCATGCCCATGATGCGTATCTAA |
| | ATGCCGTCGTTGGAACTGCTTTGATTAAGAAATATCCAAAACTTG |
| | AATCGGAGTTTGTCTATGGTGATTATAAAGTTTATGATGTTCGTAA |
| | AATGATTGCTAAGTCTGAGCAAGAAATAGGCAAAGCAACCGCAA |
| | AATATTTCTTTTACTCTAATATCATGAACTTCTTCAAAACAGAAAT |
| | TACACTTGCAAATGGAGAGATTCGCAAACGCCCTCTAATCGAAAC |
| | TAATGGGGAAACTGGAGAAATTGTCTGGGATAAAGGGCGAGATT |
| | TTGCCACAGTGCGCAAAGTATTGTCCATGCCCCAAGTCAATATTG |
| | TCAAGAAAACAGAAGTACAGACAGGCGGATTCTCCAAGGAGTCA |
| | ATTTTACCAAAAAGAAATTCGGACAAGCTTATTGCTCGTAAAAAA |
| | GACTGGGATCCAAAAAAATATGGTGGTTTTGATAGTCCAACGGTA |
| | GCTTATTCAGTCCTAGTGGTTGCTAAGGTGGAAAAAGGGAAATCG |
| | AAGAAGTTAAAATCCGTTAAAGAGTTACTAGGGATCACAATTATG |
| | GAAAGAAGTTCCTTTGAAAAAAATCCGATTGACTTTTTAGAAGCT |
| | AAAGGATATAAGGAAGTTAAAAAAGACTTAATCATTAAACTACC |
| | TAAATATAGTCTTTTTGAGTTAGAAAACGGTCGTAAACGGATGCT |
| | GGCTAGTGCCGGAGAATTACAAAAAGGAAATGAGCTGGCTCTGC |
| | CAAGCAAATATGTGAATTTTTTATATTTAGCTAGTCATTATGAAA |
| | AGTTGAAGGGTAGTCCAGAAGATAACGAACAAAAACAATTGTTT |
| | GTGGAGCAGCATAAGCATTATTTAGATGAGATTATTGAGCAAATC |
| | AGTGAATTTTCTAAGCGTGTTATTTTAGCAGATGCCAATTTAGATA |
| | AAGTTCTTAGTGCATATAACAAACATAGAGACAAACCAATACGTG |
| | AACAAGCAGAAAATATTATTCATTTATTTACGTTGACGAATCTTG |
| | GAGCTCCCGCTGCTTTTAAATATTTTGATACAACAATTGATCGTAA |
| | ACGATATACGTCTACAAAAGAAGTTTTAGATGCCACTCTTATCCA |
| | TCAATCCATCACTGGTCTTTATGAAACACGCATTGATTTGAGTCA |
| | GCTAGGAGGTGACTGA (SEQ ID NO: 58) |
| SpCas9 | MDKKYSIGLDIGTNSVGWAVITDEYKVPSKKFKVLGNTDRHSIKKNL |
| Streptococcus | IGALLFDSGETAEATRLKRTARRRYTRRKNRICYLQEIFSNEMAKVD |
| pyogenes | DSFFHRLEESFLVEEDKKHERHPIFGNIVDEVAYHEKYPTIYHLRKKL |
| M1GAS wild | VDSTDKADLRLIYLALAHMIKFRGHFLIEGDLNPDNSDVDKLFIQLV |
| type | QTYNQLFEENPINASGVDAKAILSARLSKSRRLENLIAQLPGEKKNGL |
| Encoded | FGNLIALSLGLTPNFKSNFDLAEDAKLQLSKDTYDDDLDNLLAQIGD |
| product of | QYADLFLAAKNLSDAILLSDILRVNTEITKAPLSASMIKRYDEHHQDL |
| NC_002737.2 | TLLKALVRQQLPEKYKEIFFDQSKNGYAGYIDGGASQEEFYKFIKPIL |
| (100% identical | EKMDGTEELLVKLNREDLLRKQRTFDNGSIPHQIHLGELHAILRRQE |
| to the canonical | DFYPFLKDNREKIEKILTFRIPYYVGPLARGNSRFAWMTRKSEETITP |
| Q99ZW2 | WNFEEVVDKGASAQSFIERMTNFDKNLPNEKVLPKHSLLYEYFTVY |
| wild type) | NELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDIL |
| | EDIVLTLTLFEDREMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKP |
| | ENIVIEMARENQTTQKGQKNSRERMKRIEEGIKELGSQILKEHPVENT |
| | QLQNEKLYLYYLQNGRDMYVDQELDINRLSDYDVDHIVPQSFLKDD |
| | SIDNKVLTRSDKNRGKSDNVPSEEVVKKMKNYWRQLLNAKLITQRK |
| | FDNLTKAERGGLSELDKAGFIKRQLVETRQITKHVAQILDSRMNTKY |
| | DENDKLIREVKVITLKSKLVSDFRKDFQFYKVREINNYHHAHDAYLN |
| | AVVGTALIKKYPKLESEFVYGDYKVYDVRKMIAKSEQEIGKATAKY |
| | FFYSNIMNFFKTEITLANGEIRKRPLIETNGETGEIVWDKGRDFATVR |
| | KVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPI |
| | DFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| | (SEQ ID NO: 37) |

The PE-VLPs and prime editor fusion proteins described herein may include any of the above SpCas9 sequences, or any variant thereof having at least 80%, at least 85%, at least 90%, at least 95%, or at least 99% sequence identity thereto. In other embodiments, the Cas9 protein can be a wild type Cas9 ortholog from another bacterial species different from the canonical Cas9 from S. pyogenes. For example, modified versions of the following Cas9 orthologs can be used in connection with the PE-VLPs and fusion proteins described in this specification by making mutations at positions corresponding to H840A or any other amino acids of interest in wild type SpCas9. In addition, any variant Cas9 orthologs having at least 80%, at least 85%, at least 90%, at least 95%, or at least 99% sequence identity to any of the below orthologs may also be used with the prime editors.

| Description | Sequence |
|---|---|
| LfCas9 | MKEYHIGLDIGTSSIGWAVTDSQFKLMRIKGKTAIGVRLFEEGKTAAERR |
| Lactobacillus | TFRTTRRRLKRRKWRLHYLDEIFAPHLQEVDENFLRRLKQSNIHPEDPTK |
| fermentum wild | NQAFIGKLLFPDLLKKNERGYPTLIKMRDELPVEQRAHYPVMNIYKLRE |
| type | AMINEDRQFDLREVYLAVHHIVKYRGHFLNNASVDKFKVGRIDFDKSFN |
| GenBank: | VLNEAYEELQNGEGSFTIEPSKVEKIGQLLLDTKMRKLDRQKAVAKLLE |
| SNX31424.1 1 | VKVADKEETKRNKQIATAMSKLVLGYKADFATVAMANGNEWKIDLSS |
| | ETSEDEIEKFREELSDAQNDILTEITSLFSQIMLNEIVPNGMSISESMMDRY |
| | WTHERQLAEVKEYLATQPASARKEFDQVYNKYIGQAPKERGFDLEKGL |
| | KKILSKKENWKEIDELLKAGDFLPKQRTSANGVIPHQMHQQELDRIIEKQ |
| | AKYYPWLATENPATGERDRHQAKYELDQLVSFRIPYYVGPLVTPEVQK |
| | ATSGAKFAWAKRKEDGEITPWNLWDKIDRAESAEAFIKRMTVKDTYLL |
| | NEDVLPANSLLYQKYNVLNELNNVRVNGRRLSVGIKQDIYTELFKKKKT |
| | VKASDVASLVMAKTRGVNKPSVEGLSDPKKFNSNLATYLDLKSIVGDK |
| | VDDNRYQTDLENIIEWRSVFEDGEIFADKLTEVEWLTDEQRSALVKKRY |
| | KGWGRLSKKLLTGIVDENGQRIIDLMWNTDQNFKEIVDQPVFKEQIDQL |
| | NQKAITNDGMTLRERVESVLDDAYTSPQNKKAIWQVVRVVEDIVKAVG |
| | NAPKSISIEFARNEGNKGEITRSRRTQLQKLFEDQAHELVKDTSLTEELEK |
| | APDLSDRYYFYFTQGGKDMYTGDPINFDEISTKYDIDHILPQSFVKDNSL |
| | DNRVLTSRKENNKKSDQVPAKLYAAKMKPYWNQLLKQGLITQRKFEN |
| | LTKDVDQNIKYRSLGFVKRQLVETRQVIKLTANILGSMYQEAGTEIIETR |
| | AGLTKQLREEFDLPKVREVNDYHHAVDAYLTTFAGQYLNRRYPKLRSF |
| | FVYGEYMKFKHGSDLKLRNFNFFHELMEGDKSQGKVVDQQTGELITTR |
| | DEVAKSFDRLLNMKYMLVSKEVHDRSDQLYGATIVTAKESGKLTSPIEI |
| | KKNRLVDLYGAYTNGTSAFMTIIKFTGNKPKYKVIGIPTTSAASLKRAGK |
| | PGSESYNQELHRIIKSNPKVKKGFEIVVPHVSYGQLIVDGDCKFTLASPTV |
| | QHPATQLVLSKKSLETISSGYKILKDKPAIANERLIRVFDEVVGQMNRYF |
| | TIFDQRSNRQKVADARDKFLSLPTESKYEGAKKVQVGKTEVITNLLMGL |
| | HANATQGDLKVLGLATFGFFQSTTGLSLSEDTMIVYQSPTGLFERRICLK |
| | DI (SEQ ID NO: 43) |
| SaCas9 | MDKKYSIGLDIGTNSVGWAVITDEYKVPSKKFKVLGNTDRHSIKKNLIG |
| Staphylococcus | ALLFDSGETAEATRLKRTARRRYTRRKNRICYLQEIFSNEMAKVDDSFFH |
| aureus | RLEESFLVEEDKKHERHPIFGNIVDEVAYHEKYPTIYHLRKKLVDSTDKA |
| wild type | DLRLIYLALAHMIKFRGHFLIEGDLNPDNSDVDKLFIQLVQTYNQLFEEN |
| GenBank: | PINASGVDAKAILSARLSKSRRLENLIAQLPGEKKNGLFGNLIALSLGLTP |
| AYD60528.1 | NFKSNFDLAEDAKLQLSKDTYDDDLDNLLAQIGDQYADLFLAAKNLSD |
| | AILLSDILRVNTEITKAPLSASMIKRYDEHHQDLTLLKALVRQQLPEKYK |
| | EIFFDQSKNGYAGYIDGGASQEEFYKFIKPILEKMDGTEELLVKLNREDL |
| | LRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFD |
| | KNLPNEKVLPKHSLLYEYFTVYNELTKVKYVTEGMRKPAFLSGEQKKAI |
| | VDLLFKTNRKVTVKQLKEDYFKKIECFDSVEISGVEDRFNASLGTYHDL |
| | LKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLI |
| | HDDSLTFKEDIQKAQVSGQGDSLHEHIANLAGSPAIKKGILQTVKVVDEL |
| | VKVMGRHKPENIVIEMARENQTTQKGQKNSRERMKRIEEGIKELGSQIL |
| | KEHPVENTQLQNEKLYLYYLQNGRDMYVDQELDINRLSDYDVDHIVPQ |
| | SFLKDDSIDNKVLTRSDKNRGKSDNVPSEEVVKKMKNYWRQLLNAKLI |
| | TQRKFDNLTKAERGGLSELDKAGFIKRQLVETRQITKHVAQILDSRMNT |
| | KYDENDKLIREVKVITLKSKLVSDFRKDFQFYKVREINNYHHAHDAYLN |
| | AVVGTALIKKYPKLESEFVYGDYKVYDVRKMIAKSEQEIGKATAKYFFY |
| | SNIMNFFKTEITLANGEIRKRPLIETNGETGEIVWDKGRDFATVRKVLSM |
| | PQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYK |
| | EVKKDLIIKLPKYSLFELENGRKRMLASAGELQKGNELALPSKYVNFLY |
| | LASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYT |
| | STKEVLDATLIHQSITGLYETRIDLSQLGGD (SEQ ID NO: 37) |
| SaCas9 | MGKRNYILGLDIGITSVGYGIIDYETRDVIDAGVRLFKEANVENNEGRRS |
| Staphylococcus | KRGARRLKRRRRHRIQRVKKLLFDYNLLTDHSELSGINPYEARVKGLSQ |
| aureus | KLSEEEFSAALLHLAKRRGVHNVNEVEEDTGNELSTKEQISRNSKALEE |
| | KYVAELQLERLKKDGEVRGSINRFKTSDYVKEAKQLLKVQKAYHQLDQ |
| | SFIDTYIDLLETRRTYYEGPGEGSPFGWKDIKEWYEMLMGHCTYFPEELR |
| | SVKYAYNADLYNALNDLNNLVITRDENEKLEYYEKFQIIENVFKQKKKP |
| | TLKQIAKEILVNEEDIKGYRVTSTGKPEFTNLKVYHDIKDITARKEIIENA |
| | ELLDQIAKILTIYQSSEDIQEELTNLNSELTQEEIEQISNLKGYTGTHNLSL |
| | KAINLILDELWHTNDNQIAIFNRLKLVPKKVDLSQQKEIPTTLVDDFILSP |
| | VVKRSFIQSIKVINAIIKKYGLPNDIIIELAREKNSKDAQKMINEMQKRNR |
| | QTNERIEEIIRTTGKENAKYLIEKIKLHDMQEGKCLYSLEAIPLEDLLNNP |
| | FNYEVDHIIPRSVSFDNSFNNKVLVKQEENSKKGNRTPFQYLSSSDSKISY |
| | ETFKKHILNLAKGKGRISKTKKEYLLEERDINRFSVQKDFINRNLVDTRY |
| | ATRGLMNLLRSYFRVNNLDVKVKSINGGFTSFLRRKWKFKKERNKGYK |
| | HHAEDALIIANADFIFKEWKKLDKAKKVMENQMFEEKQAESMPEIETEQ |
| | EYKEIFITPHQIKHIKDFKDYKYSHRVDKKPNRKLINDTLYSTRKDDKGN |
| | TLIVNNLNGLYDKDNDKLKKLINKSPEKLLMYHHDPQTYQKLKLIMEQ |
| | YGDEKNPLYKYYEETGNYLTKYSKKDNGPVIKKIKYYGNKLNAHLDIT |
| | DDYPNSRNKVVKLSLKPYRFDVYLDNGVYKFVTVKNLDVIKKENYYEV |
| | NSKCYEEAKKLKKISNQAEFIASFYKNDLIKINGELYRVIGVNNDLLNRIE |
| | VNMIDITYREYLENMNDKRPPHIIKTIASKTQSIKKYSTDILGNLYEVKSK |
| | KHPQIIKK (SEQ ID NO: 44) |
| StCas9 | MLFNKCIIISINLDFSNKEKCMTKPYSIGLDIGTNSVGWAVITDNYKVPSK |
| Streptococcus | KMKVLGNTSKKYIKKNLLGVLLFDSGITAEGRRLKRTARRRYTRRRNRI |
| thermophilus | LYLQEIFSTEMATLDDAFFQRLDDSFLVPDDKRDSKYPIFGNLVEEKVYH |
| UniProtKB/ | DEFPTIYHLRKYLADSTKKADLRLVYLALAHMIKYRGHFLIEGEFNSKN |
| Swiss-Prot: | NDIQKNFQDFLDTYNAIFESDLSLENSKQLEEIVKDKISKLEKKDRILKLF |
| G3ECR1.2 | PGEKNSGIFSEFLKLIVGNQADFRKCFNLDEKASLHFSKESYDEDLETLL |
| Wild type | GYIGDDYSDVFLKAKKLYDAILLSGFLTVTDNETEAPLSSAMIKRYNEH |
| | KEDLALLKEYIRNISLKTYNEVFKDDTKNGYAGYIDGKTNQEDFYVYLK |
| | NLLAEFEGADYFLEKIDREDFLRKQRTFDNGSIPYQIHLQEMRAILDKQA |
| | KFYPFLAKNKERIEKILTFRIPYYVGPLARGNSDFAWSIRKRNEKITPWNF |
| | EDVIDKESSAEAFINRMTSFDLYLPEEKVLPKHSLLYETFNVYNELTKVR |
| | FIAESMRDYQFLDSKQKKDIVRLYFKDKRKVTDKDIIEYLHAIYGYDGIE |
| | LKGIEKQFNSSLSTYHDLLNIINDKEFLDDSSNEAIIEEIIHTLTIFEDREMIK |
| | QRLSKFENIFDKSVLKKLSRRHYTGWGKLSAKLINGIRDEKSGNTILDYLI |
| | DDGISNRNFMQLIHDDALSFKKKIQKAQIIGDEDKGNIKEVVKSLPGSPAI |
| | KKGILQSIKIVDELVKVMGGRKPESIVVEMARENQYTNQGKSNSQQRLK |
| | RLEKSLKELGSKILKENIPAKLSKIDNNALQNDRLYLYYLQNGKDMYTG |
| | DDLDIDRLSNYDIDHIIPQAFLKDNSIDNKVLVSSASNRGKSDDFPSLEVV |
| | KKRKTFWYQLLKSKLISQRKFDNLTKAERGGLLPEDKAGFIQRQLVETR |
| | QITKHVARLLDEKFNNKKDENNRAVRTVKIITLKSTLVSQFRKDFELYK |
| | VREINDFHHAHDAYLNAVIASALLKKYPKLEPEFVYGDYPKYNSFRERK |
| | SATEKVYFYSNIMNIFKKSISLADGRVIERPLIEVNEETGESVWNKESDLA |
| | TVRRVLSYPQVNVVKKVEEQNHGLDRGKPKGLFNANLSSKPKPNSNEN |
| | LVGAKEYLDPKKYGGYAGISNSFAVLVKGTIEKGAKKKITNVLEFQGISI |
| | LDRINYRKDKLNFLLEKGYKDIELIIELPKYSLFELSDGSRRMLASILSTN |
| | NKRGEIHKGNQIFLSQKFVKLLYHAKRISNTINENHRKYVENHKKEFEEL |
| | FYYILEFNENYVGAKKNGKLLNSAFQSWQNHSIDELCSSFIGPTGSERKG |
| | LFELTSRGSAADFEFLGVKIPRYRDYTPSSLLKDATLIHQSVTGLYETRID |
| | LAKLGEG (SEQ ID NO: 45) |
| LcCas9 | MKIKNYNLALTPSTSAVGHVEVDDDLNILEPVHHQKAIGVAKFGEGETA |
| Lactobacillus | EARRLARSARRTTKRRANRINHYFNEIMKPEIDKVDPLMFDRIKQAGLSP |
| crispatus | LDERKEFRTVIFDRPNIASYYHNQFPTIWHLQKYLMITDEKADIRLIYWA |
| NCBI | LHSLLKHRGHFFNTTPMSQFKPGKLNLKDDMLALDDYNDLEGLSFAVA |
| Reference | NSPEIEKVIKDRSMHKKEKIAELKKLIVNDVPDKDLAKRNNKIITQIVNAI |
| Sequence: | MGNSFHLNFIFDMDLDKLTSKAWSFKLDDPELDTKFDAISGSMTDNQIGI |
| WP_ | FETLQKIYSAISLLDILNGSSNVVDAKNALYDKHKRDLNLYFKFLNTLPD |
| 133478044.1 | EIAKTLKAGYTLYIGNRKKDLLAARKLLKVNVAKNFSQDDFYKLINKEL |
| Wild type | KSIDKQGLQTRFSEKVGELVAQNNFLPVQRSSDNVFIPYQLNAITFNKILE |
| | NQGKYYDFLVKPNPAKKDRKNAPYELSQLMQFTIPYYVGPLVTPEEQV |
| | KSGIPKTSRFAWMVRKDNGAITPWNFYDKVDIEATADKFIKRSIAKDSY |
| | LLSELVLPKHSLLYEKYEVFNELSNVSLDGKKLSGGVKQILFNEVFKKTN |
| | KVNTSRILKALAKHNIPGSKITGLSNPEEFTSSLQTYNAWKKYFPNQIDNF |
| | AYQQDLEKMIEWSTVFEDHKILAKKLDEIEWLDDDQKKFVANTRLRGW |
| | GRLSKRLLTGLKDNYGKSIMQRLETTKANFQQIVYKPEFREQIDKISQAA |
| | AKNQSLEDILANSYTSPSNRKAIRKTMSVVDEYIKLNHGKEPDKIFLMFQ |
| | RSEQEKGKQTEARSKQLNRILSQLKADKSANKLESKQLADEFSNAIKKS |
| | KYKLNDKQYFYFQQLGRDALTGEVIDYDELYKYTVLHIIPRSKLTDDSQ |
| | NNKVLTKYKIVDGSVALKFGNSYSDALGMPIKAFWTELNRLKLIPKGKL |
| | LNLTTDFSTLNKYQRDGYIARQLVETQQIVKLLATIMQSRFKHTKIIEVR |
| | NSQVANIRYQFDYFRIKNLNEYYRGFDAYLAAVVGTYLYKVYPKARRL |
| | FVYGQYLKPKKTNQENQDMHLDSEKKSQGFNFLWNLLYGKQDQIFVN |
| | GTDVIAFNRKDLITKMNTVYNYKSQKISLAIDYHNGAMFKATLFPRNDR |
| | DTAKTRKLIPKKKDYDTDIYGGYTSNVDGYMLLAEIIKRDGNKQYGFYG |
| | VPSRLVSELDTLKKTRYTEYEEKLKEIIKPELGVDLKKIKKIKILKNKVPF |
| | NQVIIDKGSKFFITSTSYRWNYRQLILSAESQQTLMDLVVDPDFSNHKAR |
| | KDARKNADERLIKVYEEILYQVKNYMPMFVELHRCYEKLVDAQKTFKS |
| | LKISDKAMVLNQILILLHSNATSPVLEKLGYHTRFTLGKKHNLISENAVL |
| | VTQSITGLKENHVSIKQML (SEQ ID NO: 46) |
| PdCas9 | MTNEKYSIGLDIGTSSIGFAVVNDNNRVIRVKGKNAIGVRLFDEGKAAA |
| Pedicoccus | DRRSFRTTRRSFRTTRRRLSRRRWRLKLLREIFDAYITPVDEAFFIRLKES |
| damnosus | NLSPKDSKKQYSGDILFNDRSDKDFYEKYPTIYHLRNALMTEHRKFDVR |
| NCBI | EIYLAIHHIMKFRGHFLNATPANNFKVGRLNLEEKFEELNDIYQRVFPDE |
| Reference | SIEFRTDNLEQIKEVLLDNKRSRADRQRTLVSDIYQSSEDKDIEKRNKAV |
| Sequence: | ATEILKASLGNKAKLNVITNVEVDKEAAKEWSITFDSESIDDDLAKIEGQ |
| WP_ | MTDDGHEIIEVLRSLYSGITLSAIVPENHTLSQSMVAKYDLHKDHLKLFK |
| 062913273.1 | KLINGMTDTKKAKNLRAAYDGYIDGVKGKVLPQEDFYKQVQVNLDDS |
| Wild type | AEANEIQTYIDQDIFMPKQRTKANGSIPHQLQQQELDQIIENQKAYYPWL |
| | AELNPNPDKKRQQLAKYKLDELVTFRVPYYVGPMITAKDQKNQSGAEF |
| | AWMIRKEPGNITPWNFDQKVDRMATANQFIKRMTTTDTYLLGEDVLPA |
| | QSLLYQKFEVLNELNKIRIDHKPISIEQKQQIFNDLFKQFKNVTIKHLQDY |
| | LVSQGQYSKRPLIEGLADEKRFNSSLSTYSDLCGIFGAKLVEENDRQEDL |
| | EKIIEWSTIFEDKKIYRAKLNDLTWLTDDQKEKLATKRYQGWGRLSRKL |
| | LVGLKNSEHRNIMDILWITNENFMQIQAEPDFAKLVTDANKGMLEKTDS |
| | QDVINDLYTSPQNKKAIRQILLVVHDIQNAMHGQAPAKIHVEFARGEER |
| | NPRRSVQRQRQVEAAYEKVSNELVSAKVRQEFKEAINNKRDFKDRLFL |
| | YFMQGGIDIYTGKQLNIDQLSSYQIDHILPQAFVKDDSLTNRVLTNENQV |
| | KADSVPIDIFGKKMLSVWGRMKDQGLISKGKYRNLTMNPENISAHTENG |
| | FINRQLVETRQVIKLAVNILADEYGDSTQIISVKADLSHQMREDFELLKN |
| | RDVNDYHHAFDAYLAAFIGNYLLKRYPKLESYFVYGDFKKFTQKETKM |
| | RRFNFIYDLKHCDQVVNKETGEILWTKDEDIKYIRHLFAYKKILVSHEVR |
| | EKRGALYNQTIYKAKDDKGSGQESKKLIRIKDDKETKIYGGYSGKSLAY |
| | MTIVQITKKNKVSYRVIGIPTLALARLNKLENDSTENNGELYKIIKPQFTH |
| | YKVDKKNGEIIETTDDFKIVVSKVRFQQLIDDAGQFFMLASDTYKNNAQ |
| | QLVISNNALKAINNTNITDCPRDDLERLDNLRLDSAFDEIVKKMDKYFSA |
| | YDANNFREKIRNSNLIFYQLPVEDQWENNKITELGKRTVLTRILQGLHAN |
| | ATTTDMSIFKIKTPFGQLRQRSGISLSENAQLIYQSPTGLFERRVQLNKIK |
| | (SEQ ID NO: 47) |
| FnCas9 | MKKQKFSDYYLGFDIGTNSVGWCVTDLDYNVLRFNKKDMWGSRLFEE |
| Fusobaterium | AKTAAERRVQRNSRRRLKRRKWRLNLLEEIFSNEILKIDSNFFRRLKESSL |
| nucleatum | WLEDKSSKEKFTLFNDDNYKDYDFYKQYPTIFHLRNELIKNPEKKDIRLV |
| NCBI | YLAIHSIFKSRGHFLFEGQNLKEIKNFETLYNNLIAFLEDNGINKIIDKNNI |
| Reference | EKLEKIVCDSKKGLKDKEKEFKEIFNSDKQLVAIFKLSVGSSVSLNDLFD |
| Sequence: | TDEYKKGEVEKEKISFREQIYEDDKPIYYSILGEKIELLDIAKTFYDFMVL |
| WP_ | NNILADSQYISEAKVKLYEEHKKDLKNLKYIIRKYNKGNYDKLFKDKNE |
| 060798984.1 | NNYSAYIGLNKEKSKKEVIEKSRLKIDDLIKNIKGYLPKVEEIEEKDKAIF |
| | NKILNKIELKTILPKQRISDNGTLPYQIHEAELEKILENQSKYYDFLNYEE |
| | NGIITKDKLLMTFKFRIPYYVGPLNSYHKDKGGNSWIVRKEEGKILPWNF |
| | EQKVDIEKSAEEFIKRMTNKCTYLNGEDVIPKDTFLYSEYVILNELNKVQ |
| | VNDEFLNEENKRKIIDELFKENKKVSEKKFKEYLLVKQIVDGTIELKGVK |
| | DSFNSNYISYIRFKDIFGEKLNLDIYKEISEKSILWKCLYGDDKKIFEKKIK |
| | NEYGDILTKDEIKKINTFKFNNWGRLSEKLLTGIEFINLETGECYSSVMDA |
| | LRRTNYNLMELLSSKFTLQESINNENKEMNEASYRDLIEESYVSPSLKRAI |
| | FQTLKIYEEIRKITGRVPKKVFIEMARGGDESMKNKKIPARQEQLKKLYD |
| | SCGNDIANFSIDIKEMKNSLISYDNNSLRQKKLYLYYLQFGKCMYTGREI |
| | DLDRLLQNNDTYDIDHIYPRSKVIKDDSFDNLVLVLKNENAEKSNEYPV |
| | KKEIQEKMKSFWRFLKEKNFISDEKYKRLTGKDDFELRGFMARQLVNV |
| | RQTTKEVGKILQQIEPEIKIVYSKAEIASSFREMFDFIKVRELNDTHHAKD |
| | AYLNIVAGNVYNTKFTEKPYRYLQEIKENYDVKKIYNYDIKNAWDKEN |
| | SLEIVKKNMEKNTVNITRFIKEKKGQLFDLNPIKKGETSNEIISIKPKVYN |
| | GKDDKLNEKYGYYKSLNPAYFLYVEHKEKNKRIKSFERVNLVDVNNIK |
| | DEKSLVKYLIENKKLVEPRVIKKVYKRQVILINDYPYSIVTLDSNKLMDF |
| | ENLKPLFLENKYEKILKNVIKFLEDNQGKSEENYKFIYLKKKDRYEKNET |
| | LESVKDRYNLEFNEMYDKFLEKLDSKDYKNYMNNKKYQELLDVKEKFI |
| | KLNLFDKAFTLKSFLDLFNRKTMADFSKVGLTKYLGKIQKISSNVLSKNE |
| | LYLLEESVTGLFVKKIKL (SEQ ID NO: 48) |
| EcCas9 | RRKQRIQILQELLGEEVLKTDPGFFHRMKESRYVVEDKRTLDGKQVELP |
| Enterococcus | YALFVDKDYTDKEYYKQFPTINHLIVYLMTTSDTPDIRLVYLALHYYMK |
| cecorum | NRGNFLHSGDINNVKDINDILEQLDNVLETFLDGWNLKLKSYVEDIKNIY |
| NCBI | NRDLGRGERKKAFVNTLGAKTKAEKAFCSLISGGSTNLAELFDDSSLKEI |
| Reference | ETPKIEFASSSLEDKIDGIQEALEDRFAVIEAAKRLYDWKTLTDILGDSSS |
| Sequence: | LAEARVNSYQMHHEQLLELKSLVKEYLDRKVFQEVFVSLNVANNYPAY |
| WP_ | IGHTKINGKKKELEVKRTKRNDFYSYVKKQVIEPIKKKVSDEAVLTKLSE |
| 047338501.1 | IESLIEVDKYLPLQVNSDNGVIPYQVKLNELTRIFDNLENRIPVLRENRDK |
| Wild type | IIKTFKFRIPYYVGSLNGVVKNGKCTNWMVRKEEGKIYPWNFEDKVDLE |
| | ASAEQFIRRMTNKCTYLVNEDVLPKYSLLYSKYLVLSELNNLRIDGRPLD |
| | VKIKQDIYENVFKKNRKVTLKKIKKYLLKEGIITDDDELSGLADDVKSSL |
| | TAYRDFKEKLGHLDLSEAQMENIILNITLFGDDKKLLKKRLAALYPFIDD |
| | KSLNRIATLNYRDWGRLSERFLSGITSVDQETGELRTIIQCMYETQANLM |
| | QLLAEPYHFVEAIEKENPKVDLESISYRIVNDLYVSPAVKRQIWQTLLVIK |
| | DIKQVMKHDPERIFIEMAREKQESKKTKSRKQVLSEVYKKAKEYEHLFE |
| | KLNSLTEEQLRSKKIYLYFTQLGKCMYSGEPIDFENLVSANSNYDIDHIYP |
| | QSKTIDDSFNNIVLVKKSLNAYKSNHYPIDKNIRDNEKVKTLWNTLVSK |
| | GLITKEKYERLIRSTPFSDEELAGFIARQLVETRQSTKAVAEILSNWFPESE |
| | IVYSKAKNVSNFRQDFEILKVRELNDCHHAHDAYLNIVVGNAYHTKFTN |
| | SPYRFIKNKANQEYNLRKLLQKVNKIESNGVVAWVGQSENNPGTIATVK |
| | KVIRRNTVLISRMVKEVDGQLFDLTLMKKGKGQVPIKSSDERLTDISKY |
| | GGYNKATGAYFTFVKSKKRGKVVRSFEYVPLHLSKQFENNNELLKEYIE |
| | KDRGLTDVEILIPKVLINSLFRYNGSLVRITGRGDTRLLLVHEQPLYVSNS |
| | FVQQLKSVSSYKLKKSENDNAKLTKTATEKLSNIDELYDGLLRKLDLPIY |
| | SYWFSSIKEYLVESRTKYIKLSIEEKALVIFEILHLFQSDAQVPNLKILGLS |
| | TKPSRIRIQKNLKDTDKMSIIHQSPSGIFEHEIELTSL (SEQ ID NO: 49) |
| AhCas9 | MQNGFLGITVSSEQVGWAVINPKYELERASRKDLWGVRLFDKAETAED |
| Anaerostipes | RRMFRTNRRLNQRKKNRIHYLRDIFHEEVNQKDPNFFQQLDESNFCEDD |
| hadrus | RTVEFNFDTNLYKNQFPTVYHLRKYLMETKDKPDIRLVYLAFSKFMKN |
| NCBI | RGHFLYKGNLGEVMDFENSMKGFCESLEKFNIDFPTLSDEQVKEVRDIL |
| Reference | CDHKIAKTVKKKNIITITKVKSKTAKAWIGLFCGCSVPVKVLFQDIDEEIV |
| Sequence: | TDPEKISFEDASYDDYIANIEKGVGIYYEAIVSAKMLFDWSILNEILGDHQ |
| WP_ | LLSDAMIAEYNKHHDDLKRLQKIIKGTGSRELYQDIFINDVSGNYVCYV |
| 044924278.1 | GHAKTMSSADQKQFYTFLKNRLKNVNGISSEDAEWIDTEIKNGTLLPKQ |
| Wild type | TKRDNSVIPHQLQLREFELILDNMQEMYPFLKENREKLLKIFNFVIPYYV |
| | GPLKGVVRKGESTNWMVPKKDGVIHPWNFDEMVDKEASAECFISRMT |
| | GNCSYLFNEKVLPKNSLLYETFEVLNELNPLKINGEPISVELKQRIYEQLF |
| | LTGKKVTKKSLTKYLIKNGYDKDIELSGIDNEFHSNLKSHIDFEDYDNLS |
| | DEEVEQIILRITVFEDKQLLKDYLNREFVKLSEDERKQICSLSYKGWGNL |
| | SEMLLNGITVTDSNGVEVSVMDMLWNTNLNLMQILSKKYGYKAEIEHY |
| | NKEHEKTIYNREDLMDYLNIPPAQRRKVNQLITIVKSLKKTYGVPNKIFF |
| | KISREHQDDPKRTSSRKEQLKYLYKSLKSEDEKHLMKELDELNDHELSN |
| | DKVYLYFLQKGRCIYSGKKLNLSRLRKSNYQNDIDYIYPLSAVNDRSMN |
| | NKVLTGIQENRADKYTYFPVDSEIQKKMKGFWMELVLQGFMTKEKYFR |
| | LSRENDESKSELVSFIEREISDNQQSGRMIASVLQYYFPESKIVFVKEKLIS |
| | SFKRDFHLISSYGHNHLQAAKDAYITIVVGNVYHTKFTMDPAIYFKNHK |
| | RKDYDLNRLFLENISRDGQIAWESGPYGSIQTVRKEYAQNHIAVTKRVV |
| | EVKGGLFKQMPLKKGHGEYPLKTNDPRFGNIAQYGGYTNVTGSYFVLV |
| | ESMEKGKKRISLEYVPVYLHERLEDDPGHKLLKEYLVDHRKLNHPKILL |
| | AKVRKNSLLKIDGFYYRLNGRSGNALILTNAVELIMDDWQTKTANKISG |
| | YMKRRAIDKKARVYQNEFHIQELEQLYDFYLDKLKNGVYKNRKNNQA |
| | ELIHNEKEQFMELKTEDQCVLLTEIKKLFVCSPMQADLTLIGGSKHTGMI |
| | AMSSNVTKADFAVIAEDPLGLRNKVIYSHKGEK (SEQ ID NO: 50) |
| KvCas9 | MSQNNNKIYNIGLDIGDASVGWAVVDEHYNLLKRHGKHMWGSRLFTQ |
| Kandleria | ANTAVERRSSRSTRRRYNKRRERIRLLREIMEDMVLDVDPTFFIRLANVS |
| vitulina | FLDQEDKKDYLKENYHSNYNLFIDKDFNDKTYYDKYPTIYHLRKHLCES |
| NCBI | KEKEDPRLIYLALHHIVKYRGNFLYEGQKFSMDVSNIEDKMIDVLRQFN |
| Reference | EINLFEYVEDRKKIDEVLNVLKEPLSKKHKAEKAFALFDTTKDNKAAYK |
| Sequence: | ELCAALAGNKFNVTKMLKEAELHDEDEKDISFKFSDATFDDAFVEKQPL |
| WP_ | LGDCVEFIDLLHDIYSWVELQNILGSAHTSEPSISAAMIQRYEDHKNDLK |
| 031589969.1 | LLKDVIRKYLPKKYFEVFRDEKSKKNNYCNYINHPSKTPVDEFYKYIKK |
| Wild type | LIEKIDDPDVKTILNKIELESFMLKQNSRTNGAVPYQMQLDELNKILENQ |
| | SVYYSDLKDNEDKIRSILTFRIPYYFGPLNITKDRQFDWIIKKEGKENERIL |
| | PWNANEIVDVDKTADEFIKRMRNFCTYFPDEPVMAKNSLTVSKYEVLN |
| | EINKLRINDHLIKRDMKDKMLHTLFMDHKSISANAMKKWLVKNQYFSN |
| | TDDIKIEGFQKENACSTSLTPWIDFTKIFGKINESNYDFIEKIIYDVTVFED |
| | KKILRRRLKKEYDLDEEKIKKILKLKYSGWSRLSKKLLSGIKTKYKDSTR |
| | TPETVLEVMERTNMNLMQVINDEKLGFKKTIDDANSTSVSGKFSYAEVQ |
| | ELAGSPAIKRGIWQALLIVDEIKKIMKHEPAHVYIEFARNEDEKERKDSF |
| | VNQMLKLYKDYDFEDETEKEANKHLKGEDAKSKIRSERLKLYYTQMG |
| | KCMYTGKSLDIDRLDTYQVDHIVPQSLLKDDSIDNKVLVLSSENQRKLD |
| | DLVIPSSIRNKMYGFWEKLFNNKIISPKKFYSLIKTEFNEKDQERFINRQIV |
| | ETRQITKHVAQIIDNHYENTKVVTVRADLSHQFRERYHIYKNRDINDFHH |
| | AHDAYIATILGTYIGHRFESLDAKYIYGEYKRIFRNQKNKGKEMKKNND |
| | GFILNSMRNIYADKDTGEIVWDPNYIDRIKKCFYYKDCFVTKKLEENNG |
| | TFFNVTVLPNDTNSDKDNTLATVPVNKYRSNVNKYGGFSGVNSFIVAIK |
| | GKKKKGKKVIEVNKLTGIPLMYKNADEEIKINYLKQAEDLEEVQIGKEIL |
| | KNQLIEKDGGLYYIVAPTEIINAKQLILNESQTKLVCEIYKAMKYKNYDN |
| | LDSEKIIDLYRLLINKMELYYPEYRKQLVKKFEDRYEQLKVISIEEKCNII |
| | KQILATLHCNSSIGKIMYSDFKISTTIGRLNGRTISLDDISFIAESPTGMYSK |
| | KYKL (SEQ ID NO: 51) |
| EfCas9 | MRLFEEGHTAEDRRLKRTARRRISRRRNRLRYLQAFFEEAMTDLDENFF |
| Enterococcus | ARLQESFLVPEDKKWHRHPIFAKLEDEVAYHETYPTIYHLRKKLADSSE |
| faecalis | QADLRLIYLALAHIVKYRGHFLIEGKLSTENTSVKDQFQQFMVIYNQTFV |
| NCBI | NGESRLVSAPLPESVLIEEELTEKASRTKKSEKVLQQFPQEKANGLFGQF |
| Reference | LKLMVGNKADFKKVFGLEEEAKITYASESYEEDLEGILAKVGDEYSDVF |
| Sequence: | LAAKNVYDAVELSTILADSDKKSHAKLSSSMIVRFTEHQEDLKKFKRFIR |
| WP_ | ENCPDEYDNLFKNEQKDGYAGYIAHAGKVSQLKFYQYVKKIIQDIAGAE |
| 016631044.1 | YFLEKIAQENFLRKQRTFDNGVIPHQIHLAELQAIIHRQAAYYPFLKENQE |
| Wild type | KIEQLVTFRIPYYVGPLSKGDASTFAWLKRQSEEPIRPWNLQETVDLDQS |
| | ATAFIERMTNFDTYLPSEKVLPKHSLLYEKFMVFNELTKISYTDDRGIKA |
| | NFSGKEKEKIFDYLFKTRRKVKKKDIIQFYRNEYNTEIVTLSGLEEDQFN |
| | ASFSTYQDLLKCGLTRAELDHPDNAEKLEDIIKILTIFEDRQRIRTQLSTFK |
| | GQFSAEVLKKLERKHYTGWGRLSKKLINGIYDKESGKTILDYLVKDDGV |
| | SKHYNRNFMQLINDSQLSFKNAIQKAQSSEHEETLSETVNELAGSPAIKK |
| | GIYQSLKIVDELVAIMGYAPKRIVVEMARENQTTSTGKRRSIQRLKIVEK |
| | AMAEIGSNLLKEQPTTNEQLRDTRLFLYYMQNGKDMYTGDELSLHRLS |
| | HYDIDHIIPQSFMKDDSLDNLVLVGSTENRGKSDDVPSKEVVKDMKAY |
| | WEKLYAAGLISQRKFQRLTKGEQGGLTLEDKAHFIQRQLVETRQITKNV |
| | AGILDQRYNAKSKEKKVQIITLKASLTSQFRSIFGLYKVREVNDYHHGQD |
| | AYLNCVVATTLLKVYPNLAPEFVYGEYPKFQTFKENKATAKAIIYTNLL |
| | RFFTEDEPRFTKDGEILWSNSYLKTIKKELNYHQMNIVKKVEVQKGGFS |
| | KESIKPKGPSNKLIPVKNGLDPQKYGGFDSPVVAYTVLFTHEKGKKPLIK |
| | QEILGITIMEKTRFEQNPILFLEEKGFLRPRVLMKLPKYTLYEFPEGRRRL |
| | LASAKEAQKGNQMVLPEHLLTLLYHAKQCLLPNQSESLAYVEQHQPEF |
| | QEILERVVDFAEVHTLAKSKVQQIVKLFEANQTADVKEIAASFIQLMQFN |
| | AMGAPSTFKFFQKDIERARYTSIKEIFDATIIYQSPTGLYETRRKVVD |
| | (SEQ ID NO: 52) |
| Staphylococcus | KRNYILGLDIGITSVGYGIIDYETRDVIDAGVRLFKEANVENNEGRRSKR |
| aureus Cas9 | GARRLKRRRRHRIQRVKKLLFDYNLLTDHSELSGINPYEARVKGLSQKL |
| | SEEEFSAALLHLAKRRGVHNVNEVEEDTGNELSTKEQISRNSKALEEKY |
| | VAELQLERLKKDGEVRGSINRFKTSDYVKEAKQLLKVQKAYHQLDQSFI |
| | DTYIDLLETRRTYYEGPGEGSPFGWKDIKEWYEMLMGHCTYFPEELRSV |
| | KYAYNADLYNALNDLNNLVITRDENEKLEYYEKFQIIENVFKQKKKPTL |
| | KQIAKEILVNEEDIKGYRVTSTGKPEFTNLKVYHDIKDITARKEIIENAEL |
| | LDQIAKILTIYQSSEDIQEELTNLNSELTQEEIEQISNLKGYTGTHNLSLKAI |
| | NLILDELWHTNDNQIAIFNRLKLVPKKVDLSQQKEIPTTLVDDFILSPVVK |
| | RSFIQSIKVINAIIKKYGLPNDIIIELAREKNSKDAQKMINEMQKRNRQTN |
| | ERIEEIIRTTGKENAKYLIEKIKLHDMQEGKCLYSLEAIPLEDLLNNPFNY |
| | EVDHIIPRSVSFDNSFNNKVLVKQEENSKKGNRTPFQYLSSSDSKISYETF |
| | KKHILNLAKGKGRISKTKKEYLLEERDINRFSVQKDFINRNLVDTRYATR |
| | GLMNLLRSYFRVNNLDVKVKSINGGFTSFLRRKWKFKKERNKGYKHHA |
| | EDALIIANADFIFKEWKKLDKAKKVMENQMFEEKQAESMPEIETEQEYK |
| | EIFITPHQIKHIKDFKDYKYSHRVDKKPNRELINDTLYSTRKDDKGNTLIV |
| | NNLNGLYDKDNDKLKKLINKSPEKLLMYHHDPQTYQKLKLIMEQYGDE |
| | KNPLYKYYEETGNYLTKYSKKDNGPVIKKIKYYGNKLNAHLDITDDYP |
| | NSRNKVVKLSLKPYRFDVYLDNGVYKFVTVKNLDVIKKENYYEVNSKC |
| | YEEAKKLKKISNQAEFIASFYNNDLIKINGELYRVIGVNNDLLNRIEVNMI |
| | DITYREYLENMNDKRPPRIIKTIASKTQSIKKYSTDILGNLYEVKSKKHPQ |
| | IIKKG (SEQ ID NO: 53) |
| Geobacillus | MKYKIGLDIGITSIGWAVINLDIPRIEDLGVRIFDRAENPKTGESLALPRRL |
| thermodenitrificans | ARSARRRLRRRKHRLERIRRLFVREGILTKEELNKLFEKKHEIDVWQLRV |
| Cas9 | EALDRKLNNDELARILLHLAKRRGFRSNRKSERTNKENSTMLKHIEENQ |
| | SILSSYRTVAEMVVKDPKFSLHKRNKEDNYTNTVARDDLEREIKLIFAKQ |
| | REYGNIVCTEAFEHEYISIWASQRPFASKDDIEKKVGFCTFEPKEKRAPK |
| | ATYTFQSFTVWEHINKLRLVSPGGIRALTDDERRLIYKQAFHKNKITFHD |
| | VRTLLNLPDDTRFKGLLYDRNTTLKENEKVRFLELGAYHKIRKAIDSVY |
| | GKGAAKSFRPIDFDTFGYALTMFKDDTDIRSYLRNEYEQNGKRMENLA |
| | DKVYDEELIEELLNLSFSKFGHLSLKALRNILPYMEQGEVYSTACERAGY |
| | TFTGPKKKQKTVLLPNIPPIANPVVMRALTQARKVVNAIIKKYGSPVSIHI |
| | ELARELSQSFDERRKMQKEQEGNRKKNETAIRQLVEYGLTLNPTGLDIV |
| | KFKLWSEQNGKCAYSLQPIEIERLLEPGYTEVDHVIPYSRSLDDSYTNKV |
| | LVLTKENREKGNRTPAEYLGLGSERWQQFETFVLTNKQFSKKKRDRLLR |
| | LHYDENEENEFKNRNLNDTRYISRFLANFIREHLKFADSDDKQKVYTVN |
| | GRITAHLRSRWNFNKNREESNLHHAVDAAIVACTTPSDIARVTAFYQRR |
| | EQNKELSKKTDPQFPQPWPHFADELQARLSKNPKESIKALNLGNYDNEK |
| | LESLQPVFVSRMPKRSITGAAHQETLRRYIGIDERSGKIQTVVKKKLSEIQ |
| | LDKTGHFPMYGKESDPRTYEAIRQRLLEHNNDPKKAFQEPLYKPKKNGE |
| | LGPIIRTIKIIDTTNQVIPLNDGKTVAYNSNIVRVDVFEKDGKYYCVPIYTI |
| | DMMKGILPNKAIEPNKPYSEWKEMTEDYTFRFSLYPNDLIRIEFPREKTIK |
| | TAVGEEIKIKDLFAYYQTIDSSNGGLSLVSHDNNFSLRSIGSRTLKRFEKY |
| | QVDVLGNIYKVRGEKRVGVASSSHSKAGETIRPL (SEQ ID NO: 54) |
| ScCas9 | MEKKYSIGLDIGTNSVGWAVITDDYKVPSKKFKVLGNTNRKSIKKNLM |
| S. canis | GALLFDSGETAEATRLKRTARRRYTRRKNRIRYLQEIFANEMAKLDDSF |
| 1375 AA | FQRLEESFLVEEDKKNERHPIFGNLADEVAYHRNYPTIYHLRKKLADSPE |
| 159.2 kDa | KADLRLIYLALAHIIKFRGHFLIEGKLNAENSDVAKLFYQLIQTYNQLFEE |
| | SPLDEIEVDAKGILSARLSKSKRLEKLIAVFPNEKKNGLFGNIIALALGLTP |
| | NFKSNFDLTEDAKLQLSKDTYDDDLDELLGQIGDQYADLFSAAKNLSDA |
| | ILLSDILRSNSEVTKAPLSASMVKRYDEHHQDLALLKTLVRQQFPEKYAE |
| | IFKDDTKNGYAGYVGIGIKHRKRTTKLATQEEFYKFIKPILEKMDGAEEL |
| | LAKLNRDDLLRKQRTFDNGSIPHQIHLKELHAILRRQEEFYPFLKENREKI |
| | EKILTFRIPYYVGPLARGNSRFAWLTRKSEEAITPWNFEEVVDKGASAQS |
| | FIERMTNFDEQLPNKKVLPKHSLLYEYFTVYNELTKVKYVTERMRKPEF |
| | LSGEQKKAIVDLLFKTNRKVTVKQLKEDYFKKIECFDSVEIIGVEDRFNA |
| | SLGTYHDLLKIIKDKDFLDNEENEDILEDIVLTLTLFEDREMIEERLKTYA |
| | HLFDDKVMKQLKRRHYTGWGRLSRKMINGIRDKQSGKTILDFLKSDGF |
| | SNRNFMQLIHDDSLTFKEEIEKAQVSGQGDSLHEQIADLAGSPAIKKGIL |
| | QTVKIVDELVKVMGHKPENIVIEMARENQTTTKGLQQSRERKKRIEEGIK |
| | ELESQILKENPVENTQLQNEKLYLYYLQNGRDMYVDQELDINRLSDYDV |
| | DHIVPQSFIKDDSIDNKVLTRSVENRGKSDNVPSEEVVKKMKNYWRQLL |
| | NAKLITQRKFDNLTKAERGGLSEADKAGFIKRQLVETRQITKHVARILDS |
| | RMNTKRDKNDKPIREVKVITLKSKLVSDFRKDFQLYKVRDINNYHHAH |
| | DAYLNAVVGTALIKKYPKLESEFVYGDYKVYDVRKMIAKSEQEIGKAT |
| | AKRFFYSNIMNFFKTEVKLANGEIRKRPLIETNGETGEVVWNKEKDFAT |
| | VRKVLAMPQVNIVKKTEVQTGGFSKESILSKRESAKLIPRKKGWDTRKY |
| | GGFGSPTVAYSILVVAKVEKGKAKKLKSVKVLVGITIMEKGSYEKDPIGF |
| | LEAKGYKDIKKELIFKLPKYSLFELENGRRRMLASATELQKANELVLPQ |
| | HLVRLLYYTQNISATTGSNNLGYIEQHREEFKEIFEKIIDFSEKYILKNKV |
| | NSNLKSSFDEQFAVSDSILLSNSFVSLLKYTSFGASGGFTFLDLDVKQGRL |
| | RYQTVTEVLDATLIYQSITGLYETRTDLSQLGGD (SEQ ID NO: 55) |

The napDNAbp used in the PE-VLPs and prime editor fusion proteins described herein may include any suitable homologs and/or orthologs or naturally occurring enzymes, such as, Cas9. Cas9 homologs and/or orthologs have been described in various species, including, but not limited to, S. pyogenes and S. thermophilus. The Cas moiety may be configured (e.g., mutagenized, recombinantly engineered, or otherwise obtained from nature) as a nickase, i.e., capable of cleaving only a single strand of the target double-stranded DNA. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain; that is, the Cas9 is a nickase. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

### Reverse Transcriptase Domain

In various embodiments, the prime editors delivered by the PE-VLPs described herein comprise a reverse transcriptase domain. In some embodiments, the reverse transcriptase domain is a wild type MMLV reverse transcriptase. In some embodiments, the reverse transcriptase domain is a variant of wild type MMLV reverse transcriptase having the amino acid sequence of SEQ ID NO: 60.

For example, PE2 and PEmax comprise a variant reverse transcriptase domain of SEQ ID NO: 60, which is based on the wild type MMLV reverse transcriptase domain of SEQ ID NO: 59 (and, in particular, a Genscript codon optimized MMLV reverse transcriptase having the nucleotide sequence of SEQ ID NO: 59) and which comprises amino acid substitutions D200N T306K W313F T330P L603W relative to the wild type MMLV RT of SEQ ID NO: 60. The amino acid sequence of the variant RT of PE2 and PEmax is SEQ ID NO: 60.

The PE-VLPs and prime editors may also comprise other variant RTs as well. In various embodiments, the prime editors delivered by the VLPs described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 59, or at a corresponding amino acid position in another wild type RT polypeptide sequence.

In various embodiments, the PE-VLPs and prime editors described herein may comprise an MMLV reverse transcriptase variant in which

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes:

| | Sequence (variant substitutions relative to |
|---|---|
| Description | wild type) |
| Reverse | TLNIEDEYRLHETSKEPDVSLGSTWLSDFPQAWAETGGMGLAVRQAP |
| transcriptase | LIIPLKATSTPVSIKQYPMSQEARLGIKPHIQRLLDQGILVPCQSPWNTPL |
| (M-MLV RT) | LPVKKPGTNDYRPVQDLREVNKRVEDIHPTVPNPYNLLSGLPPSHQWY |
| wild type | TVLDLKDAFFCLRLHPTSQPLFAFEWRDPEMGISGQLTWTRLPQGFKN |
| Moloney | SPTLFDEALHRDLADFRIQHPDLILLQYVDDLLLAATSELDCQQGTRAL |
| murine | LQTLGNLGYRASAKKAQICQKQVKYLGYLLKEGQRWLTEARKETVM |
| leukemia | GQPTPKTPRQLREFLGTAGFCRLWIPGFAEMAAPLYPLTKTGTLFNWG |
| virus | PDQQKAYQEIKQALLTAPALGLPDLTKPFELFVDEKQGYAKGVLTQKL |
| Used in PE1 | GPWRRPVAYLSKKLDPVAAGWPPCLRMVAAIAVLTKDAGKLTMGQP |
| (prime editor | LVILAPHAVEALVKQPPDRWLSNARMTHYQALLLDTDRVQFGPVVAL |
| 1 fusion | NPATLLPLPEEGLQHNCLDILAEAHGTRPDLTDQPLPDADHTWYTDGS |
| protein | SLLQEGQRKAGAAVTTETEVIWAKALPAGTSAQRAELIALTQALKMA |
| disclosed | EGKKLNVYTDSRYAFATAHIHGEIYRRRGLLTSEGKEIKNKDEILALLK |
| herein) | ALFLPKRLSIIHCPGHQKGHSAEARGNRMADQAARKAAITETPDTSTLL |
| | IENSSP (SEQ ID NO: 59) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKTGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 61) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 62) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 63) |
| M-MLV RT | TLNIEDEYRLHETSKEPDVSLGSTWLSDFPQAWAETGGMGLAVRQAP |
| D200N | LIIPLKATSTPVSIKQYPMSQKARLGIKPHIQRLLDQGILVPCQSPWNTP |
| T330P | LLPVKKPGTNDYRPVQDLREVNKRVEDIHPTVPNPYNLLSGLPPSHQW |
| L603W | YTVLDLKDAFFCLRLHPTSQPLFAFEWRDPEMGISGQLTWTRLPQGFK |
| E69K | NSPTLFNEALHRDLADFRIQHPDLILLQYVDDLLLAATSELDCQQGTRA |
| | LLQTLGNLGYRASAKKAQICQKQVKYLGYLLKEGQRWLTEARKETV |
| | MGQPTPKTPRQLREFLGTAGFCRLWIPGFAEMAAPLYPLTKPGTLFNW |
| | GPDQQKAYQEIKQALLTAPALGLPDLTKPFELFVDEKQGYAKGVLTQ |
| | KLGPWRRPVAYLSKKLDPVAAGWPPCLRMVAAIAVLTKDAGKLTMG |
| | QPLVILAPHAVEALVKQPPDRWLSNARMTHYQALLLDTDRVQFGPVV |
| | ALNPATLLPLPEEGLQHNCLDILAEAHGTRPDLTDQPLPDADHTWYTD |
| | GSSLLQEGQRKAGAAVTTETEVIWAKALPAGTSAQRAELIALTQALK |
| | MAEGKKLNVYTDSRYAFATAHIHGEIYRRRGWLTSEGKEIKNKDEILA |
| | LLKALFLPKRLSIIHCPGHQKGHSAEARGNRMADQAARKAAITETPDT |
| | STLLIENSSP (SEQ ID NO: 64) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLRRFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP(SEQ ID NO: 65) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 66) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| L139P | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 67) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| L435G | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVIGAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 68) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| N454K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSKARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 69) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGKAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 70) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| W313F | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLFIPGFAEMAAPLYPLTKPGTLFNWGP |
| | DQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 71) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| D524G | SPTLFNEALHRDLADFRIQHPDLILLQYVDDLLLAATSELDCQQGTRAL |
| E562Q | LQTLGNLGYRASAKKAQICQKQVKYLGYLLKEGQRWLTEARKETVM |
| D583N | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTGGS |
| | SLLQEGQRKAGAAVTTETEVIWAKALPAGTSAQRAQLIALTQALKMA |
| | EGKKLNVYTNSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 72) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLRRFLGTAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 73) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| L139P | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 74) |
| M-MLV RT | TLNIEDEYRLHETSKEPDVSLGSTWLSDFPQAWAETGGMGLAVRQAP |
| P51L S67K | LIILLKATSTPVSIKQYPMKQEARLGIKPHIQRLLDQGILVPCQSPWNTP |
| T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFDEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P | WGPDQQKAYQEIKQALLTAPALGLPDLTKPFELFVDEKQGYAKGVLT |
| L435G | QKLGPWRRPVAYLSKKLDPVAAGWPPCLRMVAAIAVLTKDAGKLTM |
| N454K | GQPLVIGAPHAVEALVKQPPDRWLSKARMTHYQALLLDTDRVQFGPV |
| D524G | VALNPATLLPLPEEGLQHNCLDILAEAHGTRPDLTDQPLPDADHTWYT |
| D583N | GGSSLLQEGQRKAGAAVTTETEVIWAKALPAGTSAQRAELIALTQALK |
| H594Q | MAEGKKLNVYTNSRYAFATAHIQGEIYRRRGLLTSEGKEIKNKDEILA |
| D653N | LLKALFLPKRLSIIHCPGHQKGHSAEARGNRMANQAARKAAITETPDT |
| | STLLIENSSP (SEQ ID NO: 75) |
| M-MLV RT | TLNIEDEYRLHETSKEPDVSLGSTWLSDFPQAWAETGGMGLAVRQAP |
| D200N P51L | LIILLKATSTPVSIKQYPMKQEARLGIKPHIQRLLDQGILVPCQSPWNTP |
| S67K T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFNEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P L345G | WGPDQQKAYQEIKQALLTAPALGLPDLTKPFELFVDEKQGYAKGVLT |
| N454K | QKLGPWRRPVAYLSKKLDPVAAGWPPCLRMVAAIAVLTKDAGKLTM |
| D524G | GQPLVIGAPHAVEALVKQPPDRWLSKARMTHYQALLLDTDRVQFGPV |
| D583N | VALNPATLLPLPEEGLQHNCLDILAEAHGTRPDLTDQPLPDADHTWYT |
| H594Q | GGSSLLQEGQRKAGAAVTTETEVIWAKALPAGTSAQRAELIALTQALK |
| D653N | MAEGKKLNVYTNSRYAFATAHIQGEIYRRRGLLTSEGKEIKNKDEILA |
| | LLKALFLPKRLSIIHCPGHQKGHSAEARGNRMANQAARKAAITETPDT |
| | STLLIENSSP (SEQ ID NO: 76) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| in PE2 and | GQPTPKTPRQLREFLGKAGFCRLFIPGFAEMAAPLYPLTKPGTLFNWG |
| PEmax | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 60) |

In various other embodiments, the PE-VLPs and prime editors described herein (with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 59, or at a corresponding amino acid position in another wild type RT polypeptide sequence, wherein “X” can be any amino acid.

In various other embodiments, the prime editors delivered by the PE-VLPs described herein (with RT provided as either a fusion partner or in trans) can include a variant RT comprising a P51X mutation in the wild type M-MLV RT of SEQ ID NO: 59, or at a corresponding amino acid position in another wild type RT polypeptide sequence, wherein “X” can be any amino acid. In certain embodiments, X is L.

In various other embodiments, the prime editors delivered by the PE-VLPs described herein (with RT provided as either a fusion partner or in trans) can include a variant RT comprising an S67X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising an E69X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising an L139X mutation in the wild type M-MLV RT of SEQ ID NO: 59, or at a corresponding amino acid position in another wild type RT polypeptide sequence, wherein “X” can be any amino acid. In certain embodiments, X is P.

In various other embodiments, the prime editors delivered by the PE-VLPs described herein (with RT provided as either a fusion partner or in trans) can include a variant RT comprising a T197X mutation in the wild type M-MLV RT of SEQ ID NO: 59, or at a corresponding amino acid position in another wild type RT polypeptide sequence, wherein “X” can be any amino acid. In certain embodiments, X is A.

In various other embodiments, the prime editors delivered by the PE-VLPs described herein (with RT provided as either a fusion partner or in trans) can include a variant RT comprising a D200X mutation in the wild type M-MLV RT of SEQ ID NO: 59, or at a corresponding amino acid position in another wild type RT polypeptide sequence, wherein “X” can be any amino acid. In certain embodiments, X is N.

In various other embodiments, the prime editors delivered by the PE-VLPs described herein (with RT provided as either a fusion partner or in trans) can include a variant RT comprising an H204X mutation in the wild type M-MLV RT of SEQ ID NO: 59, or at a corresponding amino acid position in another wild type RT polypeptide sequence, wherein “X” can be any amino acid. In certain embodiments, X is R.

In various other embodiments, the prime editors delivered by the PE-VLPs described herein (with RT provided as either a fusion partner or in trans) can include a variant RT comprising an F209X mutation in the wild type M-MLV RT of SEQ ID NO: 59, or at a corresponding amino acid position in another wild type RT polypeptide sequence, wherein “X” can be any amino acid. In certain embodiments, X is N.

In various other embodiments, the prime editors delivered by the PE-VLPs described herein (with RT provided as either a fusion partner or in trans) can include a variant RT comprising an E302X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising an E302X mutation in the wild type M-MLV RT of SEQ ID NO: 59, or at a corresponding amino acid position in another wild type RT polypeptide sequence, wherein “X” can be any amino acid. In certain embodiments, X is R.

In various other embodiments, the prime editors delivered by the PE-VLPs described herein (with RT provided as either a fusion partner or in trans) can include a variant RT comprising a T306X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising an F309X mutation in the wild type M-MLV RT of SEQ ID NO: 59, or at a corresponding amino acid position in another wild type RT polypeptide sequence, wherein “X” can be any amino acid. In certain embodiments, X is N.

In various other embodiments, the prime editors delivered by the PE-VLPs described herein (with RT provided as either a fusion partner or in trans) can include a variant RT comprising a W313X mutation in the wild type M-MLV RT of SEQ ID NO: 59, or at a corresponding amino acid position in another wild type RT polypeptide sequence, wherein “X” can be any amino acid. In certain embodiments, X is F.

In various other embodiments, the prime editors delivered by the PE-VLPs described herein (with RT provided as either a fusion partner or in trans) can include a variant RT comprising a T330X mutation in the wild type M-MLV RT of SEQ ID NO: 59, or at a corresponding amino acid position in another wild type RT polypeptide sequence, wherein “X” can be any amino acid. In certain embodiments, X is P.

In various other embodiments, the prime editors delivered by the PE-VLPs described herein (with RT provided as either a fusion partner or in trans) can include a variant RT comprising an L345X mutation in the wild type M-MLV RT of SEQ ID NO: 59, or at a corresponding amino acid position in another wild type RT polypeptide sequence, wherein “X” can be any amino acid. In certain embodiments, X is G.

In various other embodiments, the prime editors delivered by the PE-VLPs described herein (with RT provided as either a fusion partner or in trans) can include a variant RT comprising an L435X mutation in the wild type M-MLV RT of SEQ ID NO: 59, or at a corresponding amino acid position in another wild type RT polypeptide sequence, wherein “X” can be any amino acid. In certain embodiments, X is G.

In various other embodiments, the prime editors delivered by the PE-VLPs described herein (with RT provided as either a fusion partner or in trans) can include a variant RT comprising an N454X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising a D524X mutation in the wild type M-MLV RT of SEQ ID NO: 59, or at a corresponding amino acid position in another wild type RT polypeptide sequence, wherein “X” can be any amino acid. In certain embodiments, X is G.

In various other embodiments, the prime editors delivered by the PE-VLPs described herein (with RT provided as either a fusion partner or in trans) can include a variant RT comprising an E562X mutation in the wild type M-MLV RT of SEQ ID NO: 59, or at a corresponding amino acid position in another wild type RT polypeptide sequence, wherein “X” can be any amino acid. In certain embodiments, X is Q.

In various other embodiments, the prime editors delivered by the PE-VLPs described herein (with RT provided as either a fusion partner or in trans) can include a variant RT comprising a D583X mutation in the wild type M-MLV RT of SEQ ID NO: 59, or at a corresponding amino acid position in another wild type RT polypeptide sequence, wherein “X” can be any amino acid. In certain embodiments, X is N.

In various other embodiments, the prime editors delivered by the PE-VLPs described herein (with RT provided as either a fusion partner or in trans) can include a variant RT comprising an H594X mutation in the wild type M-MLV RT of SEQ ID NO: 59, or at a corresponding amino acid position in another wild type RT polypeptide sequence, wherein “X” can be any amino acid. In certain embodiments, X is Q.

In various other embodiments, the prime editors delivered by the PE-VLPs described herein (with RT provided as either a fusion partner or in trans) can include a variant RT comprising an L603X mutation in the wild type M-MLV RT of SEQ ID NO: 59, or at a corresponding amino acid position in another wild type RT polypeptide sequence, wherein “X” can be any amino acid. In certain embodiments, X is W.

In various other embodiments, the prime editors delivered by the PE-VLPs described herein (with RT provided as either a fusion partner or in trans) can include a variant RT comprising an E607X mutation in the wild type M-MLV RT of SEQ ID NO: 59, or at a corresponding amino acid position in another wild type RT polypeptide sequence, wherein “X” can be any amino acid. In certain embodiments, X is K.

In various other embodiments, the prime editors delivered by the PE-VLPs described herein (with RT provided as either a fusion partner or in trans) can include a variant RT comprising a D653X mutation in the wild type M-MLV RT of SEQ ID NO: 59, or at a corresponding amino acid position in another wild type RT polypeptide sequence, wherein “X” can be any amino acid. In certain embodiments, X is N.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the wild-type enzymes or partial enzymes described in SEQ ID NOs: 59-76.

The prime editor (PE) system described here contemplates any publicly-available reverse transcriptase described or disclosed in any of the following U.S. patents (each of which are incorporated by reference in their entireties): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references describe reverse transcriptases in art. Each of their disclosures are incorporated herein by reference in their entireties.
Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).
Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).
Zhao, C., Liu, F. & Pyle, A. M. An ultraprocessive, accurate reverse transcriptase encoded by a metazoan group II intron. RNA 24, 183-195 (2018).
Zimmerly, S. & Wu, L. An Unexplored Diversity of Reverse Transcriptases in Bacteria. Microbiol Spectr 3, MDNA3-0058-2014 (2015).
Ostertag, E. M. & Kazazian Jr, H. H. Biology of Mammalian L1 Retrotransposons. Annual Review of Genetics 35, 501-538 (2001).
Perach, M. & Hizi, A. Catalytic Features of the Recombinant Reverse Transcriptase of Bovine Leukemia Virus Expressed in Bacteria. Virology 259, 176-189 (1999).
Lim, D. et al. Crystal structure of the Moloney murine leukemia virus RNase H domain. J. Virol. 80, 8379-8389 (2006).
Zhao, C. & Pyle, A. M. Crystal structures of a group II intron maturase reveal a missing link in spliceosome evolution. Nature Structural & Molecular Biology 23, 558-565 (2016).
Griffiths, D. J. Endogenous retroviruses in the human genome sequence. Genome Biol. 2, REVIEWS1017 (2001).
Baranauskas, A. et al. Generation and characterization of new highly thermostable and processive M-MuLV reverse transcriptase variants. Protein Eng Des Sel 25, 657-668 (2012).
Zimmerly, S., Guo, H., Perlman, P. S. & Lambowltz, A. M. Group II intron mobility occurs by target DNA-primed reverse transcription. Cell 82, 545-554 (1995).
Feng, Q., Moran, J. V., Kazazian, H. H. & Boeke, J. D. Human L1 retrotransposon encodes a conserved endonuclease required for retrotransposition. Cell 87, 905-916 (1996).
Berkhout, B., Jebbink, M. & Zsiros, J. Identification of an Active Reverse Transcriptase Enzyme Encoded by a Human Endogenous HERV-K Retrovirus. Journal of Virology 73, 2365-2375 (1999).
Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).
Arezi, B. & Hogrefe, H. Novel mutations in Moloney Murine Leukemia Virus reverse transcriptase increase thermostability through tighter binding to template-primer. Nucleic Acids Res 37, 473-481 (2009).
Blain, S. W. & Goff, S. P. Nuclease activities of Moloney murine leukemia virus reverse transcriptase. Mutants with altered substrate specificities. J. Biol. Chem. 268, 23585-23592 (1993).
Xiong, Y. & Eickbush, T. H. Origin and evolution of retroelements based upon their reverse transcriptase sequences. EMBO J 9, 3353-3362 (1990).
Herschhorn, A. & Hizi, A. Retroviral reverse transcriptases. Cell. Mol. Life Sci. 67, 2717-2747 (2010).
Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).
Liu, M. et al. Reverse Transcriptase-Mediated Tropism Switching in Bordetella Bacteriophage. Science 295, 2091-2094 (2002).
Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
Nottingham, R. M. et al. RNA-seq of human reference RNA samples using a thermostable group II intron reverse transcriptase. RNA 22, 597-613 (2016).
Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).
Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).
Nowak, E. et al. Structural analysis of monomeric retroviral reverse transcriptase in complex with an RNA/DNA hybrid. Nucleic Acids Res 41, 3874-3887 (2013).
Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).
Das, D. & Georgiadis, M. M. The Crystal Structure of the Monomeric Reverse Transcriptase from Moloney Murine Leukemia Virus. Structure 12, 819-829 (2004).
Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).
Gerard, G. F. et al. The role of template-primer in protection of reverse transcriptase from thermal inactivation. Nucleic Acids Res 30, 3118-3129 (2002).
Monot, C. et al. The Specificity and Flexibility of L1 Reverse Transcription Priming at Imperfect T-Tracts. PLOS Genetics 9, e1003499 (2013).
Mohr, S. et al. Thermostable group II intron reverse transcriptase fusion proteins and their use in cDNA synthesis and next-generation RNA sequencing. RNA 19, 958-970 (2013).

Any of the references noted above that relate to reverse transcriptases are hereby incorporated by reference in their entireties, if not already stated so.

### Nuclear Localization Sequences (NLS)

In various embodiments, the fusion proteins delivered by the PE-VLPs described herein may comprise one or more nuclear localization sequences (NLS), which help promote translocation of a protein into the cell nucleus. Such sequences are well-known in the art and can include the following examples:

| | | SEQ |
|---|---|---|
| | | ID |
| DESCRIPTION | SEQUENCE | NO: |
| NLS OF SV40 | PKKKRKV | 30 |
| LARGE | | |
| T-AG | | |
| NLS | MKRTADGSEFESPKKKRKV | 20 |
| NLS | MDSLLMNRRKFLYQFKNVRWAKG | 21 |
| | RRETYLC | |
| NLS OF | AVKRPAATKKAGQAKKKKLD | 22 |
| NUCLEOPLASMIN | | |
| NLS OF EGL-13 | MSRRRKANPTKLSENAKKLAKEV | 23 |
| | EN | |
| NLS OF C-MYC | PAAKRVKLD | 24 |
| NLS OF TUS- | KLKIKRPVK | 25 |
| PROTEIN | | |
| NLS OF POLYOMA | VSRKRPRP | 26 |
| LARGE T-AG | | |
| NLS OF | EGAPPAKRAR | 27 |
| HEPATITIS D | | |
| VIRUS ANTIGEN | | |
| NLS OF MURINE | PPQPKKKPLDGE | 28 |
| P53 | | |
| NLS OF PE1 AND | SGGSKRTADGSEFEPKKKRKV | 29 |
| PE2 | | |
| BIPARTITE SV40 | KRTADGSEFESPKKKRKV | 31 |
| NLS | | |

The NLS examples above are non-limiting. The prime editor fusion proteins delivered by the presently described PE-VLPs may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the fusion proteins, constructs encoding the fusion proteins, and PE-VLPs disclosed herein further comprise one or more, preferably, at least two nuclear localization sequences. In certain embodiments, the fusion proteins comprise at least two NLSs. In embodiments with at least two NLSs, the NLSs can be the same NLSs or they can be different NLSs. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a fusion protein (e.g., inserted between the encoded napDNAbp component (e.g., Cas9) and a polymerase domain (e.g., a reverse transcriptase).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 30), MDSLLMNRRKFLYQFKNVRWAKGRRETYLC (SEQ ID NO: 21), KRTADGSEFESPKKKRKV (SEQ ID NO: 31), or KRTADGSEFEPKKKRKV (SEQ ID NO: 77). In other embodiments, an NLS comprises the amino acid sequences

(SEQ ID NO: 78)
NLSKRPAAIKKAGQAKKKK,
(SEQ ID NO: 24)
PAAKRVKLD,
(SEQ ID NO: 80)
RQRRNELKRSF,
or
(SEQ ID NO: 80)
NQSSNFGPMKGGNFGGRSSGPYGGGGQYFAKPRNQGGY.

In one aspect of the disclosure, a prime editor or other fusion protein may be modified with one or more nuclear localization sequences (NLS), preferably at least two NLSs. In certain embodiments, the fusion proteins are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 30)); (ii) a bipartite motif consisting of two basic domains separated by a variable number of spacer amino acids and exemplified by the Xenopus nucleoplasmin NLS (KRXXXXXXXXXXKKKL (SEQ ID NO: 81)); and (iii) noncanonical sequences such as M9 of the hnRNP A1 protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form a prime editor-NLS fusion construct. In other embodiments, a fusion protein-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a prime editor and one or more NLSs, among other components.

The prime editor fusion proteins delivered by the PE-VLPs described herein may also comprise nuclear localization sequences that are linked to a prime editor through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the prime editor by any suitable strategy that effectuates forming a bond (e.g., covalent linkage, hydrogen bonding) between the prime editor and the one or more NLSs.

### Nuclear Export Sequences (NES)

In various embodiments, the fusion proteins delivered by the PE-VLPs described herein may comprise one or more nuclear export sequences (NES), which help promote translocation of a protein out of the cell nucleus. Such sequences are well-known in the art and can include the following examples:

| SEQUENCE: | SEQ ID NO: |
|---|---|
| MEELSQALASSFSV | 82 |
| PLQLPPLERLTL | 83 |
| NELALKLAGLDI | 84 |
| ERFEMFRELNEALEL | 85 |
| DHAEKVAEKLEALSV | 86 |
| QLVEELLKIICAFQL | 87 |
| TNLEALQKKLEELEL | 88 |
| DVKEEMTSALATMRV | 89 |
| STNGSLAAEFRHLQL | 90 |
| PSVQELTEQIHRLLM | 91 |
| MNFKELKDFLKELNI | 92 |
| ENFEILMKLKESLEL | 93 |
| FETVYELTKMCTIR | 94 |
| SGKASSSLGLQDFDL | 95 |
| PKYSDIDVDGLCSEL | 96 |
| VDLACTPTDVRDVDI | 97 |
| YGEKTTQRDLTELEI | 98 |
| RRIYDITNVLEGIGL | 99 |
| AKIIPYSGLLLVITV | 100 |
| LRSEEVHWLHVDMGV | 101 |
| LQSEEVHWLHLDMGV | 102 |
| LQVRKYSLDLASLIL | 103 |
| AGVEAIIRILQQLLF | 104 |
| TGVEALIRILQQLLF | 105 |
| IVLNQLCVRFFGLDL | 106 |
| SLGGFEITPPVVLRL | 107 |
| EAIQDLCLAVEEVSL | 108 |
| DELLQVLRMMVGVNI | 109 |
| SVMLAVQEGIDLLTF | 110 |
| LSSHFQELSI | 111 |
| QSTHVDIRTLEDLLM | 112 |
| ESSAEDLRTLQQLFL | 113 |
| EFSLPTHHTVRLIRV | 114 |
| MSSGYYLGEILRLAL | 115 |
| DTVLDILRDFFELRL | 116 |
| NSVNEILSEFYYVRL | 117 |
| CAFLSVKKQFEELTL | 118 |
| ISPEHVIQALESLGF | 119 |
| AHWMRQLVSFQKLKL | 120 |
| ATRELDELMASLSDF | 121 |
| YQNIELITFINALKL | 122 |
| FNATAVVRHMRKLQL | 123 |
| SGIFGLVTNLEELEV | 124 |
| EESYTLNSDLARLGV | 125 |
| EESYDLTSHLARLGV | 126 |
| GIQQAHAEQLANMRI | 127 |
| DVKEEMTSALATMRV | 89 |
| AAEPVILDLRDLFQL | 128 |
| MEGCVSNLMV | 129 |
| EGCVSNLMV | 130 |
| DMDFLRNLFSQTLSL | 131 |
| EQLLEIVHDLENLSL | 132 |
| NVMKYFTDLFDYLPL | 133 |
| KVYPIILRLGSNLSL | 134 |
| YAGFSLPHAILRIDL | 135 |
| EIVRDIKEKLCYVAL | 136 |
| EAINKLESNLRELQI | 137 |
| EAINKLENNLRELQI | 138 |
| SDQKQEQLLLKKMYL | 139 |
| KQVLWDRTFSLFQQL | 140 |
| AQLQNLTKRIDSLPL | 141 |
| NDENEHQLSLRTVSL | 142 |
| ISFTEFVKVLEKVDV | 143 |
| MESAITLWQFLLQL | 144 |
| VPKELMQQIENFEKI | 145 |
| QARFILEKIDGKIII | 146 |
| QVKFIKMIIEKELTV | 147 |
| NHRMKNLREISQLGI | 148 |
| NHRVKKLNEISKLGI | 149 |
| TEKHLQKYLRQDLRL | 150 |
| RQERKRPLLDLHIEL | 151 |
| ANMRIQDLKVSLKPL | 152 |
| ATMRVDYEQIKIKKI | 153 |
| LQGEEFVCLKSIILL | 154 |
| THYGQKAILFLPLPV | 155 |
| PSAHEITGLADSLQL | 156 |
| VRLHDVLHSDKKLTL | 157 |
| LINRNGELKLANFGL | 158 |
| LEPLKKLECLKSLDL | 159 |

The NES examples above are non-limiting. The prime editor fusion proteins delivered by the presently described PE-VLPs may comprise any known NES sequence, including any of those described in Xu, D. et al. Sequence and structural analyses of nuclear export signals in the NESdb database. Mol. Biol. Cell. 2012, 23(18), 3677-3693; Fung, H. Y. J. et al. Structural determinants of nuclear export signal orientation in binding to exportin CRM1. eLife. 2015, 4:e10034; and Kosugi, S. et al. Nuclear Export Signal Consensus Sequences Defined Using a Localization-based Yeast Selection System. Traffic. 2008, 9(12), 2053-2062, each of which are incorporated herein by reference.

In various embodiments, the fusion proteins, constructs encoding the fusion proteins, and PE-VLPs disclosed herein further comprise one or more, preferably, at least three nuclear export sequences. In certain embodiments, the fusion proteins comprise at least three NESs. In embodiments with at least three NESs, the NESs can be the same NESs or they can be different NESs. The location of the NES fusion can be at the N-terminus, the C-terminus, or within a sequence of a fusion protein (e.g., inserted between the encoded napDNAbp component (e.g., Cas9) and the gag nucleocapsid protein). In certain preferred embodiments, the NES (or multiple NESs, e.g., three NESs) are positioned between the napDNAbp and the gag nucleocapsid protein such that they can be cleaved from the napDNAbp upon delivery of the fusion protein to a target cell.

The NESs may be any known NES sequence in the art. The NESs may also be any future-discovered NESs for nuclear export. The NESs also may be any naturally-occurring NES, or any non-naturally occurring NES (e.g., an NES with one or more desired mutations).

The term “nuclear export sequence” or “NES” refers to an amino acid sequence that promotes export of a protein from the cell nucleus, for example, by nuclear transport. Nuclear export sequences are known in the art and would be apparent to the skilled artisan.

In one aspect of the disclosure, a prime editor or other fusion protein may be modified with one or more nuclear export sequences (NES), preferably at least three NESs. In certain embodiments, the fusion proteins are modified with two or more, three or more, four or more, five or more, six or more, seven or more, eight or more, nine or more, or ten or more NESs. The disclosure contemplates the use of any nuclear export sequence known in the art at the time of the disclosure, or any nuclear export sequence that is identified or otherwise made available in the state of the art after the time of the instant filing. A representative nuclear export sequence is a peptide sequence that directs the protein out of the nucleus of the cell in which the sequence is expressed. NESs commonly contain hydrophobic amino acid residues in the sequence LXXXLXXLXL, where L is a hydrophobic residue (frequently leucine), and X represents any amino acid. Nuclear export sequences often comprise leucine residues.

The fusion proteins delivered by the PE-VLPs described herein may also comprise nuclear export sequences that are linked to a prime editor through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the prime editor by any suitable strategy that effectuates forming a bond (e.g., covalent linkage, hydrogen bonding) between the prime editor and the one or more NESs. In some embodiments, the linker joining one or more NES and a prime editor is a cleavable linker, as described further herein, such that the one or more NES can be cleaved from the prime editor, e.g., upon delivery of the prime editor to a target cell.

### Linkers

The fusion proteins and PE-VLPs described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and a polymerase (e.g., a reverse transcriptase). In some embodiments, a linker joins a Cas9 nickase and a reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 164), (G)ₙ(SEQ ID NO: 165), (EAAAK)ₙ (SEQ ID NO: 166), (GGS)ₙ(SEQ ID NO: 167), (SGGS)ₙ(SEQ ID NO: 168), (XP)ₙ (SEQ ID NO: 169), or any combination thereof, wherein n is independently an integer between 1 and 30, and wherein X is any amino acid. In some embodiments, the linker comprises the amino acid sequence (GGS). (SEQ ID NO: 167), wherein n is 1, 3, or 7. In some embodiments, the linker comprises the amino acid sequence SGSETPGTSESATPES (SEQ ID NO: 170). In some embodiments, the linker comprises the amino acid sequence SGGSSGGSSGSETPGTSESATPESSGGSSGGS (SEQ ID NO: 171). In some embodiments, the linker comprises the amino acid sequence SGGSGGSGGS (SEQ ID NO: 172). In some embodiments, the linker comprises the amino acid sequence SGGS (SEQ ID NO: 162). In other embodiments, the linker comprises the amino acid sequence SGGSSGGSSGSETPGTSESATPESAGSYPYDVPDYAGSAAPAAKKKKLDGSGSGGSS GGS (SEQ ID NO: 173, 60AA). In some embodiments, the linker comprises the amino acid sequence GGS, GGSGGS (SEQ ID NO: 174), GGSGGSGGS (SEQ ID NO: 175), SGGSSGGSSGSETPGTSESATPESSGGSSGGSS (SEQ ID NO: 161), SGSETPGTSESATPES (SEQ ID NO: 170), or SGGSSGGSSGSETPGTSESATPESAGSYPYDVPDYAGSAAPAAKKKKLDGSGSGGSS GG S (SEQ ID NO: 173).

In certain embodiments, linkers may be used to link any of the peptides or peptide domains or moieties of the invention (e.g., a napDNAbp linked or fused to a reverse transcriptase domain, and/or a napDNAbp linked to one or more NESs). Any of the domains of the fusion proteins described herein may also be connected to one another through any of the presently described linkers.

In some embodiments, a linker is a cleavable linker (e.g., a linker that can be split or cut by any means). A cleavable linker may be an amino acid sequence. In some embodiments, the linker between one or more NES and the napDNAbp of the fusion proteins and PE-VLPs provided herein comprises a cleavable linker. A cleavable linker may comprise a self-cleaving peptide (e.g., a 2A peptide such as EGRGSLLTCGDVEENPGP (SEQ ID NO: 1), ATNFSLLKQAGDVEENPGP (SEQ ID NO: 2), QCTNYALLKLAGDVESNPGP (SEQ ID NO: 3), or VKQTLNFDLLKLAGDVESNPGP (SEQ ID NO: 4)). In some embodiments, a cleavable linker comprises a protease cleavage site that is cut after being contacted by a protease. For example, the present disclosure contemplates the use of cleavable linkers comprising a protease cleavage site of amino acid sequences TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8. In certain embodiments, a cleavable linker comprises an MMLV protease cleavage site or an FMLV protease cleavage site. In certain embodiments, the fusion proteins and PE-VLPs described herein comprise the cleavable linker TSTLLMENSS (SEQ ID NO: 5) joining one or more NES and a napDNAbp. In some embodiments, the linker is cleaved upon delivery of the PE-VLP/fusion protein to a target cell, releasing a free prime editor that is capable of translocating into the nucleus of the target cell.

The protease cleavage site may be any known in the art, or any sequence yet to be discovered, so long as the corresponding protease may be co-packaged in the eVLPs to allow for post-maturation cleavage within the mature eVLP particles. Such cleavage sites and their corresponding proteases include but are not limited to: (a) granzyme A, which recognizes and cleaves a sequence comprising ASPRAGGK (SEQ ID NO: 243), (b) granzyme B, which recognizes and cleaves a sequence comprising YEADSLEE (SEQ ID NO: 244), (c) granzyme K, which recognizes and cleaves a sequence comprising YQYRAL (SEQ ID NO: 246), (d) Cathepsin D, which recognizes and cleaves a sequence comprising LGVLIV (SEQ ID NO: 247). Many other combinations of specific proteases and protease cleavage sites may be used in connection with the present disclosure by co-packing a specific protease during the eVLP manufacture process. Such proteases can include, without limitation, Arg-C proteinase, Asp-N Endopeptidase, Caspase 1, Caspase 2, Caspase 3, Caspase 4, Caspase 5, Caspase 7, Caspase 8, Caspase 9, Caspase 10, Chymotrypsin, Clostripain, Enterokinase, Factor Xa, Glutamyl endopeptidase, Granzyme B, Neutrophil elastase, Pepsin, Prolyl-endopeptidase, Proteinase K, Staphylococcal peptidase I, Thermolysin, Thrombin, and Trypsin. Any protease paired with its cognate recognition sequence may be used in the present disclosure protease-sensitive linkers, including any serine protease, cysteine protease, aspartic protease, threonine protease, glutamic protease, metalloprotease, or asparagine peptide lyase (which constitute major classifications of known proteases). The specific protease cleavage sites for said enzymes are well-known in the art and may be utilized in the linkers herein to provide protease-susceptible linkers.

Group-Specific Antigen (gag) Proteins and Viral Envelope Glycoproteins

The PE-VLPs described herein include various viral envelope and capsid components, which are used to encapsulate and deliver the prime editor fusion proteins described herein. The use of viral envelope and capsid components for nucleic acid and protein delivery is known in the art, and a person of ordinary skill in the art would readily appreciate the various options known in the art that could be used or substituted for these components in the presently described PE-VLPs. The use of such viral components for nucleic acid and/or protein delivery (e.g., delivery of Cas9) is described, for example, in Mangeot et al., Nat. Commun. 10, 45 (2019); Gutkin, et al. Nat. Biotechnol. (2021); and Hamilton, J. R. et al. Cell Reports 35(9), 109207 (2021), each of which is incorporated herein by reference.

In some embodiments, the PE-VLPs described herein comprise a viral envelope glycoprotein layer as the outermost layer of the PE-VLP. Viral envelope glycoproteins are oligosaccharide-containing proteins that form a part of the viral envelope, i.e., the outermost layer of many types of viruses that protects the viral genetic materials when traveling between host cells. Glycoproteins may assist with identification and binding to receptors on a target cell membrane so that the viral envelope fuses with the membrane, allowing the contents of the viral particle (which may comprise, e.g., a fusion protein in a PE-VLP as described herein) to enter the host cell.

The viral envelope glycoproteins used in the PE-VLPs of the present disclosure may comprise any glycoprotein from an enveloped virus. In some embodiments, a viral envelope glycoprotein is an adenoviral envelope glycoprotein, an adeno-associated viral envelope glycoprotein, a retroviral envelope glycoprotein, or a lentiviral envelope glycoprotein. In certain embodiments, a viral envelope glycoprotein is a vesicular stomatitis virus G protein (VSV-G), a baboon retroviral envelope glycoprotein (BaEVRless), a FuG-B2 envelope glycoprotein, or an ecotropic murine leukemia virus (MLV) envelope glycoprotein.

Any known viral envelope glycoprotein can be used in the PE-VLPs of the present disclosure. Any viral envelope glycoprotein discovered or characterized in the future can also be used in the PE-VLPs of the present disclosure. A person of ordinary skill in the art would readily be able to find additional viral envelope glycoproteins that could be used in the PE-VLPs described herein. For example, viral envelope glycoproteins are described in Banerjee, V. and Mukhopadhyay, S. Virus Disease (2016), 27(1), 1-11 and Li, Y. et al. Front. Immunol. (2021), 12, 1-12, each of which is incorporated herein by reference.

In some embodiments, the PE-VLPs described herein further comprise an inner encapsulation layer comprising components from viral capsids. These components include gag-pro polyproteins (e.g., gag nucleocapsid proteins further comprising a viral protease linked thereto) and gag nucleocapsid proteins (e.g., proteins that make up the core structural component of the inner shell of many viruses, lacking the protease of the gag-pro polyproteins) as described herein.

Gag-pro polyproteins mediate proteolytic cleavage of gag and gag-pol polyproteins or nucleocapsid proteins during or shortly after the release of a virion from the plasma membrane. In the PE-VLPs described herein, the protease of a gag-pro polyprotein is responsible for cleaving a cleavable linker in the fusion protein to release a prime editor following delivery of the PE-VLP to a target cell. In some embodiments, a gag-pro polyprotein is an MMLV gag-pro polyprotein or an FMLV gag-pro polyprotein.

The gag nucleocapsid proteins used in the PE-VLPs of the present disclosure may be an MMLV gag nucleocapsid protein, an FMLV gag nucleocapsid protein, or a nucleocapsid protein from any other virus that produces such proteins. In some embodiments, gag nucleocapsid proteins are fused to napDNAbps (e.g., as part of a prime editor). In some embodiments, the fusion further comprises an NES as described herein. In certain embodiments, the gag nucleocapsid protein and the NES are located on one side of a cleavable linker as described herein, and the napDNAbp or prime editor is located on the other side of the cleavable linker, such that the prime editor can be released from the gag nucleocapsid protein upon cleavage of the cleavable linker by the protease of the gag-pro polyprotein following delivery of the PE-VLP to a target cell.

Both the gag-pro polyprotein and the gag nucleocapsid protein form the inner encapsulation layer of the presently described PE-VLPs. Any ratio of the gag-pro polyprotein to the gag nucleocapsid protein (i.e., as part of the fusion proteins described herein) is contemplated in the PE-VLPs of the present disclosure. In some embodiments, the ratio of the gag-pro polyprotein to the fusion protein comprising a gag nucleocapsid protein is approximately 10:1, approximately 9:1, approximately 8:1, approximately 7:1, approximately 6:1, approximately 5:1, approximately 4:1, approximately 3:1, approximately 2:1, approximately 1.5:1, approximately 1:1, or approximately 0.5:1. In certain embodiments, the ratio is approximately 3:1.

### Additional Prime Editor Domains

### A. Flap Endonucleases (e.g., FEN1)

In various embodiments, the PE fusion proteins delivered by the PE-VLPs described herein may comprise one or more flap endonucleases (e.g., FEN1), which refers to an enzyme that catalyzes the removal of 5′ single strand DNA flaps (provided in trans or fused to the PE fusion proteins). These are naturally occurring enzymes that process the removal of 5′ flaps formed during cellular processes, including DNA replication. The prime editors delivered by the PE-VLPs described herein may utilize endogenously supplied flap endonucleases or those provided in trans to remove the 5′ flap of endogenous DNA formed at the target site during prime editing. Flap endonucleases are known in the art and can are described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference). An exemplary flap endonuclease is FENi1, which can be represented by the following amino acid sequence:

| | | SEQ |
|---|---|---|
| Descrip- | | ID |
| tion | Sequence | NO: |
| FEN1 | MGIQGLAKLIADVAPSAIRENDIKSY | 176 |
| Wild | FGRKVAIDASMSIYQFLIAVRQGGDV | |
| type | LQNEEGETTSHLMGMFYRTIRMMENG | |
| (wt) | IKPVYVFDGKPPQLKSGELAKRSERR | |
| | AEAEKQLQQAQAAGAEQEVEKFTKRL | |
| | VKVTKQHNDECKHLLSLMGIPYLDAP | |
| | SEAEASCAALVKAGKVYAAATEDMDC | |
| | LTFGSPVLMRHLTASEAKKLPIQEFH | |
| | LSRILQELGLNQEQFVDLCILLGSDY | |
| | CESIRGIGPKRAVDLIQKHKSIEEIV | |
| | RRLDPNKYPVPENWLHKEAHQLFLEP | |
| | VELDPESVELKWSEPNEEELIKFMCG | |
| | EKQFSEERIRSGVKRLSKSRQGSTQG | |
| | RLDDFFKVTGSLSSAKRKEPEPKGST | |
| | KKKAKTGAAGKFKRGK | |

The flap endonucleases may also include any FEN1 variant, mutant, or other flap endonuclease ortholog, homolog, or variant. Non-limiting FEN1 variant examples are as follows:

| | | SEQ |
|---|---|---|
| Descrip- | | ID |
| tion | Sequence | NO: |
| FEN1 | MGIQGLAKLIADVAPSAIRENDIKSYFGR | 177 |
| K168R | KVAIDASMSIYQFLIAVRQGGDVLQNEEG | |
| (rela- | ETTSHLMGMFYRTIRMMENGIKPVYVFDG | |
| tive | KPPQLKSGELAKRSERRAEAEKQLQQAQA | |
| to FEN1 | AGAEQEVEKFTKRLVKVTKQHNDECKHLL | |
| wt) | SLMGIPYLDAPSEAEASCAALVRAGKVYA | |
| | AATEDMDCLTFGSPVLMRHLTASEAKKLP | |
| | IQEFHLSRILQELGLNQEQFVDLCILLGS | |
| | DYCESIRGIGPKRAVDLIQKHKSIEEIVR | |
| | RLDPNKYPVPENWLHKEAHQLFLEPEVLD | |
| | PESVELKWSEPNEEELIKFMCGEKQFSEE | |
| | RIRSGVKRLSKSRQGSTQGRLDDFFKVTG | |
| | SLSSAKRKEPEPKGSTKKKAKTGAAGKFK | |
| | RGK | |
| FEN1 | MGIQGLAKLIADVAPSAIRENDIKSYFGR | 178 |
| S187A | KVAIDASMSIYQFLIAVRQGGDVLQNEEG | |
| (rela- | ETTSHLMGMFYRTIRMMENGIKPVYVFDG | |
| tive | KPPQLKSGELAKRSERRAEAEKQLQQQAA | |
| to FEN1 | GAEQEVEKFTKRLVKVTKQHNDECKHLLS | |
| wt) | LMGIPYLDAPSEAEASCAALVKAGKVYAA | |
| | ATEDMDCLTFGAAPVLMRHLTASEAKKLP | |
| | IQEFHLSRILQELGLNQEQFVDLCILLGS | |
| | DYCESIRGIGPKRAVDLIQKHKSIEEIVR | |
| | RLDPNKYPVPENWLHKEAHQLFLEPEVLD | |
| | PESVELKWSEPNEEELIKFMCGEKQFSEE | |
| | RIRSGVKRLSKSRQGSTQGRLDDFFKVTG | |
| | SLSSAKRKEPEPKGSTKKKAKTGAAGKFK | |
| | RGK | |
| FEN1 | MGIQGLAKLIADVAPSAIRENDIKSYFGR | 179 |
| K354R | KVAIDASMSIYQFLIAVRQGGDVLQNEEG | |
| (rela- | ETTSHLMGMFYRTIRMMENGIKPVYVFDG | |
| tive | KPPQLKSGELAKRSERRAEAEKQLQQAQA | |
| to FEN1 | AGAEQEVEKFTKRLVKVTKQHNDECKHLL | |
| wt) | SLMGIPYLDAPSEAEASCAALVKAGKVYA | |
| | AATEDMDCLTFGSPVLMRHLTASEAKKLP | |
| | IQEFHLSRILQELGLNQEQFVDLCILLGS | |
| | DYCESIRGIGPKRAVDLIQKHKSIEEIVR | |
| | RLDPNKYPVPENWLHKEAHQLFLEPEVLD | |
| | PESVELKWSEPNEEELIKFMCGEKQFSEE | |
| | RIRSGVKRLSKSRQGSTQGRLDDFFKVTG | |
| | SLSSARRKEPEPKGSTKKKAKTGAAGKFK | |
| | RGK | |
| GEN1 | MGVNDLWQILEPVKQHIPLRNLGGKTIAV | 180 |
| | DLSLWVCEAQTVKKMMGSVMKPHLRNLFF | |
| | RISYLTQMDVKLVFVMEGEPPKLKADVIS | |
| | KRNQSRYGSSGKSWSQKTGRSHFKSVLRE | |
| | CLHMLECLGIPWVQAAGEAEAMCAYLNAG | |
| | GHVDGCLTNDGDTFLYGAQTVYRNFTMNT | |
| | KDPHVDCYTMSSIKSKLGLDRDALVGLAI | |
| | LLGCDYLPKGVPGVGKEQALKLIQILKGQ | |
| | SLLQRFNRWNETSCNSSPQLLVTKKLAHC | |
| | SVCSHPGSPKDHERNGCRLCKSDKYCEPH | |
| | DYEYCCPCEWHRTEHDRQLSEVENNIKKK | |
| | ACCCEGFPFHEVIQEFLLNKDKLVKVIRY | |
| | QRPDLLLFQRFTLEKMEWPNHYACEKLLV | |
| | LLTHYDMIERKLGSRNSNQLQPIRIVKTR | |
| | IRNGVHCFEIEWEKPEHYAMEDKQHGEFA | |
| | LLTIEEESLFEAAYPEIVAVYQKQKLEIK | |
| | GKKQKRIKPKENNLPEPDEVMSFQSHMTL | |
| | KPTCEIFHKQNSKLNSGISPDPTLPQESI | |
| | SASLNSLLLPKNTPCLNAQEQFMSSLRPL | |
| | AIQQIKAVSKSLISESSQPNTSSHNISVI | |
| | ADLHLSTIDWEGTSFSNSPAIQRNTFSHD | |
| | LKSEVESELSAIPDGFENIPEQLSCESER | |
| | YTANIKKVLDEDSDGISPEEHLLSGITDL | |
| | CLQDLPLKERIFTKLSYPQDNLQPDVNLK | |
| | TLSILSVKESCIANSGSDCTSHLSKDLPG | |
| | IPLQNESRDSKILKGDQLLQEDYKVNTSV | |
| | PYSVSNTVVKTCNVRPPNTALDHSRKVDM | |
| | QTTRKILMKKSVCLDRHSSDEQSAPVFGK | |
| | AKYTTQRMKHSSQKHNSSHFKESGHNKLS | |
| | SPKIHIKETEQCVRSYETAENEESCFPDS | |
| | TKSSLSSLQCHKKENNSGTCLDSPLPLRQ | |
| | RLKLRFQST | |
| ERCC5 | MGVQGLWKLLECSGRQVSPEALEGKILAV | 181 |
| | DISIWLNQALKGVRDRHGNSIENPHLLTL | |
| | FHRLCKLLFFRIRPIFVFDGDAPLLKKQT | |
| | LVKRRQRKDLASSDSRKTTEKLLKTFLKR | |
| | QAIKTAFRSKRDEALPSLTQVRRENDLYV | |
| | LPPLQEEEKHSSEEEDEKEWQERMNQKQA | |
| | LQEEFFHNPQAIDIESEDFSSLPPEVKHE | |
| | ILTDMKEFTKRRRTLFEAMPEESDDFSQY | |
| | QLKGLLKKNYLNQHIEHVQKEMNQQHSGH | |
| | IRRQYEDEGGFLKEVESRRVVSEDTSHYI | |
| | LIKGIQAKTVAEVDSESLPSSSKMHGMSF | |
| | DVKSSPCEKLKTEKEPDATPPSPRTLLAM | |
| | QAALLGSSSEEELESENRRQARGRNAPAA | |
| | VDEGSISPRTLSAIKRALDDDEDVKVCAG | |
| | DDVQTGGPGAEEMRINSSTENSDEGLKVR | |
| | DGKGIPFTATLASSSVNSAEEHVASTNEG | |
| | REPTDSVPKEQMSLVHVGTEAFPISDESM | |
| | IKDRKDRLPLESAVVRHSDAPGLPNGREL | |
| | TPASPTCTNSVSKNETHAEVLEQQNELCP | |
| | YESKFDSSLLSSDDETKCKPNSASEVIGP | |
| | VSLQETSSIVSVPSEAVDNVENVVSFNAK | |
| | EHENFLETIQEQQTTESAGQDLISIPKAV | |
| | EPMEIDSEESESDGSFIEVQSVISDEELQ | |
| | AEFPETSKPPSEQGEEELVGTREGEAPAE | |
| | SESLLRDNSERDDVDGEPQEAEKDAEDSL | |
| | HEWQDINLEELETLESNLLAQQNSLKAQK | |
| | QQQERIAATVTGQMFLESQELLRLFGIPY | |
| | IQAPMEAEAQCAILDLTDQTSGTITDDSD | |
| | IWLFGARHVYRNFFNKNKFVEYYQYVDFH | |
| | NQLGLDRNKLINLAYLLGSDYTEGIPTVG | |
| | CVTAMEILNEFPGHGLEPLLKFSEWWHEA | |
| | QKNPKIRPNPHDTKVKKKLRTLQLTPGFP | |
| | NPAVAEAYLKPVVDDSKGSFLWGKPDLDK | |
| | IREFCQRYFGWNRTKTDESLFPVLKQLDA | |
| | QQTQLRIDSFFRLAQQEKEDAKRIKSQRL | |
| | NRAVTCMLRKEKEAAASEIEAVSVAMEKE | |
| | FELLDKAKRKTQKRGITNTLEESSSLKRK | |
| | RLSDSKRKNTCGGFLGETCLSESSDGSSS | |
| | EDAESSSLMNVQRRTAAKEPKTSASDSQN | |
| | SVKEAPVKNGGATTSSSSDSDDDGGKEKM | |
| | VLVTARSVFGKKRRKLRRARGRKRKT | |

In various embodiments, the prime editor fusion proteins utilized in the methods and compositions contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences. Other endonucleases that may be utilized by the instant compositions and methods to facilitate removal of the 5′ end single strand DNA flap include, but are not limited to (1) trex 2, (2) exo1 endonuclease (e.g., Keijzers et al., Biosci Rep. 2015, 35(3): e00206)

### Trex 2

Three prime (3′) repair exonuclease 2 (TREX2) -
human
Accession No. NM_080701
(SEQ ID NO: 182)
MSEAPRAETFVFLDLEATGLPSVEPEIAELSLFAVHRSSLENPEHDESGA
LVLPRVLDKLTLCMCPERPFTAKASEITGLSSEGLARCRKAGFDGAVVRT
LQAFLSRQAGPICLVAHNGFDYDFPLLCAELRRLGARLPRDTVCLDTLPA
LRGLDRAHSHGTRARGRQGYSLGSLFHRYFRAEPSAAHSAEGDVHTLLLI
FLHRAAELLAWADEQARGWAHIEPMYLPPDDPSLEA.
Three prime (3′) repair exonuclease 2 (TREX2) -
mouse
Accession No. NM_011907
(SEQ ID NO: 183)
MSEPPRAETFVFLDLEATGLPNMDPEIAEISLFAVHRSSLENPERDDSGS
LVLPRVLDKLTLCMCPERPFTAKASEITGLSSESLMHCGKAGFNGAVVRT
LQGFLSRQEGPICLVAHNGFDYDFPLLCTELQRLGAHLPQDTVCLDTLPA
LRGLDRAHSHGTRAQGRKSYSLASLFHRYFQAEPSAAHSAEGDVHTLLLI
FLHRAPELLAWADEQARSWAHIEPMYVPPDGPSLEA.
Three prime (3′) repair exonuclease 2 (TREX2) -
rat
Accession No. NM_001107580
(SEQ ID NO: 184)
MSEPLRAETFVFLDLEATGLPNMDPEIAEISLFAVHRSSLENPERDDSGS
LVLPRVLDKLTLCMCPERPFTAKASEITGLSSEGLMNCRKAAFNDAVVRT
LQGFLSRQEGPICLVAHNGFDYDFPLLCTELQRLGAHLPRDTVCLDTLPA
LRGLDRVHSHGTRAQGRKSYSLASLFHRYFQAEPSAAHSAEGDVNTLLLI
FLHRAPELLAWADEQARSWAHIEPMYVPPDGPSLEA.

### Exo1

Human exonuclease 1 (EXO1) has been implicated in many different DNA metabolic processes, including DNA mismatch repair (MMR), micro-mediated end-joining, homologous recombination (HR), and replication. Human EXO1 belongs to a family of eukaryotic nucleases, Rad2/XPG, which also include FEN1 and GEN1. The Rad2/XPG family is conserved in the nuclease domain through species from phage to human. The EXO1 gene product exhibits both 5′ exonuclease and 5′ flap activity. Additionally, EXO1 contains an intrinsic 5′ RNase H activity. Human EXO1 has a high affinity for processing double stranded DNA (dsDNA), nicks, gaps, and pseudo Y structures and can resolve Holliday junctions using its inherit flap activity. Human EXO1 is implicated in MMR and contains conserved binding domains interacting directly with MLH1 and MSH2. EXO1 nucleolytic activity is positively stimulated by PCNA, MutSa (MSH2/MSH6 complex), 14-3-3, MRN, and 9-1-1 complex.

Exonuclease 1 (EXO1) Accession No. NM_003686
(Homo sapiens exonuclease 1 (EXO1),
transcript variant 3) - isoform A
(SEQ ID NO: 185)
MGIQGLLQFIKEASEPIHVRKYKGQVVAVDTYCWLHKGAIACAEKLAKGE
PTDRYVGFCMKFVNMLLSHGIKPILVFDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSAELSEDDLLSQYSLSFTKKTKKNSSEGN
KSLSFSEVFVPDLVNGPTNKKSVSTPPRTRNKFATFLQRKNEESGAVVVP
GTRSRFFCSSDSTDCVSNKVSIQPLDETAVTDKENNLHESEYGDQEGKRL
VDTDVARNSSDDIPNNHIPGDHIPDKATVFTDEESYSFESSKFTRTISPP
TLGTLRSCFSWSGGLGDFSRTPSPSPSTALQQFRRKSDSPTSLPENNMSD
VSQLKSEESSDDESHPLREEACSSQSQESGEFSLQSSNASKLSQCSSKDS
DSEESDCNIKLLDSQSDQTSKLRLSHFSKKDTPLRNKVPGLYKSSSADSL
STTKIKPLGPARASGLSKKPASIQKRKHHNAENKPGLQIKLNELWKNFGF
KKF.
Exonuclease 1 (EXO1) Accession No. NM_006027
(Homo sapiens exonuclease 1 (EXO1),
transcript variant 3) - isoform B
(SEQ ID NO: 186)
MGIQGLLQFIKEASEPIHVRKYKGQVVAVDTYCWLHKGAIACAEKLAKGE
PTDRYVGFCMKFVNMLLSHGIKPILVEDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSAELSEDDLLSQYSLSFTKKTKKNSSEGN
KSLSFSEVFVPDLVNGPTNKKSVSTPPRTRNKFATFLQRKNEESGAVVVP
GTRSRFFCSSDSTDCVSNKVSIQPLDETAVTDKENNLHESEYGDQEGKRL
VDTDVARNSSDDIPNNHIPGDHIPDKATVFTDEESYSFESSKFTRTISPP
TLGTLRSCFSWSGGLGDFSRTPSPSPSTALQQFRRKSDSPTSLPENNMSD
VSQLKSEESSDDESHPLREEACSSQSQESGEFSLQSSNASKLSQCSSKDS
DSEESDCNIKLLDSQSDQTSKLRLSHFSKKDTPLRNKVPGLYKSSSADSL
STTKIKPLGPARASGLSKKPASIQKRKHHNAENKPGLQIKLNELWKNFGF
KKDSEKLPPCKKPLSPVRDNIQLTPEAEEDIFNKPECGRVQRAIFQ.
Exonuclease 1 (EXO1) Accession No. NM_001319224
(Homo sapiens exonuclease 1 (EXO1),
transcript variant 4) - isoform C
(SEQ ID NO: 187)
MGIQGLLQFIKEASEPIHVRKYKGQVVAVDTYCWLHKGAIACAEKLAKGE
PTDRYVGFCMKFVNMLLSHGIKPILVFDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSELSEDDLLSQYSLSFTKKTKKNSSEGNK
SLSFSEVFVPDLVNGPTNKKSVSTPPRTRNKFATFLORKNEESGAVVVPG
TRSRFFCSSDSTDCVSNKVSIQPLDETAVTDKENNLHESEYGDQEGKRLV
DTDVARNSSDDIPNNHIPGDHIPDKATVFTDEESYSFESSKFTRTISPPT
LGTLRSCFSWSGGLGDFSRTPSPSPSTALQQFRRKSDSPTSLPENNMSDV
SQLKSEESSDDESHPLREEACSSQSQESGEFSLQSSNASKLSQCSSKDSD
SEESDCNIKLLDSQSDQTSKLRLSHFSKKDTPLRNKVPGLYKSSSADSLS
TTKIKPLGPARASGLSKKPASIQKRKHHNAENKPGLQIKLNELWKNFGFK
KDSEKLPPCKKPLSPVRDNIQLTPEAEEDIFNKPECGRVQRAIFQ.

### B. Inteins and Split-Inteins

It will be understood that in some embodiments (e.g., delivery of a prime editor in vivo), it may be advantageous to split a polypeptide (e.g., a reverse transcriptase or a napDNAbp) or a fusion protein (e.g., a prime editor) into an N-terminal half and a C-terminal half, deliver them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g., a mini-intein) split into two pieces named N-intein and C-intein, respectively. The N-intein and C-intein of a split intein can associate non-covalently to form an active intein and catalyze the splicing reaction in essentially the same way as a contiguous intein does. Split inteins have been found in nature and have also been engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N- terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues, as long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues, as long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketones, aldehydes, Cys residues, and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured ioop or intervening amino acid sequence between the−12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g., micromolar) concentrations of proteins and can be carried out under physiological conditions.

Exemplary sequences are as follows:

| NAME | SEQUENCE OF LIGAND-DEPENDENT INTEIN |
|---|---|
| 2-4 | CLAEGTRIFDPVTGTTHRIEDVVDGRKPIHVVAAAKD |
| INTEIN: | GTLLARPVVSWFDQGTRDVIGLRIAGGAIVWATPDHK |
| | VLTEYGWRAAGELRKGDRVAGPGGSGNSLALSLTADQ |
| | MVSALLDAEPPILYSEYDPTSPFSEASMMGLLTNLAD |
| | RELVHMINWAKRVPGFVDLTLHDQAHLLECAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEELRYSVIREVLPTRRARTFDLEVEELHTLVAEG |
| | VVVHNC (SEQ ID NO: 188) |
| 3-2 | CLAEGTRIFDPVTGTTHRIEDVVDGRKPIHVVAVAKD |
| INTEIN | GTLLARPVVSWFDQGTRDVIGLRIAGGAIVWATPDHK |
| | VLTEYGWRAAGELRKGDRVAGPGGSGNSLALSLTADQ |
| | MVSALLDAEPPILYSEYDPTSPFSEASMMGLLTNLAD |
| | RELVHMINWAKRVPGFVDLTLHDQAHLLECAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYTNVVPL |
| | YDLLLEMLDAHRLHAGGSGASRVQAFADALDDKFLHD |
| | MLAEELRYSVIREVLPTRRARTFDLEVEELHTLVAEG |
| | VVVHNC (SEQ ID NO: 189) |
| 30R3-1 | CLAEGTRIFDPVTGTTHRIEDVVDGRKPIHVVAAAKD |
| INTEIN | GTLLARPVVSWFDQGTRDVIGLRIAGGATVWATPDHK |
| | VLTEYGWRAAGELRKGDRVAGPGGSGNSLALSLTADQ |
| | MVSALLDAEPPIPYSEYDPTSPFSEASMMGLLTNLAD |
| | RELVHMINWAKRVPGFVDLTLHDQAHLLECAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEGLRYSVIREVLPTRRARTFDLEVEELHTLVAEG |
| | VVVHNC (SEQ ID NO: 190) |
| 30R3-2 | CLAEGTRIFDPVTGTTHRIEDVVDGRKPIHVVAAAKD |
| INTEIN | GTLLARPVVSWFDQGTRDVIGLRIAGGATVWATPDHK |
| | VLTEYGWRAAGELRKGDRVAGPGGSGNSLALSLTADQ |
| | MVSALLDAEPPILYSEYDPTSPFSEASMMGLLTNLAD |
| | RELVHMINWAKRVPGFVDLTLHDQAHLLECAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEELRYSVIREVLPTRRARTFDLEVEELHTLVAEG |
| | VVVHNC (SEQ ID NO: 191) |
| 30R3-3 | CLAEGTRIFDPVTGTTHRIEDVVDGRKPIHVVAAAKD |
| INTEIN | GTLLARPVVSWFDQGTRDVIGLRIAGGATVWATPDHK |
| | VLTEYGWRAAGELRKGDRVAGPGGSGNSLALSLTADQ |
| | MVSALLDAEPPIPYSEYDPTSPFSEASMMGLLTNLAD |
| | RELVHMINWAKRVPGFVDLTLHDQAHLLECAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEELRYSVIREVLPTRRARTFDLEVEELHTLVAEG |
| | VVVHNC (SEQ ID NO: 192) |
| 37R3-1 | CLAEGTRIFDPVTGTTHRIEDVVDGRKPIHVVAAAKD |
| INTEIN | GTLLARPVVSWFDQGTRDVIGLRIAGGATVWATPDHK |
| | VLTEYGWRAAGELRKGDRVAGPGGSGNSLALSLTADQ |
| | MVSALLDAEPPILYSEYNPTSPFSEASMMGLLTNLAD |
| | RELVHMINWAKRVPGFVDLTLHDQAHLLERAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEGLRYSVIREVLPTRRARTFDLEVEELHTLVAEG |
| | VVVHNC ((SEQ ID NO: 193) |
| 37R3-2 | CLAEGTRIFDPVTGTTHRIEDVVDGRKPIHVVAAAKD |
| INTEIN | GTLLARPVVSWFDQGTRDVIGLRIAGGAIVWATPDHK |
| | VLTEYGWRAAGELRKGDRVAGPGGSGNSLALSLTADQ |
| | MVSALLDAEPPILYSEYDPTSPFSEASMMGLLTNLAD |
| | RELVHMINWAKRVPGFVDLTLHDQAHLLERAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEGLRYSVIREVLPTRRARTFDLEVEELHTLVAEG |
| | VVVHNC (SEQ ID NO: 194) |
| 37R3-3 | CLAEGTRIFDPVTGTTHRIEDVVDGRKPIHVVAVAKD |
| INTEIN | GTLLARPVVSWFDQGTRDVIGLRIAGGATVWATPDHK |
| | VLTEYGWRAAGELRKGDRVAGPGGSGNSLALSLTADQ |
| | MVSALLDAEPPILYSEYDPTSPFSEASMMGLLTNLAD |
| | RELVHMINWAKRVPGFVDLTLHDQAHLLERAWLEILM |
| | IGLVWRSMEHPGKLLFAPNLLLDRNQGKCVEGMVEIF |
| | DMLLATSSRFRMMNLQGEEFVCLKSIILLNSGVYTFL |
| | SSTLKSLEEKDHIHRALDKITDTLIHLMAKAGLTLQQ |
| | QHQRLAQLLLILSHIRHMSNKGMEHLYSMKYKNVVPL |
| | YDLLLEMLDAHRLHAGGSGASRVQAFADALDDKFLHD |
| | MLAEELRYSVIREVLPTRRARTFDLEVEELHTLVAEG |
| | VVVHNC (SEQ ID NO: 195) |

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the art or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference. In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as two inactive fragments that subsequently undergo ligation to form a functional product.

### RNA-Protein Interaction Domain

In various embodiments, two separate protein domains (e.g., a Cas9 domain and a polymerase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (a.k.a. “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a prime editor, as well as to recruit additional functionalities to a prime editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario, a reverse transcriptase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 196).

The amino acid sequence of the MCP or MS2cp is:

(SEQ ID NO: 197)
GSASNFTQFVLVDNGGTGDVTVAPSNFANGVAEWISSNSRSQAYKVTCSV
RQSSAQNRKYTIKVEVPKVATQTVGGEELPVAGWRSYLNMELTIPIFATN
SDCELIVKAMQGLLKDGNPIPSAIAANSGIY.

### C. UGI Domain

In other embodiments, the prime editors delivered by the PE-VLPs described herein may comprise one or more uracil glycosylase inhibitor domains. The term “uracil glycosylase inhibitor (UGI)” or “UGI domain,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 198. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 198. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 198. In some embodiments, a UGI comprises an amino acid sequence homologous to the amino acid sequence set forth in SEQ ID NO: 198, or an amino acid sequence homologous to a fragment of the amino acid sequence set forth in SEQ ID NO: 198. In some embodiments, proteins comprising UGI, or fragments of UGI, homologs of UGI, or UGI fragments, are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 198. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 198. In some embodiments, the UGI comprises the following amino acid sequence: Uracil-DNA glycosylase inhibitor:

>sp|P14739|UNGI_BPPB2
(SEQ ID NO: 198)
MTNLSDIIEKETGKQLVIQESILMLPEEVEEVIGNKPESDILVHTAYDES
TDENVMLLTSDAPEYKPWALVIQDSNGENKIKML.

The prime editors utilized in the methods and compositions described herein may comprise more than one UGI domain, which may be separated by one or more linkers as described herein.

### D. Additional PE Elements

In certain embodiments, the prime editors utilized in the methods and compositions described herein may comprise an inhibitor of base repair. The term “inhibitor of base repair” or “IBR” refers to a protein that is capable in inhibiting the activity of a nucleic acid repair enzyme, for example, a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE 1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants threreof. In some embodiments, the IBR is an iBER that may be a TDG inhibitor, an MBD4 inhibitor, or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 202 (human TDG).

Some exemplary glycosylases are provided below. The catalytically inactivated variants of any of these glycosylase domains are iBERs that may be fused to the napDNAbp or polymerase domain of the prime editors utilized in the methods and compositions provided in this disclosure.

OGG (human)
(SEQ ID NO: 199)
MPARALLPRRMGHRTLASTPALWASIPCPRSELRLDLVLPSGQSFRWREQ
SPAHWSGVLADQVWTLTQTEEQLHCTVYRGDKSQASRPTPDELEAVRKYF
QLDVTLAQLYHHWGSVDSHFQEVAQKFQGVRLLRQDPIECLFSFICSSNN
NIARITGMVERLCQAFGPRLIQLDDVTYHGFPSLQALAGPEVEAHLRKLG
LGYRARYVSASARAILEEQGGLAWLQQLRESSYEEAHKALCILPGVGTKV
ADCICLMALDKPQAVPVDVHMWHIAQRDYSWHPTTSQAKGPSPQTNKELG
NFFRSLWGPYAGWAQAVLFSADLRQSRHAQEPPAKRRKGSKGPEG
MPG (human)
(SEQ ID NO: 200)
MVTPALQMKKPKQFCRRMGQKKQRPARAGQPHSSSDAAQAPAEQPHSSSD
AAQAPCPRERCLGPPTTPGPYRSIYFSSPKGHLTRLGLEFFDQPAVPLAR
AFLGQVLVRRLPNGTELRGRIVETEAYLGPEDEAAHSRGGRQTPRNRGMF
MKPGTLYVYIIYGMYFCMNISSQGDGACVLLRALEPLEGLETMRQLRSTL
RKGTASRVLKDRELCSGPSKLCQALAINKSFDQRDLAQDEAVWLERGPLE
PSEPAVVAAARVGVGHAGEWARKPLRFYVRGSPWVSVVDRVAEQDTQA
MBD4 (human)
(SEQ ID NO: 201)
MGTTGLESLSLGDRGAAPTVTSSERLVPDPPNDLRKEDVAMELERVGEDE
EQMMIKRSSECNPLLQEPIASAQFGATAGTECRKSVPCGWERVVKQRLFG
KTAGRFDVYFISPQGLKFRSKSSLANYLHKNGETSLKPEDFDFTVLSKRG
IKSRYKDCSMAALTSHLQNQSNNSNWNLRTRSKCKKDVFMPPSSSSELQE
SRGLSNFTSTHLLLKEDEGVDDVNFRKVRKPKGKVTILKGIPIKKTKKGC
RKSCSGFVQSDSKRESVCNKADAESEPVAQKSQLDRTVCISDAGACGETL
SVTSEENSLVKKKERSLSSGSNFCSEQKTSGIINKFCSAKDSEHNEKYED
TFLESEEIGTKVEVVERKEHLHTDILKRGSEMDNNCSPTRKDFTGEKIFQ
EDTIPRTQIERRKTSLYFSSKYNKEALSPPRRKAFKKWTPPRSPFNLVQE
TLFHDPWKLLIATIFLNRTSGKMAIPVLWKFLEKYPSAEVARTADWRDVS
ELLKPLGLYDLRAKTIVKFSDEYLTKQWKYPIELHGIGKYGNDSYRIFCV
NEWKQVHPEDHKLNKYHDWLWENHEKLSLS
TDG (human)
(SEQ ID NO: 202)
MEAENAGSYSLQQAQAFYTFPFQQLMAEAPNMAVVNEQQMPEEVPAPAPA
QEPVQEAPKGRKRKPRTTEPKQPVEPKKPVESKKSGKSAKSKEKQEKITD
TFKVKRKVDRFNGVSEAELLTKTLPDILTFNLDIVIIGINPGLMAAYKGH
HYPGPGNHFWKCLFMSGLSEVQLNHMDDHTLPGKYGIGFTNMVERTTPGS
KDLSSKEFREGGRILVQKLQKYQPRIAVFNGKCIYEIFSKEVFGVKVKNL
EFGLQPHKIPDTETLCYVMPSSSARCAQFPRAQDKVHYYIKLKDLRDQLK
GIERNMDVQEVQYTFDLQLAQEDAKKMAVKEEKYDPGYEAAYGGAYGENP
CSSEPCGFSSNGLIESVELRGESAFSGIPNGQWMTQSFTDQIPSFSNHCG
TQEQEEESHA

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A prime editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011, and incorporated herein by reference in its entirety.

In an aspect of the disclosure, a reporter gene that includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product that serves as a marker by which to measure the alteration or modification of expression of the gene product. In certain embodiments of the disclosure, the gene product is luciferase. In a further embodiment of the disclosure, the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments of the present disclosure, the activity of the prime editing system delivered by the presently described PE-VLPs may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the PEgRNA may deliver the guide prior to the vector encoding the PE system. In some embodiments, the vectors encoding the PE system and PEgRNA are delivered simultaneously. In certain embodiments, the simultaneously delivered vectors temporally deliver, e.g., the PE, PEgRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the PEgRNA may be self-destroyed via cleavage of a target sequence present on the vector by the PE system. The cleavage may prevent continued transcription of a PE or a PEgRNA from the vector. Although transcription may occur on the linearized vector for some amount of time, the expressed transcripts or proteins subject to intracellular degradation will have less time to produce off-target effects without continued supply from expression of the encoding vectors.

Delivery of MMR Inhibitors with PE-VLPs

In some embodiments, the present disclosure contemplates delivery of an inhibitor of the mismatch repair (MMR) pathway using the PE-VLPs described herein alongside a prime editor to enhance the efficiency of prime editing. Thus, the present disclosure contemplates any suitable means to inhibit MMR. In one embodiment, the disclosure embraces administering an effective amount of an inhibitor of the MMR pathway. In various embodiments, the MMR pathway may be inhibited by inhibiting, blocking, or inactivating any one or more MMR proteins or variants at the genetic level (e.g., in the gene encoding the one or more MMR proteins, such as introducing a mutation that inactivates the MMR protein or variant thereof), transcriptional level (e.g., by transcript knockdown), translational level (e.g., by blocking translation of one or more MMR proteins from their cognate transcripts), or at the protein level (e.g., application of an inhibitor (e.g., small molecule, antibody, dominant negative protein partner) or by targeted protein degradation (e.g., PROTAC-based degradation). The present disclosure also contemplates methods of prime editing using the PE-VLPs described herein which are designed to install modifications to a nucleic acid molecule that evade correction by the MMR pathway, without the need to provide an MMR inhibitor. Delivering an MMR inhibitor alongside the prime editor using the presently described PE-VLPs, or installing modifications to a nucleic acid molecule that avoid correction by the MMR pathway, results in increased editing efficiency and reduced indel formation. As used herein, “during” prime editing can embrace any suitable sequence of events, such that the prime editing step can be applied before, at the same time, or after the step of blocking, inhibiting, or inactivating the MMR pathway (e.g., by targeting the inhibition of MLH1). For example, in some embodiments, an inhibitor of the MMR pathway may be delivered at the same time as the prime editor, either in the same PE-VLP, or in separate PE-VLPs. In some embodiments, an inhibitor of the MMR pathway may be delivered before delivery of the prime editor, or after delivery of the prime editor.

In some embodiments, a prime editing system component, e.g., a pegRNA, is designed to install modifications in the target nucleic acid which evade the MMR system, without the need to provide an inhibitor. In certain embodiments, the DNA mismatch repair (MMR) system can be inhibited, blocked, or otherwise inactivated by inhibiting one or more proteins of the MMR system, including, but not limited to MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA.

Thus, in one aspect, the present disclosure provides a method for editing a nucleotide molecule (e.g., a genome) by delivering an inhibitor of the MMR pathway and a prime editor using the PE-VLPs described herein.

In another aspect, the present disclosure provides a method for editing a nucleotide molecule (e.g., a genome) by delivering an inhibitor of the MMR system, e.g., MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA, and a prime editor using the PE-VLPs described herein.

In one aspect, the present disclosure delivery of a prime editor and an inhibitor of MLH1 or a variant thereof using the PE-VLPs described herein. Without being bound by theory, MLH1 is a key MMR protein that heterodimerizes with PMS2 to form MutL alpha, a component of the post-replicative DNA mismatch repair system (MMR). DNA repair is initiated by MutS alpha (MSH2-MSH6) or MutS beta (MSH2-MSH3) binding to a dsDNA mismatch, then MutL alpha is recruited to the heteroduplex. Assembly of the MutL-MutS-heteroduplex ternary complex in presence of RFC and PCNA is sufficient to activate endonuclease activity of PMS2. It introduces single-strand breaks near the mismatch and thus generates new entry points for the exonuclease EXO1 to degrade the strand containing the mismatch. DNA methylation would prevent cleavage and therefore assure that only the newly mutated DNA strand is going to be corrected. MutL alpha (MLH1-PMS2) interacts physically with the clamp loader subunits of DNA polymerase III, suggesting that it may play a role to recruit the DNA polymerase III to the site of the MMR. Also implicated in DNA damage signaling, a process which induces cell cycle arrest and can lead to apoptosis in case of major DNA damages. MLH1 also heterodimerizes with MLH3 to form MutL gamma which plays a role in meiosis. The “canonical” human MLH1 amino acid sequence is represented by:

>sp|P40692|MLH1_HUMAN DNA mismatch repair
protein Mlh1 OS = Homo sapiens
OX = 9606 GN = MLH1 PE = 1 SV = 1
(SEQ ID NO: 9)
MSFVAGVIRRLDETVVNRIAAGEVIQRPANAIKEMIENCLDAKSTSIQVI
VKEGGLKLIQ
IQDNGTGIRKEDLDIVCERFTTSKLQSFEDLASISTYGFRGEALASISHV
AHVTITTKTADGKCAYRASYSDGKLKAPPKPCAGNQGTQITVEDLFYNIA
TRRKALKNPSEEYGKILEVVGRYSVHNAGISFSVKKQGETVADVRTLPNA
STVDNIRSIFGNAVSRELIEIGCEDKTLAF
KMNGYISNANYSVKKCIFLLFINHRLVESTSLRKAIETVYAAYLPKNTHP
FLYLSLEISP
QNVDVNVHPTKHEVHFLHEESILERVQQHIESKLLGSNSSRMYFTQTLLP
GLAGPSGEMVKSTTSLTSSSTSGSSDKVYAHQMVRTDSREQKLDAFLQPL
SKPLSSQPQAIVTEDKTDIS
SGRARQQDEEMLELPAPAEVAAKNQSLEGDTTKGTSEMSEKRGPTSSNPR
KRHREDSDVEMVEDDSRKEMTAACTPRRRIINLTSVLSLQEEINEQGHEV
LREMLHNHSFVGCVNPQWALAQHQTKLYLLNTTKLSEELFYQILIYDFAN
FGVLRLSEPAPLFDLAMLALDSPESGWTEEDGPKEGLAEYIVEFLKKKAE
MLADYFSLEIDEEGNLIGLPLLIDNYVPPLEGLPIFILRLATEVNWDEEK
ECFESLSKECAMFYSIRKQYISEESTLSGQQSEVPGSIPNSWKWTVEHIV
YKALRSHILPPKHFTEDGNILQLANLPDLYKVFERC

MLH1 also may include other human isoforms, including P40692-2, which differs from the canonical sequence in that residues 1-241 of the canonical sequence are missing:

>sp|P40692-2|MLH1_HUMAN Isoform 2 of DNA
mismatch repair protein Mlh1 OS = Homo
sapiens OX = 9606 GN = MLH1
(SEQ ID NO: 10)
MNGYISNANYSVKKCIFLLFINHRLVESTSLRKAIETVYAAYLPKNTHPF
LYLSLEISPQ
NVDVNVHPTKHEVHFLHEESILERVQQHIESKLLGSNSSRMYFTQTLLPG
LAGPSGEMVKSTTSLTSSSTSGSSDKVYAHQMVRTDSREQKLDAFLQPLS
KPLSSQPQAIVTEDKTDISS
GRARQQDEEMLELPAPAEVAAKNQSLEGDTTKGTSEMSEKRGPTSSNPRK
RHREDSDVEMVEDDSRKEMTAACTPRRRIINLTSVLSLQEEINEQGHEVL
REMLHNHSFVGCVNPQWALAQHQTKLYLLNTTKLSEELFYQILIYDFANF
GVLRLSEPAPLFDLAMLALDSPESGWTEEDGPKEGLAEYIVEFLKKKAEM
LADYFSLEIDEEGNLIGLPLLIDNYVPPLEGLPIFILRLATEVNWDEEKE
CFESLSKECAMFYSIRKQYISEESTLSGQQSEVPGSIPNSWKWTVEHIVY
KALRSHILPPKHFTEDGNILQLANLPDLYKVFERC
MLH1 also may include a third known isoform
known as P40692-3, which differs from the
canonical sequence in that residues 1-101
(of MSFVAGVIRR . . . ASISTYGFRG (SEQ ID
NO: 9)) are replaced with MAF:
>sp|P40692-3|MLH1_HUMAN Isoform 3 of DNA
mismatch repair protein Mlh1 OS = Homo
sapiens OX = 9606 GN = MLH1
(SEQ ID NO: 12)
MAFEALASISHVAHVTITTKTADGKCAYRASYSDGKLKAPPKPCAGNQGT
QITVEDLFYNIATRRKALKNPSEEYGKILEVVGRYSVHNAGISFSVKKQG
ETVADVRTLPNASTVDNIRSIFGNAVSRELIEIGCEDKTLAFKMNGYISN
ANYSVKKCIFLLFINHRLVESTSLRKAIET
VYAAYLPKNTHPFLYLSLEISPQNVDVNVHPTKHEVHFLHEESILERVQQ
HIESKLLGSN
SSRMYFTQTLLPGLAGPSGEMVKSTTSLTSSSTSGSSDKVYAHQMVRTDS
REQKLDAFLQPLSKPLSSQPQAIVTEDKTDISSGRARQQDEEMLELPAPA
EVAAKNQSLEGDTTKGTSEMSEKRGPTSSNPRKRHREDSDVEMVEDDSRK
EMTAACTPRRRIINLTSVLSLQEEINEQGHEVLREMLHNHSFVGCVNPQW
ALAQHQTKLYLLNTTKLSEELFYQILIYDFANFGVLRLSEPAPLFDLAML
ALDSPESGWTEEDGPKEGLAEYIVEFLKKKAEMLADYFSLEIDEEGNLIG
LPLLIDNYVPPLEGLPIFILRLATEVNWDEEKECFESLSKECAMFYSIRK
QYISEESTLS
GQQSEVPGSIPNSWKWTVEHIVYKALRSHILPPKHFTEDGNILQLANLPD
LYKVFERC.

The disclosure contemplates that inhibitors of any of the following proteins may be delivered using the PE-VLPs described herein to inhibit the MMR pathway during prime editing. In addition, such exemplary proteins may also be used to engineer or otherwise make a dominant negative variant that may be used as a type of inhibitor when administered in an effective amount which blocks, inactivates, or inhibits the MMR. Without being bound by theory, it is believed that MLH1 dominant negative mutants can saturate binding of MutS. Exemplary MLH1 proteins include the following amino acid sequences, or amino acid sequences having at least 70%, at least 75%, at least 80%, at least 85%, at least 90%, at least 95%, at least 96%, at least 97%, at least 98%, at least 99%, or up to 100% sequence identity with any of the following sequences:

| | | SEQ |
|---|---|---|
| Descrip- | | ID |
| tion | Sequence | NO: |
| MLH1 | MSFVAGVIRRLDETVVNRIAAGEVIQRPAN | 9 |
| Homo | AIKEMIENCLDAKSTSIQVIVKEGGLKLIQ | |
| sapiens | IQDNGTGIRKEDLDIVCERFTTSKLQSFED | |
| SwissProt | LASISTYGFRGEALASISHVAHVTITTKTA | |
| Accession | DGKCAYRASYSDGKLKAPPKPCAGNQGTQI | |
| No. | TVEDLFYNIATRRKALKNPSEEYGKILEVV | |
| P40692 | GRYSVHNAGISFSVKKQGETVADVRTLPNA | |
| Wild | STVDNIRSIFGNAVSRELIEIGCEDKTLAF | |
| type | KMNGYISNANYSVKKCIFLLFINHRLVEST | |
| | SLRKAIETVYAAYLPKNTHPFLYLSLEISP | |
| | QNVDVNVHPTKHEVHFLHEESILERVQQHI | |
| | ESKLLGSNSSRMYFTQTLLPGLAGPSGEMV | |
| | KSTTSLTSSSTSGSSDKVYAHQMVRTDSRE | |
| | QKLDAFLQPLSKPLSSQPQAIVTEDKTDIS | |
| | SGRARQQDEEMLELPAPAEVAAKNQSLEGD | |
| | TTKGTSEMSEKRGPTSSNPRKRHREDSDVE | |
| | MVEDDSRKEMTAACTPRRRIINLTSVLSLQ | |
| | EEINEQGHEVLREMLHNHSFVGCVNPQWAL | |
| | AQHQTKLYLLNTTKLSEELFYQILIYDFAN | |
| | FGVLRLSEPAPLFDLAMLALDSPESGWTEE | |
| | DGPKEGLAEYIVEFLKKKAEMLADYFSLEI | |
| | DEEGNLIGLPLLIDNYVPPLEGLPIFILRL | |
| | ATEVNWDEEKECFESLSKECAMFYSIRKQY | |
| | ISEESTLSGQQSEVPGSIPNSWKWTVEHIV | |
| | YKALRSHILPPKHFTEDGNILQLANLPDLY | |
| | KVFERC | |
| MLH1 | MAFVAGVIRRLDETVVNRIAAGEVIQRPAN | 203 |
| Mus | AIKEMIENCLDAKSTNIQVVVKEGGLKLIQ | |
| musculus | IQDNGTGIRKEDLDIVCERFTTSKLQTFED | |
| SwissProt | LASISTYGFRGEALASISHVAHVTITTKTA | |
| Accession | DGKCAYRASYSDGKLQAPPKPCAGNQGTLI | |
| No. | TVEDLFYNIITRRKALKNPSEEYGKILEVV | |
| Q9JK91 | GRYSIHNSGISFSVKKQGETVSDVRTLPNA | |
| Wild | TTVDNIRSIFGNAVSRELIEVGCEDKTLAF | |
| type | KMNGYISNANYSVKKCIFLLFINHRLVESA | |
| | ALRKAIETVYAAYLPKNTHPFLYLSLEISP | |
| | QNVDVNVHPTKHEVHFLHEESILQRVQQHI | |
| | ESKLLGSNSSRMYFTQTLLPGLAGPSGEAA | |
| | RPTTGVASSSTSGSGDKVYAYQMVRTDSRE | |
| | QKLDAFLQPVSSLGPSQPQDPAPVRGARTE | |
| | GSPERATREDEEMLALPAPAEAAAESENLE | |
| | RESLMETSDAAQKAAPTSSPGSSRKRHRED | |
| | SDVEMVENASGKEMTAACYPRRRIINLTSV | |
| | LSLQEEISERCHETLREMLRNHSFVGCVNP | |
| | QWALAQHQTKLYLLNTTKLSEELFYQILIY | |
| | DFANFGVLRLSEPAPLFDLAMLALDSPESG | |
| | WTEDDGPKEGLAEYIVEFLKKKAEMLADYF | |
| | SVEIDEEGNLIGLPLLIDSYVPPLEGLPIF | |
| | ILRLATEVNWDEEKECFESLSKECAMFYSI | |
| | RKQYILEESTLSGQQSDMPGSTSKPWKWTV | |
| | EHIIYKAFRSHLLPPKHFTEDGNVLQLANL | |
| | PDLYKVFERC | |
| MLH1 | MSFVAGVIRRLDETVVNRIAAGEVIQRPAN | 204 |
| Rattus | AIKEMTENCLDAKSTNIQVIVREGGLKLIQ | |
| norvegicus | IQDNGTGIRKEDLDIVCERFTTSKLQTFED | |
| SwissProt | LAMISTYGFRGEALASISHVAHVTITTKTA | |
| Accession | DGKCAYRASYSDGKLQAPPKPCAGNQGTLI | |
| No. | TVEDLFYNIITRKKALKNPSEEYGKILEVV | |
| P97679 | GRYSIHNSGISFSVKKQGETVSDVRTLPNA | |
| Wild | TTVDNIRSIFGNAVSRELIEVGCEDKTLAF | |
| type | KMNGYISNANYSVKKCIFLLFINHRLVESA | |
| | ALKKAIEAVYAAYLPKNTHPFLYLILEISP | |
| | QNVDVNVHPTKHEVHFLHEESILERVQQHI | |
| | ESKLLGSNSSRMYFTQTLLPGLAGPSGEAV | |
| | KSTTGIASSSTSGSGDKVHAYQMVRTDSRD | |
| | QKLDAFMQPVSRRLPSQPQDPVPGNRTEGS | |
| | PEKAMQKDQEISELPAPMEAAADSASLERE | |
| | SVIGASEVVAPQRHPSSPGSSRKRHPEDSD | |
| | VEMMENDSRKEMTAACYPRRRIINLTSVLS | |
| | LQEEINDRGHETLREMLRNHTFVGCVNPQW | |
| | ALAQHQTKLYLLNTTKLSEELFYQILIYDF | |
| | ANFGVLRLPEPAPLFDFAMLALDSPESGWT | |
| | EEDGPKEGLAEYIVEFLKKKAKMLADYFSV | |
| | EIDEEGNLIGLPLLIDSYVPPLEGLPIFIL | |
| | RLATEVNWDEEECFESLSKECAVFYSIRKQ | |
| | YILEESALSGQQSDMPGSPSKPWKWTVEHI | |
| | IYKAFRSHLLPPKHFTEDGNVLQLANLPDL | |
| | CKVFERC | |
| MLH1 | MSLVAGVIRRLDETVVNRIAAGEVIQRPAN | 205 |
| Bos taurus | AIKEMIENCLDAKSTSIQVVVKEGGLKLIQ | |
| SwissProt | IQDNGTGIRKEDLEIVCERFTTSKLQSFED | |
| Accession | LAHISTYGFRGEALASISHVAHVTITTKTA | |
| No. | DGKCAYRAHYSDGKLKAPPKPCAGNQGTQI | |
| F1MPGO | TVEDLFYNISTRRKALKNPSEEYGKILEVV | |
| Wild | GRYAVHNSGIGFSVKKQGETVADVRTLPNA | |
| type | TTVDNIRSIFGNAVSRELIEVECEDKTLAF | |
| | KMNGYISNANYSVKKCIFLLFINHRLVESA | |
| | SLRKAIETVYAAYLPKSTHPFLYLSLEISP | |
| | QNVDVNVHPTKHEVHFLHEDSILERLQQHI | |
| | ESRLLGSNASRTYFTQTLLPGLPGPSGEAV | |
| | KSTASVTSSSTAGSGDRVYAHQMVRTDCRE | |
| | QKLDAFLQPVSKALSSQPQAVVPEHRTDAS | |
| | SSGTRQQDEEMLELPAPAAVAAKSQALEDD | |
| | ATMRAADLAEKRGPSSSPENPRKRPREDSD | |
| | VEMVEDASRKEMTAACTPRRRIINLTSVLS | |
| | LQEEINERGHETLREMLHNHSFVGCVNPQW | |
| | ALAQHQTKLYLLNTTRLSEELFYQILVYDF | |
| | ANFGVLRLSEPAPLFDLAMLALDSPESGWT | |
| | EEDGPKEGLAEYIVEFLKKKAEMLADYFSL | |
| | EIDEEGNLVGLPLLIDNYVPPLEGLPIFIL | |
| | RLATEVNWDEEKECFESLSKECAMFYSIRK | |
| | QYVSAESTLSGQQSEVPGSTANPWKWTVEH | |
| | VIYKAFRSHLLPPKHFTEDGNILQLANLPD | |
| | LYKVFERC | |

The PE-VLPs described herein may be used to deliver MLH1 mutants or truncated variants. In some embodiments, the mutants and truncated variants of the human MLH1 wild-type protein are utilized.

In one aspect, a truncated variant of human MLH1 is delivered using the PE-VLPs of the present disclosure. In some embodiments, amino acids 754-756 of the wild-type human MLH1 protein are truncated (Δ754-756, hereinafter referred to as MLH1dn). In some embodiments, a truncated variant of human MLH1 comprising only the N-terminal domain (amino acids 1-335) is provided (hereinafter referred to as MLH1dn^{NTD}). In various embodiments, the following MLH1 variants are provided in this disclosure:

| Description | Sequence | SEQ ID NO: |
|---|---|---|
| MLH1 | MSFVAGVIRRLDETVVNRIAAGEVIQRPANAIKAMIEN | 13 |
| E34A | CLDAKSTSIQVIVKEGGLKLIQIQDNGTGIRKEDLDIVC | |
| | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLLGSNSSRMYFTQTL | |
| | LPGLAGPSGEMVKSTTSLTSSSTSGSSDKVYAHQMVRT | |
| | DSREQKLDAFLQPLSKPLSSQPQAIVTEDKTDISSGRAR | |
| | QQDEEMLELPAPAEVAAKNQSLEGDTTKGTSEMSEKR | |
| | GPTSSNPRKRHREDSDVEMVEDDSRKEMTAACTPRRR | |
| | IINLTSVLSLQEEINEQGHEVLREMLHNHSFVGCVNPQ | |
| | WALAQHQTKLYLLNTTKLSEELFYQILIYDFANFGVLR | |
| | LSEPAPLFDLAMLALDSPESGWTEEDGPKEGLAEYIVE | |
| | FLKKKAEMLADYFSLEIDEEGNLIGLPLLIDNYVPPLEG | |
| | LPIFILRLATEVNWDEEKECFESLSKECAMFYSIRKQYIS | |
| | EESTLSGQQSEVPGSIPNSWKWTVEHIVYKALRSHILPP | |
| | KHFTEDGNILQLANLPDLYKVFERC | |
| MLH1 | MSFVAGVIRRLDETVVNRIAAGEVIQRPANAIKEMIEN | 14 |
| Δ756 | CLDAKSTSIQVIVKEGGLKLIQIQDNGTGIRKEDLDIVC | |
| | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLLGSNSSRMYFTQTL | |
| | LPGLAGPSGEMVKSTTSLTSSSTSGSSDKVYAHQMVRT | |
| | DSREQKLDAFLQPLSKPLSSQPQAIVTEDKTDISSGRAR | |
| | QQDEEMLELPAPAEVAAKNQSLEGDTTKGTSEMSEKR | |
| | GPTSSNPRKRHREDSDVEMVEDDSRKEMTAACTPRRR | |
| | IINLTSVLSLQEEINEQGHEVLREMLHNHSFVGCVNPQ | |
| | WALAQHQTKLYLLNTTKLSEELFYQILIYDFANFGVLR | |
| | LSEPAPLFDLAMLALDSPESGWTEEDGPKEGLAEYIVE | |
| | FLKKKAEMLADYFSLEIDEEGNLIGLPLLIDNYVPPLEG | |
| | LPIFILRLATEVNWDEEKECFESLSKECAMFYSIRKQYIS | |
| | EESTLSGQQSEVPGSIPNSWKWTVEHIVYKALRSHILPP | |
| | KHFTEDGNILQLANLPDLYKVFER[-] | |
| MLH1 | MSFVAGVIRRLDETVVNRIAAGEVIQRPANAIKEMIEN | 15 |
| Δ754-756 | CLDAKSTSIQVIVKEGGLKLIQIQDNGTGIRKEDLDIVC | |
| | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLLGSNSSRMYFTQTL | |
| | LPGLAGPSGEMVKSTTSLTSSSTSGSSDKVYAHQMVRT | |
| | DSREQKLDAFLQPLSKPLSSQPQAIVTEDKTDISSGRAR | |
| | QQDEEMLELPAPAEVAAKNQSLEGDTTKGTSEMSEKR | |
| | GPTSSNPRKRHREDSDVEMVEDDSRKEMTAACTPRRR | |
| | IINLTSVLSLQEEINEQGHEVLREMLHNHSFVGCVNPQ | |
| | WALAQHQTKLYLLNTTKLSEELFYQILIYDFANFGVLR | |
| | LSEPAPLFDLAMLALDSPESGWTEEDGPKEGLAEYIVE | |
| | FLKKKAEMLADYFSLEIDEEGNLIGLPLLIDNYVPPLEG | |
| | LPIFILRLATEVNWDEEKECFESLSKECAMFYSIRKQYIS | |
| | EESTLSGQQSEVPGSIPNSWKWTVEHIVYKALRSHILPP | |
| | KHFTEDGNILQLANLPDLYKVF[- - -] | |
| MLH1 | MSFVAGVIRRLDETVVNRIAAGEVIQRPANAIKAMIEN | 16 |
| E34A Δ754- | CLDAKSTSIQVIVKEGGLKLIQIQDNGTGIRKEDLDIVC | |
| 756 | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLLGSNSSRMYFTQTL | |
| | LPGLAGPSGEMVKSTTSLTSSSTSGSSDKVYAHQMVRT | |
| | DSREQKLDAFLQPLSKPLSSQPQAIVTEDKTDISSGRAR | |
| | QQDEEMLELPAPAEVAAKNQSLEGDTTKGTSEMSEKR | |
| | GPTSSNPRKRHREDSDVEMVEDDSRKEMTAACTPRRR | |
| | IINLTSVLSLQEEINEQGHEVLREMLHNHSFVGCVNPQ | |
| | WALAQHQTKLYLLNTTKLSEELFYQILIYDFANFGVLR | |
| | LSEPAPLFDLAMLALDSPESGWTEEDGPKEGLAEYIVE | |
| | FLKKKAEMLADYFSLEIDEEGNLIGLPLLIDNYVPPLEG | |
| | LPIFILRLATEVNWDEEKECFESLSKECAMFYSIRKQYIS | |
| | EESTLSGQQSEVPGSIPNSWKWTVEHIVYKALRSHILPP | |
| | KHFTEDGNILQLANLPDLYKVF[- - -] | |
| MLH1 1- | MSFVAGVIRRLDETVVNRIAAGEVIQRPANAIKEMIEN | 17 |
| 335 | CLDAKSTSIQVIVKEGGLKLIQIQDNGTGIRKEDLDIVC | |
| | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLL | |
| MLH1 1- | MSFVAGVIRRLDETVVNRIAAGEVIQRPANAIKAMIEN | 18 |
| 335 E34A | CLDAKSTSIQVIVKEGGLKLIQIQDNGTGIRKEDLDIVC | |
| | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLL | |
| MLH1 1- | MSFVAGVIRRLDETVVNRIAAGEVIQRPANAIKEMIEN | 19 |
| 335 | CLDAKSTSIQVIVKEGGLKLIQIQDNGTGIRKEDLDIVC | |
| NLS^{SV40} | ERFTTSKLQSFEDLASISTYGFRGEALASISHVAHVTITT | |
| | KTADGKCAYRASYSDGKLKAPPKPCAGNQGTQITVED | |
| | LFYNIATRRKALKNPSEEYGKILEVVGRYSVHNAGISFS | |
| | VKKQGETVADVRTLPNASTVDNIRSIFGNAVSRELIEIG | |
| | CEDKTLAFKMNGYISNANYSVKKCIFLLFINHRLVESTS | |
| | LRKAIETVYAAYLPKNTHPFLYLSLEISPQNVDVNVHP | |
| | TKHEVHFLHEESILERVQQHIESKLLPKKKRKV | |
| MLH1 501- | INLTSVLSLQEEINEQGHEVLREMLHNHSFVGCVNPQW | 206 |
| 756 | ALAQHQTKLYLLNTTKLSEELFYQILIYDFANFGVLRLS | |
| | EPAPLFDLAMLALDSPESGWTEEDGPKEGLAEYIVEFL | |
| | KKKAEMLADYFSLEIDEEGNLIGLPLLIDNYVPPLEGLP | |
| | IFILRLATEVNWDEEKECFESLSKECAMFYSIRKQYISE | |
| | ESTLSGQQSEVPGSIPNSWKWTVEHIVYKALRSHILPPK | |
| | HFTEDGNILQLANLPDLYKVFERC | |
| MLH1 501- | INLTSVLSLQEEINEQGHEVLREMLHNHSFVGCVNPQW | 207 |
| 753 | ALAQHQTKLYLLNTTKLSEELFYQILIYDFANFGVLRLS | |
| | EPAPLFDLAMLALDSPESGWTEEDGPKEGLAEYIVEFL | |
| | KKKAEMLADYFSLEIDEEGNLIGLPLLIDNYVPPLEGLP | |
| | IFILRLATEVNWDEEKECFESLSKECAMFYSIRKQYISE | |
| | ESTLSGQQSEVPGSIPNSWKWTVEHIVYKALRSHILPPK | |
| | HFTEDGNILQLANLPDLYKVF[- - -] | |
| MLH1 461- | KRGPTSSNPRKRHREDSDVEMVEDDSRKEMTAACTPR | 208 |
| 756 | RRIINLTSVLSLQEEINEQGHEVLREMLHNHSFVGCVNP | |
| | QWALAQHQTKLYLLNTTKLSEELFYQILIYDFANFGVL | |
| | RLSEPAPLFDLAMLALDSPESGWTEEDGPKEGLAEYIV | |
| | EFLKKKAEMLADYFSLEIDEEGNLIGLPLLIDNYVPPLE | |
| | GLPIFILRLATEVNWDEEKECFESLSKECAMFYSIRKQY | |
| | ISEESTLSGQQSEVPGSIPNSWKWTVEHIVYKALRSHIL | |
| | PPKHFTEDGNILQLANLPDLYKVFERC | |
| MLH1 461- | KRGPTSSNPRKRHREDSDVEMVEDDSRKEMTAACTPR | 209 |
| 753 | RRIINLTSVLSLQEEINEQGHEVLREMLHNHSFVGCVNP | |
| | QWALAQHQTKLYLLNTTKLSEELFYQILIYDFANFGVL | |
| | RLSEPAPLFDLAMLALDSPESGWTEEDGPKEGLAEYIV | |
| | EFLKKKAEMLADYFSLEIDEEGNLIGLPLLIDNYVPPLE | |
| | GLPIFILRLATEVNWDEEKECFESLSKECAMFYSIRKQY | |
| | ISEESTLSGQQSEVPGSIPNSWKWTVEHIVYKALRSHIL | |
| | PPKHFTEDGNILQLANLPDLYKVF[- - -] | |
| NLS^{SV40} | PKKKRKVINLTSVLSLQEEINEQGHEVLREMLHNHSF | 210 |
| MLH1 501- | VGCVNPQWALAQHQTKLYLLNTTKLSEELFYQILIYDF | |
| 753 | ANFGVLRLSEPAPLFDLAMLALDSPESGWTEEDGPKEG | |
| | LAEYIVEFLKKKAEMLADYFSLEIDEEGNLIGLPLLIDN | |
| | YVPPLEGLPIFILRLATEVNWDEEKECFESLSKECAMFY | |
| | SIRKQYISEESTLSGQQSEVPGSIPNSWKWTVEHIVYKA | |
| | LRSHILPPKHFTEDGNILQLANLPDLYKVF[- - -] | |
| NLS^{SV40} | PKKKRKVKRGPTSSNPRKRHREDSDVEMVEDDSRKE | 211 |
| MLH1 461- | MTAACTPRRRIINLTSVLSLQEEINEQGHEVLREMLHN | |
| 753 | HSFVGCVNPQWALAQHQTKLYLLNTTKLSEELFYQILI | |
| | YDFANFGVLRLSEPAPLFDLAMLALDSPESGWTEEDGP | |
| | KEGLAEYIVEFLKKKAEMLADYFSLEIDEEGNLIGLPLL | |
| | IDNYVPPLEGLPIFILRLATEVNWDEEKECFESLSKECA | |
| | MFYSIRKQYISEESTLSGQQSEVPGSIPNSWKWTVEHIV | |
| | YKALRSHILPPKHFTEDGNILQLANLPDLYKVF[- - -] | |

In still another aspect, the present disclosure contemplates the delivery of an inhibitor of MLH1 using the PE-VLPs described herein. In various embodiments, the inhibitor can be a small molecule inhibitor. In other embodiments, the inhibitor can be an anti-MLH1 antibody, e.g., a neutralizing antibody that inactivates MLH1. In still other embodiments, the inhibitor can be a dominant negative mutant of MLH1. In still other embodiments, the inhibitor can be targeted at the level of transcription of MLH1, e.g., an siRNA or other nucleic acid agent that knocks down the level of a transcript encoding MLH1.

In still other aspects, the present disclosure provides methods for prime editing whereby correction by the MMR pathway of the alterations introduced into a target nucleic acid molecule is evaded, without the need to provide an inhibitor of the MMR pathway. pegRNAs designed with consecutive nucleotide mismatches compared to a target site on the target nucleic acid, for example, pegRNAs that have three or more consecutive mismatching nucleotides, can evade correction by the MMR pathway and may be delivered using the PE-VLPs described herein, resulting in an increase in prime editing efficiency and/or a decrease in the frequency of indel formation compared to the introduction of a single nucleotide mismatch using prime editing. In addition, insertions and deletions of 10 or more nucleotides in length introduced by prime editing may also evade correction by the MMR pathway, resulting in an increase in prime editing efficiency and/or a decrease in the frequency of indel formation compared to the introduction of an insertion or deletion of less than 10 nucleotides in length using prime editing.

Thus, in one aspect, the present disclosure provides methods for editing a nucleic acid molecule by prime editing comprising delivering a prime editor using a PE-VLP described herein and a pegRNA comprising a DNA synthesis template on its extension arm comprising three or more consecutive nucleotide mismatches relative to a target site on the nucleic acid molecule. At least one of the consecutive nucleotide mismatches results in an alteration in the amino acid sequence of a protein expressed from the nucleic acid molecule. In some embodiments, more than one of the consecutive nucleotide mismatches results in an alteration in the amino acid sequence of a protein expressed from the nucleic acid molecule. On the other hand, at least one of the remaining nucleotide mismatches (i.e., those that do not result in an alteration in the amino acid sequence of a protein expressed from the nucleic acid molecule) are silent mutations. The silent mutations may be present in coding regions of the target nucleic acid molecule or in non-coding regions of the target nucleic acid molecule. When the silent mutations are present in a coding region, they introduce into the nucleic acid molecule one or more alternate codons encoding the same amino acid as the unedited nucleic acid molecule. Alternatively, when the silent mutations are in a non-coding region, the silent mutations may be present in a region of the nucleic acid molecule that does not influence splicing, gene regulation, RNA lifetime, or other biological properties of the target site on the nucleic acid molecule.

Any number of consecutive nucleotide mismatches of three or more can be used to achieve the benefits of evading correction by the MMR pathway. In some embodiments, the DNA synthesis template of the extension arm on the pegRNA comprises 3, 4, 5, 6, 7, 8, 9, 10, 11, 12, 13, 14, 15, 16, 17, 18, 19, 20, 21, 22, 23, 24, or 25 consecutive nucleotide mismatches relative to the endogenous sequence of a target site in the nucleic acid molecule edited by prime editing. In some embodiments, the DNA synthesis template of the extension arm on the pegRNA comprises 3, 4, or 5 consecutive nucleotide mismatches relative to the endogenous sequence of a target site in the nucleic acid molecule edited by prime editing. In some embodiments, the DNA synthesis template of the extension arm on the pegRNA comprises 6, 7, 8, 9, 10, 11, 12, 13, 14, 15, 16, 17, 18, 19, or 20 consecutive nucleotide mismatches relative to the endogenous sequence of a target site in the nucleic acid molecule edited by prime editing. In some embodiments, the DNA synthesis template of the extension arm on the pegRNA comprises four or more, five or more, six or more, seven or more, eight or more, nine or more, or ten or more consecutive nucleotide mismatches relative to a target site on the nucleic acid molecule.

In another aspect, the present disclosure provides methods for editing a nucleic acid molecule by prime editing comprising delivering a prime editor using a PE-VLP as described herein and a pegRNA comprising a DNA synthesis template on its extension arm comprising an insertion or deletion of 10 or more nucleotides relative to a target site on the nucleic acid molecule. Insertions and deletions of 10 or more nucleotides in length evade correction by the MMR pathway when introduced by prime editing and thus can benefit from the inhibition of the MMR pathway without the need to provide an inhibitor of MMR. Insertions and deletions of any length greater than 10 nucleotides can be used to achieve the benefits of naturally evading correction by the MMR pathway. In some embodiments, the DNA synthesis template comprises an insertion or deletion of 10, 11, 12, 13, 14, 15, 16, 17, 18, 19, 20, 21, 22, 23, 24, or 25 nucleotides relative to the endogenous sequence at a target site of the nucleic acid molecule edited by prime editing. In some embodiments, the DNA synthesis template comprises an insertion or deletion of 11 or more nucleotides, 12 or more nucleotides, 13 or more nucleotides, 14 or more nucleotides, 15 or more nucleotides, 16 or more nucleotides, 17 or more nucleotides, 18 or more nucleotides, 19 or more nucleotides, 20 or more nucleotides, 21 or more nucleotides, 22 or more nucleotides, 23 or more nucleotides, 24 or more nucleotides, or 25 or more nucleotides relative to a target site on a nucleic acid molecule. In certain embodiments, the DNA synthesis template comprises an insertion or deletion of 15 or more nucleotides relative to a target site on the nucleic acid molecule.

### PEgRNAs

The prime editing system delivered by the PE-VLPs described herein contemplates the use of any suitable PEgRNAs.

### PEgRNA Architecture

In some embodiments, an extended guide RNA is used in the prime editing system delivered using the PE-VLPs disclosed herein whereby a traditional guide RNA includes a ˜20 nt protospacer sequence and a gRNA core region, which binds with the napDNAbp. In some embodiments, the guide RNA includes an extended RNA segment at the 5′ end, i.e., a 5′ extension. In some embodiments, the 5′ extension includes a reverse transcription template sequence, a reverse transcription primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA usable in the prime editing system is used in the methods and compositions disclosed herein wherein a traditional guide RNA includes a ˜20 nt protospacer sequence and a gRNA core, which binds with the napDNAbp. In some embodiments, the guide RNA includes an extended RNA segment at the 3′ end, i.e., a 3′ extension. In some embodiments, the 3′ extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA usable in the prime editing system is used in the methods and compositions disclosed herein wherein a traditional guide RNA includes a ˜20 nt protospacer sequence and a gRNA core, which binds with the napDNAbp. In some embodiments, the guide RNA includes an extended RNA segment at an intermolecular position within the gRNA core, i.e., an intramolecular extension. In some embodiments, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the protospacer sequence of the guide RNA. In another embodiment, the position of the intermolecular RNA extension in the gRNA core. In still another embodiment, the position of the intermolecular RNA extension is anywhere within the guide RNA molecule except within the protospacer sequence, or at a position which disrupts the protospacer sequence. In one embodiment, the intermolecular RNA extension is inserted downstream from the 3′ end of the protospacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides downstream of the 3′ end of the protospacer sequence.

In other embodiments, the intermolecular RNA extension is inserted into the gRNA, which refers to the portion of the guide RNA corresponding or comprising the tracrRNA, which binds and/or interacts with the Cas9 protein or equivalent thereof (i.e., a different napDNAbp). Preferably the insertion of the intermolecular RNA extension does not disrupt or minimally disrupts the interaction between the tracrRNA portion and the napDNAbp.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The one or more nucleotide changes may include one or more single-base nucleotide changes, one or more deletions, and/or one or more insertions.

The synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand that becomes displaced as the 5′ flap species and that overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs maybe naturally occurring, expressed from an encoding nucleic acid, or synthesized chemically. Methods are well known in the art for obtaining or otherwise synthesizing guide RNAs, and for determining the appropriate sequence of the guide RNA, including the protospacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor to a target sequence may be assessed by any suitable assay. For example, the components of a prime editor, including the guide sequence to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything). A unique target sequence in a genome may include an S. pyogenes Cas9 target site of the form MMMMMMMMMNNNNNNNNNNNXGG where NNNNNNNNNNNXGG (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW where NNNNNNNNNNNNXXAGAAW (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW where NNNNNNNNNNNXXAGAAW (N is A, G, T, or C; X can be anything; and W is A or T). For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything). A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything). In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080, incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 212)
NNNNNNNNGTTTTTGTACTCTCAAGATTTAGAAATAAATCTTGCAGAAGC
TACAAAGATAAGGCTTCATGCCGAAATCAACACCCTGTCATTTTATGGCA
GGGTGTTTTCGTTATTTAATTTTTT;
(2)
(SEQ ID NO: 213)
NNNNNNNNNNNNNNNNNNGTTTTTGTACTCTCAGAAATGCAGAAGCTACA
AAGATAAGGCTTCATGCCGAAATCAACACCCTGTCATTTTATGGCAGGGT
GTTTTCGTTATTTAATTTTTT;
(3)
(SEQ ID NO: 214)
NNNNNNNNNNNNNNNNNNNNGTTTTTGTACTCTCAGAAATGCAGAAGCTA
CAAAGATAAGGCTTCATGCCGAAATCAACACCCTGTCATTTTATGGCAGG
GTGTTTTTT;
(4)
(SEQ ID NO: 215)
NNNNNNNNNNNNNNNNNNNNGTTTTAGAGCTAGAAATAGCAAGTTAAAAT
AAGGCTAGTCCGTTATCAACTTGAAAAAGTGGCACCGAGTCGGTGCTTTT
TT;
(5)
(SEQ ID NO: 216)
NNNNNNNNNNNNNNNNNNNNGTTTTAGAGCTAGAAATAGCAAGTTAAAAT
AAGGCTAGTCCGTTATCAACTTGAAAAAGTGTTTTTTT;
AND
(6)
(SEQ ID NO: 217)
NNNNNNNNNNNNNNNNNNNNGTTTTAGAGCTAGAAATAGCAAGTTAAAAT
AAGGCTAGTCCGTTATCATTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-GUUUUAGAGCUAGAAAUAGCAAGUUAAAAUAAAGGCUAGUCCGUUAUCAACU UGAAAAAGUGGCACCGAGUCGGUGCUUUUU-3′ (SEQ ID NO: 218), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic acid sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editors utilized in the methods and compositions described herein.

In some embodiments, a PEgRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an edit template (B), and a homology arm (C). In addition, the PEgRNA may comprise an optional 3′ end modifier region (e1) and an optional 5′ end modifier region (e2). Still further, the PEgRNA may comprise a transcriptional termination signal at the 3′ end of the PEgRNA. These structural elements are further defined herein. The depiction of the structure of the PEgRNA is not meant to be limiting and embraces variations in the arrangement of the elements. For example, the optional sequence modifiers (el) and (e2) could be positioned within or between any of the other regions shown, and not limited to being located at the 3′ and 5′ ends.

### PEgRNA Modifications

The PEgRNAs may also include additional design modifications that may alter the properties and/or characteristics of PEgRNAs, thereby improving the efficacy of prime editing. In various embodiments, these modifications may belong to one or more of a number of different categories, including but not limited to: (1) designs to enable efficient expression of functional PEgRNAs from non-polymerase III (pol III) promoters, which would enable the expression of longer PEgRNAs without burdensome sequence requirements; (2) modifications to the core, Cas9-binding PEgRNA scaffold, which could improve efficacy; (3) modifications to the PEgRNA to improve RT processivity, enabling the insertion of longer sequences at targeted genomic loci; and (4) addition of RNA motifs to the 5′ or 3′ termini of the PEgRNA that improve PEgRNA stability, enhance RT processivity, prevent misfolding of the PEgRNA, or recruit additional factors important for genome editing.

In one embodiment, PEgRNA could be designed with polIlI promoters to improve the expression of longer-length PEgRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a PEgRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed PEgRNA, which has been shown to result in markedly reduced Cas9:sgRNA activity in a site-dependent manner. Additionally, while pol III-transcribed PEgRNAs can simply terminate in a run of 6-7 U's, PEgRNAs transcribed from pol II or pol I would require a different termination signal. Often such signals also result in polyadenylation, which would result in undesired transport of the PEgRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA- (lncRNA) tagged sgRNAs. These platforms include RNAs expressed from pCMV and that terminate in the ENE element from the MALATI ncRNA from humans, the PAN ENE element from KSHV, or the 3′ box from U1 snRNA. Notably, the MALATI ncRNA and PAN ENEs form triple helices protecting the polyA-tail. These constructs could also enhance RNA stability. It is contemplated that these expression systems will also enable the expression of longer PEgRNAs.

In addition, a series of methods have been designed for the cleavage of the portion of the pol II promoter that would be transcribed as part of the PEgRNA, adding either a self-cleaving ribozyme such as the hammerhead, pistol, hatchet, hairpin, VS, twister, or twister sister ribozymes, or other self-cleaving elements to process the transcribed guide, or a hairpin that is recognized by Csy4 and also leads to processing of the guide. Also, it is hypothesized that incorporation of multiple ENE motifs could lead to improved PEgRNA expression and stability, as previously demonstrated for the KSHV PAN RNA and element. It is also anticipated that circularizing the PEgRNA in the form of a circular intronic RNA (ciRNA) could also lead to enhanced RNA expression and stability, as well as nuclear localization.

In various embodiments, the PEgRNA may include various above elements, as exemplified by the following sequences.

Non-limiting example 1 - PEgRNA expression
platform consisting of pCMV, Csy4 hairpin,
the PEgRNA, and MALAT1 ENE
(SEQ ID NO: 219)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTAGGGTCATGAAGGTT
TTTCTTTTCCTGAGAAAACAACACGTATTGTTTTCTCAGGTTTTGCTTTT
TGGCCTTTTTCTAGCTTAAAAAAAAAAAAAGCAAAAGATGCTGGTGGTTG
GCACTCCTGGTTTCCAGGACGGGGTTCAAATCCCTGCGGCGTCTTTGCTT
TGACT
Non-limiting example 2 - PERNA expression
platform consisting of pCMV, Csy4 hairpin,
the PEgRNA, and PAN ENE
(SEQ ID NO: 220)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAA
Non-limiting example 3 - PEgRNA expression
platform consisting of pCMV, Csy4 hairpin,
the PEgRNA, and 3xPAN ENE
(SEQ ID NO: 221)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAAACACACTG
TTTTGGCTGGGTTTTTCCTTGTTCGCACCGGACACCTCCAGTGACCAGAC
GGCAAGGTTTTTATCCCAGTGTATATTGGAAAAACATGTTATACTTTTGA
CAATTTAACGTGCCTAGAGCTCAAATTAAACTAATACCATAACGTAATGC
AACTTACAACATAAATAAAGGTCAATGTTTAATCCATAAAAAAAAAAAAA
AAAAAATCTCTCTGTTTTGGCTGGGTTTTTCCTTGTTCGCACCGGACACC
TCCAGTGACCAGACGGCAAGGTTTTTATCCCAGTGTATATTGGAAAAACA
TGTTATACTTTTGACAATTTAACGTGCCTAGAGCTCAAATTAAACTAATA
CCATAACGTAATGCAACTTACAACATAAATAAAGGTCAATGTTTAATCCA
TAAAAAAAAAAAAAAAAAAA
Non-limiting example 4 - PERNA expression
platform consisting of pCMV, Csy4 hairpin,
the PEgRNA, and 3′ box
(SEQ ID NO: 222)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTGTTTCAAAAGTAGACT
GTACGCTAAGGGTCATATCTTTTTTTGTTTGGTTTGTGTCTTGGTTGGCG
TCTTAAA
Non-limiting example 5 - PEgRNA expression
platform consisting of pU1, Csy4 hairpin,
the PEgRNA, and 3′ box
(SEQ ID NO: 223)
CTAAGGACCAGCTTCTTTGGGAGAGAACAGACGCAGGGGGGGGAGGGAAA
AAGGGAGAGGCAGACGTCACTTCCCCTTGGCGGCTCTGGCAGCAGATTGG
TCGGTTGAGTGGCAGAAAGGCAGACGGGGACTGGGCAAGGCACTGTCGGT
GACATCACGGACAGGGCGACTTCTATGTAGATGAGGCAGCGCAGAGGCTG
CTGCTTCGCCACTTGCTGCTTCACCACGAAGGAGTTCCCGTGCCCTGGGA
GCGGGTTCAGGACCGCTGATCGGAAGTGAGAATCCCAGCTGTGTGTCAGG
GCTGGAAAGGGCTCGGGAGTGCGCGGGGCAAGTGACCGTGTGTGTAAAGA
GTGAGGCGTATGAGGCTGTGTCGGGGCAGAGGCCCAAGATCTCAGTTCAC
TGCCGTATAGGCAGGGCCCAGACTGAGCACGTGAGTTTTAGAGCTAGAAA
TAGCAAGTTAAAATAAGGCTAGTCCGTTATCAACTTGAAAAAGTGGGACC
GAGTCGGTCCTCTGCCATCAAAGCGTGCTCAGTCTGTTTCAGCAAGTTCA
GAGAAATCTGAACTTGCTGGATTTTTGGAGCAGGGAGATGGAATAGGAGC
TTGCTCCGTCCACTCCACGCATCGACCTGGTATTGCAGTACCTCCAGGAA
CGGTGCACCCACTTTCTGGAGTTTCAAAAGTAGACTGTACGCTAAGGGTC
ATATCTTTTTTTGTTTGGTTTGTGTCTTGGTTGGCGTCTTAAA.

In various other embodiments, the PEgRNA may be improved by introducing modifications to the scaffold or core sequences. The core, Cas9-binding PEgRNA scaffold can likely be improved to enhance PE activity. Several such approaches have already been demonstrated. For instance, the first pairing element of the scaffold (P1) contains a GTTTT-AAAAC (SEQ ID NO: 231) pairing element. Such runs of Ts have been shown to result in pol III pausing and premature termination of the RNA transcript. Rational mutation of one of the T-A pairs to a G-C pair in this portion of P1 has been shown to enhance sgRNA activity, suggesting this approach would also be feasible for PEgRNAs. Additionally, increasing the length of P1 has also been shown to enhance sgRNA folding and lead to improved activity, suggesting it as another avenue for the modification of PEgRNA activity. Example modifications to the core can include:

PEgRNA containing a 6 nt extension to P1
(SEQ ID NO: 224)
GGCCCAGACTGAGCACGTGAGTTTTAGAGCTAGCTCATGAAAATGAGCTA
GCAAGTTAAAATAAGGCTAGTCCGTTATCAACTTGAAAAAGTGGGACCGA
GTCGGTCCTCTGCCATCAAAGCGTGCTCAGTCTGTTTTTTT
PERNA containing a T-A to G-C mutation
within P1
(SEQ ID NO: 225)
GGCCCAGACTGAGCACGTGAGTTTGAGAGCTAGAAATAGCAAGTTTAAAT
AAGGCTAGTCCGTTATCAACTTGAAAAAGTGGGACCGAGTCGGTCCTCTG
CCATCAAAGCGTGCTCAGTCTGTTTTTTT

In various other embodiments, the PEgRNA may be modified at the edit template region. As the size of the insertion templated by the PEgRNA increases, it is more likely to be degraded by endonucleases, undergo spontaneous hydrolysis, or fold into secondary structures unable to be reverse-transcribed by the RT, or that disrupt folding of the PEgRNA scaffold and subsequent Cas9-RT binding. Accordingly, it is likely that modification to the template of the PEgRNA might be necessary to affect large insertions, such as the insertion of whole genes. Some strategies to do so include the incorporation of modified nucleotides within a synthetic or semi-synthetic PEgRNA that render the RNA more resistant to degradation or hydrolysis or less likely to adopt inhibitory secondary structures. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the PEgRNA to enhance stability and activity. Alternatively, or additionally, the template of the PEgRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two separate PEgRNAs. In such a design, a PE would be used to initiate transcription, and also to recruit a separate template RNA to the targeted site via an RNA-binding protein fused to Cas9 or an RNA recognition element on the PEgRNA itself such as the MS2 aptamer. The RT could either directly bind to this separate template RNA, or initiate reverse transcription on the original PEgRNA before swapping to the second template. Such an approach could enable long insertions by both preventing misfolding of the PEgRNA upon addition of the long template, and also by not requiring dissociation of Cas9 from the genome for long insertions to occur, which could possibly inhibit PE-based long insertions.

In still other embodiments, the PEgRNA may be modified by introducing additional RNA motifs at the 5′ and 3′ termini of the PEgRNAs, or even at positions therein between (e.g., in the gRNA core region, or the spacer). Several such motifs—such as the PAN ENE from KSHV and the ENE from MALATI were discussed above as possible means to terminate expression of longer PEgRNAs from non-pol III promoters. These elements form RNA triple helices that engulf the polyA tail, resulting in their being retained within the nucleus. However, by forming complex structures at the 3′ terminus of the PEgRNA that occlude the terminal nucleotide, these structures would also likely help prevent exonuclease-mediated degradation of PEgRNAs.

Other structural elements inserted at the 3′ terminus could also enhance RNA stability, albeit without enabling termination from non-pol III promoters. Such motifs could include hairpins or RNA quadruplexes that would occlude the 3′ terminus, or self-cleaving ribozymes such as HDV that would result in the formation of a 2′-3′-cyclic phosphate at the 3′ terminus, and also potentially render the PEgRNA less likely to be degraded by exonucleases. Inducing the PEgRNA to cyclize via incomplete splicing—to form a ciRNA—could also increase PEgRNA stability and result in the PEgRNA being retained within the nucleus.

Additional RNA motifs could also improve RT processivity or enhance PEgRNA activity by enhancing RT binding to the DNA-RNA duplex. Addition of the native sequence bound by the RT in its cognate retroviral genome could enhance RT activity. This could include the native primer binding site (PBS), polypurine tract (PPT), or kissing loops involved in retroviral genome dimerization and initiation of transcription.

Addition of dimerization motifs—such as kissing loops or a GNRA tetraloop/tetraloop receptor pair—at the 5′ and 3′ termini of the PEgRNA could also result in effective circularization of the PEgRNA, improving stability. Additionally, it is envisioned that addition of these motifs could enable the physical separation of the PEgRNA spacer and primer, preventing occlusion of the spacer, which would hinder PE activity. Short 5′ extensions or 3′ extensions to the PEgRNA that form a small toehold hairpin in the spacer region or along the primer binding site could also compete favorably against the annealing of intracomplementary regions along the length of the PEgRNA, e.g., the interaction between the spacer and the primer binding site that can occur. Finally, kissing loops could also be used to recruit other template RNAs to the genomic site and enable swapping of RT activity from one RNA to the other. A number of secondary RNA structures may be engineered into any region of the PEgRNA, including in the terminal portions of the extension arm (i.e., e1 and e2), as shown.

Example modifications include, but are not limited to:

PEgRNA-HDV fusion
(SEQ ID NO: 226)
GGCCCAGACTGAGCACGTGAGTTTTAGAGCTAGAAATAGCAAGTTAAAAT
AAGGCTAGTCCGTTATCAACTTGAAAAAGTGGGACCGAGTCGGTCCTCTG
CCATCAAAGCGTGCTCAGTCTGGGCCGGCATGGTCCCAGCCTCCTCGCTG
GCGCCGGCTGGGCAACATGCTTCGGCATGGCGAATGGGACTTTTTTT
PEgRNA-MMLV kissing loop
(SEQ ID NO: 227)
GGTGGGAGACGTCCCACCGGCCCAGACTGAGCACGTGAGTTTTAGAGCTA
GAAATAGCAAGTTAAAATAAGGCTAGTCCGTTATCAACTTGAAAAAGTGG
GACCGAGTCGGTCCTCTGCCATCAAAGCTTCGACCGTGCTCAGTCTGGTG
GGAGACGTCCCACCTTTTTTT
PEgRNA-VS ribozyme kissing loop
(SEQ ID NO: 228)
GAGCAGCATGGCGTCGCTGCTCACGGCCCAGACTGAGCACGTGAGTTTTA
GAGCTAGAAATAGCAAGTTAAAATAAGGCTAGTCCGTTATCAACTTGAAA
AAGTGGGACCGAGTCGGTCCTCTGCCATCAAAGCTTCGACCGTGCTCAGT
CTCCATCAGTTGACACCCTGAGGTTTTTTT
PEgRNA-GNRA tetraloop/tetraloop receptor
(SEQ ID NO: 229)
GCAGACCTAAGTGGUGACATATGGTCTGGGCCCAGACTGAGCACGTGAGT
TTTAGAGCTAUACGTAGCAAGTTAAAATAAGGCTAGTCCGTTATCAACTT
UACGAAGTGGGACCGAGTCGGTCCTCTGCCATCAAAGCTTCGACCGTGCT
CAGTCTGCATGCGATTAGAAATAATCGCATGTTTTTTT
PEgRNA template switching secondary
RNA-HDV fusion
(SEQ ID NO: 230)
TCTGCCATCAAAGCTGCGACCGTGCTCAGTCTGGTGGGAGACGTCCCACC
GGCCGGCATGGTCCCAGCCTCCTCGCTGGCGCCGGCTGGGCAACATGCTT
CGGCATGGCGAATGGGACTTTTTTT

PEgRNA scaffolds could be further improved via directed evolution, in an analogous fashion to how SpCas9 and prime editors (PE) have been improved. Directed evolution could enhance PEgRNA recognition by Cas9 or evolved Cas9 variants. Additionally, it is likely that different PEgRNA scaffold sequences would be optimal at different genomic loci, either enhancing PE activity at the site in question, reducing off-target activities, or both. Finally, evolution of PEgRNA scaffolds to which other RNA motifs have been added would almost certainly improve the activity of the fused PEgRNA relative to the unevolved, fusion RNA. For instance, evolution of allosteric ribozymes composed of c-di-GMP-I aptamers and hammerhead ribozymes led to dramatically improved activity, suggesting that evolution would improve the activity of hammerhead-PEgRNA fusions as well. In addition, while Cas9 currently does not generally tolerate 5′ extension of the sgRNA, directed evolution will likely generate enabling mutations that mitigate this intolerance, allowing additional RNA motifs to be utilized.

The present disclosure contemplates any such ways to further improve the efficacy of the prime editing systems utilized in the methods and compositions disclosed here.

In various embodiments, it may be advantageous to limit the appearance of a consecutive sequence of Ts from the extension arm, as consecutive series of T's may limit the capacity of the PEgRNA to be transcribed. For example, strings of at least three consecutive T's, at least four consecutive T's, at least five consecutive T's, at least six consecutive T's, at least seven consecutive T's, at least eight consecutive T's, at least nine consecutive T's, at least ten consecutive T's, at least eleven consecutive T's, at least twelve consecutive T's, at least thirteen consecutive T's, at least fourteen consecutive T's, or at least fifteen consecutive T's should be avoided when designing the PEgRNA, or should be at least removed from the final designed sequence. In one embodiment, one can avoid the inclusion of unwanted strings of consecutive T's in PEgRNA extension arms by avoiding target sites that are rich in consecutive A:T nucleobase pairs.

### Methods of Producing PE-VLPs

In one aspect, the present disclosure relates to methods for producing the eVLPs described herein. In some embodiments, a method for producing the presently described eVLPs comprises transfecting, transducing, electroporating, or otherwise inserting into a producer cell one or more polynucleotides that together encode all the components of the eVLPs (e.g., any of the pluralities of polynucleotides described herein, or any of the vectors described herein). In some embodiments, the present disclosure provides one or more vectors comprising one, two, three, or all four of the plurality of polynucleotides provided herein. In certain embodiments, each of the first, second, third, and fourth polynucleotides are on separate vectors. In certain embodiments, one or more of the first, second, third, and fourth polynucleotides are on the same vector.

In some embodiments, once the producer cell expresses the polynucleotides, the various components of the eVLPs self-assemble spontaneously within the producer cells. Assembly of the eVLPs relies on multimerization of the gag polyproteins encoded on the polynucleotides as described above. The gag polyproteins (some of which are fused to a gene editing agent, such as a prime editor) multimerize at the cell membrane of a producer cell and are subsequently released into the producer cell supernatant spontaneously. Thus, PE-eVLPs may be produced by transient transfection of producer cells (for example, Gesicle Producer 293T cells) as described in the Examples herein. All of the polynucleotides required for production of the eVLPs may be transfected into the producer cells simultaneously, or each polynucleotide needed may be transfected one at a time. In some embodiments, a single polynucleotide encodes all the components needed to produce the eVLPs described herein. Following transfection and incubation of the producer cells (e.g., for about 2 hours, about 3 hours, about 4 hours, about 5 hours, about 6 hours, about 7 hours, about 8 hours, about 9 hours, about 10 hours, about 15 hours, about 24 hours, about 36 hours, about 48 hours, or more than 48 hours), producer cell supernatant may be harvested, and eVLPs may be purified therefrom.

Any cell capable of expressing a foreign polynucleotide may be used to produce the eVLPs described herein. For example, the present disclosure contemplates the use of any of the cells listed in the Kits and Cells section herein for production of the eVLPs, or any other cell known in the art capable of expressing a foreign polynucleotide.

### Pharmaceutical Compositions

Other aspects of the present disclosure relate to pharmaceutical compositions comprising any of the PE-VLPs, fusion proteins, and polynucleotides/pluralities of polynucleotides described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits and Cells

The fusion proteins, PE-VLPs, and compositions of the present disclosure may be assembled into kits. In some embodiments, the kit comprises polynucleotides for expression and assembly of the PE-VLPs described herein. In other embodiments, the kit further comprises appropriate guide nucleotide sequences or nucleic acid vectors for the expression of such guide nucleotide sequences, to target the Cas9 protein of the prime editors being delivered by the PE-VLPs to the desired target sequence.

The kits described herein may include one or more containers housing components for performing the methods described herein, and optionally instructions for use. Any of the kits described herein may further comprise components needed for performing the prime editing methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the PE-VLPs described herein (e.g., including, but not limited to, the napDNAbps, reverse transcriptase domains, gag proteins, gRNAs, and viral envelope glycoproteins). In some embodiments, the nucleotide sequence(s) comprises a heterologous promoter (or more than a single promoter) that drives expression of the PE-VLP system components.

Other aspects of this disclosure provide kits comprising one or more nucleic acid constructs encoding the various components of the PE-VLP system described herein, e.g., a nucleotide sequence encoding the components of the PE-VLP system capable of delivering a prime editor to a target cell. In some embodiments, the nucleotide sequence comprises a heterologous promoter that drives expression of the PE-VLP system components.

Cells that may contain any of the PE-VLPs, fusion proteins, and compositions described herein include prokaryotic cells and eukaryotic cells. The methods described herein may be used to deliver a base into a eukaryotic cell (e.g., a mammalian cell, such as a human cell). In some embodiments, the cell is in vitro (e.g., cultured cell). In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, PE-VLPs are delivered into human embryonic kidney (HEK) cells (e.g., HEK 293 or HEK 293T cells). In some embodiments, PE-VLPs are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells, are used in assessing one or more test compounds.

### EXAMPLES

### Example 1. Virus-Like Particle (VLP)-Mediated Delivery of Prime Editor and Guide RNA

Virus-like particles (VLPs) were engineered to package prime editors (PE), the associated prime editor guide RNAs (pegRNAs), and other components to enable efficient prime editing. To produce the initial version of PE2 VLPs, plasmids for expressing the following components were transfected into gesicle cells: VSV-G envelope glycoprotein, MMLV-Gag-pol, prime editor, and pegRNA. To facilitate cargo packaging, three major components were adopted in this system: (1) gag-cargo fusion to promote the trafficking of the editor components to the site of particle formation; (2) 3 copies of nuclear export signal (NES) to facilitate proper localization of the editor at the cytoplasm of the producer cells; and (3) a protease cleavage site to allow the release of the editor from the gag into the target cells. In the initial version of PE VLP, the prime editor was split into a Cas9 half and a reverse transcriptase (RT) half, and each half was fused to an intein. Thus, the assembly of the functional prime editor depends on the intein splicing event.

Several experiments were conducted to optimize the PE2 VLP system. First, a single-particle system where two halves of the PE were packaged in a single particle was compared to a two-particle system where each half of PE was packaged individually into separate particles. This comparison showed that the single-particle system displayed higher editing efficiency. Next, nuclear localization signals (NLSs) were added at each end of the editor halves. It was hypothesized that the additional NLS may facilitate editor localization to the nucleus of the target cells. Indeed, the experiments showed that having two copies of NLS, one at each end of the prime editor, was more efficient than having one copy.

The system was further improved by identifying major bottlenecks in the initial system. First, it was hypothesized that lower binding affinity of pegRNA to Cas9 as compared to sgRNA might have impaired the packaging of pegRNA in the VLPs. This hypothesis was confirmed by showing in the dual transfection-transduction experiment that supplementing pegRNA to the target cells doubles the editing efficiency of PE VLPs. The same experiment also showed that the supplementation of sgRNA does not affect base editor (BE) eVLP editing efficiency, further confirming that efficient pegRNA packaging is a unique challenge to PE VLPs. Therefore, the F+E scaffold developed by Chen, B. et al. was adopted, which has been shown to improve guide RNA binding to Cas9 and avoid premature transcription termination. This modification led to an improvement in the editing efficiency for PE VLPs.

Next, the system was upgraded by packaging the PEmax—a prime editor harboring several modifications that demonstrates more robust activity (Chen, P. et al.). The resulting PE2max VLP provided an improvement in the editing efficiency across all sites tested.

PE3max VLPs were then developed, in which an additional nicking guide was packaged in the VLP for nicking of the unedited strand. An all-in-one particle system was first compared to a separate-particle system, in which the nicking guide RNA (ngRNA) was packaged separately from the pegRNA. The results showed that the all-in-one particle system had higher editing efficiency. Then, a range of pegRNA to ngRNA ratios was screened in the all-in-one particle system, and it was found that 30% of ngRNA among the total mass of guide RNA transfected was the most optimal. This PE3max VLP system offered an additional 3.5-fold improvement over the PE2max VLP system.

The effect of evading the mismatch-repair pathway, which has been shown to adversely affect editing efficiency, was then explored in the context of PE VLPs. In order to assess the effect, the editing efficiency for +5 G>C edit and +1 T>A edit at the HEK3 site was compared. The G>C edit is considered a mismatch repair-privileged edit, which evades MMR pathway efficiently. Indeed, the data suggested that such an edit that evades MMR has much higher editing efficiency. Therefore, evading the MMR pathway that reverts the installed edit is an important strategy to improve PE VLP editing efficiency, especially because PE is packaged as a transiently expressing RNP form and thus has a limited lifetime. Two strategies for evading MMR have been studied: first, Chen et al. have shown that in vitro co-transfection of MLH1dn with PE improves editing efficiency by suppressing MMR. Packaging of MLH1dn protein into the VLP was accomplished using the Gag-fusion strategy. Both the all-in-one particle and the separate-particle systems, where Gag-MLH1dn fusion protein was packaged in a separate particle from the PE, were tested, and the separate-particle system showed more promise. A dual transfection-transduction experiment showed that MLH1dn plasmid transfection offers significant improvement to PE2max VLP editing efficiency, again showing that evading MMR has a significant role in improving VLP PE editing. The experiment further showed that MLH1dn is indeed being packaged in the particle. Another strategy to evade MMR is to install silent mutations next to the desired edit. To verify this strategy, the addition of three or four contiguous mutations next to the desired +1 T>A edit at the HEK3 locus was tested. The results showed that adding contiguous mutations improves the editing efficiency of the desired edit, and the efficiency was even comparable to that of lipofectamine plasmid transfection.

Finally, the editor construct was further optimized because the initial split design was susceptible to inefficient PE assembly by intein splicing and the potential for the Cas9 half alone binding to the target edit site. Four additional split constructs and three full-length constructs were tested. Among all, the most optimal construct was the full-length editor with a deletion in the last six amino acids of RT. The 10 amino acids at the C-terminus of RT encode an endogenous protease site that may be recognized by the protease being expressed in the system and thus may lead to the cleavage of the NLS at the C-terminus of RT. Therefore, the deletion may increase the amount of prime editor with an NLS at the C-terminus.

Overall, the all-in-one particle system in which full-length (6 aa deleted RT) PE is packaged along with pegRNA and ngRNA shows the highest editing efficiency.

### Example 2. Further Optimized VLP-Mediated Delivery of Prime Editor and Guide RNA

VLPs packaging prime editors and the associated guide RNAs as described above were optimized further.

### Editor Construct Engineering

Several editor constructs were engineered and screened to further optimize the initial split-editor construct for the delivery of functional PE (FIG. 32). Among all constructs tested, two main modifications resulted in improvement over the initial construct. First, the full-length editor offered 1.3-fold improvement in editing efficiency over the split-editor construct, likely because intein trans-splicing is no longer required to reconstitute a functional editor. Second, the six amino acids at the C-terminus of MMLV RT were removed to eliminate the endogenous protease cleavage site. The rationale for this engineering was that the MMLV protease may recognize this cleavage site and cleave off the nuclear localization signal (NLS), which is critical for localizing the editor to the target cell nuclei. Overall, these engineering efforts facilitated the proper assembly of a functional prime editor and resulted in enhanced PE-eVLP efficiencies.

### VLP Architecture Engineering

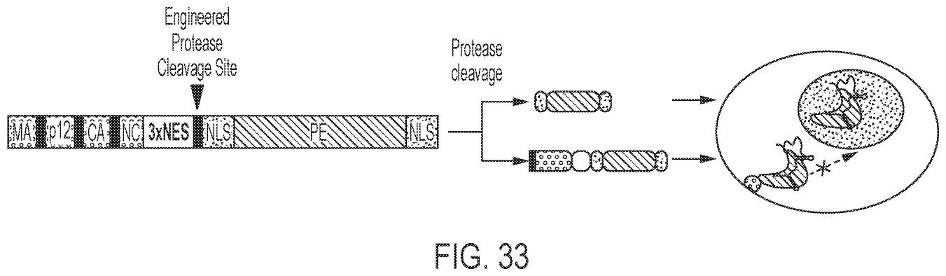

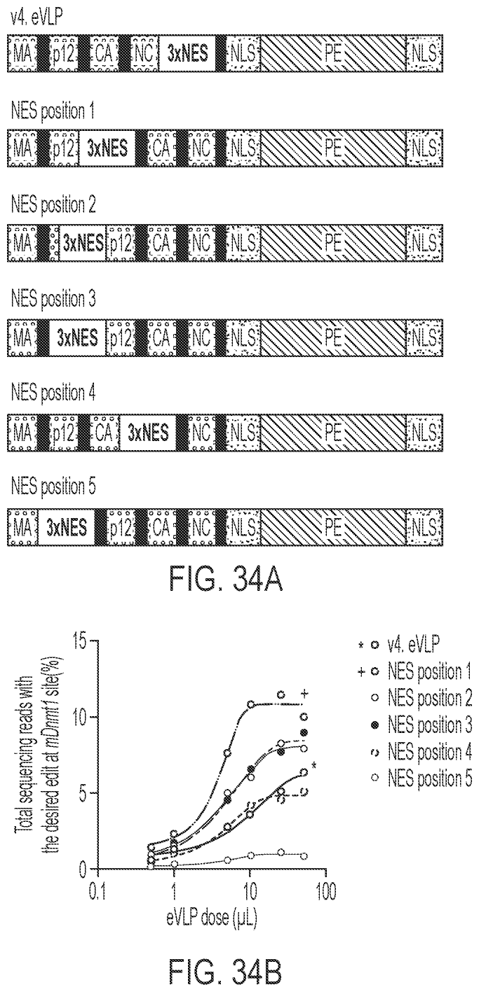

NES is instrumental to the localization of the Gag-editor fusion prior to proteolytic cleavage. After cleavage, however, the editors need to be separated from the NES for transport to target cell nuclei. In the v4 eVLP architecture design, the 3×NES was placed in front of the engineered protease cleavage site to facilitate proper cleavage of the editors from Gag and NES. In this design, the MMLV Gag protein has several endogenous protease cleavage sites that direct natural proteolytic processing. Therefore, a fraction of editors may still retain NES after the protease cleavage, thus potentially interfering with the proper localization of the editors (FIG. 33). Screens were therefore performed to identify a site within the Gag protein that could tolerate NES insertion (FIG. 34A). Among the five new explored sites, several showed improved editing over the v4 eVLP (FIG. 34B).

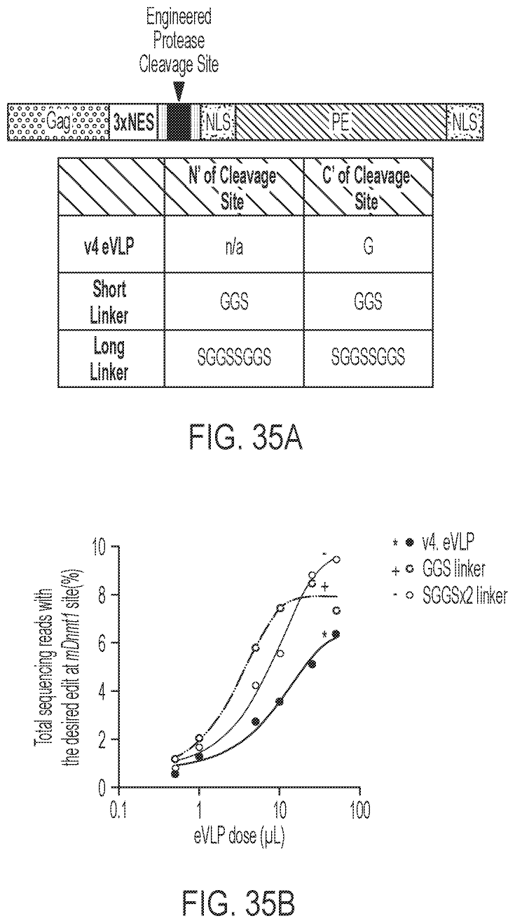

Another parameter to potentially optimize was the linkers flanking the engineered protease cleavage site. Because the delivery of functional RNP relies on proteolytic cleavage at the intended site, inserting linker sequences may better expose the site for protease recognition (FIG. 35A). Both short and long linkers tested showed higher editing compared to the original construct, and the shorter linker sequence was chosen in the eVLP designs moving forward (FIG. 35B).

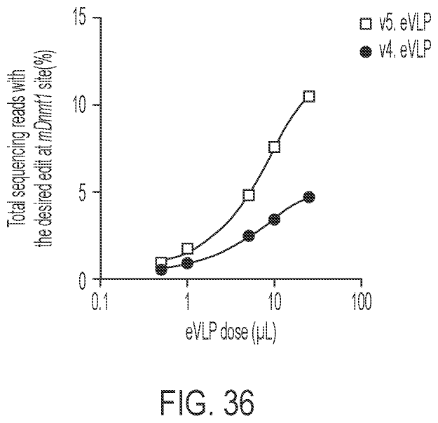

The optimized NES location was further combined with the optimal linker sequence. Overall, this optimized v5 eVLP architecture resulted in substantially improved editing efficiency compared to the original v4 eVLP (FIG. 36).

### Strategy to Evade MMR

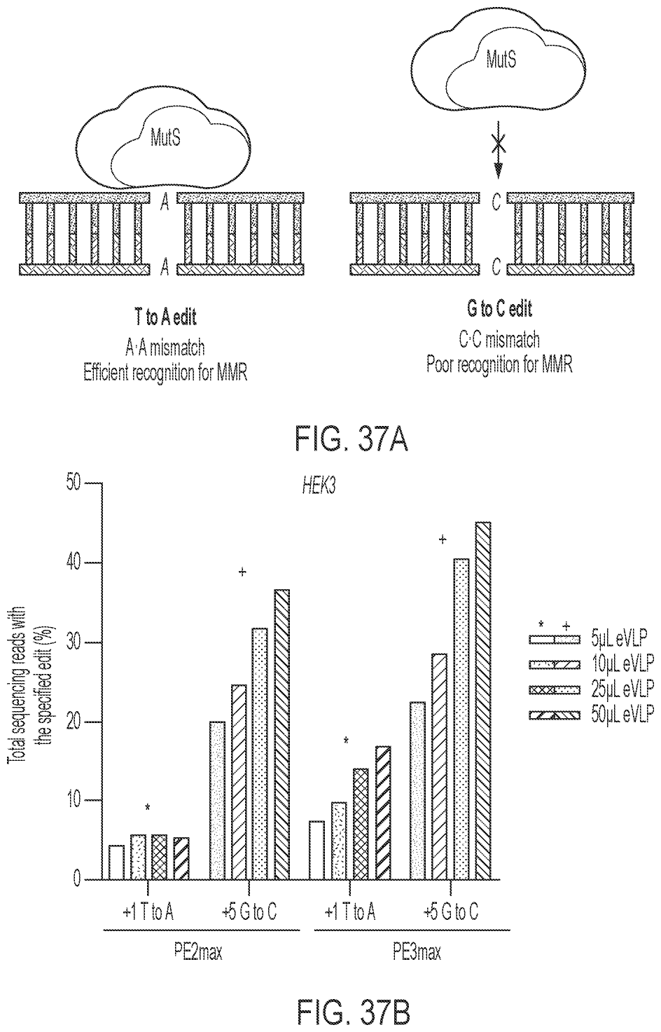

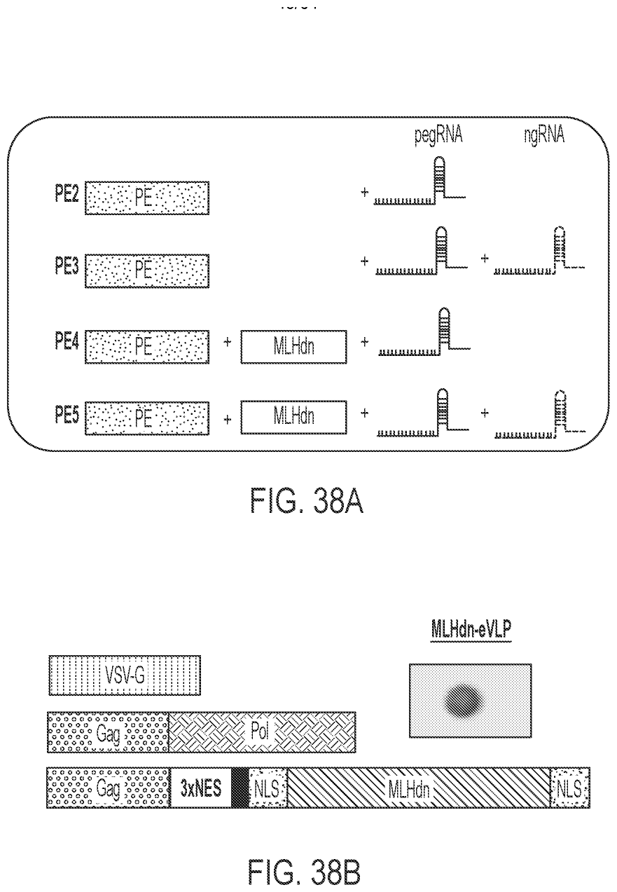

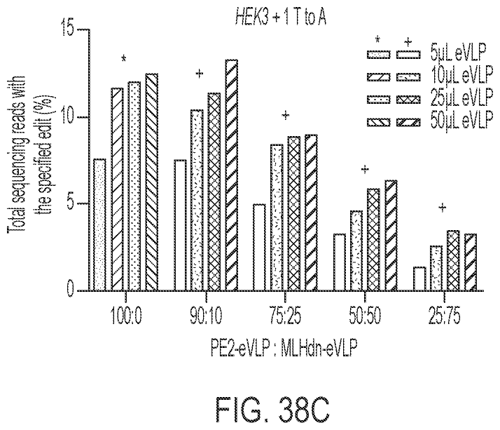

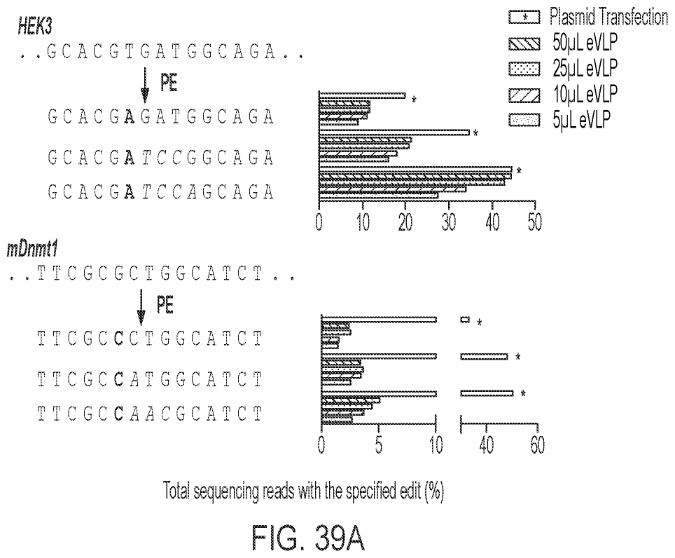

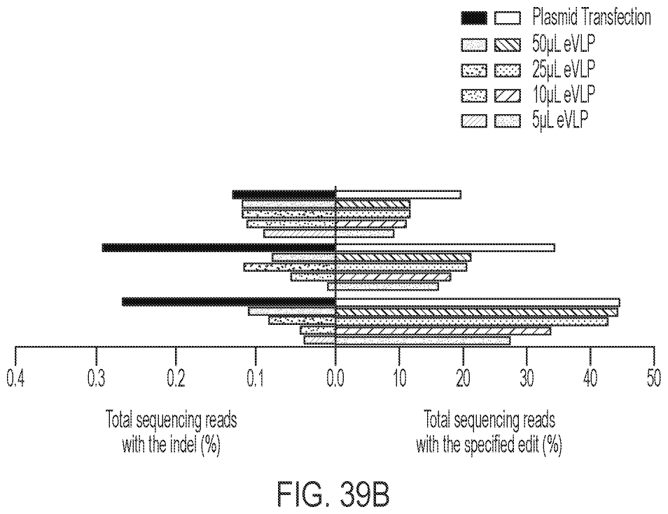

It has been shown that the installation of additional contiguous mutations in addition to the desired correction of the mutation can increase the chance that the edit will avoid reversion by the mismatch repair (MMR) pathway, which can adversely affect prime editing outcomes (FIGS. 37A-37B, 38A-38C). Such a strategy may be advantageous as no additional components need to be packaged in the eVLP. Additional contiguous mutations were installed for edits at the HEK3 site and the mDnmt1 site (FIG. 39A). Here, editing was substantially improved when additional mutations were encoded in the pegRNA. For the mDnmt1 site edit, a modest improvement was achieved, and for the HEK3 site edit, PE-eVLP transduction showed comparable editing to the plasmid transfection. Additionally, the number of insertion-deletion byproducts generated from eVLP transduction was substantially lower than the plasmid transfection, confirming the advantages of the system (FIG. 39B).

Optimization of pegRNA Packaging

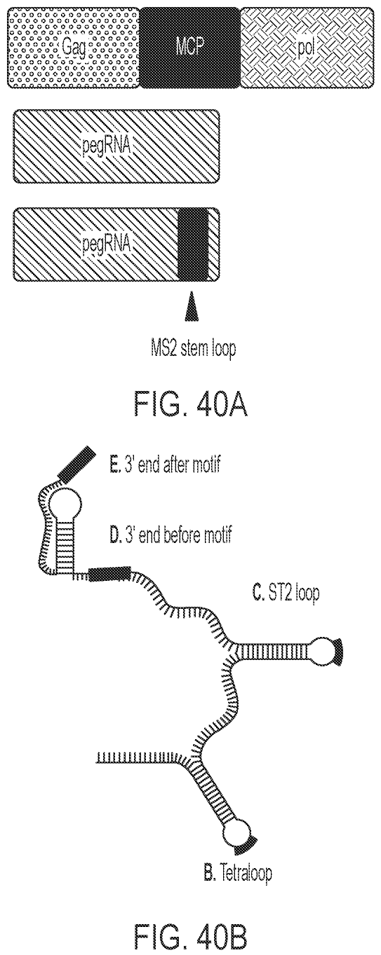

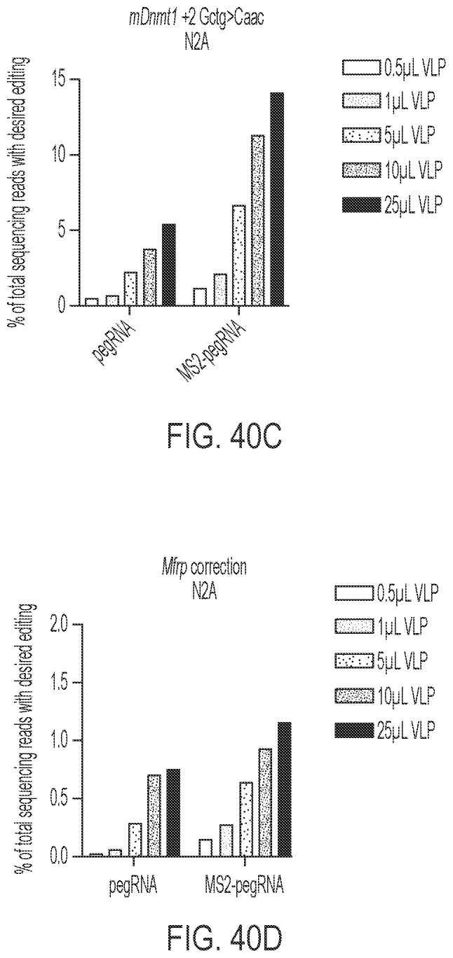

To improve pegRNA packaging in the VLP, MS2 and MS2-coat protein (MCP) interactions were analyzed (FIG. 40A). The MS2 stem loop was inserted in various regions of the pegRNA and ngRNA, and MCP was fused to Gag-pol (FIG. 40B). MS2 stem loop inserted in the ST2 loop region of the guide RNA scaffold was found to be optimal. Furthermore, various strategies for MCP fusion to Gag-pol were tested, and MCP insertion at the C-terminus of the Gag-NC domain was found to be optimal. This MS2-MCP strategy resulted in significantly improved editing efficiency at multiple sites (FIGS. 40C-40D).

Optimization of ngRNA Packaging

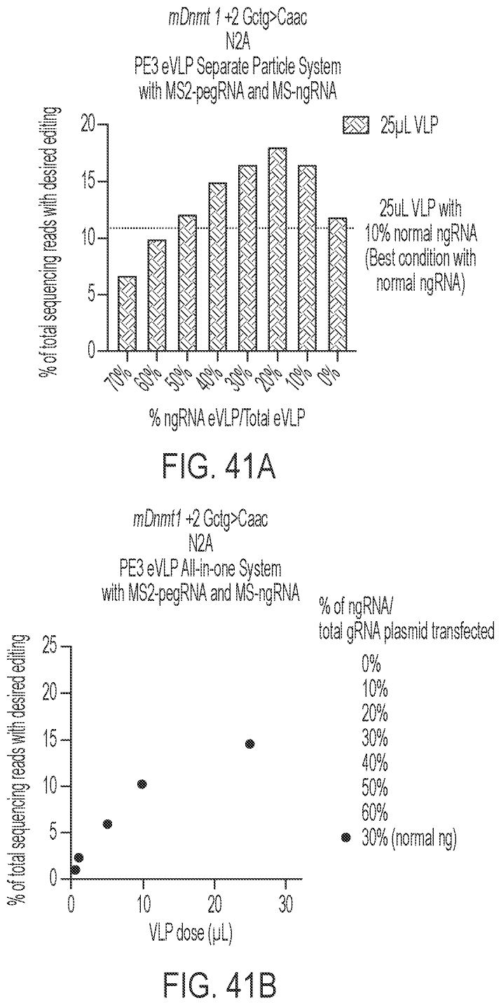

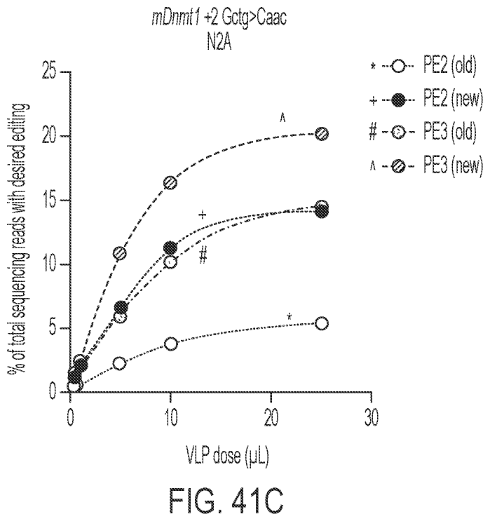

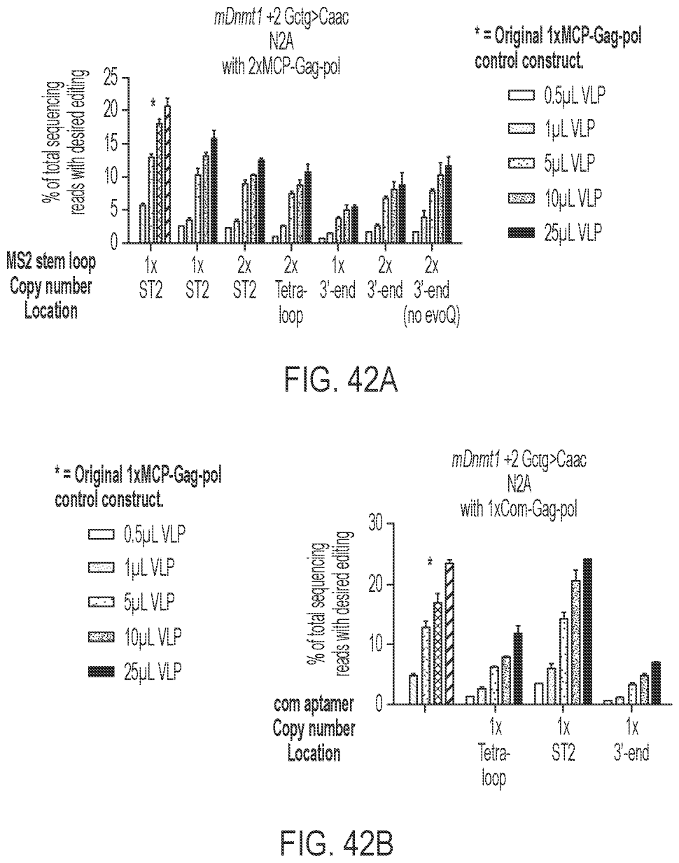

Insertions of the MS2 stem loop into the nicking guide RNA (ngRNA) to improve PE3 delivery by VLP were also tested. Both the separate particle system, in which the MS2-pegRNA and the MS2-ngRNA are packaged in different particles, and the all-in-one particle system, in which both the MS2-pegRNA and the MS2-ngRNA are packaged into the same particle, have been tested (FIGS. 41A-41C). It was confirmed that use of MS2-ngRNA resulted in significantly improved editing efficiency. Furthermore, given the smaller size of the Com protein compared to MCP, use of the Com protein and com aptamer instead of MCP-MS2 was also tested (FIGS. 42A-42B). The results suggest that this strategy is comparable to the MCP-MS2 strategy.

### Stoichiometry Optimization

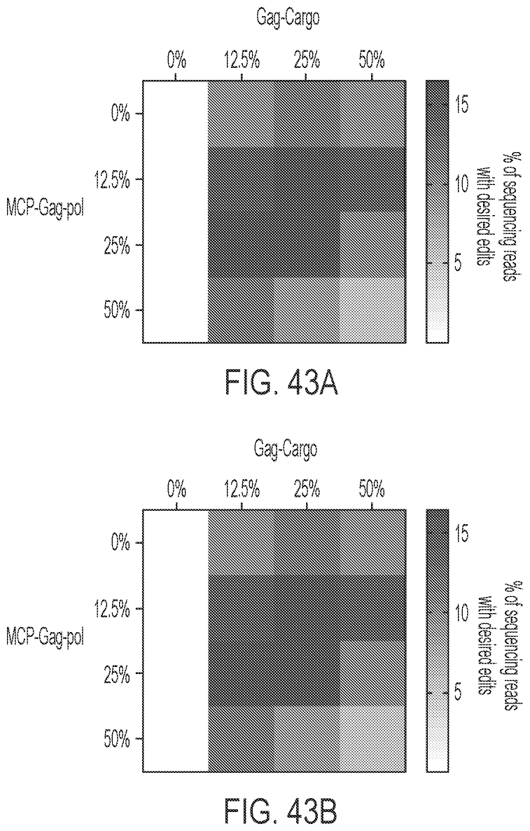

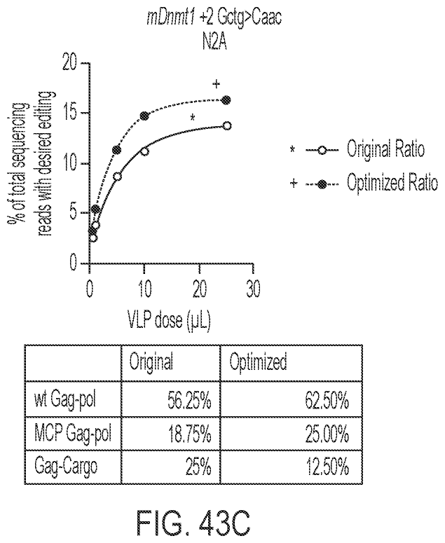

Screens were performed to determine the optimal ratio for various plasmid components to produce VLPs (FIGS. 43A-43B). The new optimized ratio showed higher editing efficiency compared to the previous ratio adopted from v4 ABE eVLP (FIG. 43C).

### Coiled-Coil Peptide for Editor Recruitment

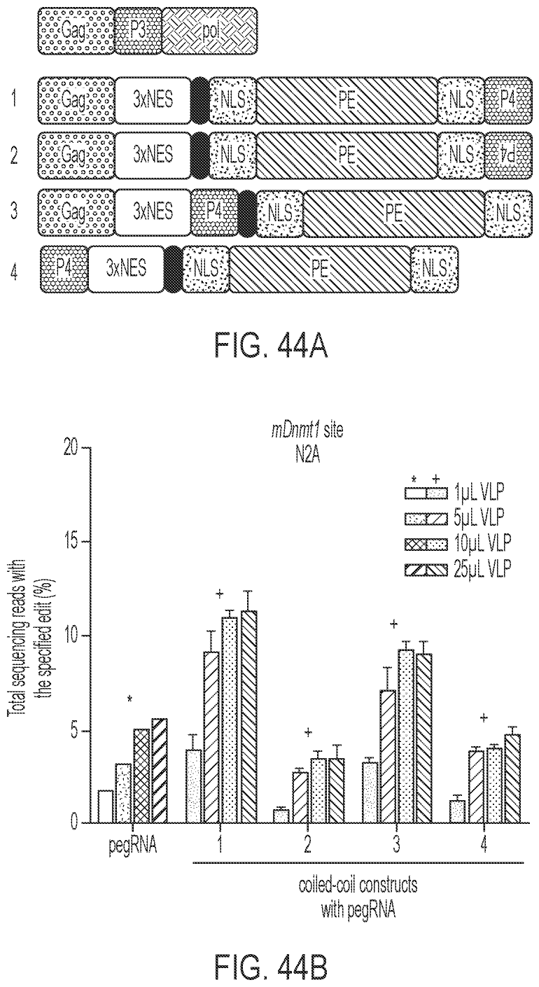

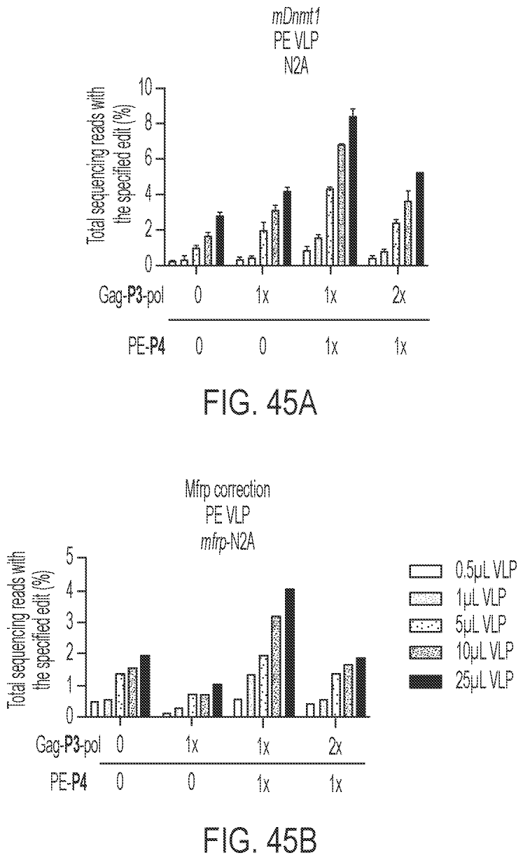

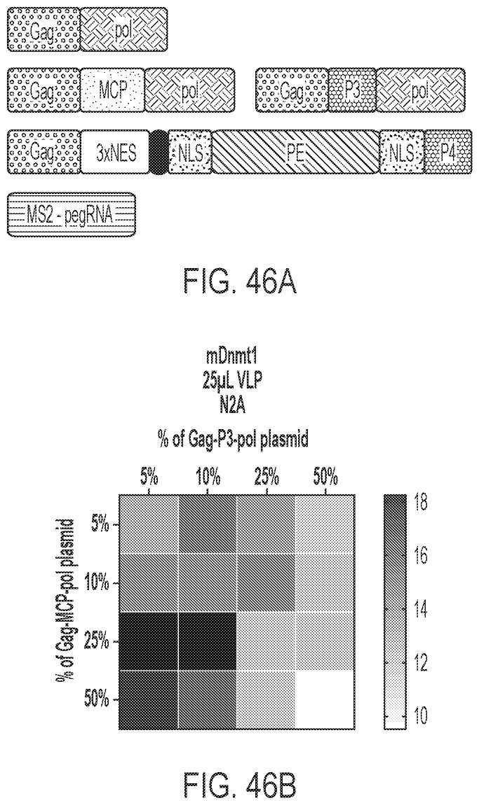

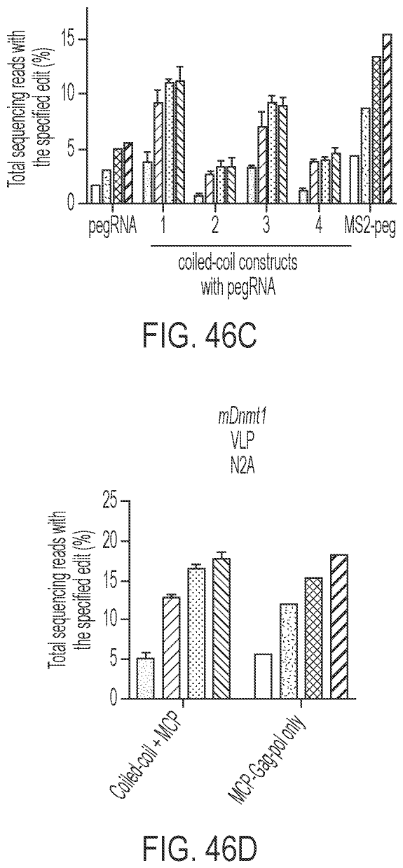

Coiled-coil peptides form a strong heterodimeric interaction and have been fused to proteins to recruit two distinct domains in proximity. In order to further improve prime editor packaging into the VLP, P3 peptide was fused to Gag-pol, and P4 peptide was fused to various positions of the prime editor construct (FIG. 44A). With regard to the first construct in FIG. 44A, where the P4 peptide is fused to the C-terminus of the Gag-PE fusion, the editing efficiency almost doubled (FIG. 44B). Therefore, it is likely that the coiled-coil peptide interaction acts as an additional mechanism for the editor recruitment in VLP. In construct 2 in FIG. 44A, an anti-parallel arrangement of the coiled-coil peptide was tested. With regard to construct 4 in FIG. 44A, it is also worth noting that the Gag-fusion has been deleted and the prime editor recruitment only depends on the coiled-coil peptide. This construct led to editing efficiency comparable to that of the Gag-PE fusion construct, confirming that the coiled-coil peptides do facilitate the editor packaging (FIG. 44B). This was further validated with an additional control condition and at an additional locus, with an additional P3 peptide fused to the construct (FIGS. 45A-45B). The results suggest that with one copy of P3, and P4 fused to the C-terminus of the Gag-PE, editing efficiency significantly improves (FIGS. 45A-45B). The strategy described further above utilizing Gag-MCP-Pol and MS2-pegRNA to facilitate pegRNA packaging still shows higher editing efficiency than the coiled-coil peptide strategy. In order to stack (i.e., combine) the benefits of these two strategies, in addition to wild type Gag-pol, Gag-MCP-pol and Gag-P3-pol need to be transfected into the producer cell (FIG. 46A). A 4×4 matrix was screened by varying the ratio of the three components (FIG. 46B). The best coiled-coil plus MCP strategy was comparable to the MCP-gag-pol only construct, and screening of various ratios revealed that it is preferable to utilize only Gag-MCP-pol and wt Gag-pol (FIGS. 46C-46D).

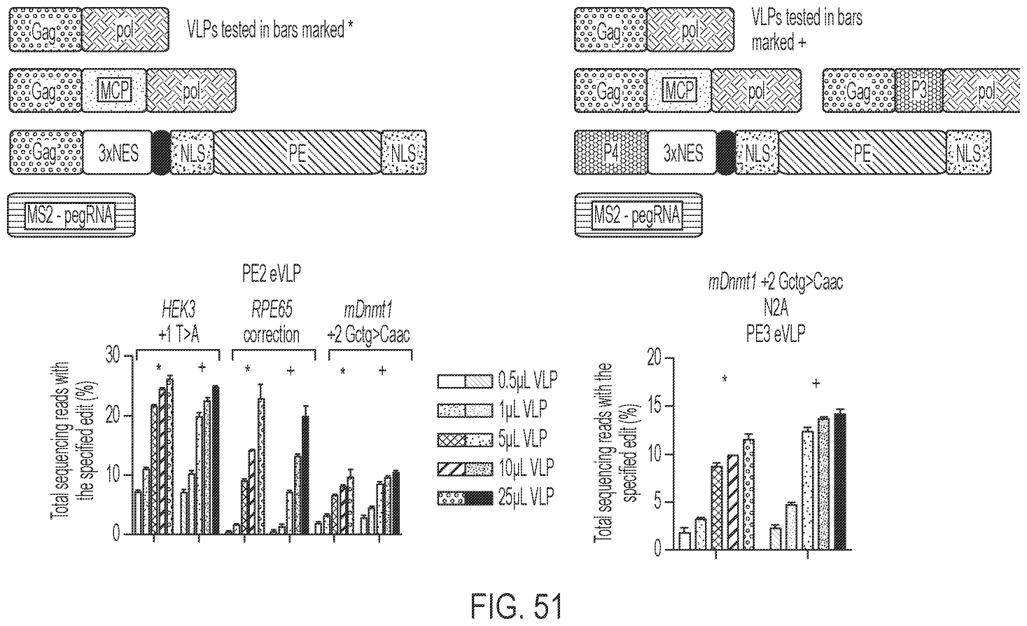

Additional strategies were tested for recruitment of prime editors into eVLPs using coiled-coil peptides (FIG. 51). P3 and p4 are a pair of coiled-coil peptides that are known to form a strong heteromeric interaction, which may be able to help with recruitment of prime editors to eVLPs. P3 peptide was fused to Gag-pol, and the Gag fused to PE was replaced with p4 peptide. With an optimized ratio, the coiled-coil strategy of packaging the prime editor was found to be nearly comparable to the optimized v5 eVLP. Furthermore, the coiled-coil strategy was found to work comparably or even better than the v5 eVLP in the context of delivering PE3. In this strategy, recruitment of prime editor no longer depends on the covalent linkage to the fused Gag domain and instead happens via non-covalent protein-protein interactions. Any strong protein-protein interaction can therefore be used to help recruit prime editors into VLPs.

### Use of Tf1 Reverse Transcriptase in PE-eVLPs

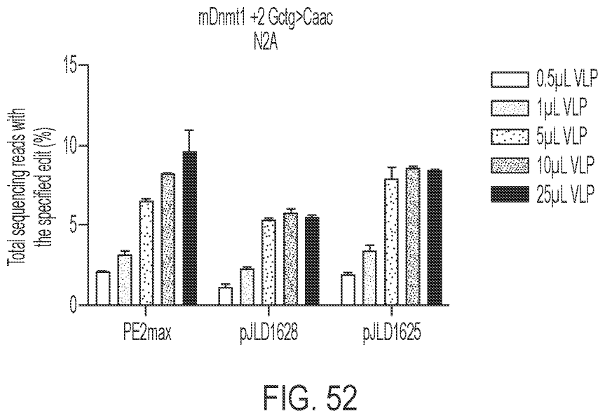

pJLD1628 and pJLD1625 are prime editors that utilize an evolved small reverse transcriptase (Tfl). The use of these prime editors in eVLPs shows that the RT of the prime editor can be modularly switched in the PE-eVLPs (FIG. 52).

### Example 3. Testing of PE VLPs In Vivo

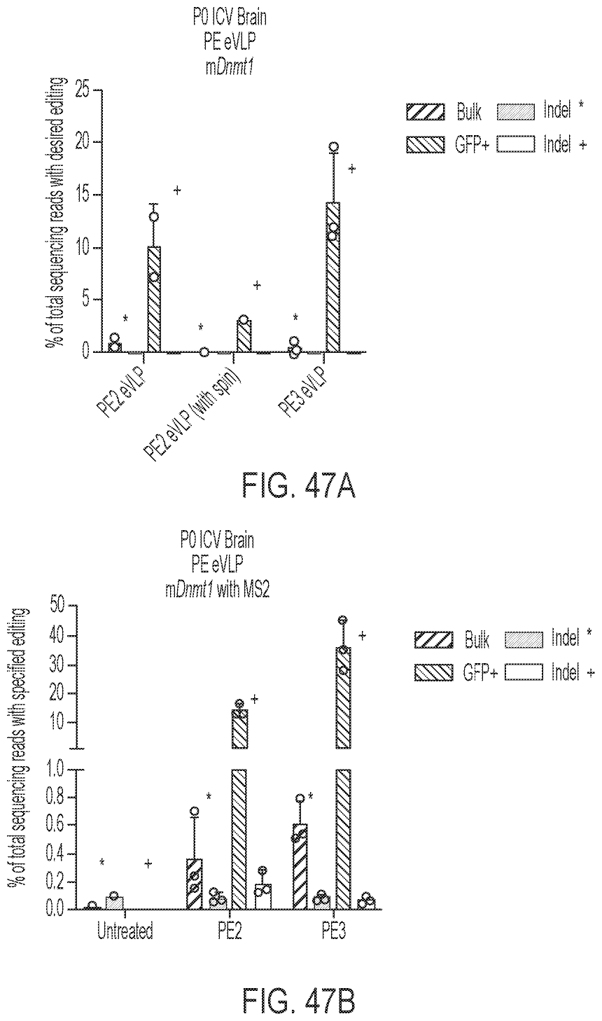

Intracranial injection (ICV) was performed on P0 mice with PE eVLP co-injected with Lenti-GFP:KASH pseudotyped with VSV-G (FIGS. 47A-47B). Among the GFP positive population, which are cell types transducible by VSV-G, the editing efficiency was significantly improved using the MCP-MS2 system, showing up to 45% editing.

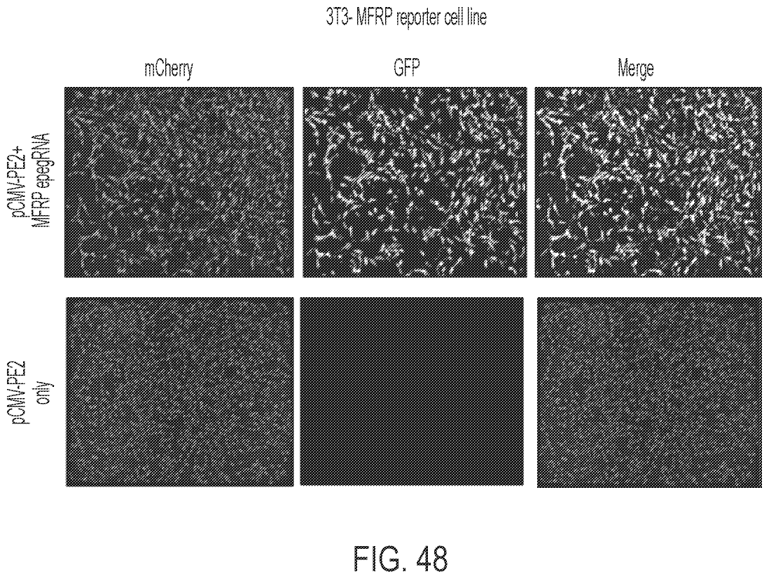

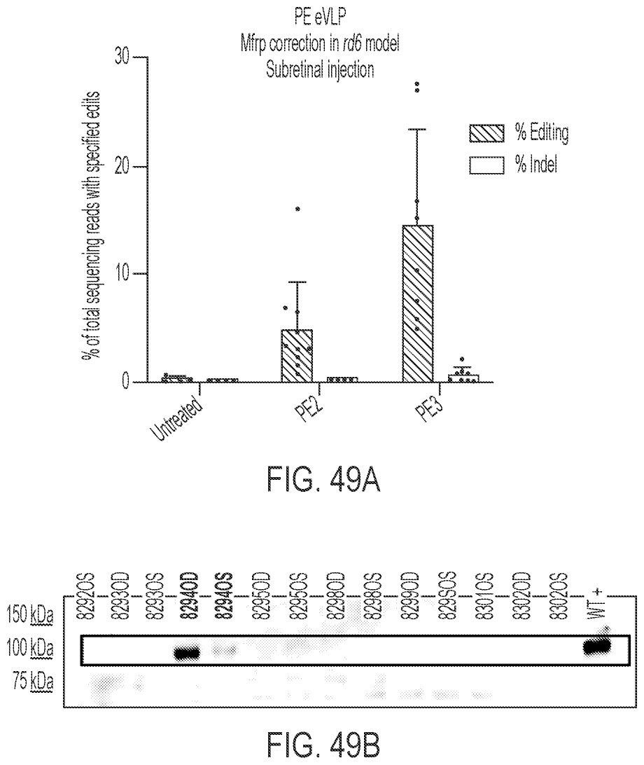

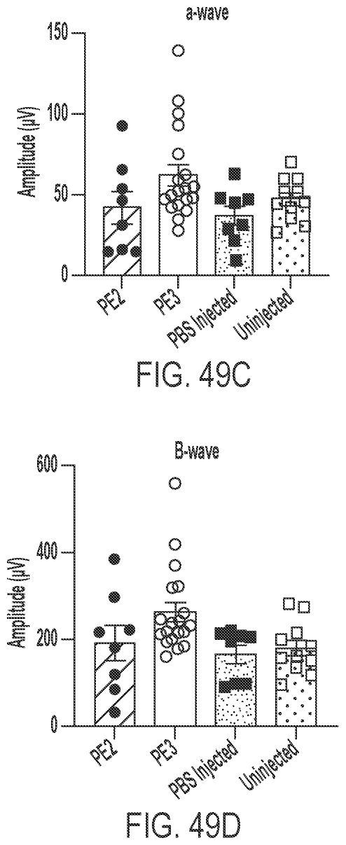

Prime editing strategies for the correction of retinal disease in an rd6 mouse model, which harbors a 4 bp deletion in the splice donor of the membrane-type frizzled-related protein (Mfrp) gene that results in the skipping of exon 4, were screened and optimized (FIG. 48). Skipping of exon 4 results in small, white retinal spots and progressive photoreceptor degeneration. This leads to reinitis pigmentosa and other diseases with mutations in the human homolog. Mfrp is expressed mainly in RPE cells and the ciliary epithelium of retina. With the optimal pegRNA, robust correction of the gene in the reporter cell line was achieved using prime editors delivered by PE VLPs. PE VLPs were used to achieve up to 5% and 15% on average editing with PE2 and PE3 system, respectively (FIGS. 49A-49D). Restoration of protein via western blot was also observed (FIG. 49B).

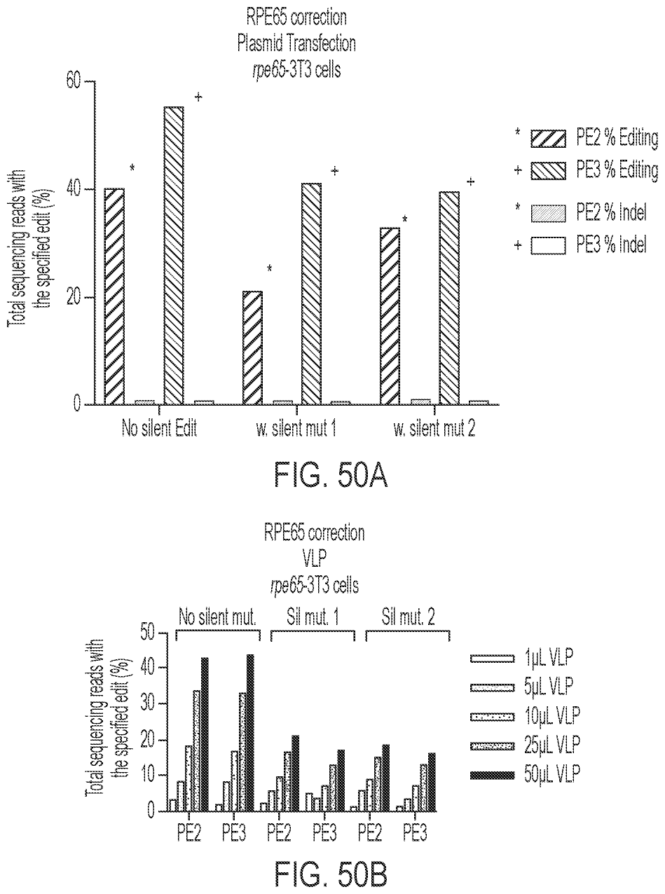

The prime editing strategy for gene correction in the rdl2 model mouse was further optimized (FIGS. 50A-50B). Use of prime editing (delivered by VLPs) allows for cleaner edits and fewer off-target edits compared to other editing strategies. With the optimized pegRNA and ngRNA, over 40% editing in cell culture was achieved using PE VLP.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A virus-like particle (VLP) comprising a group-specific antigen (gag) protease (pro) polyprotein and one or more fusion proteins, wherein the gag-pro polyprotein and the one or more fusion proteins are encapsulated by a lipid membrane and a viral envelope glycoprotein, and wherein each of the one or more fusion proteins comprises:
(i) a gag nucleocapsid protein;
(ii) a nuclear export sequence (NES);
(iii) a cleavable linker; and
(iv) a nucleic acid programmable DNA binding protein (napDNAbp) and/or a domain comprising an RNA-dependent DNA polymerase activity.

2. A VLP comprising (i) a group-specific antigen (gag) protease (pro) polyprotein, (ii) a prime editor comprising a napDNAbp and a domain comprising an RNA-dependent DNA polymerase activity, and (iii) a fusion protein comprising a gag nucleocapsid protein and a nuclear export sequence (NES), encapsulated by a lipid membrane and a viral envelope glycoprotein.

3. The VLP of claim 1 or 2, wherein the napDNAbp is a Cas9 protein.

4. The VLP of claim 3, wherein the Cas9 protein is a Cas9 nickase.

5. The VLP of claim 3, wherein the Cas9 protein is a nuclease-inactivated Cas9 (dCas9).

6. The VLP of any one of claims 1-5, wherein the domain comprising an RNA-dependent DNA polymerase activity is a reverse transcriptase.

7. The VLP of claim 6, wherein the reverse transcriptase is an MMLV reverse transcriptase.

8. The VLP of claim 7, wherein the MMLV reverse transcriptase comprises a C-terminal amino acid truncation to remove the endogenous MMLV protease cleavage site.

9. The VLP of claim 8, wherein the C-terminal amino acid truncation is about 1-180, about 1-170, about 1-160, about 1-150, about 1-140, about 1-130, about 1-120, about 1-110, about 1-100, about 1-90, about 1-80, about 1-70, about 1-60, about 1-50, about 1-40, about 1-30, about 1-20, or about 1-10 amino acids in length.

10. The VLP of claim 8 or 9, wherein the C-terminal amino acid truncation is about six amino acids in length.

11. The VLP of any one of claims 1-10, wherein the napDNAbp is bound to a prime editing guide RNA (pegRNA).

12. The VLP of any one of claims 1 or 3-11, wherein the one or more fusion proteins comprise a prime editor, or a portion thereof.

13. The VLP of claim 2 or 12, wherein the prime editor comprises PE2, PE3, PE4, PE5, PE2max, PE3max, PE4max, or PE5max.

14. The VLP of claim 13, wherein PE3 and PE3max comprise a second strand nicking guide RNA (ngRNA).

15. The VLP of claim 14, wherein the ratio of the ngRNA to the pegRNA is approximately 30:100.

16. The VLP of any one of claims 1 or 3-15, wherein the one or more fusion proteins each comprises two NES, three NES, four NES, five NES, six NES, seven NES, eight NES, nine NES, or ten NES.

17. The VLP of any one of claims 2-15, wherein the fusion protein comprises two NES, three NES, four NES, five NES, six NES, seven NES, eight NES, nine NES, or ten NES.

18. The VLP of any one of claims 1-17, wherein the NES, or multiple NES, are inserted within the gag nucleocapsid protein.

19. The VLP of claim 18, wherein the NES, or multiple NES, are inserted between the p12 and CA domains of the gag nucleocapsid protein, within the p12 domain of the gag nucleocapsid protein, or between the p12 and MA domains of the gag nucleocapsid protein.

20. The VLP of any one of claims 1 or 3-19, wherein the one or more fusion proteins further comprise a nuclear localization sequence (NLS).

21. The VLP of claim 20, wherein the one or more fusion proteins further comprise two NLS.

22. The VLP of claim 21, wherein the one or more fusion proteins comprise a first NLS at the N-terminus of the napDNAbp and a second NLS at the C-terminus of the domain comprising an RNA-dependent DNA polymerase activity.

23. The VLP of claim 2, wherein the prime editor further comprises an NLS.

24. The VLP of claim 23, wherein the prime editor further comprises two NLS.

25. The VLP of claim 24, wherein the prime editor comprises a first NLS at the N-terminus of the napDNAbp and a second NLS at the C-terminus of the domain comprising an RNA-dependent DNA polymerase activity.

26. The VLP of claim 2, wherein the prime editor and the fusion protein were previously fused via a cleavable linker, and the cleavable linker has subsequently been cleaved by the protease of the gag-pro-polyprotein.

27. The VLP of any one of claims 1 or 3-26, wherein the cleavable linker is located between the napDNAbp and the NES.

28. The VLP of any one of claims 1-27, wherein the cleavable linker comprises a protease cleavage site.

29. The VLP of claim 28, wherein the protease cleavage site is a Moloney murine leukemia virus (MMLV) protease cleavage site or a Friend murine leukemia virus (FMLV) protease cleavage site.

30. The VLP of claim 28 or 29, wherein the protease cleavage site comprises the amino acid sequence TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8.

31. The VLP of any one of claims 1 or 3-30, wherein one or more additional linkers are inserted N′ and/or C′ to the cleavable linker.

32. The VLP of claim 31, wherein a linker comprising the amino acid sequence G is inserted C′ to the cleavable linker.

33. The VLP of claim 31, wherein linkers comprising the amino acid sequence GGS are inserted N′ and/or C′ to the cleavable linker.

34. The VLP of claim 31, wherein linkers comprising the amino acid sequence SGGSSGGS (SEQ ID NO: 163) are inserted N′ and/or C′ to the cleavable linker.

35. The VLP of any one of claims 1-34, wherein the gag-pro polyprotein comprises an MMLV gag-pro polyprotein or an FMLV gag-pro polyprotein.

36. The VLP of any one of claims 1-35, wherein the gag nucleocapsid protein comprises an MMLV gag nucleocapsid protein or an FMLV gag nucleocapsid protein.

37. The VLP of any one of claims 1 or 3-36, wherein the napDNAbp and the domain comprising an RNA-dependent DNA polymerase activity are included on the same fusion protein.

38. The VLP of claim 37, wherein the fusion protein comprises the structure:
[gag nucleocapsid protein]-[napDNAbp]-[domain comprising RNA-dependent DNA polymerase activity], wherein]-[comprises an optional linker.

39. The VLP of claim 37, wherein the fusion protein comprises the structure:
[gag nucleocapsid protein]-[1×-3×NES]-[cleavable linker]-[NLS]-[napDNAbp]-[domain comprising RNA-dependent DNA polymerase activity]-[NLS], wherein]-[comprises an optional linker.

40. The VLP of any one of claims 1 or 3-36, wherein the napDNAbp and the domain comprising an RNA-dependent DNA polymerase activity are included on two different fusion proteins, and wherein each of the fusion proteins comprises a split intein to facilitate fusion of the napDNAbp and the domain comprising an RNA-dependent DNA polymerase activity.

41. The VLP of claim 40, wherein the two fusion proteins comprise the structures:
[gag nucleocapsid protein]-[napDNAbp]-[split intein]; and
[gag nucleocapsid protein]-[split intein]-[domain comprising RNA-dependent DNA polymerase activity], wherein]-[comprises an optional linker.

42. The VLP of claim 40, wherein the two fusion proteins comprise the structures:
[gag nucleocapsid protein]-[first portion of napDNAbp]-[split intein]; and
[gag nucleocapsid protein]-[split intein]-[second portion of napDNAbp]-[domain comprising RNA-dependent DNA polymerase activity], wherein]-[comprises an optional linker.

43. The VLP of any one of claims 2-36, wherein the fusion protein comprises the structure:
[gag nucleocapsid protein]-[1×-3×NES], wherein]-[comprises an optional linker.

44. The VLP of any one of claims 2-36 or 43, wherein the prime editor comprises the structure:
[NLS]-[domain comprising RNA-dependent DNA polymerase activity]-[napDNAbp]-[NLS], wherein]-[comprises an optional linker.

45. The VLP of any one of claims 1-44, wherein the viral envelope glycoprotein is an adenoviral envelope glycoprotein, an adeno-associated viral envelope glycoprotein, a retroviral envelope glycoprotein, or a lentiviral envelope glycoprotein.

46. The VLP of claim 45, wherein the viral envelope glycoprotein is a retroviral envelope glycoprotein.

47. The VLP of claim 46, wherein the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein.

48. The VLP of any one of claims 1-47, wherein the VLP further comprises an inhibitor of the DNA mismatch repair (MMR) pathway.

49. The VLP of claim 48, wherein the inhibitor of MMR comprises MLH1dn.

50. The VLP of claim 48 or 49, wherein the inhibitor of MMR is fused to a gag nucleocapsid protein, and wherein the MMR inhibitor-gag nucleocapsid protein fusion is encapsulated by a viral envelope glycoprotein.

51. The VLP of claim 50, wherein the MMR inhibitor-gag nucleocapsid protein fusion further comprises one or more NES.

52. The VLP of claim 50 or 51, wherein the MMR inhibitor-gag nucleocapsid protein fusion further comprises a cleavable linker.

53. The VLP of any one of claims 50-52, wherein the MMR inhibitor-gag nucleocapsid protein fusion comprises the structure:
[gag nucleocapsid protein]-[1×-3×NES]-[cleavable linker]-[MMR inhibitor], wherein]-[comprises an optional linker.

54. The VLP of any one of claims 11-53, wherein the pegRNA comprises one or more silent mutations to increase editing efficiency by facilitating evasion of the MMR pathway.

55. The VLP of any one of claims 11-54, wherein the pegRNA and/or ngRNA structure comprises an aptamer, and wherein the gag-pro polyprotein is fused to a target molecule that binds the aptamer, thereby facilitating packaging of the pegRNA and/or ngRNA into the VLP.

56. The VLP of claim 55, wherein the aptamer is inserted into the pegRNA backbone sequence and/or the ngRNA backbone sequence.

57. The VLP of claim 55 or 56, wherein the target molecule that binds the aptamer is inserted into the gag-pro polyprotein.

58. The VLP of any one of claims 55-57, wherein the aptamer comprises the MS2 stem loop, and wherein the target molecule that binds the aptamer comprises the MS2 coat protein.

59. The VLP of any one of claims 55-57, wherein the aptamer comprises the Com aptamer, and wherein the target molecule that binds the aptamer comprises the Com protein.

60. The VLP of any one of claims 55-59, wherein the ratio of wild type gag-pro polyprotein to target molecule-modified gag-pro polyprotein to one or more fusion proteins in the VLP is approximately 5:2:1.

61. The VLP of any one of claims 1-60, wherein the Gag-pro polyprotein is fused to a first coiled-coil peptide and the one or more fusion proteins are fused to a second coiled-coil peptide, wherein interaction of the first and second coiled-coil peptides with one another facilitates the assembly of the VLP.

62. The VLP of claim 61, wherein the first coiled-coil peptide is inserted into the gag-pro polyprotein.

63. The VLP of claim 61 or 62, wherein the second coiled-coil peptide is fused to the N-terminus of the one or more fusion proteins, the C-terminus of the one or more fusion proteins, or at an internal position within the one or more fusion proteins.

64. The VLP of claim 63, wherein the second coiled-coil peptide is fused to the C-terminus of the one or more fusion proteins.

65. The VLP of any one of claims 61-64, wherein one of the first or the second coiled-coil peptides comprises the P3 peptide, and the other of the first or the second coiled-coil peptides comprises the P4 peptide.

66. The VLP of any one of claims 61-65, wherein the first coiled-coil peptide comprises the P3 peptide.

67. The VLP of any one of claims 61-66, wherein the second coiled-coil peptide comprises the P4 peptide.

68. A cell comprising the VLP of any one of claims 1-67.

69. A plurality of polynucleotides comprising:
(i) a first polynucleotide comprising a nucleic acid sequence encoding a viral envelope glycoprotein;
(ii) a second polynucleotide comprising a nucleic acid sequence encoding a group-specific antigen (gag) protease (pro) polyprotein;
(iii) a third polynucleotide comprising a nucleic acid sequence encoding one or more fusion proteins, wherein each of the one or more fusion proteins comprises:
(a) a gag nucleocapsid protein;
(b) a nuclear export sequence (NES);
(c) a cleavable linker; and
(d) a nucleic acid programmable DNA binding protein (napDNAbp) and/or a domain comprising an RNA-dependent DNA polymerase activity; and
(iv) a fourth polynucleotide comprising a nucleic acid sequence encoding a guide RNA (gRNA), wherein the gRNA binds to the napDNAbp of the one or more fusion proteins encoded by the third polynucleotide.

70. The plurality of polynucleotides of claim 69, wherein the ratio of the second polynucleotide to the third polynucleotide is approximately 10:1, approximately 9:1, approximately 8:1, approximately 7:1, approximately 6:1, approximately 5:1, approximately 4:1, approximately 3:1, approximately 2:1, approximately 1.5:1, approximately 1:1, or approximately 0.5:1.

71. The plurality of polynucleotides of claim 70, wherein the ratio of the second polynucleotide to the third polynucleotide is approximately 3:1.

72. The plurality of polynucleotides of any one of claims 69-71, wherein the napDNAbp is a Cas9 protein.

73. The plurality of polynucleotides of claim 72, wherein the Cas9 protein is a Cas9 nickase.

74. The plurality of polynucleotides of claim 72, wherein the Cas9 protein is a nuclease-inactive Cas9 (dCas9).

75. The plurality of polynucleotides of any one of claims 69-74, wherein the domain comprising an RNA-dependent DNA polymerase activity is a reverse transcriptase.

76. The plurality of polynucleotides of claim 75, wherein the reverse transcriptase is an MMLV reverse transcriptase.

77. The plurality of polynucleotides of claim 76, wherein the MMLV reverse transcriptase comprises a C-terminal amino acid truncation to remove the endogenous MMLV protease cleavage site.

78. The plurality of polynucleotides of claim 77, wherein the C-terminal amino acid truncation is about 1-180, about 1-170, about 1-160, about 1-150, about 1-140, about 1-130, about 1-120, about 1-110, about 1-100, about 1-90, about 1-80, about 1-70, about 1-60, about 1-50, about 1-40, about 1-30, about 1-20, or about 1-10 amino acids in length.

79. The plurality of polynucleotides of claim 78, wherein the C-terminal amino acid truncation is about six amino acids in length.

80. The plurality of polynucleotides of any one of claims 69-79, wherein the gRNA is a prime editing guide RNA (pegRNA).

81. The plurality of polynucleotides of any one of claims 69-80, wherein the one or more fusion proteins comprise a prime editor, or a portion thereof.

82. The plurality of polynucleotides of claim 81, wherein the prime editor comprises PE2, PE3, PE4, PE5, PE2max, PE3max, PE4max, or PE5max.

83. The plurality of polynucleotides of claim 82, wherein PE3 and PE3max comprise a second strand nicking guide RNA (ngRNA).

84. The plurality of polynucleotides of claim 83, wherein the ratio of the ngRNA to the pegRNA is approximately 30:100.

85. The plurality of polynucleotides of any one of claims 69-84, wherein the one or more fusion proteins each comprises two NES, three NES, four NES, five NES, six NES, seven NES, eight NES, nine NES, or ten NES.

86. The plurality of polynucleotides of any one of claims 69-85, wherein the NES, or multiple NES, are inserted within the gag nucleocapsid protein.

87. The plurality of polynucleotides of claim 86, wherein the NES, or multiple NES, are inserted between the p12 and CA domains of the gag nucleocapsid protein, within the p12 domain of the gag nucleocapsid protein, or between the p12 and MA domains of the gag nucleocapsid protein.

88. The plurality of polynucleotides of any one of claims 69-87, wherein the one or more fusion proteins further comprise a nuclear localization sequence (NLS).

89. The plurality of polynucleotides of claim 88, wherein the one or more fusion proteins further comprise two NLS.

90. The plurality of polynucleotides of claim 89, wherein the one or more fusion proteins comprise a first NLS at the N-terminus of the napDNAbp and a second NLS at the C-terminus of the domain comprising an RNA-dependent DNA polymerase activity.

91. The plurality of polynucleotides of any one of claims 69-90, wherein the cleavable linker is located between the napDNAbp and the NES.

92. The plurality of polynucleotides of any one of claims 69-91, wherein the cleavable linker comprises a protease cleavage site.

93. The plurality of polynucleotides of claim 92, wherein the protease cleavage site is a Moloney murine leukemia virus (MMLV) protease cleavage site or a Friend murine leukemia virus (FMLV) protease cleavage site.

94. The plurality of polynucleotides of claim 92 or 93, wherein the protease cleavage site comprises the amino acid sequence TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8.

95. The plurality of polynucleotides of any one of claims 69-94, wherein one or more additional linkers are inserted N′ and/or C′ to the cleavable linker.

96. The plurality of polynucleotides of claim 95, wherein a linker comprising the amino acid sequence G is inserted C′ to the cleavable linker.

97. The plurality of polynucleotides of claim 95, wherein linkers comprising the amino acid sequence GGS are inserted N′ and/or C′ to the cleavable linker.

98. The plurality of polynucleotides of claim 95, wherein linkers comprising the amino acid sequence SGGSSGGS (SEQ ID NO: 163) are inserted N′ and/or C′ to the cleavable linker.

99. The plurality of polynucleotides of any one of claims 69-98, wherein the gag-pro polyprotein comprises an MMLV gag-pro polyprotein or an FMLV gag-pro polyprotein.

100. The plurality of polynucleotides of any one of claims 69-99, wherein the gag nucleocapsid protein comprises an MMLV gag nucleocapsid protein or an FMLV gag nucleocapsid protein.

101. The plurality of polynucleotides of any one of claims 69-100, wherein the napDNAbp and the domain comprising an RNA-dependent DNA polymerase activity are included on the same fusion protein.

102. The plurality of polynucleotides of claim 101, wherein the fusion protein comprises the structure:
[gag nucleocapsid protein]-[napDNAbp]-[domain comprising RNA-dependent DNA polymerase activity], wherein]-[comprises an optional linker.

103. The plurality of polynucleotides of claim 101, wherein the fusion protein comprises the structure:
[gag nucleocapsid protein]-[1×-3×NES]-[cleavable linker]-[NLS]-[napDNAbp]-[domain comprising RNA-dependent DNA polymerase activity]-[NLS], wherein]-[comprises an optional linker.

104. The plurality of polynucleotides of any one of claims 69-103, wherein the napDNAbp and the domain comprising an RNA-dependent DNA polymerase activity are included on two different fusion proteins, and wherein each of the fusion proteins comprises a split intein to facilitate fusion of the napDNAbp and the domain comprising an RNA-dependent DNA polymerase activity.

105. The plurality of polynucleotides of claim 104, wherein the two fusion proteins comprise the structures:
[gag nucleocapsid protein]-[napDNAbp]-[split intein]; and
[gag nucleocapsid protein]-[split intein]-[domain comprising RNA-dependent DNA polymerase activity], wherein]-[comprises an optional linker.

106. The plurality of polynucleotides of any one of claims 69-105, wherein the viral envelope glycoprotein is an adenoviral envelope glycoprotein, an adeno-associated viral envelope glycoprotein, a retroviral envelope glycoprotein, or a lentiviral envelope glycoprotein.

107. The plurality of polynucleotides of claim 106, wherein the viral envelope glycoprotein is a retroviral envelope glycoprotein.

108. The plurality of polynucleotides of claim 107, wherein the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein.

109. The plurality of polynucleotides of any one of claims 69-108 further comprising a fifth polynucleotide encoding an inhibitor of the DNA mismatch repair (MMR) pathway.

110. The plurality of polynucleotides of claim 109, wherein the inhibitor of MMR comprises MLH1dn.

111. The plurality of polynucleotides of claim 109 or 110, wherein the inhibitor of MMR is fused to a gag nucleocapsid protein, and wherein the MMR inhibitor-gag nucleocapsid protein fusion is encapsulated by a viral envelope glycoprotein.

112. The plurality of polynucleotides of claim 111, wherein the MMR inhibitor-gag nucleocapsid protein fusion further comprises one or more NES.

113. The plurality of polynucleotides of claim 111 or 112, wherein the MMR inhibitor-gag nucleocapsid protein fusion further comprises a cleavable linker.

114. The plurality of polynucleotides of any one of claims 111-113, wherein the MMR inhibitor-gag nucleocapsid protein fusion comprises the structure:
[gag nucleocapsid protein]-[1×-3×NES]-[cleavable linker]-[MMR inhibitor], wherein]-[comprises an optional linker.

115. The plurality of polynucleotides of any one of claims 80-114, wherein the pegRNA comprises one or more silent mutations to increase editing efficiency by facilitating evasion of the MMR pathway.

116. The plurality of polynucleotides of any one of claims 80-115, wherein the pegRNA and/or ngRNA structure comprises an aptamer, and wherein the gag-pro polyprotein is fused to a target molecule that binds the aptamer, thereby facilitating packaging of the pegRNA and/or ngRNA into the VLP.

117. The plurality of polynucleotides of claim 116, wherein the aptamer is inserted into the pegRNA backbone sequence and/or the ngRNA backbone sequence.

118. The plurality of polynucleotides of claim 116 or 117, wherein the target molecule that binds the aptamer is inserted into the gag-pro polyprotein.

119. The plurality of polynucleotides of any one of claims 116-118, wherein the aptamer comprises the MS2 stem loop, and wherein the target molecule that binds the aptamer comprises the MS2 coat protein.

120. The plurality of polynucleotides of any one of claims 116-118, wherein the aptamer comprises the Com aptamer, and wherein the target molecule that binds the aptamer comprises the Com protein.

121. The plurality of polynucleotides of any one of claims 116-118, wherein the ratio of wild type gag-pro polyprotein to target molecule-modified gag-pro polyprotein to one or more fusion proteins in the VLP encoded by the plurality of polynucleotides is approximately 5:2:1.

122. The plurality of polynucleotides of any one of claims 69-121, wherein the Gag-pro polyprotein is fused to a first coiled-coil peptide and the one or more fusion proteins are fused to a second coiled-coil peptide, wherein interaction of the first and second coiled-coil peptides with one another facilitates the assembly of the VLP encoded by the plurality of polynucleotides.

123. The plurality of polynucleotides of claim 122, wherein the first coiled-coil peptide is inserted into the gag-pro polyprotein.

124. The plurality of polynucleotides of claim 122 or 123, wherein the second coiled-coil peptide is fused to the N-terminus of the one or more fusion proteins, the C-terminus of the one or more fusion proteins, or at an internal position within the one or more fusion proteins.

125. The plurality of polynucleotides of claim 124, wherein the second coiled-coil peptide is fused to the C-terminus of the one or more fusion proteins.

126. The plurality of polynucleotides of any one of claims 122-125, wherein one of the first or the second coiled-coil peptides comprises the P3 peptide, and the other of the first or the second coiled-coil peptides comprises the P4 peptide.

127. The plurality of polynucleotides of any one of claims 122-126, wherein the first coiled-coil peptide comprises the P3 peptide.

128. The plurality of polynucleotides of any one of claims 122-127, wherein the second coiled-coil peptide comprises the P4 peptide.

129. One or more vectors comprising the plurality of polynucleotides of any one of claims 69-128.

130. The one or more vectors of claim 129, wherein each of the first, second, third, and fourth polynucleotides are on separate vectors.

131. The one or more vectors of claim 129, wherein one or more of the first, second, third, and fourth polynucleotides are on the same vector.

132. A cell comprising the plurality of polynucleotides of any one of claims 60-118 or the one or more vectors of any one of claims 129-131.

133. A method of making a virus-like particle (VLP) for delivering a prime editor fusion protein comprising transfecting the plurality of polynucleotides of any one of claims 60-118 or the one or more vectors of any one of claims 129-131 into a cell.

134. A pharmaceutical composition comprising a virus-like particle (VLP) comprising a group-specific antigen (gag) protease (pro) polyprotein and one or more fusion proteins, wherein the gag-pro polyprotein and the one or more fusion proteins are encapsulated by a lipid membrane and a viral envelope glycoprotein, and wherein each of the one or more fusion proteins comprises:
(i) a gag nucleocapsid protein;
(ii) a nuclear export sequence (NES);
(iii) a cleavable linker; and
(iv) a nucleic acid programmable DNA binding protein (napDNAbp) and/or a domain comprising an RNA-dependent DNA polymerase activity.

135. A pharmaceutical composition comprising a VLP comprising (i) a group-specific antigen (gag) protease (pro) polyprotein, (ii) a prime editor comprising a napDNAbp and a domain comprising an RNA-dependent DNA polymerase activity, and (iii) a fusion protein comprising a gag nucleocapsid protein and a nuclear export sequence (NES), encapsulated by a lipid membrane and a viral envelope glycoprotein.

136. The pharmaceutical composition of claim 134 or 135, wherein the napDNAbp is a Cas9 protein.

137. The pharmaceutical composition of claim 136, wherein the Cas9 protein is a Cas9 nickase.

138. The pharmaceutical composition of claim 136, wherein the Cas9 protein is a nuclease-inactive Cas9 (dCas9).

139. The pharmaceutical composition of any one of claims 134-138, wherein the domain comprising an RNA-dependent DNA polymerase activity is a reverse transcriptase.

140. The pharmaceutical composition of claim 139, wherein the reverse transcriptase is an MMLV reverse transcriptase.

141. The pharmaceutical composition of claim 140, wherein the MMLV reverse transcriptase comprises a C-terminal amino acid truncation to remove the endogenous MMLV protease cleavage site.

142. The pharmaceutical composition of claim 141, wherein the C-terminal amino acid truncation is about 1-180, about 1-170, about 1-160, about 1-150, about 1-140, about 1-130, about 1-120, about 1-110, about 1-100, about 1-90, about 1-80, about 1-70, about 1-60, about 1-50, about 1-40, about 1-30, about 1-20, or about 1-10 amino acids in length.

143. The pharmaceutical composition of claim 141 or 142, wherein the C-terminal amino acid truncation is about six amino acids in length.

144. The pharmaceutical composition of any one of claims 134 or 136-143, wherein the napDNAbp is bound to a prime editing guide RNA (pegRNA).

145. The pharmaceutical composition of any one of claims 134 or 136-144, wherein the fusion protein comprises a prime editor, or a portion thereof.

146. The pharmaceutical composition of claim 135 or 145, wherein the prime editor comprises PE2, PE3, PE4, PE5, PE2max, PE3max, PE4max, or PE5max.

147. The pharmaceutical composition of claim 146, wherein PE3 and PE3max comprise a second strand nicking guide RNA (ngRNA).

148. The pharmaceutical composition of claim 147, wherein the ratio of the ngRNA to the pegRNA is approximately 30:100.

149. The pharmaceutical composition of any one of claims 134 or 136-148, wherein the one or more fusion proteins each comprises two NES, three NES, four NES, five NES, six NES, seven NES, eight NES, nine NES, or ten NES.

150. The pharmaceutical composition of any one of claims 135-149, wherein the fusion protein comprises two NES, three NES, four NES, five NES, six NES, seven NES, eight NES, nine NES, or ten NES.

151. The pharmaceutical composition of any one of claims 134-150, wherein the NES, or multiple NES, are inserted within the gag nucleocapsid protein.

152. The pharmaceutical composition of claim 151, wherein the NES, or multiple NES, are inserted between the p12 and CA domains of the gag nucleocapsid protein, within the p12 domain of the gag nucleocapsid protein, or between the p12 and MA domains of the gag nucleocapsid protein.

153. The pharmaceutical composition of any one of claims 134 or 136-152, wherein the one or more fusion proteins further comprise a nuclear localization sequence (NLS).

154. The pharmaceutical composition of claim 153, wherein the one or more fusion proteins further comprise two NLS.

155. The pharmaceutical composition of claim 154, wherein the one or more fusion proteins comprise a first NLS at the N-terminus of the napDNAbp and a second NLS at the C-terminus of the domain comprising an RNA-dependent DNA polymerase activity.

156. The pharmaceutical composition of claim 135, wherein the prime editor further comprises an NLS.

157. The pharmaceutical composition of claim 156, wherein the prime editor further comprises two NLS.

158. The pharmaceutical composition of claim 157, wherein the prime editor comprises a first NLS at the N-terminus of the napDNAbp and a second NLS at the C-terminus of the domain comprising an RNA-dependent DNA polymerase activity.

159. The pharmaceutical composition of claim 135, wherein the prime editor and the fusion protein were previously fused via a cleavable linker, and the cleavable linker has subsequently been cleaved by the protease of the gag-pro-polyprotein.

160. The pharmaceutical composition of any one of claims 134 or136-159, wherein the cleavable linker is located between the napDNAbp and the NES.

161. The pharmaceutical composition of any one of claims 134-160, wherein the cleavable linker comprises a protease cleavage site.

162. The pharmaceutical composition of claim 161, wherein the protease cleavage site is a Moloney murine leukemia virus (MMLV) protease cleavage site or a Friend murine leukemia virus (FMLV) protease cleavage site.

163. The pharmaceutical composition of claim 161 or 162, wherein the protease cleavage site comprises the amino acid sequence TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8.

164. The pharmaceutical composition of any one of claims 134 or 136-163, wherein one or more additional linkers are inserted N′ and/or C′ to the cleavable linker.

165. The pharmaceutical composition of claim 164, wherein a linker comprising the amino acid sequence G is inserted C′ to the cleavable linker.

166. The pharmaceutical composition of claim 164, wherein linkers comprising the amino acid sequence GGS are inserted N′ and/or C′ to the cleavable linker.

167. The pharmaceutical composition of claim 164, wherein linkers comprising the amino acid sequence SGGSSGGS (SEQ ID NO: 163) are inserted N′ and/or C′ to the cleavable linker.

168. The pharmaceutical composition of any one of claims 134-167, wherein the gag-pro polyprotein comprises an MMLV gag-pro polyprotein or an FMLV gag-pro polyprotein.

169. The pharmaceutical composition of any one of claims 134-168, wherein the gag nucleocapsid protein comprises an MMLV gag nucleocapsid protein or an FMLV gag nucleocapsid protein.

170. The pharmaceutical composition of any one of claims 134 or 136-169, wherein the napDNAbp and the domain comprising an RNA-dependent DNA polymerase activity are included on the same fusion protein.

171. The pharmaceutical composition of claim 170, wherein the fusion protein comprises the structure:
[gag nucleocapsid protein]-[napDNAbp]-[domain comprising RNA-dependent DNA polymerase activity], wherein]-[comprises an optional linker.

172. The pharmaceutical composition of claim 170, wherein the fusion protein comprises the structure:
[gag nucleocapsid protein]-[1×-3×NES]-[cleavable linker]-[NLS]-[napDNAbp]-[domain comprising RNA-dependent DNA polymerase activity]-[NLS], wherein]-[comprises an optional linker.

173. The pharmaceutical composition of any one of claims 134 or 136-172, wherein the napDNAbp and the domain comprising an RNA-dependent DNA polymerase activity are included on two different fusion proteins, and wherein each of the fusion proteins comprises a split intein to facilitate fusion of the napDNAbp and the domain comprising an RNA-dependent DNA polymerase activity.

174. The pharmaceutical composition of claim 173, wherein the two fusion proteins comprise the structures:
[gag nucleocapsid protein]-[napDNAbp]-[split intein]; and
[gag nucleocapsid protein]-[split intein]-[domain comprising RNA-dependent DNA polymerase activity], wherein]-[comprises an optional linker.

175. The pharmaceutical composition of claim 173, wherein the two fusion proteins comprise the structures:
[gag nucleocapsid protein]-[first portion of napDNAbp]-[split intein]; and
[gag nucleocapsid protein]-[split intein]-[second portion of napDNAbp]-[domain comprising RNA-dependent DNA polymerase activity], wherein]-[comprises an optional linker.

176. The pharmaceutical composition of any one of claims 135-169, wherein the fusion protein comprises the structure:
[gag nucleocapsid protein]-[1×-3×NES], wherein]-[comprises an optional linker.

177. The pharmaceutical composition of any one of claims 135-169 or 176, wherein the prime editor comprises the structure:
[NLS]-[reverse transcriptase domain]-[napDNAbp]-[NLS], wherein]-[comprises an optional linker.

178. The pharmaceutical composition of any one of claims 134-177, wherein the viral envelope glycoprotein is an adenoviral envelope glycoprotein, an adeno-associated viral envelope glycoprotein, a retroviral envelope glycoprotein, or a lentiviral envelope glycoprotein.

179. The pharmaceutical composition of claim 178, wherein the viral envelope glycoprotein is a retroviral envelope glycoprotein.

180. The pharmaceutical composition of claim 179, wherein the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein.

181. The pharmaceutical composition of any one of claims 134-180 further comprising an inhibitor of the DNA mismatch repair (MMR) pathway.

182. The pharmaceutical composition of claim 181, wherein the inhibitor of MMR comprises MLH1dn.

183. The pharmaceutical composition of claim 181 or 182, wherein the inhibitor of MMR is fused to a gag nucleocapsid protein, and wherein the MMR inhibitor-gag nucleocapsid protein fusion is encapsulated by a viral envelope glycoprotein.

184. The pharmaceutical composition of claim 183, wherein the MMR inhibitor-gag nucleocapsid protein fusion further comprises one or more NES.

185. The pharmaceutical composition of claim 183 or 184, wherein the MMR inhibitor-gag nucleocapsid protein fusion further comprises a cleavable linker.

186. The pharmaceutical composition of any one of claims 134-185, wherein the MMR inhibitor-gag nucleocapsid protein fusion comprises the structure:
[gag nucleocapsid protein]-[lX-3×NES]-[cleavable linker]-[MMR inhibitor], wherein]-[comprises an optional linker.

187. The pharmaceutical composition of any one of claims 144-186, wherein the pegRNA comprises one or more silent mutations to increase editing efficiency by facilitating evasion of the MMR pathway.

188. The pharmaceutical composition of any one of claims 144-187, wherein the pegRNA and/or ngRNA structure comprises an aptamer, and wherein the gag-pro polyprotein is fused to a target molecule that binds the aptamer, thereby facilitating packaging of the pegRNA and/or ngRNA into the VLP.

189. The pharmaceutical composition of claim 188, wherein the aptamer is inserted into the pegRNA backbone sequence and/or the ngRNA backbone sequence.

190. The pharmaceutical composition of claim 188 or 189, wherein the target molecule that binds the aptamer is inserted into the gag-pro polyprotein.

191. The pharmaceutical composition of any one of claims 188-190, wherein the aptamer comprises the MS2 stem loop, and wherein the target molecule that binds the aptamer comprises the MS2 coat protein.

192. The pharmaceutical composition of any one of claims 188-190, wherein the aptamer comprises the Com aptamer, and wherein the target molecule that binds the aptamer comprises the Com protein.

193. The pharmaceutical composition of any one of claims 188-192, wherein the ratio of wild type gag-pro polyprotein to target molecule-modified gag-pro polyprotein to one or more fusion proteins in the VLP is approximately 5:2:1.

194. The pharmaceutical composition of any one of claims 134-193, wherein the Gag-pro polyprotein is fused to a first coiled-coil peptide and the one or more fusion proteins are fused to a second coiled-coil peptide, wherein interaction of the first and second coiled-coil peptides with one another facilitates the assembly of the VLP.

195. The pharmaceutical composition of claim 194, wherein the first coiled-coil peptide is inserted into the gag-pro polyprotein.

196. The pharmaceutical composition of claim 194 or 195, wherein the second coiled-coil peptide is fused to the N-terminus of the one or more fusion proteins, the C-terminus of the one or more fusion proteins, or at an internal position within the one or more fusion proteins.

197. The pharmaceutical composition of claim 196, wherein the second coiled-coil peptide is fused to the C-terminus of the one or more fusion proteins.

198. The pharmaceutical composition of any one of claims 194-197, wherein one of the first or the second coiled-coil peptides comprises the P3 peptide, and the other of the first or the second coiled-coil peptides comprises the P4 peptide.

199. The pharmaceutical composition of any one of claims 194-197, wherein the first coiled-coil peptide comprises the P3 peptide.

200. The pharmaceutical composition of any one of claims 194-199, wherein the second coiled-coil peptide comprises the P4 peptide.

201. A method for editing a nucleic acid molecule in a target cell by prime editing comprising contacting the target cell with the VLP of any one of claims 1-67 or the pharmaceutical composition of any one of claims 134-200, thereby installing one or more modifications to the nucleic acid molecule at a target site.

202. The method of claim 201, wherein the target cell is a mammalian cell.

203. The method of claim 201 or 202, wherein the target cell is a human cell.

204. The method of any one of claims 201-203, wherein the cell is in a subject.

205. The method of claim 204, wherein the subject is a human.

206. The method of any one of claims 201-205, wherein the one or more modifications to the nucleic acid molecule are associated with reducing, relieving, or preventing the symptoms of a disease or disorder.

207. The method of any one of claims 201-206 further comprising contacting the target cell with additional pegRNA molecules.

208. The method of claim 207, wherein contacting the target cell with additional pegRNA molecules increases the prime editing efficiency.

209. The method of any one of claims 201-208, wherein the extension arm of the pegRNA comprises a DNA synthesis template comprising three or more consecutive nucleotide mismatches relative to the endogenous sequence of the target site on the nucleic acid molecule.

210. The method of claim 209, wherein at least one of the three consecutive nucleotide mismatches results in an alteration in the amino acid sequence of a protein expressed from the nucleic acid molecule, and wherein at least one of the remaining three or more consecutive nucleotide mismatches are silent mutations.

211. The method of claim 210, wherein the silent mutations are in a coding region of the nucleic acid molecule.

212. The method of claim 211, wherein the silent mutations introduce into the nucleic acid molecule one or more alternate codons encoding the same amino acid as the unedited nucleic acid molecule.

213. The method of claim 210, wherein the silent mutations are in a non-coding region of the nucleic acid molecule.

214. The method of claim 213, wherein the silent mutations are in a region of the nucleic acid molecule that does not influence splicing, gene regulation, RNA lifetime, or other biological properties of the target site on the nucleic acid molecule.

215. The method of any one of claims 209-214, wherein the extension arm of the pegRNA comprises four or more, five or more, six or more, seven or more, eight or more, nine or more, or ten or more consecutive nucleotide mismatches relative to the endogenous sequence of the target site on the nucleic acid molecule.

216. The method of any one of claims 209-215, wherein the three or more consecutive nucleotide mismatches evade correction by the DNA mismatch repair pathway.

217. A fusion protein comprising:
(i) a gag nucleocapsid protein;
(ii) a nuclear export sequence (NES);
(iii) a cleavable linker;
(iv) a nucleic acid programmable DNA binding protein (napDNAbp); and/or a domain comprising an RNA-dependent DNA polymerase activity.

218. The fusion protein of claim 217, wherein the napDNAbp is a Cas9 protein.

219. The fusion protein of claim 218, wherein the Cas9 protein is a Cas9 nickase.

220. The fusion protein of claim 218, wherein the Cas9 protein is a nuclease-inactivated Cas9 protein.

221. The fusion protein of any one of claims 217-220, wherein the domain comprising an RNA-dependent DNA polymerase activity is a reverse transcriptase.

222. The fusion protein of claim 221, wherein the reverse transcriptase is an MMLV reverse transcriptase.

223. The fusion protein of claim 222, wherein the MMLV reverse transcriptase comprises a C-terminal amino acid truncation to remove the endogenous MMLV protease cleavage site.

224. The fusion protein of claim 223, wherein the C-terminal amino acid truncation is about 1-180, about 1-170, about 1-160, about 1-150, about 1-140, about 1-130, about 1-120, about 1-110, about 1-100, about 1-90, about 1-80, about 1-70, about 1-60, about 1-50, about 1-40, about 1-30, about 1-20, or about 1-10 amino acids in length.

225. The fusion protein of claim 223 or 224, wherein the C-terminal amino acid truncation is about six amino acids in length.

226. The fusion protein of any one of claims 217-225, wherein the napDNAbp is bound to a prime editing guide RNA (pegRNA).

227. The fusion protein of any one of claims 217-226, wherein the fusion protein comprises a prime editor, or a portion thereof.

228. The fusion protein of claim 227, wherein the prime editor comprises PE2, PE3, PE4, PE5, PE2max, PE3max, PE4max, or PE5max.

229. The fusion protein of claim 228, wherein PE3 and PE3max comprise a second strand nicking guide RNA (ngRNA).

230. The fusion protein of any one of claims 217-229, wherein the fusion protein comprises two NES, three NES, four NES, five NES, six NES, seven NES, eight NES, nine NES, or ten NES.

231. The fusion protein of any one of claims 217-230, wherein the NES, or multiple NES, are inserted within the gag nucleocapsid protein.

232. The fusion protein of claim 231, wherein the NES, or multiple NES, are inserted between the p12 and CA domains of the gag nucleocapsid protein, within the p12 domain of the gag nucleocapsid protein, or between the p12 and MA domains of the gag nucleocapsid protein.

233. The fusion protein of any one of claims 217-232, wherein the fusion protein further comprises a nuclear localization sequence (NLS).

234. The fusion protein of claim 233, wherein the fusion protein further comprises two NLS.

235. The fusion protein of claim 234, wherein the fusion protein comprises a first NLS at the N-terminus of the napDNAbp and a second NLS at the C-terminus of the domain comprising an RNA-dependent DNA polymerase activity.

236. The fusion protein of any one of claims 217-235, wherein the cleavable linker is located between the napDNAbp and the NES.

237. The fusion protein of any one of claims 217-236, wherein the cleavable linker comprises a protease cleavage site.

238. The fusion protein of claim 237, wherein the protease cleavage site is a Moloney murine leukemia virus (MMLV) protease cleavage site or a Friend murine leukemia virus (FMLV) protease cleavage site.

239. The fusion protein of claim 237 or 238, wherein the protease cleavage site comprises the amino acid sequence TSTLLMENSS (SEQ ID NO: 5), PRSSLYPALTP (SEQ ID NO: 6), VQALVLTQ (SEQ ID NO: 7), PLQVLTLNIERR (SEQ ID NO: 8), or an amino acid sequence at least 90% identical to any one of SEQ ID NOs: 5-8.

240. The fusion protein of any one of claims 217-239, wherein one or more additional linkers are inserted N′ and/or C′ to the cleavable linker.

241. The fusion protein of claim 240, wherein a linker comprising the amino acid sequence G is inserted C′ to the cleavable linker.

242. The fusion protein of claim 240, wherein linkers comprising the amino acid sequence GGS are inserted N′ and/or C′ to the cleavable linker.

243. The fusion protein of claim 240, wherein linkers comprising the amino acid sequence SGGSSGGS (SEQ ID NO: 163) are inserted N′ and/or C′ to the cleavable linker.

244. The fusion protein of any one of claims 217-243, wherein the gag-pro polyprotein comprises an MMLV gag-pro polyprotein or an FMLV gag-pro polyprotein.

245. The fusion protein of any one of claims 217-244, wherein the gag nucleocapsid protein comprises an MMLV gag nucleocapsid protein or an FMLV gag nucleocapsid protein.

246. The fusion protein of any one of claims 217-245, wherein the fusion protein comprises both the napDNAbp and the domain comprising an RNA-dependent DNA polymerase activity.

247. The fusion protein of claim 246, wherein the fusion protein comprises the structure:
[gag nucleocapsid protein]-[lX-3×NES]-[cleavable linker]-[NLS]-[napDNAbp]-[domain comprising RNA-dependent DNA polymerase activity]-[NLS], wherein]-[comprises an optional linker.

248. The fusion protein of claim 246, wherein the fusion protein comprises the structure:
[gag nucleocapsid protein]-[1×-3×NES]-[cleavable linker]-[NLS]-[napDNAbp]-[domain comprising RNA-dependent DNA polymerase activity]-[NLS], wherein]-[comprises an optional linker.

249. A composition comprising a first fusion protein of any one of claims 217-245, wherein the first fusion protein comprises a napDNAbp, and a second fusion protein of any one of claims 217-245, wherein the second fusion protein comprises a domain comprising an RNA-dependent DNA polymerase activity.

250. The composition of claim 249, wherein the first and the second fusion proteins comprise the structures:
[gag nucleocapsid protein]-[napDNAbp]-[split intein]; and
[gag nucleocapsid protein]-[split intein]-[domain comprising RNA-dependent DNA polymerase activity], wherein]-[comprises an optional linker.

251. The composition of claim 249, wherein the first and the second fusion proteins comprise the structures:
[gag nucleocapsid protein]-[first portion of napDNAbp]-[split intein]; and
[gag nucleocapsid protein]-[split intein]-[second portion of napDNAbp]-[domain comprising RNA-dependent DNA polymerase activity], wherein]-[comprises an optional linker.

252. A polynucleotide encoding the fusion protein of any one of claims 217-248.

253. A vector comprising the polynucleotide of claim 252.

254. A cell comprising the fusion protein of any one of claims 217-248, the polynucleotide of claim 252, or the vector of claim 253.

255. A kit comprising the virus-like particle of any one of claims 1-67, the plurality of polynucleotides of any one of claims 69-128, the one or more vectors of any one of claims 129-131, or the fusion protein of any one of claims 217-248.

256. A virus-like particle of any one of claims 1-67 produced by transfecting, transducing, electroporating, or otherwise inserting the plurality of polynucleotides of any one of claims 69-128 or the one or more vectors of any one of claims 129-131 into a cell and expressing the components of the virus-like particle from the plurality of polynucleotides or one or more vectors in the cell, thereby allowing the virus-like particle to spontaneously assemble in the cell.
